(19) United States
(12) Patent Application Publication
(10) Pub. No.: US 2023/0272474 A1
(43) Pub. Date: Aug. 31, 2023

(54) METHYLATION MARKERS FOR MELANOMA AND USES THEREOF

(71) Applicant: The University of North Carolina at Chapel Hill, Chapel Hill, NC (US)

(72) Inventors: Kathleen C. DORSEY, Pittsboro, NC (US); Nancy E. THOMAS, Durham, NC (US); Sharon N. EDMISTON, Chapel Hill, NC (US); Pamela A. GROBEN, Mebane, NC (US); Joel S. PARKER, Apex, NC (US)

(21) Appl. No.: 16/963,063

(22) PCT Filed: Jan. 18, 2019
(86) PCT No.: PCT/US2019/014375

§ 371 Date: Jul. 17, 2020

Related U.S. Application Data
(60) Provisional application No. 62/619,334, filed on Jan. 19, 2018.

(51) Int. Cl. C12Q 1/6886

(52) U.S. Cl. CPC C12Q 1/6886; C12Q 2600/154; C12Q 1/6806

(57) ABSTRACT

This disclosure is directed to a method for detecting melanoma in a tissue sample by measuring a level of methylation of one or more regulatory elements differentially methylated in melanoma and benign nevi. The invention provides methods for detecting melanoma, related kits, and methods of screening for compounds to prevent or treat melanoma.

### CROSS REFERENCE TO RELATED APPLICATIONS

This application claims the benefit of U.S. Provisional Appn. No. 62/619,334 filed 19 Jan. 2018, Dorsey et al., entitled “METHYLATION MARKERS FOR MELANOMA AND USES THEREOF”, Atty. Dkt. No. 150-25-PROV which is hereby incorporated by reference in its entirety.

### STATEMENT REGARDING FEDERALLY SPONSORED RESEARCH OR DEVELOPMENT

This invention was made with government support under Grant Nos. CA134368, CA160138, and CA199487 awarded by the National Institutes of Health. The United States Government has certain rights in the invention.

### 1. FIELD

The present disclosure provides a diagnostic for melanoma and uses thereof. The disclosure provides methods for detecting melanoma by a panel of methylated elements, related kits, and methods of screening for compounds to prevent or treat melanoma.

### 2. BACKGROUND

### 2.1. Introduction Skin Cancer and Melanoma

Skin cancer is the most common form of cancer. There are two major types of skin cancer, keratinocyte cancers (basal and squamous cell carcinomas) and melanoma. Though melanoma is less than five percent of the skin cancers, it is the seventh most common malignancy in the U.S. and is responsible for most of the skin cancer related deaths. Specifically, the American Cancer Society estimates that in the U.S. alone 87,000 new cases of melanoma, will be diagnosed in 2017 and almost 9,700 people will die of melanoma (American Cancer Society Cancer Facts and Figures 2017). The WHO estimates that 65,000 people die worldwide of melanoma every year (Lucas, R., Global Burden of Disease of Solar Ultraviolet Radiation, Environmental Burden of Disease Series, Jul. 25, 2006; No. 13. News release, World Health Organization).

As with many cancers, the clinical outcome for melanoma depends on the stage at the time of the initial diagnosis. When melanoma is diagnosed early, the prognosis is good. However, if diagnosed in late stages, it is a deadly disease. In particular, in 2010 the ACS reports that the 5-year survival rate is 98% for melanoma diagnosed when small and localized, stage IA or IB. However, when the melanoma has spread beyond the original area of skin and nearby lymph nodes, the 5-year survival rate drops to 18% for distant metastatic disease, or stage IV melanoma (American Cancer Society Cancer Facts and Figures 2017, Table 8). It is therefore imperative to diagnose melanoma in its earliest form.

### 2.2. Issues with Melanoma Diagnosis

Early diagnosis is difficult due to the overlap in clinical and histopathological features of early melanomas and benign nevi, especially benign atypical nevi (Strauss et al., 2007, Br. J. Dermatol. 157, 758-764). Moreover, there is a sizeable disagreement amongst pathologists regarding the diagnosis of melanoma and benign diseases such as compound melanocytic nevi or Spitz nevi. One study reported a 15% discordance (Shoo et al. 2010, J. Am. Acad. Dermatol. 62(5), 751-756). An earlier study of over 1000 melanocytic lesions reported that an expert panel found a 14% rate of false positives, misclassifying benign lesions as invasive melanoma; and a 17% rate of false negatives, misclassifying malignant melanoma as benign (Veenhuizen et al. 1997, J. Pathol. 182, 266-272). In one study where an expert panel interpreted lesions as melanoma, a group of general pathologists mistakenly diagnosed dysplastic nevi in 12% of the readings (Brochez et al., 2002, J. Pathol. 196, 459-466). In fact, many nevi, especially atypical or dysplastic nevi, are difficult to distinguish from melanoma, even by expert pathologists (Farmer et al., 1996, Hum. Pathol. 27, 528-531). This results in a quandary for clinicians who not only biopsy but re-excise with margins large numbers of benign atypical nevi in the population (Fung, 2003, Arch. Dermatol. 139, 1374-1375), at least, in part, due to lack of confidence in the histopathologic diagnosis. The numbers involved are substantial in the U.S. alone. One study estimated that with 1,500,000 to 4,500,000 annual biopsies of melanocytic neoplasms, 200,000 to 650,000 discordant cases would result annually (Shoo et al. 2010, J. Am. Acad. Dermatol. 62(5), 751-756). This high rate of misdiagnosis is problematic on many levels. The false positives lead to unnecessary costly medical interventions, e.g., overly large excisions, sentinel lymph node biopsy, high-dose interleukin-2 or interferon alpha, adjuvant trials with new agents, and needless stress for the patients. The false negatives mean increased likelihood of a presentation with more severe disease, which as discussed above, dramatically increases the risk of a poor clinical outcome and death.

Furthermore, current guidelines recommend wide excisional biopsy with 1.0 to 3.0 mm margins for patients presenting with primary melanoma (NCCN, Clin. Pract. Guidelines in Oncology—v.1.2017: Melanoma, Nov. 10, 2016, page ME-B). However, excisional biopsy with such broad margins would not be appropriate, as biopsied histologically benign nevi would typically either not be excised or excised with conservative margins (2-5 mm) for certain dysplastic nevi, Spitz nevi, and atypical blue nevi.

### 2.3. Standard of Care for Melanoma

For suspicious pigmented lesions, current guidelines recommend excisional biopsy with 1-3 mm margins and rebiopsy if the sample is inadequate for diagnosis or microstaging. Pathologists typically assess Breslow's depth or thickness, ulceration, mitotic rate, margin status and Clark's level (based on the skin layer penetrated). A positive diagnosis for melanoma may lead to an evaluation for potential spread to the lymph nodes or other organs. Patients with stage I or II melanoma often are further staged with sentinel lymph node biopsy (SLNB) including immunohistochemical (IHC) staining. IHC can be used as an adjunct to the standard histopathologic examination (hematoxylin and eosin (H&E) staining, etc.) for melanocytic lesions or to determine the tumor of origin. Antibodies such as 5100, HMB-45 and MART-1/Melan-A or cocktails of all three may be used for staining (Ivan & Prieto, 2010, Future Oncol. 6(7), 1163-1175). Follow up may include cross sectional imaging (CT, MRI, PET). For patients suspected with stage III disease, with clinically positive lymph nodes, guidelines recommend fine needle aspiration or open biopsy of the enlarged lymph node and imaging for baseline staging. For patients with distant metastases, stage IV, serum lactate dehydrogenase (LDH) may have a prognostic role (NCCN ver. 1.2017).

As discussed above, wide excision is recommended for primary melanoma. For patients with lymph node involvement, stage III, complete lymph node dissection may be indicated. For patients with resected stage IIB or III melanoma, studies have shown that adjuvant high-dose interferon alfa-2b and peginterferon alfa-2b have led to longer disease-free survival. Davar & Kirkwood Adjuvant Therapy of Melanoma, 2016, Cancer Treat Res 167:181-208. High dose ipilimumab was FDA approved in 2015 as an adjuvant therapy for patients with Stage III melanoma based on lower recurrence-free survival in the treated group but has substantial toxicity. Eggermont et al., Adjuvant ipilimumab versus placebo after complete resection of high-risk stage III melanoma (EORTC 18071): a randomised, double-blind, phase 3 trial. The lancet oncology 2015; 16(5):522-30. In 2017, the anti-PD-1 antibody nivolumab was FDA approved for patients with completely resected stage IIIB, IIIC, or IV melanoma based on findings that adjuvant therapy with nivolumab resulted in significantly longer recurrence-free survival and a lower rate of grade 3 or 4 adverse events than adjuvant therapy with ipilimumab. Weber et al., 2017, Adjuvant Nivolumab versus Ipilimumab in Resected Stage III or IV Melanoma. N Engl J Med 377(19):1824-1835. For metastatic or unresectable melanoma first line systemic treatments include: immunotherapy such as anti-PD-1 monotherapy with pembrolizumab or nivolumab or combination therapy with nivolumab and ipilimumab; BRAF/MEK inhibitor combination therapy (dabrafenib/trametinib or vemurafenib/cobimetinib) BRAF V600 targeted therapy. Second line therapy may include systemic treatment with conventional chemotherapies such as albumin-bound paclitaxel, carboplatin, dacarbazine, IL-2, interferon alfa-2b, nitrosourea, temozolomide, vinblastine and combinations thereof (NCCN ver. 1.2017 ME-G).

### 2.4. Current Diagnostic Challenges

Cutaneous melanoma is a potentially aggressive malignancy with a propensity to metastasize early, and there is a pronounced survival difference between localized and metastatic disease (Siegel et al, 2014). Despite newly available targeted and immunomodulatory agents for melanoma (Chapman et al, 2011; Hamid et al, 2013; Hauschild et al, 2012; Hodi et al, 2010; Robert et al, 2015), systemic therapies lead to cures in a relatively small number of patients. Therefore, early detection is crucial for favorable outcomes, yet early definitive diagnosis can be difficult due to the overlap in clinical and histologic appearances of melanomas and highly prevalent benign melanocytic nevi (moles) (Strauss et al, 2007). Histopathologic review is considered the ‘gold standard’ for melanoma diagnosis; however, numerous studies have reported interobserver discordance in the diagnosis of melanocytic lesions even by expert dermatopathologists (Brochez et al, 2002; Shoo et al, 2010; Veenhuizen et al, 1997). In one study (Farmer et al, 1996), review of 40 benign and malignant melanocytic lesions by eight dermatopathologists produced discordant diagnoses in 38% of cases. Moreover, certain nevus subtypes, especially dysplastic nevi, Spitz nevi, and atypical blue nevi can be particularly difficult to distinguish from melanoma (Brochez et al, 2002; Gerami et al, 2014). The difficulty in accurately diagnosing melanoma presents a quandary for clinicians who not only biopsy but often re-excise with margins large numbers of dysplastic nevi in the population (Fung, 2003) due in part to lack of confidence in the histopathologic diagnosis. A critical need exists for improving diagnostic methods to avoid under- and over-treatment of melanocytic lesions. Yet the small size of melanocytic lesions and early melanomas, which are typically submitted in their entirety in formalin for diagnosis, present particular challenges as any new diagnostic test needs to be robust enough to perform reliably from small formalin-fixed paraffin-embedded (FFPE) specimens.

Prior studies have shown that melanomas differ from benign nevi at the molecular level, exhibiting variations in mRNA expression (Clarke et al, 2015; Talantov et al, 2005; Koh et al, 2008; Haqq et al, 2005; Alexandrescu et al, 2010), gene copy number (Shain et al, 2015; North et al, 2014; Bastian et al, 2003; Bauer and Bastian, 2006; Gerami et al, 2009; Bastian et al, 2000), protein expression (Ivan and Prieto, 2010; Uguen et al, 2015; Busam, 2013), and DNA methylation (Conway et al, 2011; Gao et al, 2013), indicating that certain molecular biomarkers could provide valuable tools for melanoma diagnosis, alone or in conjunction with histopathology. However, due to the practical limitations of typically small FFPE specimens as well as technical challenges or the labor intensity in the performance and implementation of some assays, few of these molecular differences have been translated to the clinic for melanoma diagnosis.

DNA methylation is a relatively stable epigenetic modification to the DNA that does not alter the nucleotide sequence but is associated with variation in gene expression (Plass, 2002). Changes in methylation at CpG dinucleotides in the upstream regulatory regions of genes are often among the earliest events observed during neoplastic progression of precancerous lesions (Arai and Kanai, 2010), and hypermethylation of CpG islands in tumor suppressor gene promoters is a common mechanism of gene silencing in human cancer (Arai and Kanai, 2010; Jones, 2012; Herman and Baylin, 2003). Moreover, aberrant DNA methylation occurs widely in melanomas (Thomas et al, 2014; Furuta et al, 2004; Tanemura et al, 2009; TCGA, 2015), and (Conway et al, 2011) and others (Gao et al, 2013) have reported differences in DNA methylation between primary melanomas and benign nevi, supporting the use of epigenetic biomarkers for early melanoma diagnosis.

### 3. SUMMARY OF THE DISCLOSURE

In embodiment (1) the present disclosure provides a method for detecting melanoma in a tissue sample which comprises: (a) measuring a level of methylation of a plurality of regulatory elements differentially methylated in melanoma and benign nevi; and (b) determining whether melanoma is present or absent in the tissue sample if there is (i) hypermethylation of at least one regulatory element associated with a gene encoding ALX3, CCDC140, CCDC19, DYNC1I1, FLJ22536, HOXD12, LIPC, NBLA00301/HAND2, NRXN1, ONECUT1, PAX3/CCDC140, PROM1, RASGEF1C, SGEF, SHANK3, SHOX2, SIX6, TBX5, TLX3, and ZBTB38, and (ii) hypomethylation of at least one regulatory element associated with a gene encoding ANKH, C3AR1, C5orf56, CACNA1C, CYTIP, EPB41L4A, FAIM3, GIMAP7, GOLIM4, KREMEN1, MAS1L, MBP, MYT1L, OPCML, SORCS2, TLR1, and VOPP1. The disclosure also provides a method which consists of, or consists essentially of, measuring regulatory elements associated with these genes.

In embodiment (2) the present disclosure provides a method for detecting melanoma in a tissue sample which comprises: (a) measuring a level of methylation of a plurality of regulatory elements differentially methylated in melanoma and benign nevi; and (b) determining whether melanoma is present or absent in the tissue sample if there is (i) hypermethylation of at least one regulatory element associated with a gene encoding ALX3, C22orf9, CBFA2T3, CCDC140, DEFB128, EFCAB1, ESRRG, FAM134B, FAM193A, GFI1, GNG7, HIPK2, HOXD12, HOXD13, MREG, MYADML, NRXN1, PAX3/CCDC140, PROM1, RASGEF1C, SEMA4B, SHOX2, SIGIRR, SIX6, TBX5, TLX3, and ZBTB38, and (ii) hypomethylation of at least one regulatory element associated with a gene encoding ANKH, ANXA2, C3AR1, CACNA1C, ELSPBP1, EPB41L4A, FAIM3, GOLIM4, IGDCC4, KIAA1609, LAMAS, MBP, MKKS, MYOM2, PDS5B, PKHD1, PPIAL4B; PPIAL4A, PTPN22, RASGEF1C, ROBO1, SORBS2, SORCS2, TARM1, TLR1, TMEM132B, and VOPP1. The disclosure also provides a method which consists of, or consists essentially of, measuring regulatory elements associated with these genes.

In embodiment (3) the present disclosure provides the method of any of embodiments (1) or (2) wherein the level of methylation is measured at single CpG site resolution.

In embodiment (5) the present disclosure provides a method for detecting melanoma in a tissue sample which comprises: (a) measuring a level of methylation of a plurality of regulatory elements differentially methylated in melanoma and benign nevi; and (b) determining whether melanoma is present or absent in the tissue sample if there is (i) hypermethylation of a CpG site cg01725872, cg02192204, cg02936049, cg03874199, cg04131969, cg05787556, cg06215569, cg07817686, cg08258526, cg08657228, cg08697503, cg08898055, cg09935388, cg10119160, cg11523712, cg12072972, cg12515659, cg12983971, cg12993163, cg13019491, cg13164157, cg13782322, cg14064356, cg14405813, cg16325502, cg18077971, cg18689332, cg19352038, cg22322562, cg24874003, cg25790133, and cg25975621, and (ii) hypomethylation of a CpG site cg00295418, cg00387964, cg00916635, cg01975505, cg02468320, cg02585849, cg03315407, cg04499514, cg05208607, cg05594873, cg07637837, cg08331829, cg08337633, cg08757862, cg09120722, cg09785377, cg11033617, cg15158847, cg15536663, cg16113793, cg18098839, cg18694313, cg21966754, cg23350716, cg24107163, cg26579713, and cg26820259. The disclosure also provides a method which consists of, or consists essentially of, measuring the methylation or lack thereof of these CpG sites.

In embodiment (4) the present disclosure provides a method for detecting melanoma in a tissue sample which comprises: (a) measuring a level of methylation of a plurality of regulatory elements differentially methylated in melanoma and benign nevi; and (b) determining whether melanoma is present or absent in the tissue sample if there is (i) hypermethylation of a CpG site cg02744046, cg02936049, cg03874199, cg05787556, cg06215569, cg06573459, cg07553475, cg07569216, cg08697503, cg08898055, cg09476130, cg12993163, cg13019491, cg13164157, cg14064356, cg16325502, cg16919569, cg17889682, cg18077971, cg18689332, cg18851100, cg19352038, and cg22322562, and (ii) hypomethylation of a CpG site cg00387964, cg02468320, cg03315407, cg03653573, cg04499514, cg07230581, cg07637837, cg08337633, cg08757862, cg10559416, cg12423733, cg15158847, cg15536663, cg15849098, cg17918270, cg18098839, and cg26967305. The disclosure also provides a method which consists of, or consists essentially of, measuring the methylation or lack thereof of these CpG sites.

In embodiment (6) the present disclosure provides the method of any of embodiments (1)-(5) further comprising measuring at least one DNA mutation in a TERT gene promoter region. The DNA mutation in the TERT gene promoter may be 103C>T, 105_106CC>TT, 124C>T, 138_139CC>TT, 146C>T, 148C>T, or 156C>T.

In embodiment (7) the present disclosure provides the method of embodiment (6), wherein the measuring at least one DNA mutation in the TERT gene promoter and the measuring of a level of methylation of a plurality of regulatory elements are performed sequentially. Alternatively, the measurement of the DNA mutation in the TERT gene promoter and the measurement of the level of methylation are done together.

In embodiment (8) the present disclosure provides the method of embodiment (7), wherein the DNA mutation in the TERT gene promoter is measured before measuring the level of methylation of a plurality of regulatory elements.

In embodiment (9) the present disclosure provides a method of detecting biomarkers in a tissue sample obtained from a human patient, the method comprising measuring a methylation state of each site in a plurality of classifier elements at a nucleic acid level wherein the plurality of classifier elements are selected from at least one regulatory element associated with a gene encoding ALX3, ANKH, C3AR1, C5orf56, CACNA1C, CCDC140, CCDC19, CYTIP, DYNC1I1, EPB41L4A, FAIM3, FLJ22536, GIMAP7, GOLIM4, HOXD12, KREMEN1, LIPC, MAS1L, MBP, MYT1L, NBLA00301; HAND2, NRXN1, ONECUT1, OPCML, PAX3; CCDC140, PROM1, RASGEF1C, SGEF, SHANK3, SHOX2, SIX6, SORCS2, TBX5, TLR1, TLX3, VOPP1, and ZBTB38.

In embodiment (10) the present disclosure provides the method of embodiment (9) further comprising measuring at least one DNA mutation in a TERT gene promoter region.

In embodiment (11) the present disclosure provides a method of detecting biomarkers in a tissue sample obtained from a human patient, the method comprising measuring a methylation state of each site in a plurality of classifier elements at a nucleic acid level wherein the plurality of classifier elements are selected from at least one regulatory element associated with a gene encoding ALX3, ANKH, ANXA2, C22orf9, C3AR1, CACNA1C, CBFA2T3, CCDC140, DEFB128, EFCAB1, ELSPBP1, EPB41L4A, ESRRG, FAIM3, FAM134B, FAM193A, GFI1, GNG7, GOLIM4, HIPK2, HOXD12, HOXD13, IGDCC4, KIAA1609, LAMAS, MBP, MKKS, MREG, MYADML, MYOM2, NRXN1, PAX3; CCDC140, PDS5B, PKHD1, PPIAL4B; PPIAL4A, PROM1, PTPN22, RASGEF1C, ROBO1, SEMA4B, SHOX2, SIGIRR, SIX6, SORBS2, SORCS2, TARM1, TBX5, TLR1, TLX3, TMEM132B, VOPP1, and ZBTB38.

In embodiment (12) the present disclosure provides the method of embodiment (11) further comprising measuring DNA mutation(s) in a TERT gene promoter region.

In embodiment (13) the present disclosure provides the method of embodiment (9) or (11), where the DNA mutation(s) in the TERT gene promoter are 103C>T, 105_106CC>TT, 124C>T, 138_139CC>TT, 146C>T, 148C>T, or 156C>T.

In embodiment (14) the present disclosure provides the method of any of embodiments (9)-(13), which further comprises comparing the detected methylation levels of the plurality of classifier elements to the expression levels of the plurality of classifier elements in at least one sample training set(s), wherein one of the sample training set(s) comprise methylation level data of the plurality of classifier elements from a melanoma sample and one of the sample training set(s) comprise methylation level data of the plurality of classifier elements from a normal nevus sample, and the comparing step comprises applying a statistical algorithm which comprises determining a correlation between the methylation level data obtained from the human tissue sample and the methylation level data from the melanoma and the normal nevus training set(s).

In embodiment (15) the present disclosure provides the method of any of embodiments 1-14, wherein the tissue sample is a common nevi sample.

In embodiment (16) the present disclosure provides the method of any of embodiments 1-14, wherein the tissue sample is a dysplastic nevi sample.

In embodiment (17) the present disclosure provides the method of any of embodiments 1-14, wherein the tissue sample is a benign atypical nevi sample.

In embodiment (18) the present disclosure provides the method of any of embodiments 1-14, wherein the tissue sample is a melanocytic lesion of unknown potential.

In embodiment (19) the present disclosure provides the method of any of embodiments 1-14, wherein the tissue sample is a formalin-fixed, paraffin-embedded sample.

In embodiment (20) the present disclosure provides the method of any of embodiments 1-14, wherein the tissue sample is a fresh-frozen sample.

In embodiment (21) the present disclosure provides the method of any of embodiments 1-14, wherein the tissue sample is a fresh tissue sample.

In embodiment (22) the present disclosure provides the method of any of embodiments 1-14, wherein the tissue sample is a dissected tissue, an excision biopsy, a needle biopsy, a punch biopsy, a shave biopsy, or a skin biopsy sample.

In embodiment (23) the present disclosure provides the method of any of embodiments 1-14, wherein the tissue sample is a lymph node biopsy sample.

In embodiment (24) the present disclosure provides the method of any of embodiments 1-14, wherein the level of methylation is measured using a bisulfate conversion-based microarray assay.

In embodiment (25) the present disclosure provides the method of any of embodiments 1-14, wherein the level of methylation is measured using a methylation specific polymerase chain reaction assay.

In embodiment (26) the present disclosure provides the method of any of embodiments 1-14, wherein the level of methylation is measured using a mass spectrometry assay.

In embodiment (27) the present disclosure provides the method of any of embodiments 1-14, wherein a plurality of regulatory elements differentially methylated are measured, and together they have a sensitivity of greater than 95% more preferably greater than 97%.

In embodiment (28) the present disclosure provides a method for treating a patient with a suspicious melanocytic lesion, the method comprising the steps of: determining whether the suspicious lesion is a melanoma by obtaining, or having obtained a biological sample from the patient, and performing, or having performed, a test the biological sample to determine if there is (i) hypermethylation of at least on regulatory element associated with a gene encoding ALX3, CCDC140, CCDC19, DYNC1I1, FLJ22536, HOXD12, LIPC, NBLA00301/HAND2, NRXN1, ONECUT1, PAX3/CCDC140, PROM1, RASGEF1C, SGEF, SHANK3, SHOX2, SIX6, TBX5, TLX3, and ZBTB38, and (ii) hypomethylation of at least one regulatory element associated with a gene encoding ANKH, C3AR1, C5orf56, CACNA1C, CYTIP, EPB41L4A, FAIM3, GIMAP7, GOLIM4, KREMEN1, MAS1L, MBP, MYT1L, OPCML, SORCS2, TLR1, and VOPP1; if the suspicious lesion is determined to be a melanoma treating the patient.

In embodiment (29) the present disclosure provides the method of embodiment 28 further comprising measuring at least one DNA mutation in a TERT gene promoter region. The DNA mutation in the TERT gene promoter may be 103C>T, 105_106CC>TT, 124C>T, 138_139CC>TT, 146C>T, 148C>T, or 156C>T.

In embodiment (30) the present disclosure provides the method of embodiments 28 or 29, wherein the treatment is wide surgical excision (≥1 cm) of the suspicious melanocytic lesion.

In embodiment (31) the present disclosure provides a kit comprising: (a) at least one reagent selected from the group consisting of: (i) a series of 40 nucleic acid probes or 59 nucleic acid probes capable of specifically hybridizing with an element differentially methylated in melanoma and benign nevi; (ii) a series of nucleic acid primers capable of PCR amplification of an element differentially methylated in melanoma and benign nevi; and (iii) a series of methylation specific antibodies and probes capable of specifically hybridizing with 40 elements differentially methylated in melanoma and benign nevi; and (b) instructions for use in measuring a level of methylation of 40 or 59 elements in a tissue sample from a subject suspected of having melanoma.

In embodiment (32) the present disclosure provides a method of identifying a compound that prevents or treats melanoma progression, the method comprising the steps of: (a) contacting a compound with a sample comprising a cell or a tissue; (b) measuring a level of methylation of 40 or more regulatory elements differentially methylated in melanoma and benign nevi; and (c) determining a functional effect of the compound on the level of methylation; thereby identifying a compound that prevents or treats melanoma.

### 4. BRIEF DESCRIPTION OF THE FIGURES

FIG. 1A-1D. Performance of the 40-CpG diagnostic methylation signature for melanoma in training and test sets. The training set consisted of 60 melanomas and 48 nevi, while the validation set included 29 melanomas and 25 nevi. The 40 diagnostic probes were identified from the model that analyzed annotated probes with IQR>0.2 beta between melanomas and nevi. (FIG. 1A) Heatmap showing methylation at diagnostic signature probes in melanomas (black) and nevi (white) from the combined training (white) and test (black) sets. Darker gray represents highly methylated and lighter represents unmethylated. (FIG. 1B) Contribution of each probe to the signature as indicated by weight score. (FIG. 1C) ROC plot showing diagnostic accuracy in the test set. (FIG. 1D) PCA showing the segregation of melanoma and nevus samples based on the 40-probe signature.

FIG. 2A-2D. Diagnostic methylation signature calls on uncertain melanocytic samples versus histologically-confirmed melanomas and nevi. Interobserver dermatopathologic review identified 89 melanomas, 73 nevi, and 41 diagnostically uncertain samples. (FIG. 2A) Supervised heatmap, ordered left to right from lowest to highest diagnostic prediction score, showing methylation levels at the 40 diagnostic CpGs in melanomas (black) or nevi (white) from the training (white) or test sets (black), or uncertain samples (lighter gray). (FIG. 2B) Waterfall plot of prediction scores, ordered as in the heatmap, and color-coded for diagnosis. (FIG. 2C) Boxplots of prediction scores for each sample type, with the median and interquartile range encompassed by the box. The broken line indicates the prediction score threshold for distinguishing melanomas from nevi. (FIG. 2D) PCA plot shows sample segregation based on the 40-CpG signature.

FIG. 3A-3E. Independent validation of differential methylation at genes diagnostic for melanoma. TCGA melanoma 450K methylation data were obtained from Broad Institute (FIG. 3A, FIG. 3B) (TCGA, 2015), while the 27K methylation dataset of Gao et al (2013) was downloaded from GEO (accession number GSE45266) (FIG. 3C-FIG. 3E). (FIG. 3A) Heatmap of 40-CpG methylation and waterfall plot of diagnostic prediction scores in 105 primary melanomas from TCGA (black), and 89 melanomas and 73 nevi from UNC (lighter gray). (FIG. 3B) Boxplots showing diagnostic prediction scores for TCGA primary or metastatic melanomas and UNC primary melanomas and nevi. (FIG. 3C) Heatmap illustrating 27K methylation at 44 CpGs in 38 diagnostic genes in 24 melanomas and 5 nevi from the study of Gao et al (2013). (FIG. 3D) Methylation-based PCA plot showing separation of melanomas from nevi. (FIG. 3E) Boxplots showing differential methylation at 2 CpGs (HOXD12, cg3874199 and PAX3, cg19352038) exactly matching diagnostic 450K probes.

FIG. 4A-4D. Comparative performance of diagnostic methylation models tested in primary melanomas and benign nevi in the training set. The training set (67% of samples, randomly selected) consisted of 60 melanomas and 48 nevi for which all 3 dermatopathologic reviews and the initial pathology report were in complete agreement. An exception was that one nevoid melanoma based on the pathology report had two expert reviews as a melanoma and one as a nevus, but was allowed to remain in the data as a melanoma as the patient had had visceral metastases and died of disease. Area under the receiver operating characteristic curves (AUC) versus number of probes are shown for each diagnostic model tested. The broken line in all plots indicates AUC of 0.98. Eight diagnostic models were tested in panels A and B that contained as starting probe sets either (FIG. 4A) all available 450K probes (overlapping probes on the EPIC 850K methylation array) or (FIG. 4B) 450K probes associated with candidate genes differentially methylated between melanomas and nevi in our prior Illumina Cancer Panel I methylation study (Conway et al. 2011). The eight models tested within each of the two probe sets were as follows: (1) all 450K or ‘candidate gene’ probes (- - - -), (2) probes filtered for IQR>0.2 β (∘ ∘ ∘ ∘), (3) model adjusted for age (age-adjusted) (˜ ˜ ˜ ˜), (4) model adjusted for age (age-adjusted), and probes filtered for IQR>0.2 β (∘ ∘ ∘ ∘), (5) age-associated probes removed from model (age-independent) (˜ ˜ ˜ ˜), (6) age-associated probes removed from model (age-independent), and probes filtered for IQR>0.2 β (∘ ∘ ∘ ∘), (7) age-associated probes removed, and model adjusted for age (age-independent, age-adjusted) (˜ ˜ ˜ ˜), (8) age-associated probes removed, and model adjusted for age (age-independent, age-adjusted), and probes filtered for IQR>0.2 β (∘ ∘ ∘ ∘). Models that did not account for age (models 1 or 2) provided the highest diagnostic accuracy with fewest probes. (FIG. 4C) Comparison of models derived from all 450K/IQR>0.2 β versus candidate/IQR>0.2 β. (FIG. 4D) Comparison of models derived from all 450K/IQR>0.2 β versus 450K/IQR>0.2 β and restricted to probes with Illumina gene annotation.

FIG. 5A-FIG. 5B. Performance of the 40-CpG diagnostic methylation signature according to patient age. (FIG. 5A) Area Under the Receiver Operating Curve (AUC), sensitivity, and specificity for all histopathology-confirmed melanoma and nevus patients younger (≤50 years of age) (left plot) or older (>50 years of age) (right plot) at diagnosis. (FIG. 5B) Scatter plot of 40-CpG diagnostic prediction score (y axis) versus patient age for all melanoma, nevus, and diagnostically uncertain specimens (x axis).

FIG. 6A-6B. Independent validation of differential mRNA expression at genes diagnostic for melanoma. The Affymetrix Hu133A gene expression dataset from Talantov et al (2005) was obtained from GEO (accession number GSE3189). (FIG. 6A) Heatmap illustrating mRNA expression for 25 (of 38) diagnostic genes in 45 primary melanomas and 18 nevi. (FIG. 6B) mRNA expression-based PCA plot showing separation of melanomas from nevi.

FIG. 7A-7C Development of the 59 CpG age-adjusted methylation signature for melanoma diagnosis and its performance in the validation set. The signature was derived from the training set (89 melanomas, 73 nevi) using the same method as the 40 CpG signature (BMIQ normalization, probes restricted to those on both the Illumina 450K and 850K arrays, probes filtered for IQR) but was additionally adjusted for age at diagnosis. (FIG. 7A) The final age-adjusted signature included 59 CpGs plus age and was derived from the bmiq.anno.850.Iqr.AGE model. (FIG. 7B) Contributions of each feature (59 probes+age) to the bmiq.anno.850K.iqr2.AGE model. (FIG. 7C) Diagnostic performance of the 59 CpG age-adjusted signature in the validation set (29 melanomas, 25 nevi).

FIG. 8A-8D. Performance of the 40-CpG melanoma classifier in training and/or validation sets. Specimens in the training (60 melanomas and 48 nevi) and validation (29 melanomas and 25 nevi) sets had diagnostic consensus on interobserver review. The 40 diagnostic probes were identified from the model that analyzed annotated probes with IQR>0.2 β between melanomas and nevi. FIG. 8A Heatmap showing methylation at 40 classifier probes in melanomas (black) and nevi (white) from the combined training (white) and validation sets (black). Black represents highly methylated and white represents unmethylated. FIG. 8B Boxplots of classifier scores for histological subtypes of nevi and melanomas. FIG. 8C ROC plot showing diagnostic accuracy in the validation set. FIG. 8D PCA showing the segregation of melanoma and nevus samples based on the 40 CpG classifier.

FIG. 9A-9D. Independent validation of differential methylation at classifier CpG loci. Validation of the diagnostic classifier was conducted in three public datasets. FIG. 9A 40-CpG methylation heatmap and waterfall plot of classifier scores in 105 primary melanomas from TCGA (TCGA, 2015) (Black) compared with 89 melanomas and 73 nevi from UNC/UR (gray). FIG. 9B Boxplots showing classifier scores for TCGA primary or metastatic melanomas and UNC/UR primary melanomas and nevi. FIG. 9C Boxplots showing classifier scores for 33 primary and 28 metastatic melanomas, and 14 nevi, and ROC plot showing the diagnostic accuracy of the 40 CpG classifier comparing nevi to primary melanomas in the GSE86355 450K methylation dataset. In the GSE45266 27K methylation dataset, FIG. 9D PCA of methylation at 44 CpGs associated with diagnostic classifier genes illustrates segregation of 24 primary melanomas from 5 nevi, and boxplots showing methylation differences at the 2 CpG loci (cg3874199 and cg19352038) directly matching 450K probes in the diagnostic classifier.

FIG. 10A-10D. Diagnostic 40-CpG melanoma classifier calls on melanomas, nevi, and diagnostically uncertain samples. Interobserver dermatopathologic review identified 89 melanomas, 73 nevi, and 41 uncertain samples. FIG. 10A Supervised heatmap, ordered left to right from lowest to highest diagnostic classifier score, showing methylation levels at the 40 diagnostic CpGs in melanomas (black) or nevi (white) from the training (white) or validation sets (black), or uncertain samples (lighter gray). FIG. 10B Waterfall plot of classifier scores, ordered as in the heatmap, and color-coded for diagnosis. FIG. 10C Boxplots of classifier scores for each diagnostic category, with median and interquartile range encompassed by each box. The broken lines indicate the classifier score threshold for distinguishing melanomas from nevi. FIG. 10D PCA plot shows sample segregation based on the 40 CpG classifier.

FIG. 11A-11B. Boxplots illustrating differential methylation at the 40 classifier and neighboring CpGs in melanomas and nevi. Boxplots show methylation at classifier CpGs (gray) and, if present, nearby CpGs (black) within 500 base pairs upstream or downstream of the classifier CpGs. P values were determined by the Wilcoxon test. FIG. 11A Classifier CpGs hypermethylated in melanomas compared with nevi. FIG. 11B Classifier CpGs hypomethylated in melanomas compared with nevi.

FIG. 12. Heatmaps showing methylation at the 40 classifier CpGs in the primary melanomas and nevi in the UNC/UR training and validation sets. The clinical and pathologic characteristics of the samples are annotated.

FIG. 13. Boxplots of classifier scores according to clinical staging features in the primary melanomas in the UNC/UR training and validation sets. The median and interquartile range are encompassed by each box. The broken line indicates the classifier score threshold for distinguishing melanomas from nevi.

FIG. 14. PCA plots showing separation of melanomas, nevi and diagnostically uncertain samples by different probe sets. Uncertain melanocytic samples fell among pathologically-confirmed nevi or between melanomas and nevi in PCA plots when using: FIG. 14A 40 classifier probes, or FIG. 14B 41,448 probes obtained after filtering for IQR>0.2 β and Illumina gene annotation.

FIG. 15. Diagnostic calls by pathologists versus the 40-CpG classifier for the 41 uncertain melanocytic samples. Diagnostic calls by pathologists were nevus (dark gray), melanoma (gray with X box) or uncertain (light gray) (top panel). The original pathology classification was based on the initial pathology report. Interobserver review was subsequently conducted by three expert dermatopathologists. The 41 uncertain samples lacked consensus among these four levels of pathology review. 40-CpG classifier scores and diagnostic calls for the 41 uncertain samples are shown, ordered from lowest to highest (lower panel).

FIG. 16. Superficial spreading malignant melanoma, measuring 1.6 mm in Breslow thickness, without ulceration. This melanoma harbored a hotspot −124C>T TERT promoter mutation (hematoxylin and eosin; ×4.9 magnification).

FIG. 17. Lentigo maligna melanoma, 3.0-mm Breslow thickness, nonulcerated. No TERT promoter mutation was identified (hematoxylin and eosin; ×13 magnification).

FIG. 18A-18B Benign, predominantly intradermal melanocytic nevus with a congenital pattern. This nevus was found to harbor a hotspot −124C>T TERT promoter mutation. No mitotic figures were present (hematoxylin and eosin; 18A ×3.8 and 18B ×40 magnification, respectively).

FIG. 19A-19D. Compound melanocytic neoplasm with severe architectural and cytological atypia. This indeterminate case was found to harbor a −124C>T TERT promoter mutation. FIG. 19A, A compound melanocytic neoplasm fills and expands the papillary dermis forming a dome-shaped lesion (hematoxylin and eosin; ×3.1). FIG. 19B, The junctional component of the tumor has discrete nesting of melanocytes without confluence or pagetoid spread of cells (hematoxylin and eosin; ×200). FIG. 19C, Areas within the dermal component have expansive groupings of epithelioid melanocytes with vesicular chromatin patterns and prominent nucleoli, and there are lymphocytes present (hematoxylin and eosin; 200). FIG. 19D, Mitotic figures (arrow) were rarely found in the dermal component of the melanocytic tumor (hematoxylin and eosin; ×400).

FIG. 20. Combination TERT promoter and the DNA methylation assay screening algorithm for primary melanocytic proliferations. In a preferred embodiment, the sample is also screened for TERT promoter mutations. In one embodiment, the TERT promoter mutations are determined first. If there is a de novo ETS/TCF binding site that is created, then the lesion is called a positive (a melanoma). If the TERT promoter assay is negative or fails the assay then the DNA methylation assay is run. If it is positive in the methylation assay then the lesion is called positive (a melanoma). If it is negative after both assay, then it is called a nevus. *Noted are the number of samples in our dataset that were screened by each assay using this algorithm.

FIG. 21. Diagnostic Algorithm Showing the Decision Pathway for a Clinician using the DNA methylation test.

### 5. DETAILED DESCRIPTION OF THE DISCLOSURE

### 5.1. Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or there below. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “melanoma” refers to malignant neoplasms of melanocytes, which are pigment cells present normally in the epidermis, in adnexal structures including hair follicles, and sometimes in the dermis. Sometimes it is referred to as “cutaneous melanoma” or “malignant melanoma.” There are at least four types of cutaneous melanoma: lentigo maligna melanoma (LMM), superficial spreading melanoma (SSM), nodular melanoma (NM), and acral lentiginous melanoma (ALM). Cutaneous melanoma typically starts as a proliferation of single melanocytes, e.g., at the junction of the epidermis and the dermis. The cells first grow in a horizontal manner and settle in an area of the skin that can vary from a few millimeters to several centimeters. As noted above, in most instances the transformed melanocytes usually, but not always, produce increased amounts of pigment so that the area involved can be seen by the clinician.

The terms “nucleic acid” and “nucleic acid molecule” may be used interchangeably throughout the disclosure. The terms refer to nucleic acids of any composition, such as DNA (e.g., complementary DNA (cDNA), genomic DNA (gDNA) and the like), RNA (e.g., messenger RNA (mRNA), short inhibitory RNA (siRNA), ribosomal RNA (rRNA), tRNA, microRNA, RNA highly expressed by the melanoma or nevi, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. Examples of nucleic acids are SEQ ID NO: 1-40 shown in Supp. TABLE S4; SEQ ID NO: 41-80 in Supp. TABLE S5; SEQ ID NO: 81-139 in Supp. TABLE S6; SEQ ID NO: 140-198 in Supp. TABLE S7; and SEQ ID NO: 199-480, which may be methylated or unmethylated at any CpG site present in the sequence, including the CpG sites shown in brackets on some sequences. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses methylated forms, conservatively modified variants thereof (e.g., degenerate codon substitutions), alleles, orthologs, single nucleotide polymorphisms (SNPs), and complementary sequences as well as the sequence explicitly indicated. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil.

A “methylated regulatory element” as used herein refers to a segment of DNA sequence at a defined location in the genome of an individual. Typically, a “methylated regulatory element” is at least 15 nucleotides in length and contains at least one cytosine. It may be at least 18, 20, 25, 30, 50, 80, 100, 150, 200, 250, or 300 nucleotides in length and contain 1 or 2, 5, 10, 15, 20, 25, or 30 cytosines. For any one “methylated regulatory element” at a given location, e.g., within a region centering around a given genetic locus, nucleotide sequence variations may exist from individual to individual and from allele to allele even for the same individual. Typically, such a region centering around a defined genetic locus (e.g., a CpG island) contains the locus as well as upstream and/or downstream sequences. Each of the upstream or downstream sequence (counting from the 5′ or 3′ boundary of the genetic locus, respectively) can be as long as 10 kb, in other cases may be as long as 5 kb, 2 kb, 1 kb, 500 bp, 200 bp, or 100 bp. Furthermore, a “methylated regulatory element” may modulate expression of a nucleotide sequence transcribed into a protein or not transcribed for protein production (such as a non-coding mRNA). The “methylated regulatory element” may be an inter-gene sequence, intra-gene sequence (intron), protein-coding sequence (exon), a non protein-coding sequence (such as a transcription promoter or enhancer), or a combination thereof.

As used herein, a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring, however, for purposes herein, thymine is not considered a methylated nucleotide when present in DNA since thymine is a typical nucleotide base of DNA. Typical nucleoside bases for DNA are thymine, adenine, cytosine and guanine. Typical bases for RNA are uracil, adenine, cytosine and guanine. Correspondingly a “methylation site” is the location in the target gene nucleic acid region where methylation has, or has the possibility of occurring. For example, a location containing CpG is a methylation site wherein the cytosine may or may not be methylated.

As used herein, a “CpG site” or “methylation site” is a nucleotide within a nucleic acid that is susceptible to methylation either by natural occurring events in vivo or by an event instituted to chemically methylate the nucleotide in vitro.

As used herein, a “methylated nucleic acid molecule” refers to a nucleic acid molecule that contains one or more nucleotides that is/are methylated.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745).

The term “epigenetic state” or “epigenetic status” as used herein refers to any structural feature at a molecular level of a nucleic acid (e.g., DNA or RNA) other than the primary nucleotide sequence. For instance, the epigenetic state of a genomic DNA may include its secondary or tertiary structure determined or influenced by, e.g., its methylation pattern or its association with cellular proteins.

The term “methylation profile” “methylation state” or “methylation status,” as used herein to describe the state of methylation of a genomic sequence, refers to the characteristics of a DNA segment at a particular genomic locus relevant to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles. The term “methylation” profile” or “methylation status” also refers to the relative or absolute concentration of methylated C or unmethylated C at any particular stretch of residues in a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., sample nucleic acid) are methylated as compared to another sequence from a different region or from a different individual (e.g., relative to normal nucleic acid), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”, and more specifically, when the methylation status differs between melanoma and benign or healthy moles, the sequences are considered “differentially methylated in melanoma and benign nevi”. Measurement of the levels of differential methylation may be done by a variety of ways known to those skilled in the art. One method is to measure the methylation level of individual interrogated CpG sites determined by the β-value, defined as the ratio of fluorescent signal from the methylated allele to the sum of the fluorescent signals of both the methylated and unmethylated alleles and calculated as β=max(Cy5,0)/(|Cy5|+|Cy3|+100). β values ranged from 0 in the case of completely unmethylated to 1 in the case of fully methylated DNA. (Bibikova et al., 2006) The difference in the ratios between methylated and unmethylated sequences in melanoma and benign nevi may be 0.1, 0.15, 0.2, 0.25, 0.3, 0.4, 0.5, 0.55, 0.6, 0.65, 0.7, 0.8, or 0.9. In non-limiting embodiments, the difference in the ratios is between 0.2 and 0.65, or between 0.2 and 0.4.

The term “bisulfite” as used herein encompasses any suitable type of bisulfite, such as sodium bisulfite, or other chemical agent that is capable of chemically converting a cytosine (C) to a uracil (U) without chemically modifying a methylated cytosine and therefore can be used to differentially modify a DNA sequence based on the methylation status of the DNA, e.g., U.S. Pat. Pub. US 2010/0112595 (Menchen et al.). As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C→U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

The terms “non-bisulfite-based method” and “non-bisulfite-based quantitative method” as used herein refer to any method for quantifying methylated or non-methylated nucleic acid that does not require the use of bisulfite. The terms also refer to methods for preparing a nucleic acid to be quantified that do not require bisulfite treatment. Examples of non-bisulfite-based methods include, but are not limited to, methods for digesting nucleic acid using one or more methylation sensitive enzymes and methods for separating nucleic acid using agents that bind nucleic acid based on methylation status. The terms “methyl-sensitive enzymes” and “methylation sensitive restriction enzymes” are DNA restriction endonucleases that are dependent on the methylation state of their DNA recognition site for activity. For example, there are methyl-sensitive enzymes that cleave or digest at their DNA recognition sequence only if it is not methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. As used herein, the terms “cleave”, “cut” and “digest” are used interchangeably.

The term “target nucleic acid” as used herein refers to a nucleic acid examined using the methods disclosed herein to determine if the nucleic acid is melanoma associated. The term “control nucleic acid” as used herein refers to a nucleic acid used as a reference nucleic acid according to the methods disclosed herein to determine if the nucleic acid is associated with melanoma. The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Primers” as used herein refer to oligonucleotides that can be used in an amplification method, such as a polymerase chain reaction (PCR), to amplify a nucleotide sequence based on the polynucleotide sequence corresponding to a particular genomic sequence, e.g., one specific for a particular CpG site. At least one of the PCR primers for amplification of a polynucleotide sequence is sequence-specific for the sequence.

The term “template” refers to any nucleic acid molecule that can be used for amplification in the technology. RNA or DNA that is not naturally double stranded can be made into double stranded DNA so as to be used as template DNA. Any double stranded DNA or preparation containing multiple, different double stranded DNA molecules can be used as template DNA to amplify a locus or loci of interest contained in the template DNA.

The term “amplification reaction” as used herein refers to a process for copying nucleic acid one or more times. In embodiments, the method of amplification includes, but is not limited to, polymerase chain reaction, self-sustained sequence reaction, ligase chain reaction, rapid amplification of cDNA ends, polymerase chain reaction and ligase chain reaction, Q-β replicase amplification, strand displacement amplification, rolling circle amplification, or splice overlap extension polymerase chain reaction. In some embodiments, a single molecule of nucleic acid may be amplified.

The term “sensitivity” as used herein refers to the number of true positives divided by the number of true positives plus the number of false negatives, where sensitivity (sens) may be within the range of 0<sens<1. Ideally, method embodiments herein have the number of false negatives equaling zero or close to equaling zero, so that no subject is wrongly identified as not having melanoma when they indeed have melanoma. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where sensitivity (spec) may be within the range of 0<spec<1. Ideally, the methods described herein have the number of false positives equaling zero or close to equaling zero, so that no subject is wrongly identified as having melanoma when they do not in fact have melanoma. Hence, a method that has both sensitivity and specificity equaling one, or 100%, is preferred.

“RNAi molecule” or “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, siRNA refers to a nucleic acid that has substantial or complete identity to a target gene and forms a double stranded siRNA. The sequence of the siRNA can correspond to the full-length target gene, or a sub-sequence of the full-length target gene. Typically, the siRNA is at least about 15-50 nucleotides in length (e.g., each complementary sequence of the double stranded siRNA is 15-50 nucleotides in length, and the double stranded siRNA is about 15-50 base pairs in length, preferably about 20-30 base nucleotides, preferably about 20-25 nucleotides in length, e.g., 20, 21, 22, 23, 24, 25, 26, 27, 28, 29, or 30 nucleotides in length.

An “antisense” polynucleotide is a polynucleotide that is substantially complementary to a target polynucleotide and has the ability to specifically hybridize to the target polynucleotide. Ribozymes are enzymatic RNA molecules capable of catalyzing specific cleavage of RNA. The composition of ribozyme molecules preferably includes one or more sequences complementary to a target mRNA, and the well-known catalytic sequence responsible for mRNA cleavage or a functionally equivalent sequence (see, e.g., U.S. Pat. No. 5,093,246 (Cech et al.); U.S. Pat. No. 5,766,942 (Haseloff et al.); U.S. Pat. No. 5,856,188 (Hampel et al.) which are incorporated herein by reference in their entirety). Ribozyme molecules designed to catalytically cleave target mRNA transcripts can also be used to prevent translation of genes associated with the progression of melanoma. These genes may be genes found to be differentially methylated in melanoma.

The phrase “functional effects” in the context of assays for testing means compounds that modulate a methylation of a regulatory region of a gene associated with melanoma. This may also be a chemical or phenotypic effect such as altered transcriptional activity of a gene differentially methylated in melanoma, or altered activities and the downstream effects of proteins encoded by these genes. A functional effect may include transcriptional activation or repression, the ability of cells to proliferate, expression in cells during melanoma progression, and other characteristics of melanoma cells. “Functional effects” include in vitro, in vivo, and ex vivo activities. By “determining the functional effect” is meant assaying for a compound that increases or decreases the transcription of genes or the translation of proteins that are indirectly or directly under the influence of a gene differentially methylated in melanoma. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of the marker; measuring changes in enzymatic activity; the ability to increase or decrease cellular proliferation, apoptosis, cell cycle arrest, measuring changes in cell surface markers. Validation of the functional effect of a compound on melanoma progression can also be performed using assays known to those of skill in the art such as metastasis of melanoma cells by tail vein injection of melanoma cells in mice. The functional effects can be evaluated by many means known to those skilled in the art, e.g., microscopy for quantitative or qualitative measures of alterations in morphological features, measurement of changes in RNA or protein levels for other genes expressed in melanoma cells, measurement of RNA stability, identification of downstream or reporter gene expression (CAT, luciferase, β-gal, GFP and the like), e.g., via chemiluminescence, fluorescence, colorimetric reactions, antibody binding, inducible markers, etc.

“Inhibitors,” “activators,” and “modulators” of the markers are used to refer to activating, inhibitory, or modulating molecules identified using in vitro and in vivo assays of the methylation state, the expression of genes differentially methylated in melanoma or the translation proteins encoded thereby. Inhibitors, activators, or modulators also include naturally occurring and synthetic ligands, antagonists, agonists, antibodies, peptides, cyclic peptides, nucleic acids, antisense molecules, ribozymes, RNAi molecules, small organic molecules and the like. Such assays for inhibitors and activators include, e.g., (1)(a) measuring methylation states, (b) the mRNA expression, or (c) proteins expressed by genes differentially methylated in melanoma in vitro, in cells, or cell extracts; (2) applying putative modulator compounds; and (3) determining the functional effects on activity, as described above.

Samples or assays comprising genes differentially methylated in melanoma are treated with a potential activator, inhibitor, or modulator are compared to control samples without the inhibitor, activator, or modulator to examine the extent of inhibition. Control samples (untreated with inhibitors) are assigned a relative activity value of 100%. Inhibition of methylation, expression, or proteins encoded by genes differentially methylated in melanoma is achieved when the activity value relative to the control is about 80%, preferably 50%, more preferably 25-0%. Activation of methylation, expression, or proteins encoded by genes differentially methylated in melanoma is achieved when the activity value relative to the control (untreated with activators) is 110%, more preferably 150%, more preferably 200-500% (i.e., two to five-fold higher relative to the control), more preferably 1000-3000% higher. While many changes in methylation will be associated with changes in activity or functional effects, some changes in methylation may not. Nonetheless, changes in the 40 CpG or 59 CpG methylation signature described herein are indicative of increased likelihood of melanoma.

The term “test compound” or “drug candidate” or “modulator” or grammatical equivalents as used herein describes any molecule, either naturally occurring or synthetic, e.g., protein, oligopeptide, small organic molecule, polysaccharide, peptide, circular peptide, lipid, fatty acid, siRNA, polynucleotide, oligonucleotide, etc., to be tested for the capacity to directly or indirectly modulate genes differentially methylated in melanoma. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis. The compound may be “small organic molecule” that is an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

As used herein, the verb “comprise” in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### 5.2. Tissue Samples

The tissue sample may be from a patient suspected of having melanoma or from a patient diagnosed with melanoma, e.g., for confirmation of diagnosis or establishing a clear margin or for the detection of melanoma cells in other tissues such as lymph nodes. The biological sample may also be from a subject with an ambiguous diagnosis in order to clarify the diagnosis. The sample may be obtained for the purpose of differential diagnosis, e.g., a subject with a histopathologically benign lesion to confirm the diagnosis. The sample may also be obtained for the purpose of prognosis, i.e., determining the course of the disease and selecting primary treatment options. Tumor staging and grading are examples of prognosis. The sample may also be evaluated to select or monitor therapy, selecting likely responders in advance from non-responders or monitoring response in the course of therapy. In addition, the sample may be evaluated as part of post-treatment ongoing surveillance of patients who have had melanoma. The sample may also be obtained to differentiate dysplastic nevi from other benign nevi. The sample may be a melanoma sample such as a superficial spreading melanoma, nodular melanoma, lentigo maligna melanoma, acral lentiginous melanoma, unclassifiable or other (spitzoid/desmoplastic/nevoid/spindle cell) melanoma. The sample may be normal skin, a benign nevus, a melanoma-in-situ (MIS), or a high-grade dysplastic nevus (HGDN).

Biological samples may be obtained using any of a number of methods in the art. Examples of biological samples comprising potential melanocytic lesions include those obtained from excised skin biopsies, such as punch biopsies, shave biopsies, fine needle aspirates (FNA), or surgical excisions; or biopsy from non-cutaneous tissues such as lymph node tissue, mucosa, conjuctiva, or uvea, other embodiments. The biological sample may be obtained by shaving, waxing, or stripping the region of interest on the skin. A non-limiting example of a product for stripping skin for RNA recovery is the EGIR™ tape strip product (DermTech International, La Jolla, Calif., see also, Wachsman et al., 2011, Brit. J. Dern. 164 797-806). Representative biopsy techniques include, but are not limited to, excisional biopsy, incisional biopsy, needle biopsy, surgical biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy may require a “core-needle biopsy” of the tumor mass, or a “fine-needle aspiration biopsy” which generally contains a suspension of cells from within the tumor mass. The biological sample may be a microdissected sample, such as a PALM-laser (Carl Zeiss MicroImaging GmbH, Germany) capture microdissected sample.

A sample may also be a sample of muscosal surfaces, blood and blood fractions or products (e.g., serum, plasma, platelets, red blood cells, white blood cells, circulating tumor cells isolated from blood, free DNA isolated from blood, and the like), sputum, lymph and tongue tissue, cultured cells, e.g., primary cultures, explants, and transformed cells, stool, urine, etc. The sample may also be vascular tissue or cells from blood vessels such as microdissected blood vessel cells of endothelial origin. A sample is typically obtained from a eukaryotic organism, most preferably a mammal such as a primate e.g., chimpanzee or human, cow, dog, cat; or a rodent, e.g., guinea pig, rat, mouse, rabbit.

A sample can be treated with a fixative such as formaldehyde and embedded in paraffin (FFPE) and sectioned for use in the methods of the invention. Alternatively, fresh or frozen tissue may be used. These cells may be fixed, e.g., in alcoholic solutions such as 100% ethanol or 3:1 methanol:acetic acid. Nuclei can also be extracted from thick sections of paraffin-embedded specimens to reduce truncation artifacts and eliminate extraneous embedded material. Typically, biological samples, once obtained, are harvested and processed prior to nucleic acid analysis using standard methods known in the art. Such processing typically includes protease treatment and additional fixation in an aldehyde solution such as formaldehyde.

### 5.3. Techniques for Measuring Methylation

A variety of methylation analysis procedures are known in the art and may be used to practice the invention. These assays allow for determination of the methylation state of one or a plurality of CpG sites within a tissue sample. In addition, these methods may be used for absolute or relative quantification of methylated nucleic acids. Another embodiment of the invention are methods of detecting melanoma based on differentially methylated sites found in tissue analysis described herein, and not differentially methylated in cultured melanocytes and/or melanoma cell lines. Such methylation assays may involve, among other techniques, two major steps. The first step is a methylation specific reaction or separation, such as (i) bisulfite treatment, (ii) methylation specific binding, or (iii) methylation specific restriction enzymes. The second major step involves (i) amplification and detection, or (ii) direct detection, by a variety of methods such as (a) PCR (sequence-specific amplification) such as Taqman®, (b) DNA sequencing of untreated and bisulfite-treated DNA, (c) sequencing by ligation of dye-modified probes (including cyclic ligation and cleavage), (d) pyrosequencing, (e) single-molecule sequencing, (f) mass spectroscopy, or (g) Southern blot analysis.

Additionally, restriction enzyme digestion of PCR products amplified from bisulfite-converted DNA may be used, e.g., the method described by Sadri & Hornsby (1996, Nucl. Acids Res. 24:5058-5059), or COBRA (Combined Bisulfite Restriction Analysis) (Xiong & Laird, 1997, Nucleic Acids Res. 25:2532-2534). COBRA analysis is a quantitative methylation assay useful for determining DNA methylation levels at specific gene loci in small amounts of genomic DNA. Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Frommer et al., 1992, Proc. Nat. Acad. Sci. USA, 89, 1827-1831). PCR amplification of the bisulfite converted DNA is then performed using primers specific for the CpG sites of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligomer; control hybridization oligomer; kinase labeling kit for oligomer probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

5.3.1. Methylation-Specific PCR (MSP)

Methylation-Specific PCR (MSP) allows for assessing the methylation status of virtually any group of CpG sites within a CpG island, independent of the use of methylation-sensitive restriction enzymes (Herman et al., 1996, Proc. Nat. Acad. Sci. USA, 93, 9821-9826; U.S. Pat. Nos. 5,786,146, 6,017,704, 6,200,756, 6,265,171 (Herman & Baylin) U.S. Pat. Pub. No. 2010/0144836 (Van Engeland et al.); which are hereby incorporated by reference in their entirety). Briefly, DNA is modified by sodium bisulfite converting unmethylated, but not methylated cytosines to uracil, and subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes. The ColoSure™ test is a commercially available test for colon cancer based on the MSP technology and measurement of methylation of the vimentin gene (Itzkowitz et al., 2007, Clin Gastroenterol. Hepatol. 5(1), 111-117). Alternatively, one may use quantitative multiplexed methylation specific PCR (QM-PCR), as described by Fackler et al. Fackler et al., 2004, Cancer Res. 64(13) 4442-4452; or Fackler et al., 2006, Clin. Cancer Res. 12(11 Pt 1) 3306-3310.

5.3.2. MethyLight and Heavy Methyl Methods

The MethyLight and Heavy Methyl assays are a high-throughput quantitative methylation assay that utilizes fluorescence-based real-time PCR (Taq Man®) technology that require no further manipulation after the PCR step (Eads, C. A. et al., 2000, Nucleic Acid Res. 28, e 32; Cottrell et al., 2007, J. Urology 177, 1753, U.S. Pat. No. 6,331,393 (Laird et al.), the contents of which are hereby incorporated by reference in their entirety). Briefly, the MethyLight process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both. The MethyLight assay may be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites. Typical reagents (e.g., as might be found in a typical MethyLight-based kit) for MethyLight analysis may include, but are not limited to: PCR primers for specific gene (or methylation-altered DNA sequence or CpG island); TaqMan® probes; optimized PCR buffers and deoxynucleotides; and Taq polymerase. The MethyLight technology is used for the commercially available tests for lung cancer (epi proLung BL Reflex Assay); colon cancer (epi proColon assay and mSEPT9 assay) (Epigenomics, Berlin, Germany) PCT Pub. No. WO 2003/064701 (Schweikhardt and Sledziewski), the contents of which is hereby incorporated by reference in its entirety.

Quantitative MethyLight uses bisulfite to convert genomic DNA and the methylated sites are amplified using PCR with methylation independent primers. Detection probes specific for the methylated and unmethylated sites with two different fluorophores provides simultaneous quantitative measurement of the methylation. The Heavy Methyl technique begins with bisulfate conversion of DNA. Next specific blockers prevent the amplification of unmethylated DNA. Methylated genomic DNA does not bind the blockers and their sequences will be amplified. The amplified sequences are detected with a methylation specific probe. (Cottrell et al., 2004, Nuc. Acids Res. 32, e10, the contents of which is hereby incorporated by reference in its entirety).

The Ms-SNuPE technique is a quantitative method for assessing methylation differences at specific CpG sites based on bisulfite treatment of DNA, followed by single-nucleotide primer extension (Gonzalgo & Jones, 1997, Nucleic Acids Res. 25, 2529-2531). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites. Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery reagents or kit (e.g., precipitation, ultrafiltration, affinity column); desulfonation buffer; and DNA recovery components.

5.3.3. Differential Binding-based Methylation Detection Methods

For identification of differentially methylated regions, one approach is to capture methylated DNA. This approach uses a protein, in which the methyl binding domain of MBD2 is fused to the Fc fragment of an antibody (MBD-FC) (Gebhard et al., 2006, Cancer Res. 66:6118-6128; and PCT Pub. No. WO 2006/056480 A2 (Relhi), the contents of which are hereby incorporated by reference in their entirety). This fusion protein has several advantages over conventional methylation specific antibodies. The MBD FC has a higher affinity to methylated DNA and it binds double stranded DNA. Most importantly the two proteins differ in the way they bind DNA. Methylation specific antibodies bind DNA stochastically, which means that only a binary answer can be obtained. The methyl binding domain of MBD-FC, on the other hand, binds DNA molecules regardless of their methylation status. The strength of this protein—DNA interaction is defined by the level of DNA methylation. After binding genomic DNA, eluate solutions of increasing salt concentrations can be used to fractionate non-methylated and methylated DNA allowing for a more controlled separation (Gebhard et al., 2006, Nucleic Acids Res. 34 e82). Consequently, this method, called Methyl-CpG immunoprecipitation (MCIP), not only enriches, but also fractionates genomic DNA according to methylation level, which is particularly helpful when the unmethylated DNA fraction should be investigated as well.

Alternatively, one may use 5-methyl cytidine antibodies to bind and precipitate methylated DNA. Antibodies are available from Abcam (Cambridge, Mass.), Diagenode (Sparta, N.J.) or Eurogentec (c/o AnaSpec, Fremont, Calif.). Once the methylated fragments have been separated they may be sequenced using microarray based techniques such as methylated CpG-island recovery assay (MIRA) or methylated DNA immunoprecipitation (MeDIP) (Pelizzola et al., 2008, Genome Res. 18, 1652-1659; O'Geen et al., 2006, BioTechniques 41(5), 577-580, Weber et al., 2005, Nat. Genet. 37, 853-862; Horak and Snyder, 2002, Methods Enzymol., 350, 469-83; Lieb, 2003, Methods Mol. Biol., 224, 99-109). Another technique is methyl-CpG binding domain column/segregation of partly melted molecules (MBD/SPM, Shiraishi et al., 1999, Proc. Natl. Acad. Sci. USA 96(6):2913-2918).

5.3.4. Methylation Specific Restriction Enzymatic Methods

For example, there are methylation-sensitive enzymes that preferentially or substantially cleave or digest at their DNA recognition sequence if it is non-methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. Methylation-sensitive enzymes that digest unmethylated DNA suitable for use in methods of the technology include, but are not limited to, Hpall, Hhal, Maell, BstUI and Acil. An enzyme that can be used is Hpall that cuts only the unmethylated sequence CCGG. Another enzyme that can be used is Hhal that cuts only the unmethylated sequence GCGC. Both enzymes are available from New England BioLabs®, Inc. Combinations of two or more methyl-sensitive enzymes that digest only unmethylated DNA can also be used. Suitable enzymes that digest only methylated DNA include, but are not limited to, Dpnl, which only cuts at fully methylated 5′-GATC sequences, and McrBC, an endonuclease, which cuts DNA containing modified cytosines (5-methylcytosine or 5-hydroxymethylcytosine or N4-methylcytosine) and cuts at recognition site 5′ . . . Pu^{m}C(N₄₀₋₃₀₀₀) Pu^{m}C . . . 3′ (New England BioLabs, Inc., Beverly, Mass.). Cleavage methods and procedures for selected restriction enzymes for cutting DNA at specific sites are well known to the skilled artisan. For example, many suppliers of restriction enzymes provide information on conditions and types of DNA sequences cut by specific restriction enzymes, including New England BioLabs, Pro-Mega Biochems, Boehringer-Mannheim, and the like. Sambrook et al. (See Sambrook et al. Molecular Biology: A Laboratory Approach, Cold Spring Harbor, N.Y. 1989) provide a general description of methods for using restriction enzymes and other enzymes.

The methylated CpG island amplification (MCA) technique is a method that can be used to screen for altered methylation patterns in genomic DNA, and to isolate specific sequences associated with these changes (Toyota et al., 1999, Cancer Res. 59, 2307-2312, U.S. Pat. No. 7,700,324 (Issa et al.) the contents of which are hereby incorporated by reference in their entirety). Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligomers or probes; control hybridization oligomers or probes.

5.3.5. Methylation-Sensitive High Resolution Melting (HRM)

Wojdacz et al. reported methylation-sensitive high resolution melting as a technique to assess methylation. (Wojdacz and Dobrovic, 2007, Nuc. Acids Res. 35(6) e41; Wojdacz et al. 2008, Nat. Prot. 3(12) 1903-1908; Balic et al., 2009 J. Mol. Diagn. 11 102-108; and US Pat. Pub. No. 2009/0155791 (Wojdacz et al.), the contents of which are hereby incorporated by reference in their entirety). A variety of commercially available real time PCR machines have HRM systems including the Roche LightCycler480, Corbett Research RotorGene6000, and the Applied Biosystems 7500. HRM may also be combined with other techniques such as pyrosequencing as described by Candiloro et al. (Candiloro et al., 2011, Epigenetics 6(4) 500-507), QPCR or MSP. In one embodiment, HRM is performed on the Roche LightCycler with MSP assays using SYBR green instead of TaqMan probes. Any of SEQ ID NO 1-480, or portions thereof, may be used in a HRM assay.

5.3.6. Mass Spectroscopic Detection Methods

Another method for analyzing methylation sites is a primer extension assay, including an optimized PCR amplification reaction that produces amplified targets for analysis using mass spectrometry. The assay can also be done in a multiplex format. Mass spectrometry is a particularly effective method for the detection of polynucleotides associated with the differentially methylated regulatory elements. The presence of the polynucleotide sequence is verified by comparing the mass of the detected signal with the expected mass of the polynucleotide of interest. The relative signal strength, e.g., mass peak on a spectra, for a particular polynucleotide sequence indicates the relative population of a specific allele, thus enabling calculation of the allele ratio directly from the data. This method is described in detail in PCT Pub. No. WO 2005/012578A1 (Beaulieu et al.) which is hereby incorporated by reference in its entirety. For methylation analysis, the assay may be adopted to detect bisulfite introduced methylation dependent C to T sequence changes. These methods are particularly useful for performing multiplexed amplification reactions and multiplexed primer extension reactions (e g., multiplexed homogeneous primer mass extension (hME) assays) in a single well to further increase the throughput and reduce the cost per reaction for primer extension reactions.

For a review of mass spectrometry methods using Sequenom® standard iPLEX™ assay and MassARRAY® technology, see Jurinke et al., 2004, Mol. Biotechnol. 26, 147-164. For methods of detecting and quantifying target nucleic acids using cleavable detector probes that are cleaved during the amplification process and detected by mass spectrometry, see PCT Pub. Nos. WO 2006/031745 (Van Der Boom and Boecker); WO 2009/073251 A1 (Van Den Boom et al.); WO 2009/114543 A2 (Oeth et al.); and WO 2010/033639 A2 (Ehrich et al.); which are hereby incorporated by reference in their entirety.

5.3.7. Additional Methods for Methylation Analysis

Other methods for DNA methylation analysis include restriction landmark genomic scanning (RLGS, Costello et al., 2002, Meth. Mol. Biol., 200, 53-70), methylation-sensitive-representational difference analysis (MS-RDA, Ushijima and Yamashita, 2009, Methods Mol Biol. 507, 117-130). Comprehensive high-throughput arrays for relative methylation (CHARM) techniques are described in WO 2009/021141 (Feinberg and Irizarry). The Roche® NimbleGen® microarrays including the Chromatin Immunoprecipitation-on-chip (ChIP-chip) or methylated DNA immunoprecipitation-on-chip (MeDIP-chip). These tools have been used for a variety of cancer applications including melanoma, liver cancer and lung cancer (Koga et al., 2009, Genome Res., 19, 1462-1470; Acevedo et al., 2008, Cancer Res., 68, 2641-2651; Rauch et al., 2008, Proc. Nat. Acad. Sci. USA, 105, 252-257). Others have reported bisulfate conversion, padlock probe hybridization, circularization, amplification and next generation or multiplexed sequencing for high throughput detection of methylation (Deng et al., 2009, Nat. Biotechnol. 27, 353-360; Ball et al., 2009, Nat. Biotechnol. 27, 361-368; U.S. Pat. No. 7,611,869 (Fan)). As an alternative to bisulfate oxidation, Carrell et al. have reported selective oxidants that oxidize 5-methylcytosine, without reacting with thymidine, which are followed by PCR or pyrosequencing (WO 2009/049916 (Carrell et al.). These references for these techniques are hereby incorporated by reference in their entirety.

5.3.8. Polynucleotide Sequence Amplification and Determination

Following reaction or separation of nucleic acid in a methylation specific manner, the nucleic acid may be subjected to sequence-based analysis. Furthermore, once it is determined that one particular melanoma genomic sequence is differentially methylated compared to the benign counterpart, the amount of this genomic sequence can be determined. Subsequently, this amount can be compared to a standard control value and serve as an indication for the melanoma. In many instances, it is desirable to amplify a nucleic acid sequence using any of several nucleic acid amplification procedures which are well known in the art. Specifically, nucleic acid amplification is the chemical or enzymatic synthesis of nucleic acid copies which contain a sequence that is complementary to a nucleic acid sequence being amplified (template). The methods and kits of the invention may use any nucleic acid amplification or detection methods known to one skilled in the art, such as those described in U.S. Pat. No. 5,525,462 (Takarada et al.); U.S. Pat. No. 6,114,117 (Hepp et al.); U.S. Pat. No. 6,127,120 (Graham et al.); U.S. Pat. No. 6,344,317 (Urnovitz); U.S. Pat. No. 6,448,001 (Oku); U.S. Pat. No. 6,528,632 (Catanzariti et al.); and PCT Pub. No. WO 2005/111209 (Nakajima et al.); all of which are incorporated herein by reference in their entirety.

In some embodiments, the nucleic acids may be amplified by PCR amplification using methodologies known to one skilled in the art. One skilled in the art will recognize, however, that amplification can be accomplished by other known methods, such as ligase chain reaction (LCR), Qβ-replicase amplification, rolling circle amplification, transcription amplification, self-sustained sequence replication, nucleic acid sequence-based amplification (NASBA), each of which provides sufficient amplification. Branched-DNA technology may also be used to qualitatively demonstrate the presence of a sequence of the technology, which represents a particular methylation pattern, or to quantitatively determine the amount of this particular genomic sequence in a sample. Nolte reviews branched-DNA signal amplification for direct quantitation of nucleic acid sequences in clinical samples (Nolte, 1998, Adv. Clin. Chem. 33:201-235).

The PCR process is well known in the art and is thus not described in detail herein. For a review of PCR methods and protocols, see, e.g., Innis et al., eds., PCR Protocols, A Guide to Methods and Application, Academic Press, Inc., San Diego, Calif. 1990; U.S. Pat. No. 4,683,202 (Mullis); which are incorporated herein by reference in their entirety. PCR reagents and protocols are also available from commercial vendors, such as Roche Molecular Systems. PCR may be carried out as an automated process with a thermostable enzyme. In this process, the temperature of the reaction mixture is cycled through a denaturing region, a primer annealing region, and an extension reaction region automatically. Machines specifically adapted for this purpose are commercially available.

Amplified sequences may also be measured using invasive cleavage reactions such as the Invader® technology (Zou et al., 2010, Association of Clinical Chemistry (AACC) poster presentation on Jul. 28, 2010, “Sensitive Quantification of Methylated Markers with a Novel Methylation Specific Technology,” available at www.exactsciences.com; and U.S. Pat. No. 7,011,944 (Prudent et al.) which are incorporated herein by reference in their entirety).

5.3.9. High Throughput and Single Molecule Sequencing Technology

Suitable next generation sequencing technologies are widely available. Examples include the 454 Life Sciences platform (Roche, Branford, Conn.) (Margulies et al. 2005 Nature, 437, 376-380); Illumina's Genome Analyzer, Illumina's MiSeq System, Illumina's NextSeq System, Illumina's MiniSeq System, GoldenGate Methylation Assay, or Infinium Methylation Assays, i.e., Illumina Infinium MethylationEPIC BeadChip (850K array), Illumina Infinium HumanMethylation450 BeadChip, or Infinium HumanMethylation 27K BeadArray (Illumina, San Diego, Calif.; Bibkova et al., 2006, Genome Res. 16, 383-393; U.S. Pat. Nos. 6,306,597 and 7,598,035 (Macevicz); U.S. Pat. No. 7,232,656 (Balasubramanian et al.)); or DNA Sequencing by Ligation, SOLiD System (Applied Biosystems/Life Technologies; U.S. Pat. Nos. 6,797,470, 7,083,917, 7,166,434, 7,320,865, 7,332,285, 7,364,858, and 7,429,453 (Barany et al.); or the Helicos True Single Molecule DNA sequencing technology (Harris et al., 2008 Science, 320, 106-109; U.S. Pat. Nos. 7,037,687 and 7,645,596 (Williams et al.); 7,169,560 (Lapidus et al.); 7,769,400 (Harris)), the single molecule, real-time (SMRT™) technology of Pacific Biosciences, and sequencing (Soni and Meller, 2007, Clin. Chem. 53, 1996-2001) which are incorporated herein by reference in their entirety. These systems allow the sequencing of many nucleic acid molecules isolated from a specimen at high orders of multiplexing in a parallel fashion (Dear, 2003, Brief Funct. Genomic Proteomic, 1(4), 397-416 and McCaughan and Dear, 2010, J. Pathol., 220, 297-306). Each of these platforms allow sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, (iii) targeted next-generation sequencing from bisulfite treated DNA and (iv) single-molecule sequencing.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Study nucleic acids may be immobilized to a solid support, hybridized with a sequencing primer, incubated with DNA polymerase, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphosulfate, fueling the luciferin reaction, which produces a chemiluminescent signal allowing sequence determination. Machines for pyrosequencing and methylation specific reagents are available from Qiagen, Inc. (Valencia, Calif.). See also Tost and Gut, 2007, Nat. Prot. 2 2265-2275. An example of a system that can be used by a person of ordinary skill based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano et al., 2003, J. Biotech. 102, 117-124). Such a system can be used to exponentially amplify amplification products generated by a process described herein, e.g., by ligating a heterologous nucleic acid to the first amplification product generated by a process described herein.

Next-generation sequencing (NGS) is a nucleic acid sequencing method based on sequencing by synthesis, where fluorescently labeled deoxyribonucleotide triphosphates (dNTPs) catalyzed by DNA polymerase are incorporated into a DNA temple through cycles of DNA synthesis and nucleotides are identified by fluorophore excitation at each incorporation step. NGS allows this process to take place in a multiplex reaction across millions of DNA fragments in parallel. Generally, sequencing by synthesis involves synthesizing, one nucleotide at a time, a DNA strand complimentary to the strand whose sequence is being sought. Study nucleic acids may be immobilized to a solid support, hybridized with a sequencing primer, and incubated with DNA polymerase in the presence of fluorescently labeled dNTPS. After each cycle, the image is scanned and the emission wavelength and intensity are recorded and used to identify the base incorporated. This process is repeated multiple times to create a specific read length of bases. Such a system can be used to exponentially amplify amplification products generated by a process described herein, e.g., by sequencing bisulfite-treated DNA to identify methylated or unmethylated CpGs included in our diagnostic model.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing or detection, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each other for energy transfer to occur successfully. Bailey et al. recently reported a highly sensitive (15 pg methylated DNA) method using quantum dots to detect methylation status using fluorescence resonance energy transfer (MS-qFRET) (Bailey et al. 2009, Genome Res. 19(8), 1455-1461, which is incorporated herein by reference in its entirety).

An example of a system that can be used based on single-molecule sequencing generally involves hybridizing a primer to a study nucleic acid to generate a complex; associating the complex with a solid phase; iteratively extending the primer by a nucleotide tagged with a fluorescent molecule; and capturing an image of fluorescence resonance energy transfer signals after each iteration (e.g., Braslavsky et al., PNAS 100(7): 3960-3964 (2003); U.S. Pat. No. 7,297,518 (Quake et al.) which are incorporated herein by reference in their entirety). Such a system can be used to directly sequence amplification products generated by processes described herein. In some embodiments, the released linear amplification product can be hybridized to a primer that contains sequences complementary to immobilized capture sequences present on a solid support, a bead or glass slide for example. Hybridization of the primer-released linear amplification product complexes with the immobilized capture sequences, immobilizes released linear amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer-released linear amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

The technology described herein may be practiced with digital PCR. Digital PCR was developed by Kalinina and colleagues (Kalinina et al., 1997, Nucleic Acids Res. 25; 1999-2004) and further developed by Vogelstein and Kinzler (1999, Proc. Natl. Acad. Sci. U.S.A. 96; 9236-9241). The application of digital PCR is described by Cantor et al. (PCT Pub. Nos. WO 2005/023091A2 (Cantor et al.); WO 2007/092473 A2, (Quake et al.)), which are hereby incorporated by reference in their entirety. Digital PCR takes advantage of nucleic acid (DNA, cDNA or RNA) amplification on a single molecule level, and offers a highly sensitive method for quantifying low copy number nucleic acid. Fluidigm® Corporation offers systems for the digital analysis of nucleic acids.

In some embodiments, nucleotide sequencing may be by solid phase single nucleotide sequencing methods and processes. Solid phase single nucleotide sequencing methods involve contacting sample nucleic acid and solid support under conditions in which a single molecule of sample nucleic acid hybridizes to a single molecule of a solid support. Such conditions can include providing the solid support molecules and a single molecule of sample nucleic acid in a “microreactor.” Such conditions also can include providing a mixture in which the sample nucleic acid molecule can hybridize to solid phase nucleic acid on the solid support. Single nucleotide sequencing methods useful in the embodiments described herein are described in PCT Pub. No. WO 2009/091934 (Cantor).

In certain embodiments, nanopore sequencing detection methods include (a) contacting a nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected.

A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

The invention encompasses methods known in the art for enhancing the sensitivity of the detectable signal in such assays, including, but not limited to, the use of cyclic probe technology (Bakkaoui et al., 1996, BioTechniques 20: 240-8, which is incorporated herein by reference in its entirety); and the use of branched probes (Urdea et al., 1993, Clin. Chem. 39, 725-6; which is incorporated herein by reference in its entirety). The hybridization complexes are detected according to well-known techniques in the art.

Reverse transcribed or amplified nucleic acids may be modified nucleic acids. Modified nucleic acids can include nucleotide analogs, and in certain embodiments include a detectable label and/or a capture agent. Examples of detectable labels include, without limitation, fluorophores, radioisotopes, colorimetric agents, light emitting agents, chemiluminescent agents, light scattering agents, enzymes and the like. Examples of capture agents include, without limitation, an agent from a binding pair selected from antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) pairs, and the like. Modified nucleic acids having a capture agent can be immobilized to a solid support in certain embodiments.

Next generation sequencing techniques may be applied to measure expression levels or count numbers of transcripts using RNA-seq or whole transcriptome shotgun sequencing. See, e.g., Mortazavi et al. 2008 Nat Meth 5(7) 621-627 or Wang et al. 2009 Nat Rev Genet 10(1) 57-63. Nucleic acids in the invention may be counted using methods known in the art. In one embodiment, NanoString's nCounter® system may be used (Seattle, Wash.). Geiss et al. 2008 Nat Biotech 26(3) 317-325; U.S. Pat. No. 7,473,767 (Dimitrov). In addition, NanoString's Digital Spatial Profiling (DSP) platform may be used for nucleic acid or protein detection. Blank et al., 2018 Nature Medicine 24 1655-1661; Amaria et al., 2018 Nature Medicine 24 1649-1654. Alternatively, Fluidigm's Dynamic Array system may be used (South San Francisco, Calif.). Byrne et al. 2009 PLoS ONE 4 e7118; Helzer et al. 2009 Can Res 69 7860-7866. For reviews, see also Zhao et al. 2011 Sci China Chem 54(8) 1185-1201 and Ozsolak and Milos 2011 Nat Rev Genet 12 87-98.

### 5.4. Next-Generation Bisulfite Sequencing Method (NGBS)

(250-500 ng of Genomic DNA)

Standardized tissue microdissection Each melanocytic lesion encircled by the pathologist will be measured, have the dimensions recorded and the area calculated. Manual microdissection will be performed on lesions having a cross-sectional area of >2 mm2 by superimposing a non-stained tissue section over the H&E-stained slide and removing the tumor tissue within the pathologist's marked boundaries using a sterile needle. If a melanoma has an associated nevus, only melanoma cells will be selectively removed. Lesional tissues will be pooled from multiple sections and used for DNA isolation.

Laser capture microdissection (LCM) If the lesion is very small (<2 mm2) or intermixed with a large proportion of non-melanocytic cells as judged by the pathologist, LCM will be performed to capture the encircled lesional cells. LCM will be performed under the supervision of a dermatopathologist using an ArcturusXT Laser Capture Microdissection System (ThermoFisher Scientific, Waltham, Mass.) or other similar system. The entire area(s) of the lesion of interest can be encircled and lifted off the slide in a single pass. Importantly, LCM using the ArcturusXT system can be performed on 5 μm-thick FFPE specimens that have previously been mounted on either charged or uncharged slides, enabling the use of banked tissue sections. If a melanoma has a contiguous nevus, melanoma cells will be microdissected away from the remaining nevus cells.

DNA preparation and quality assessment DNA will be isolated using our standard proteinase K-based technique or another commercially available FFPE nucleic acid isolation protocol. DNA quality and quantity will be assessed using Quant-IT PicoGreen dsDNA assay (ThermoFisher Scientific), Illumina FFPE QC assay, and a multiplex PCR reaction of housekeeping genes (i.e. β-actin).

Bisulfite modification of DNA & controls for bisulfite conversion and methylation assays Sodium bisulfite treatment of 250-500 ng DNA from each sample or control will be performed using the EZ DNA methylation, EZ DNA Methylation-Gold or EZ DNA Methylation-Lightning Kit (Zymo Research, Irvine, Calif.) according to the manufacturer's protocol. (Sodium bisulfite chemistry converts nonmethylated cytosines to uracils, which are then converted to thymines in the PCR). After bisulfite treatment, DNA quantity will be determined using a Nanodrop spectrophotometer (ThermoFisher Scientific). Human HCT116 DKO Non-methylated DNA and Human HCT116 DKO Methylated DNA (Zymo Research) will serve as control DNAs, and together with PCR using a set of specially-designed primers (Zymo Research), will be used to assess the efficiency of bisulfite-mediated conversion of DNA.

Description of targeted NGBS A targeted NGBS assay will be developed for simultaneously measuring DNA methylation at the diagnostic CpGs plus control loci (unmethylated and fully methylated controls, bisulfite conversion controls) in FFPE specimens using NGS on a MiniSeq or MiSeq sequencer (Illumina). A custom target-enrichment assay used to create libraries for NGBS includes gene-specific primers designed for bisulfite treated DNA, molecular barcodes, and index adaptors recognized by Illumina sequencers. Genomic DNA sites in 40 or 59 CpGs plus controls will amplified in a multiplex reaction by PCR using bisulfite-converted gDNA as a template with Kapa HiFi HotStart Uracil+ ReadyMix (Kapa Biosystems) (Wilmington, Mass.), PfuTurbo Cx HotStart DNA polymerase (Agilent) or Phusion Hot Start Flex DNA Polymerase (New England Biolabs, NEB, Ipswich, Mass.). Unique molecular barcodes and Illumina's index adaptors will be added by ligation or PCR. Samples will be processed using a dual strand protocol with a mirrored complementary set of amplicons on both DNA strands to eliminate amplification errors sometimes occurring with FFPE derived DNAs. After amplification and library clean-up, the DNA will be visualized using the Agilent Tape Station to determine quantity and fragment size. The library DNA will be denatured and diluted to the proper concentration, normalized samples will be pooled for multiplexed sequencing (150 bp paired-end reads), combined with a PhiX control (10%), and loaded onto the flow cell in the MiniSeq or MiSeq for NGS using Illumina's sequencing by synthesis technology. Sequencing depth of ˜1000× has been found to be sufficient for a precise measurement of DNA methylation levels, and increasing sequencing depth does not further improve the accuracy⁵⁴. Sequencing analysis will be viewed in Local Run Manager and will be aligned using an automated bioinformatics pipeline. This workflow generates the raw sequence data to identify variants based on cytosine methylation (or not) at the target CpG site.

### 5.5. Additional Methods

5.5.1. Antibody Staining/Detection

In some embodiments, the invention may encompass detecting and/or quantitating using antibodies either alone or in conjunction with measurement of methylation levels. Antibodies are already used in current practice in the classification and/or diagnosis of melanocytic lesions (Alonso et al., 2004, Am. J. Pathol. 164(1) 193-203; Ivan & Prieto, 2010, Future Oncol. 6(7), 1163-1175; Linos et al., 2011, Biomarkers Med. 5(3) 333-360; and Rothberg et al., 2009 J. Nat. Canc. Inst. 101(7) 452-474, the contents of which are hereby incorporated by reference in their entireties). Examples of antibodies that are used include HMB45/gp100 (Abcam; AbD Serotec; BioGenex, San Ramon, Calif.; Biocare Medical, Concord, Calif.); MART-1/Melan-A (Abcam; AbD Serotec; BioGenex; Thermo Scientific Pierce Abs., Rockford, Ill.); Microphthalmia transcription factor/MITF-1 (Invitrogen); NKI/C3 (Melanoma Associated Antigen 100+/7 kDa)(Abcam; Thermo Scientific Pierce Abs.); p75NTR/neurotrophin receptor (Abcam; AbD Serotec; Promega, Madison, Wis.); S100 (Abcam; AbD Serotec, Raleigh, N.C.; BioGenex); Tyrosinase (Abcam; AbD Serotec; Thermo Scientific Pierce Abs.). In one embodiment a cocktail of S100, HMB-45 and MART-1/Melan-A is used. Antibodies may also be used to detect the gene products of the methylated genes described herein. Specifically, genes hypomethylated would be expected to show over-expression and genes hypermethylated would be expected to show under-expression. Staining markers of tumor vascular formation may also be used in conjunction with the present invention (Bhati et al., 2008, Am. J. Pathol. 172(5), 1381-1390, including Table 1 on page 1387, the contents of which are incorporated herein by reference in their entirety).

Antibody reagents can be used in assays to detect expression levels in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., 1996, Curr. Opin. Biotechnol., 7, 60-65. The term immunoassay encompasses techniques including, without limitation, enzyme immunoassays (EIA) such as enzyme multiplied immunoassay technique (EMIT), enzyme-linked immunosorbent assay (ELISA), Enzyme-Linked ImmunoSpot assay (ELISPOT), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., 1997, Electrophoresis, 18, 2184-2193; Bao, 1997, J. Chromatogr. B. Biomed. Sci., 699, 463-480. Liposome immunoassays, such as flow-injection liposome immunoassays and liposome immunosensors, are also suitable for use in the present invention. See, e.g., Rongen et al., 1997, J. Immunol. Methods, 204, 105-133. In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.) and can be performed using a Behring Nephelometer Analyzer (Fink et al., 1989, J. Clin. Chem. Clin. Biochem., 27, 261-276).

Specific immunological binding of the antibody to nucleic acids can be detected directly or indirectly. Direct labels include fluorescent or luminescent tags, metals, dyes, radionuclides, and the like, attached to the antibody. An antibody labeled with iodine-125¹²⁵I can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-/3-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of 125I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

Proteins or nucleic acids described herein also may be visualized using advanced technology such as Hyperion Imaging System from Fluidym, Inc. See “Simultaneous Multiplexed Imaging of mRNA and Proteins with Subcellular Resolution in Breast Cancer Tissue Samples by Mass Cytometry” Schulz et al. 2018 Cell Systems 25-36; “Multiplex protein detection on circulating tumor cells from liquid biopsies using imaging mass cytometry” Gerdtsson et al. Convergent Science Physical Oncology (2018): 015002; “Imaging Mass Cytometry” Chang, Q., et al. 2017 Cytometry Part A 160-169; “Highly multiplexed imaging of tumor tissues with subcellular resolution by mass cytometry” Giesen, C., et al. 2014 Nature Methods 417-422. In addition, NanoString's Digital Spatial Profiling (DSP) platform may be used for nucleic acid or protein detection. Blank et al., 2018 Nature Medicine 24 1655-1661; Amaria et al., 2018 Nature Medicine 24 1649-1654.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot. The antibodies may be in an array of one or more antibodies, single or double stranded nucleic acids, proteins, peptides or fragments thereof, amino acid probes, or phage display libraries. Many protein/antibody arrays are described in the art. These include, for example, arrays produced by Ciphergen Biosystems (Fremont, Calif.), Packard BioScience Company (Meriden Conn.), Zyomyx (Hayward, Calif.) and Phylos (Lexington, Mass.). Examples of such arrays are described in the following patents: U.S. Pat. No. 6,225,047 (Hutchens and Yip); U.S. Pat. No. 6,537,749 (Kuimelis and Wagner); and U.S. Pat. No. 6,329,209 (Wagner et al.), all of which are incorporated herein by reference in their entirety.

### 5.5.2. Fluorescence In Situ Hybridization (FISH) and Comparative Genomic Hybridization (CGH)

In some embodiments, the invention may further encompass detecting and/or quantitating using fluorescence in situ hybridization (FISH) in a sample, preferably a tissue sample, obtained from a subject in accordance with the methods of the invention. FISH is a common methodology used in the art, especially in the detection of specific chromosomal aberrations in tumor cells, for example, to aid in diagnosis and tumor staging. As applied in the methods of the invention, it can be used in conjunction with detecting methylation. For reviews of FISH methodology, see, e. g., Weier et al., 2002, Expert Rev. Mol. Diagn. 2 (2): 109-119; Trask et al., 1991, Trends Genet. 7 (5): 149-154; and Tkachuk et al., 1991, Genet. Anal. Tech. Appl. 8: 676-74; U.S. Pat. No. 6,174,681 (Halling et al.); for multi-color FISH specific to melanoma, see Gerami et al., 2009, Am. J. Surg. Pathol. 33(8) 1146-1156; and PCT Pub. No. WO 2007/028031 A2 (Bastian et al.); all of which are incorporated herein by reference in their entirety. Alternatively, comparative genomic hybridization (CGH) also may be used as part of the methods disclosed herein. Specifically, Bastian et al. describe CGH as a means to find patterns of chromosomal aberrations associated with melanoma (Bastian et al., 2003, Am. J. Pathol. 163(5) 1765-1770).

In alternative embodiments, the invention encompasses use of additional melanoma specific gene expression and/or antibody assays either in situ, i.e., directly upon tissue sections (fixed and/or frozen) of patient tissue obtained from biopsies or resections, such that no nucleic acid purification is necessary; or based on extracted and/or amplified nucleic acids. Targets for such assays are disclosed in Haqq et al. 2005, Proc. Nat. Acad. Sci. USA, 102(17), 6092-6097; Riker et al., 2008, BMC Med. Genomics, 1, 13, pub. 28 Apr. 2008; Hoek et al., 2004, Can. Res. 64, 5270-5282; PCT Pub. Nos. WO 2008/030986 and WO 2009/111661 (Kashani-Sabet & Haqq); U.S. Pat. No. 7,247,426 (Yakhini et al.), all of which are incorporated herein by reference in their entirety. Several researchers have reported the use of microRNAs (miRNA) for cancer or melanoma detection. These methods could be used in combination with the methylation methods described herein (see Mueller et al., 2009, J. Invest. Dermatol., 129, 1740-1751; Leidinger et al., 2010, BMC Cancer, 10, 262; U.S. Pat. Pub. 2009/0220969 (Chiang and Shi); PCT Pub. No. WO 2010/068473 (Reynolds and Siva); which are hereby incorporated by reference in their entirety). Alternatively, the methylated nucleic acids may be detected in blood either as free DNA or in circulating tumor cells. For in situ procedures see, e.g., Nuovo, G. J., 1992, PCR In Situ Hybridization: Protocols And Applications, Raven Press, NY, which is incorporated herein by reference in its entirety.

Methods for making nucleic acid microarrays are known to the skilled artisan and are described, for example, in Lockhart et al., 1996, Nat. Biotech. 14, 1675-1680, 1996 Schena et al., 1996, Proc. Natl. Acad. Sci. USA, 93, 10614-10619, U.S. Pat. No. 5,837,832 (Ghee et al.) and PCT Pub. No. WO 00/56934 (Englert et al.), herein incorporated by reference. To produce a nucleic acid microarray, oligonucleotides may be synthesized or bound to the surface of a substrate using a chemical coupling procedure and an ink jet application apparatus, as described U.S. Pat. No. 6,015,880 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

The measurement of differentially methylated elements associated with melanoma may alone, or in conjunction with other melanoma detection tools discussed above (antibody staining, PCR, CGH, FISH) may have several other non-limiting uses. Amongst these uses are: (i) reclassifying specimens that were indeterminate or difficult to identify in a pathology laboratory; (ii) deciding to follow up with a lymph node examination (SLNB) and/or PET/CAT/MRI or other imaging methods; (iii) determining the frequency of follow up visits; or (iv) initiating other investigatory analysis such as a blood draw and evaluation for circulating tumor cells. Furthermore, the differentially methylated elements associated with melanoma may help to determine which patients would benefit from adjuvant treatment after surgical resection.

Methods for Next-generation Bisulfite Sequencing (NGBS) can be utilized to measure methylated or non-methylated CpGs as described, for example in Wen et. al., 2014, Genome Biology. 15:R49; Lee et. al., 2015, MEX. 115:1-7; and Farlik et. al., 2015, Cell Reports 10, 1386-1397. DNA is treated by sodium bisulfite to convert nonmethylated cytosines to uracils, which are then converted to thymines in PCR or sequencing. Generally, sodium bisulfite treated DNA undergoes end-repair, is hybridized to specific primers for amplification, and has molecular barcodes and index adaptors ligated in incorporated during PCR. The amplified DNA is quantitated, sized, normalized, and combined for multiplexed NGS sequencing.

### 5.6. Compositions and Kits

The invention provides compositions and kits measuring methylation of polynucleotides at the differentially methylated elements described herein using DNA methylation specific assays, antibodies or other reagents specific for the nucleic acids specific for the polynucleotides. Kits for carrying out the diagnostic assays of the invention typically include, in suitable container means, (i) a reagent for methylation specific reaction or separation, (ii) a probe that comprises an antibody or nucleic acid sequence that specifically binds to the marker polynucleotides of the invention, (iii) a label for detecting the presence of the probe and (iv) instructions for how to measure the level of methylation of the polynucleotide. The kits may include several antibodies or polynucleotide sequences encoding polypeptides of the invention, e.g., a first antibody and/or second and/or third and/or additional antibodies that recognize a gene differentially methylated in melanoma. In one embodiment the nucleic acids in the kit are the forward and reverse PCR primers for the 40 CpG assay (SEQ ID NO: 81-160). In another embodiment, the nucleic acids in the kit are forward and reverse PCR primers for the 59 CpG assay (SEQ ID NO: 379-496). In yet another embodiment, nucleic acids for detecting mutations in the TERT promoter such as SEQ ID NO: 497-500 are included with the nucleic acids or either the 40 CpG assay or the 59 CpG assay. The container means of the kits will generally include at least one vial, test tube, flask, bottle, syringe and/or other container into which a first antibody specific for one of the polypeptides or a first nucleic acid specific for one of the polynucleotides of the present invention may be placed and/or suitably aliquoted. Where a second and/or third and/or additional component is provided, the kit will also generally contain a second, third and/or other additional container into which this component may be placed. Alternatively, a container may contain a mixture of more than one antibody or nucleic acid reagent, each reagent specifically binding a different marker in accordance with the present invention. The kits of the present invention will also typically include means for containing the antibody or nucleic acid probes in close confinement for commercial sale. Such containers may include injection and/or blow-molded plastic containers into which the desired vials are retained.

The kits may further comprise positive and negative controls, as well as instructions for the use of kit components contained therein, in accordance with the methods of the present invention.

### 5.7. In Vivo Imaging

The various markers of the invention also provide reagents for in vivo imaging such as, for instance, the imaging of metastasis of melanoma to regional lymph nodes using labeled reagents that detect (i) DNA methylation associated with melanoma, (ii) a polypeptide or polynucleotide regulated by the differentially methylated elements. In vivo imaging techniques may be used, for example, as guides for surgical resection or to detect the distant spread of melanoma. For in vivo imaging purposes, reagents that detect the presence of these proteins or genes, such as antibodies, may be labeled with a positron-emitting isotope (e.g., 18F) for positron emission tomography (PET), gamma-ray isotope (e.g., 99mTc) for single photon emission computed tomography (SPECT), a paramagnetic molecule or nanoparticle (e.g., Gd³⁺ chelate or coated magnetite nanoparticle) for magnetic resonance imaging (MRI), a near-infrared fluorophore for near-infra red (near-IR) imaging, a luciferase (firefly, bacterial, or coelenterate), green fluorescent protein, or other luminescent molecule for bioluminescence imaging, or a perfluorocarbon-filled vesicle for ultrasound. Fluorodeoxyglucose (FDG)-PET metabolic uptake alone or in combination with MRI is particularly useful.

Furthermore, such reagents may include a fluorescent moiety, such as a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are discussed, for example, in U.S. Pat. No. 4,452,720 (Harada et al.); U.S. Pat. No. 5,227,487 (Haugland and Whitaker); and U.S. Pat. No. 5,543,295 (Bronstein et al.). Other fluorescent labels suitable for use in the practice of this invention include a fluorescein dye. Typical fluorescein dyes include, but are not limited to, 5-carboxyfluorescein, fluorescein-5-isothiocyanate and 6-carboxyfluorescein; examples of other fluorescein dyes can be found, for example, in U.S. Pat. No. 4,439,356 (Khanna and Colvin); U.S. Pat. No. 5,066,580 (Lee), U.S. Pat. No. 5,750,409 (Hermann et al.); and U.S. Pat. No. 6,008,379 (Benson et al.). The kits may include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®, and other rhodamine dyes. Other rhodamine dyes can be found, for example, in U.S. Pat. No. 5,936,087 (Benson et al.), U.S. Pat. No. 6,025,505 (Lee et al.); U.S. Pat. No. 6,080,852 (Lee et al.). The kits may include a cyanine dye, such as, for example, Cy3, Cy3B, Cy3.5, Cy5, Cy5.5, Cy7. Phosphorescent compounds including porphyrins, phthalocyanines, polyaromatic compounds such as pyrenes, anthracenes and acenaphthenes, and so forth, may also be used.

### 5.8. Methods to Identify Compounds

A variety of methods may be used to identify compounds that modulate DNA methylation and prevent or treat melanoma progression. Typically, an assay that provides a readily measured parameter is adapted to be performed in the wells of multi-well plates in order to facilitate the screening of members of a library of test compounds as described herein. Thus, in one embodiment, an appropriate number of cells can be plated into the cells of a multi-well plate, and the effect of a test compound on the expression of a gene differentially methylated in melanoma can be determined. The compounds to be tested can be any small chemical compound, or a macromolecule, such as a protein, sugar, nucleic acid or lipid. Typically, test compounds will be small chemical molecules and peptides. Essentially any chemical compound can be used as a test compound in this aspect of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods are used which involve providing a combinatorial chemical or peptide library containing a large number of potential therapeutic compounds. Such “combinatorial chemical libraries” or “ligand libraries” are then screened in one or more assays, as described herein, to identify those library members (particular chemical species or subclasses) that display a desired characteristic activity. In this instance, such compounds are screened for their ability to modulate the expression of genes differentially methylated in melanoma. A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries are well known to those of skill in the art. Such combinatorial chemical libraries include, but are not limited to, peptide libraries (see, e.g., U.S. Pat. No. 5,010,175 (Rutter and Santi), Furka, 1991, Int. J. Pept. Prot. Res., 37:487-493; and Houghton et al., 1991, Nature, 354:84-88). Other chemistries for generating chemical diversity libraries can also be used. Such chemistries include, but are not limited to: U.S. Pat. No. 6,075,121 (Bartlett et al.) peptoids; U.S. Pat. No. 6,060,596 (Lerner et al.) encoded peptides; U.S. Pat. No. 5,858,670 (Lam et al.) random bio-oligomers; U.S. Pat. No. 5,288,514 (Ellman) benzodiazepines; U.S. Pat. No. 5,539,083 (Cook et al.) peptide nucleic acid libraries; U.S. Pat. No. 5,593,853 (Chen and Radmer) carbohydrate libraries; U.S. Pat. No. 5,569,588 (Ashby and Rine) isoprenoids; U.S. Pat. No. 5,549,974 (Holmes) thiazolidinones and metathiazanones; U.S. Pat. No. 5,525,735 (Takarada et al.) and U.S. Pat. No. 5,519,134 (Acevado and Hebert) pyrrolidines; 5,506,337 (Summerton and Weller) morpholino compounds; U.S. Pat. No. 5,288,514 (Ellman) benzodiazepines; diversomers such as hydantoins, benzodiazepines and dipeptides (Hobbs et al., 1993, Proc. Nat. Acad. Sci. USA, 90, 6909-6913), vinylogous polypeptides (Hagihara et al., 1992, J. Amer. Chem. Soc., 114, 6568), nonpeptidal peptidomimetics with glucose scaffolding (Hirschmann et al., 1992, J. Amer. Chem. Soc., 114, 9217-9218), analogous organic syntheses of small compound libraries (Chen et al., 1994, J. Amer. Chem. Soc., 116:2661 (1994)), oligocarbamates (Cho et al., 1993, Science, 261, 1303 (1993)), and/or peptidyl phosphonates (Campbell et al., 1994, J. Org. Chem., 59:658), nucleic acid libraries (see Ausubel, Berger and Sambrook, all supra); antibody libraries (see, e.g., Vaughn et al., 1996, Nat. Biotech., 14(3):309-314, carbohydrate libraries, e.g., Liang et al., 1996, Science, 274:1520-1522, small organic molecule libraries (see, e.g., benzodiazepines, Baum, 1993, C&EN, January 18, page 33. Devices for the preparation of combinatorial libraries are commercially available (see, e.g., 357 MPS, 390 MPS, Advanced Chem Tech, Louisville Ky., Symphony, Rainin, Woburn, Mass., 433 A Applied Biosystems, Foster City, Calif., 9050 Plus, Millipore, Bedford, Mass.). In addition, numerous combinatorial libraries are themselves commercially available (see, e.g., ComGenex (Princeton, N.J.), Asinex (Moscow, RU), Tripos, Inc. (St. Louis, Mo.), ChemStar, Ltd., (Moscow, RU), 3D Pharmaceuticals (Exton, Pa.), Martek Biosciences (Columbia, Md.), etc.).

Methylation modifiers are known and have been the basis for several approved drugs. Major classes of enzymes are DNA methyl transferases (DNMTs), histone deacetylases (HDACs), histone methyl transferases (HMTs), and histone acetylases (HATs). DNMT inhibitors azacitidine (Vidaza®) and decitabine have been approved for myelodysplastic syndromes (for a review see Musolino et al., 2010, Eur. J. Haematol. 84, 463-473; Issa, 2010, Hematol. Oncol. Clin. North Am. 24(2), 317-330; Howell et al., 2009, Cancer Control, 16(3) 200-218; which are hereby incorporated by reference in their entirety). HDAC inhibitor, vorinostat (Zolinza®, SAHA) has been approved by FDA for treating cutaneous T-cell lymphoma (CTCL) for patients with progressive, persistent, or recurrent disease (Marks and Breslow, 2007, Nat. Biotech. 25(1), 84-90). Specific examples of compound libraries include: DNA methyl transferase (DNMT) inhibitor libraries available from Chem Div (San Diego, Calif.); cyclic peptides (Nauman et al., 2008, Chem Bio Chem 9, 194-197); natural product DNMT libraries (Medina-Franco et al, 2010, Mol. Divers., Springer, published online 10 Aug. 2010); HDAC inhibitors from a cyclic cop-tetrapeptide library (Olsen and Ghadiri, 2009, J. Med. Chem. 52(23), 7836-7846); HDAC inhibitors from chlamydocin (Nishino et al., 2006, Amer. Peptide Symp. 9(7), 393-394).

### 5.9. Methods of Inhibition Using Nucleic Acids

A variety of nucleic acids, such as antisense nucleic acids, siRNAs or ribozymes, may be used to inhibit the function of the markers of this invention. Ribozymes that cleave mRNA at site-specific recognition sequences can be used to destroy target mRNAs, particularly through the use of hammerhead ribozymes. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

The following Examples further illustrate the disclosure and are not intended to limit the scope. In particular, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### 6. EXAMPLES

### 6.1. Introduction Section 6.1-6.4

Early diagnosis improves melanoma survival, yet the histologic diagnosis of cutaneous melanoma can be exceedingly challenging even for expert dermatopathologists. Analysis of epigenetic alterations, such as DNA methylation, that occur in melanoma can aid in its early diagnosis. Using a genome-wide methylation screen, we assessed CpG methylation in a diverse series of 89 formalin-fixed paraffin-embedded primary melanomas, 73 benign nevi, and 41 melanocytic samples with uncertain diagnoses identified from inter-observer review by three dermatopathologists. Melanomas and nevi were split into training and validation sets. Predictive modeling in the training set using ElasticNet identified a 40-CpG methylation signature distinguishing 60 melanomas from 48 nevi. High diagnostic accuracy (AUC=0.996, sensitivity=96.6%, specificity=100.0%) was confirmed in the independent validation set (29 melanomas, 25 nevi). The diagnostic signature included homeobox transcription factors and genes with roles in stem cell pluripotency or the nervous system. Differential methylation of diagnostic genes was also validated in published series of primary melanomas and nevi. Application of the 40-CpG diagnostic predictor to diagnostically uncertain samples assigned melanoma or nevus status, potentially offering diagnostic clarity to many of these samples. In summary, the robust, highly-accurate DNA methylation signature described here offers a promising assay for improving the diagnosis of primary melanoma.

Our initial study using a methylation array that targeted cancer-related genes provided proof-of-principle that DNA methylation differences could distinguish invasive primary melanomas from benign nevi in small FFPE samples (Conway et al, 2011). In the present study, we extend this work by identifying and independently validating a highly accurate diagnostic methylation signature that distinguishes primary melanomas from a broad histologic spectrum of benign nevi within a series of melanocytic samples reviewed by a panel of expert dermatopathologists. These findings could translate to a robust melanoma diagnostic test ideal for use in FFPE melanocytic specimens.

### 6.2. Results

Patient and specimen characteristics Illumina 450K methylation analysis was successfully performed on 97% of samples, including 89 FFPE primary melanomas with median Breslow thickness of 1.85 mm (range of 0.37-17.00 mm), balanced for AJCC tumor stages and histologic subtypes, 73 benign nevi (including intradermal, common acquired, dysplastic, Spitz, and blue nevi), and 41 melanocytic lesions with uncertain diagnosis. Melanomas and nevi (excluding samples with uncertain diagnoses) were divided into training (67% of samples; 60 melanomas and 48 nevi) and validation (33%; 29 melanomas and 25 nevi) sets (TABLE 1); these did not differ significantly in patient age, sex or other clinical or pathologic characteristics. Melanoma patients in both the training and test sets were older than nevus patients. The diagnoses assigned to the uncertain samples are listed in Supp. TABLE 51. A lack of diagnostic consensus between any of the dermatopathologists, or between any dermatopathologist and the original pathology report, or a call of ‘uncertain’ by any pathologist or the pathology report resulted in a sample being assigned to the uncertain category.

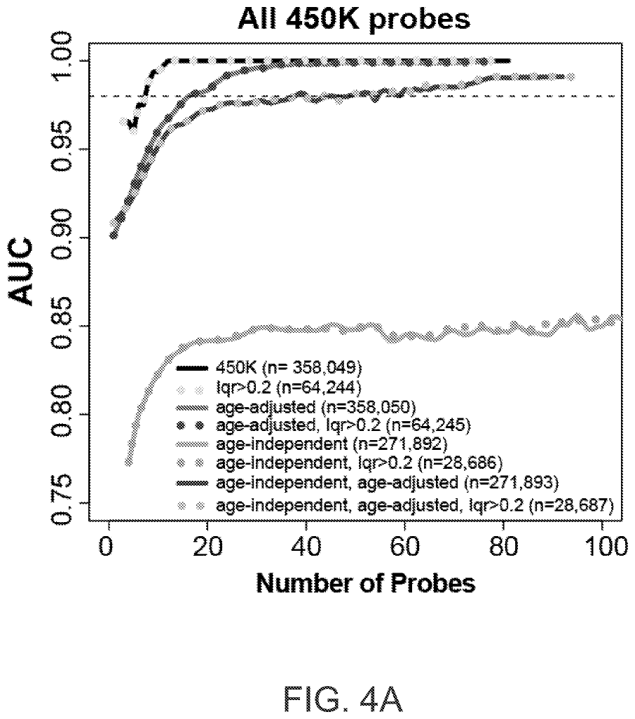

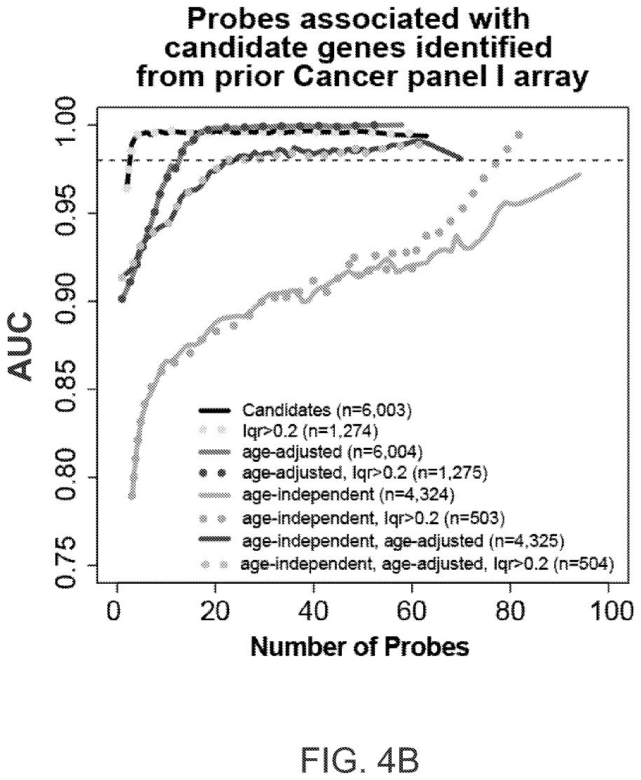

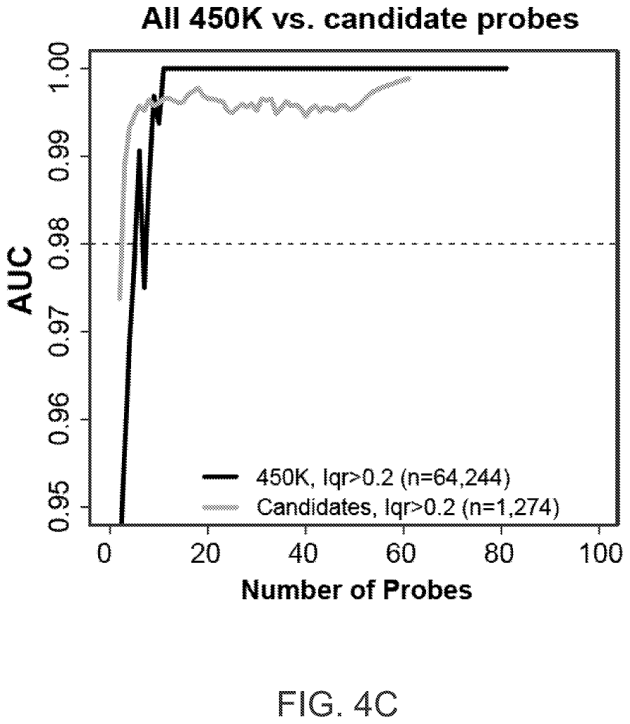

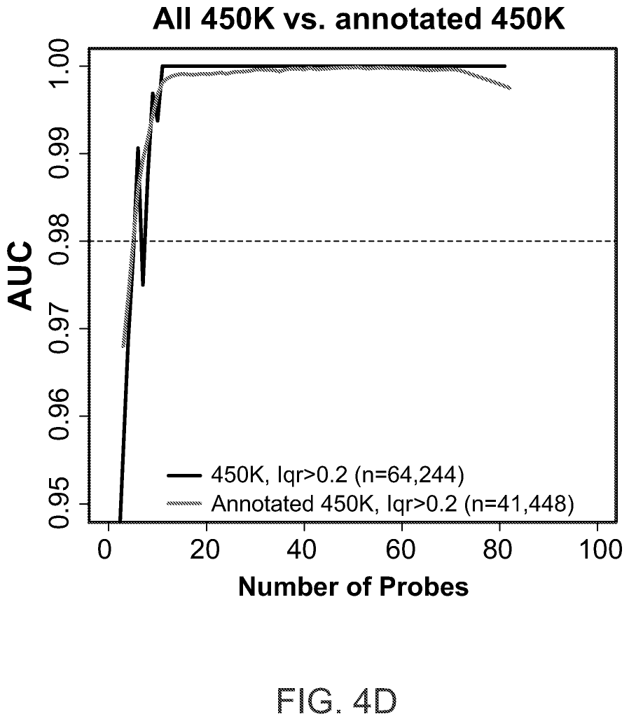

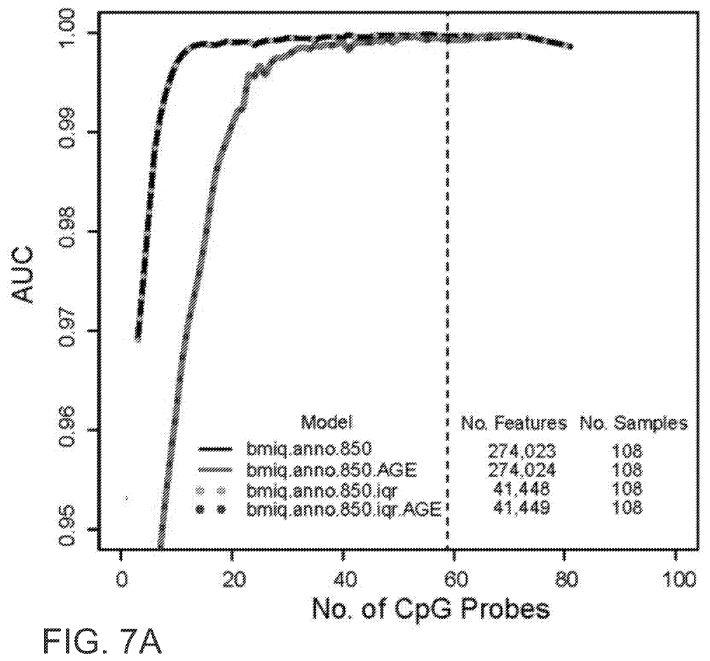

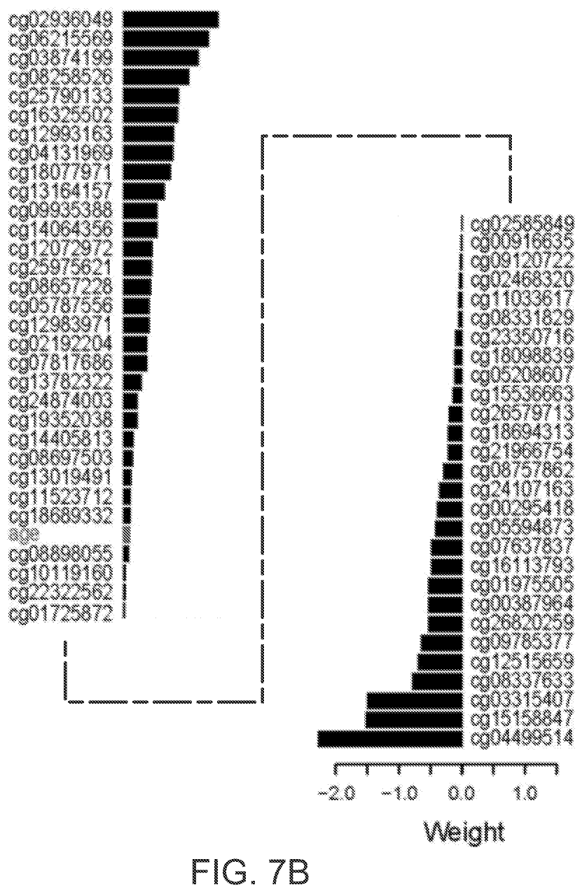

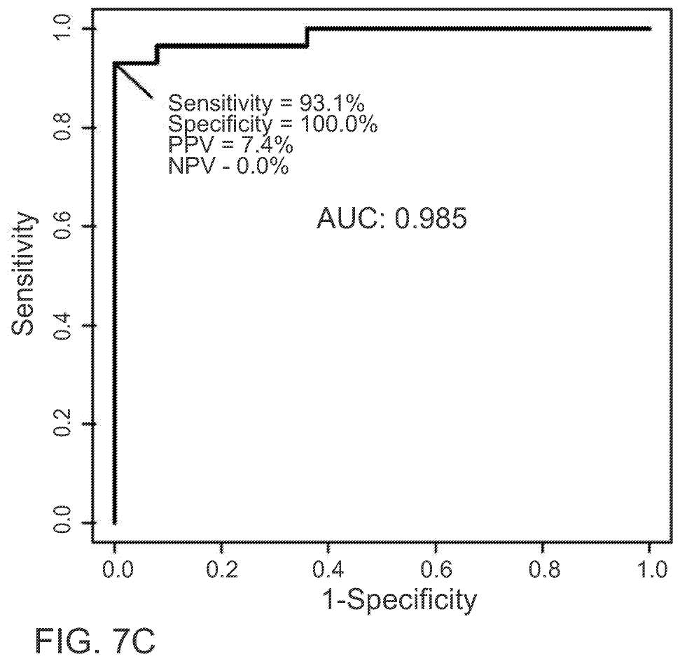

Development and independent validation of a diagnostic methylation signature for melanoma ElasticNet cross validation was used to develop and compare the diagnostic accuracy of CpG signatures derived from multiple 450K probe sets in the training set. Inclusion of all CpG probes provided slightly better diagnostic accuracy than a limited set of probes associated with candidate genes identified from our prior study (Conway et al, 2011) (FIGS. 4A-4D). Accounting for age differences in the models by removing age-associated probes or adjusting for age, or both, all resulted in prediction models with inferior diagnostic discrimination; however, this could be overcome by increasing the number of features in the age-adjusted models. Restricting the models to probes showing larger methylation differences between melanomas and nevi (FIGS. 4A and 4B) and/or to probes with Illumina gene annotation (FIG. 4D) produced results that were very comparable to the more complete probe sets. Based on comparative performance of the various models, we identified a 40-CpG signature associated with 38 genes for further characterization derived from the probe set filtered for IQR>0.2 beta and with gene annotation (n=41,448 probes; FIG. 4C). CpGs contributing to the diagnostic predictor were both hypermethylated (n=23) or hypomethylated (n=17) in melanomas relative to nevi, and the majority were located in the upstream regulatory regions of genes (TSS200, TSS1500, 5′UTR), including one-third in enhancer regions (TABLE 2). A second model adjusted for age and filtered for probes with IQR>0.2 β (n=64,245 probes; FIG. 4A) produced an accurate 59 CpG diagnostic signature, with AUC=0.985, sensitivity=93.1%, and specificity=100.0% (FIG. 7A-7C).

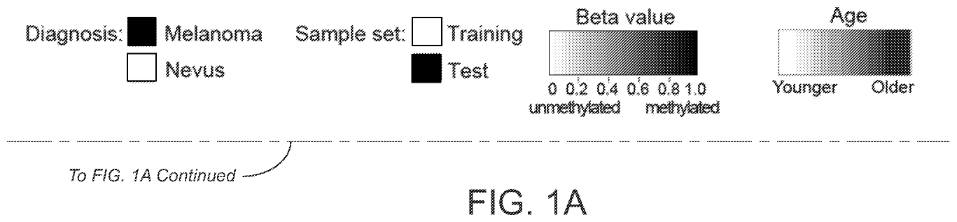

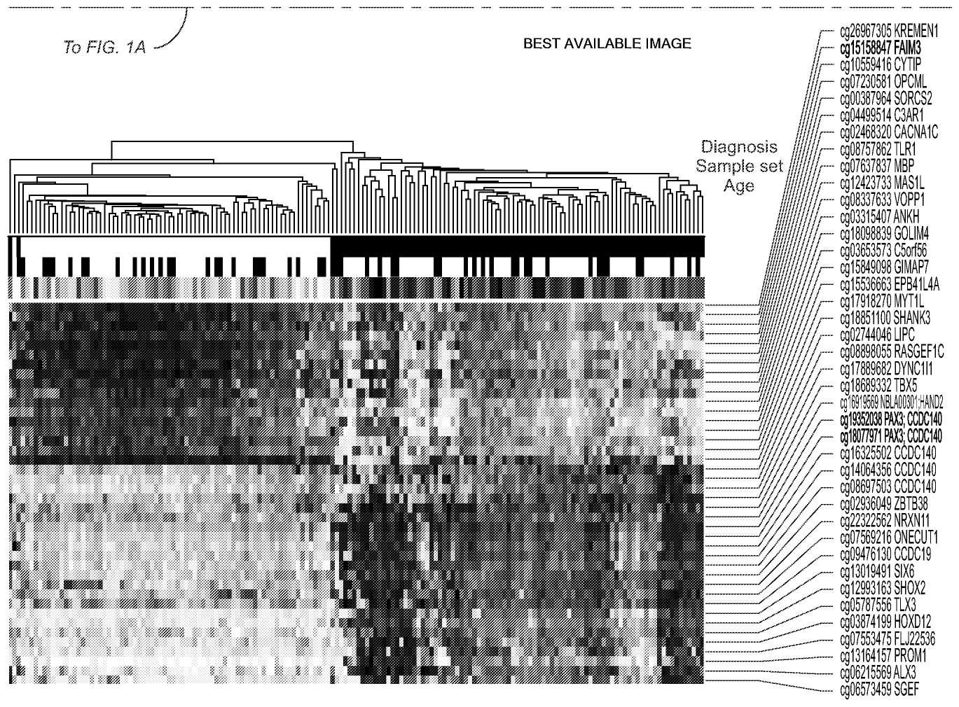

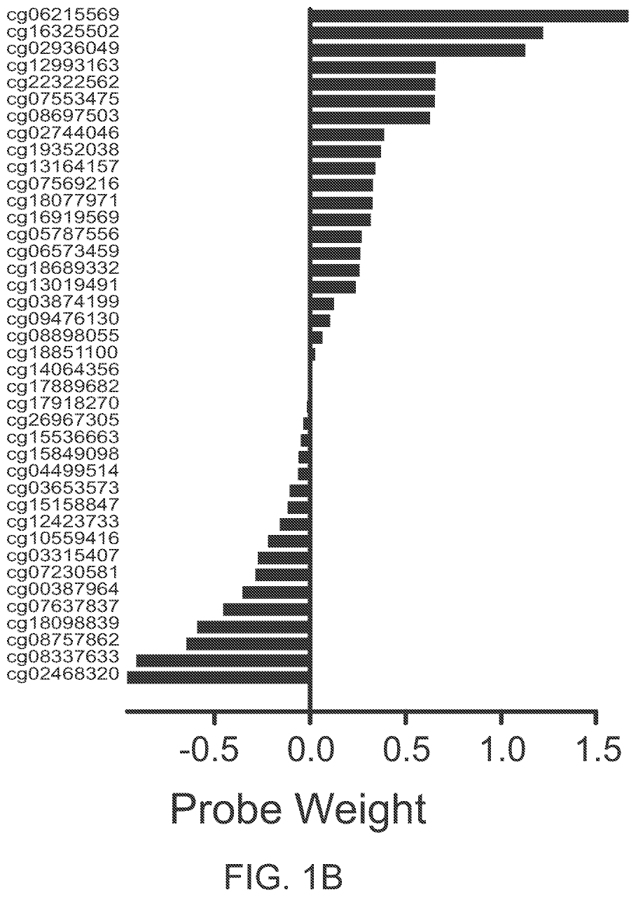

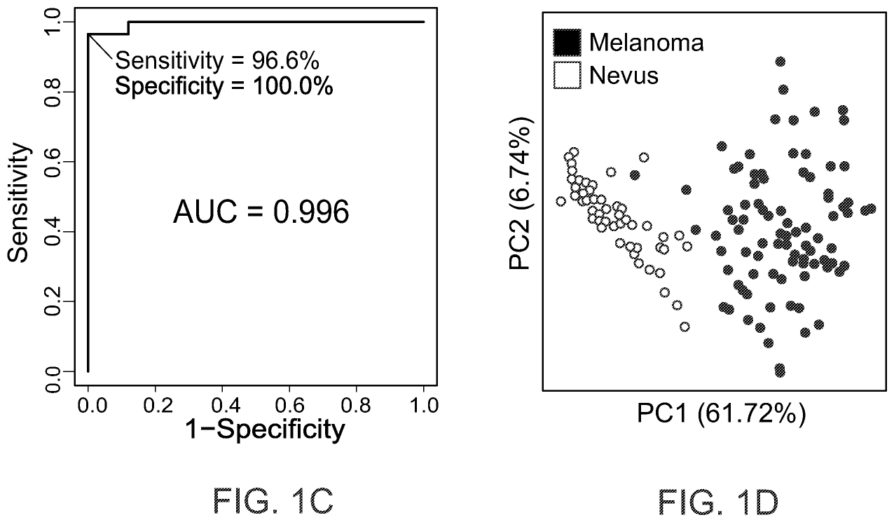

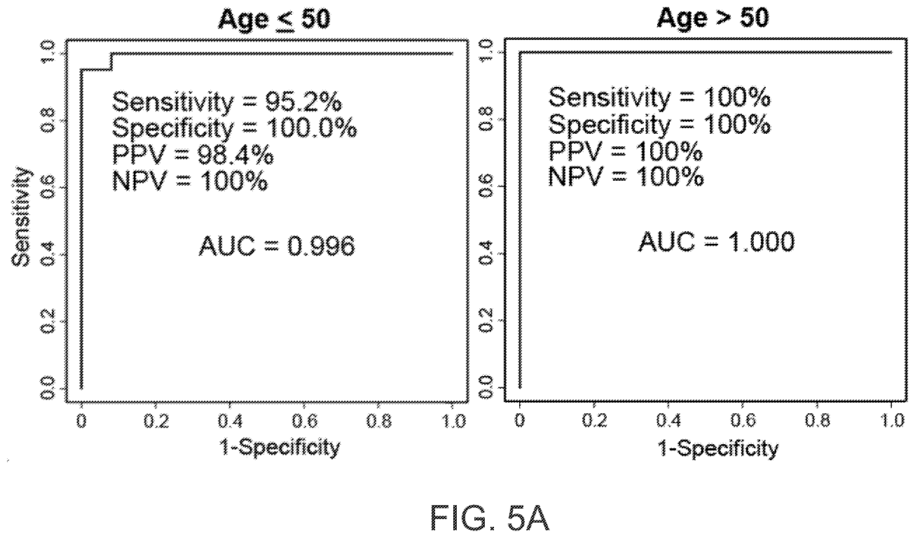

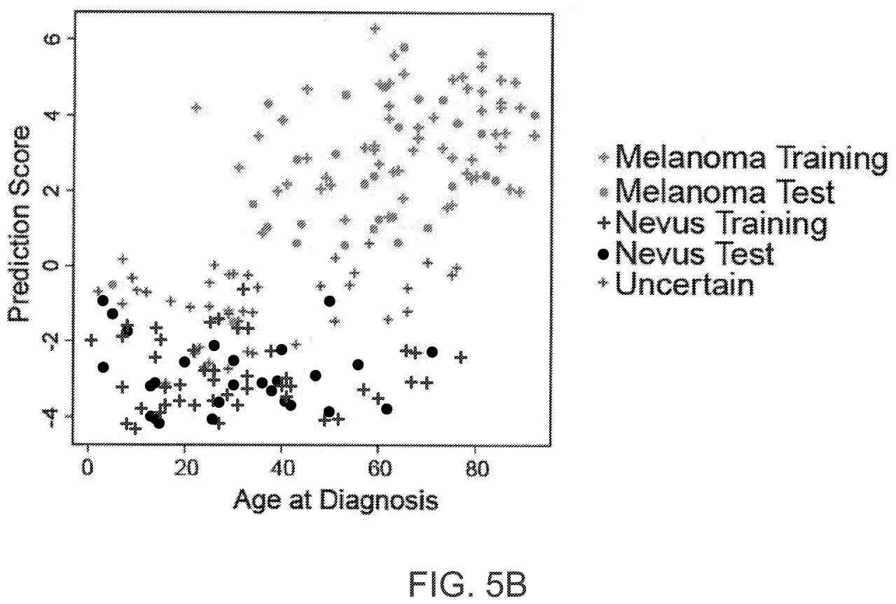

The heatmap in FIG. 1A illustrates methylation levels at the 40 CpG-diagnostic signature probes in primary melanomas and nevi in the training and test sets, and the bar plot in FIG. 1B shows the relative contribution of each probe to the signature. The diagnostic accuracy of the predictor for melanoma in the independent validation set was high (AUC=0.996), with a sensitivity of 96.7%, specificity of 100%, positive predictive value (PPV) of 96.2%, and negative predictive value (NPV) of 100% (FIG. 1C). PCA confirmed the segregation of melanomas from nevi based on the 40-probe signature (FIG. 1D). Despite the age difference between melanoma and nevus patients and age-associated CpGs being retained in the model, the 40-CpG diagnostic predictor performed similarly in differentiating melanomas from nevi among both younger (≤50 years; AUC=0.996) and older patients (>50 years; AUC=1.00) (FIG. 5A-5B). The accuracy of the 40-CpG diagnostic classifier was also high irrespective of patient sex, anatomic site of the lesion, lesion pigmentation, the degree of solar elastosis in surrounding skin, and technical factors such as institutional source of tissues, percent melanocytic cells or the presence of lymphocytes, or the Illumina methylation array used (Supp. TABLE S2). Only 2 samples (of 89; 2.2%) were molecularly misclassified between the training and validation sets; both were melanomas misclassified as nevi. One (sample 691) was a thin superficial spreading melanoma (Breslow thickness=0.54 mm) and the other (sample 848) was a nodular melanoma (Breslow thickness=6.86 mm) from a 5-year old child. DAVID gene ontology analysis, described in the Supplemental Methods, indicated that the diagnostic signature was enriched in homeobox genes that play roles in embryonic development and differentiation (e.g., PAX3, TLX3, SHOX2, ALX3, SIX6, HOXD12, ONECUT1), other transcriptional regulatory genes (HAND2, TBX5, ZBTB38), and genes involved in neurological processes (NRXN1, SHANK3, HAND2, MBP, OPCML, SORCS2) (Supp. TABLE S3).

Diagnostic Signature Calls in Histologically Uncertain Samples

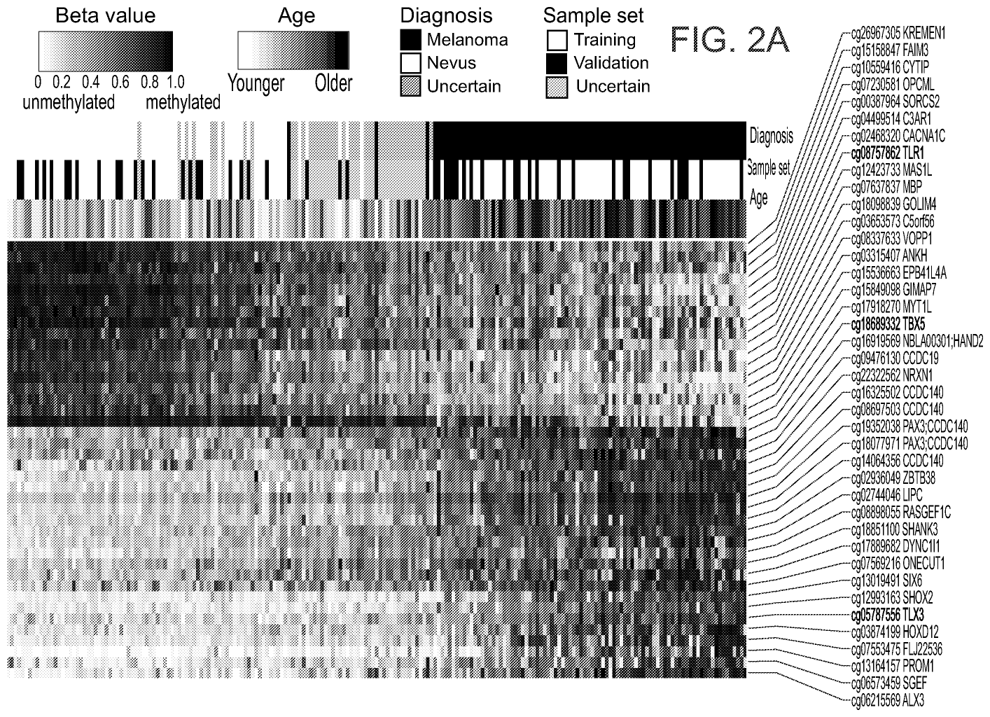

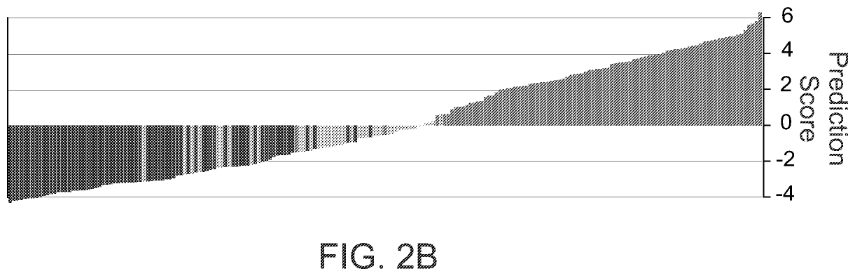

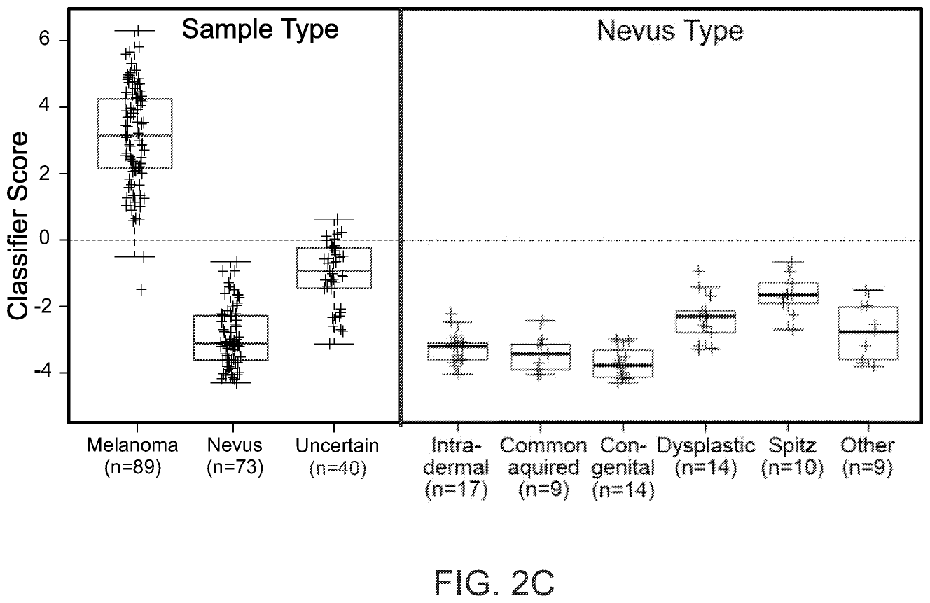

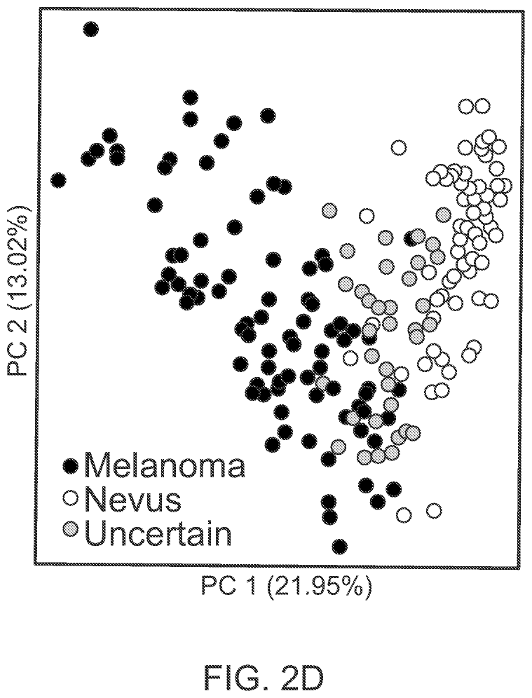

For the 41 melanocytic specimens lacking a clear diagnostic consensus, we applied the methylation predictor to derive a diagnostic prediction score for a call of melanoma or nevus. The heatmap in FIG. 2A illustrates methylation levels at the 40 diagnostic CpGs in the complete sample series, ordered from lowest (negative for nevi) to highest prediction scores (positive for melanoma). Uncertain samples largely resided between the histologically-confirmed benign nevi and primary invasive melanomas, with about half clustered in a zone of intermediate methylation around the prediction score threshold (scores between −1.5 and 0.5). In total, 36 were called nevus and 5 were called melanoma by the prediction score, as shown in the waterfall plot (FIG. 2B). According to the original pathology report (rather than the inter-observer review), the 5 uncertain samples epigenetically diagnosed as melanomas included one superficial spreading melanoma, one atypical Spitz tumor, one atypical Spitz tumor or melanoma (favored diagnosis), one atypical epitheliod blue nevus/pigmented epitheliod melanocytoma, and one atypical dysplastic nevus or thin melanoma. Among the 36 ‘uncertain’ samples molecularly diagnosed as nevi, 3 were identified as melanoma by the pathology report, and most others were histopathologically challenging lesions that included Spitz tumors, blue nevi, and dysplastic nevi or potentially thin melanomas. Boxplots illustrating the range of 40-CpG prediction scores by diagnostic class or nevus subtype show that Spitz nevi fall closest to the diagnostic threshold (FIG. 2C). PCA confirms the segregation of melanomas from nevi, with the uncertain samples falling among the nevi or residing at the interface between nevi and melanomas (FIG. 2D).

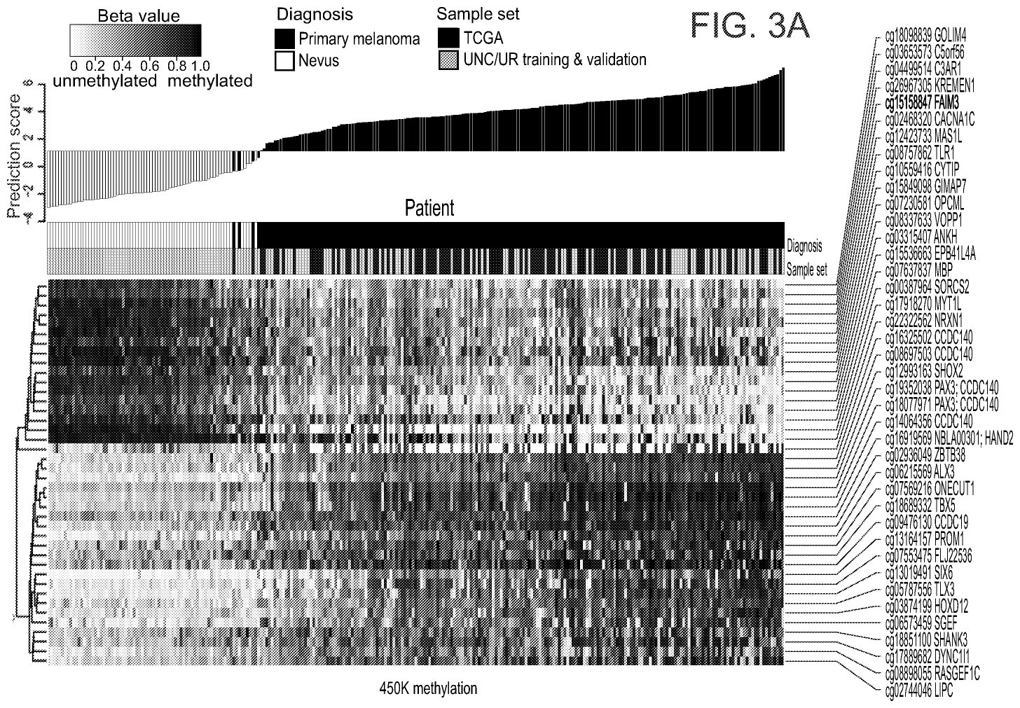

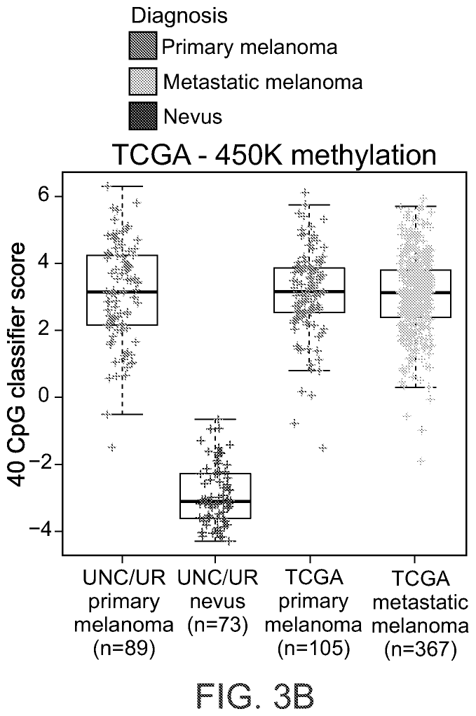

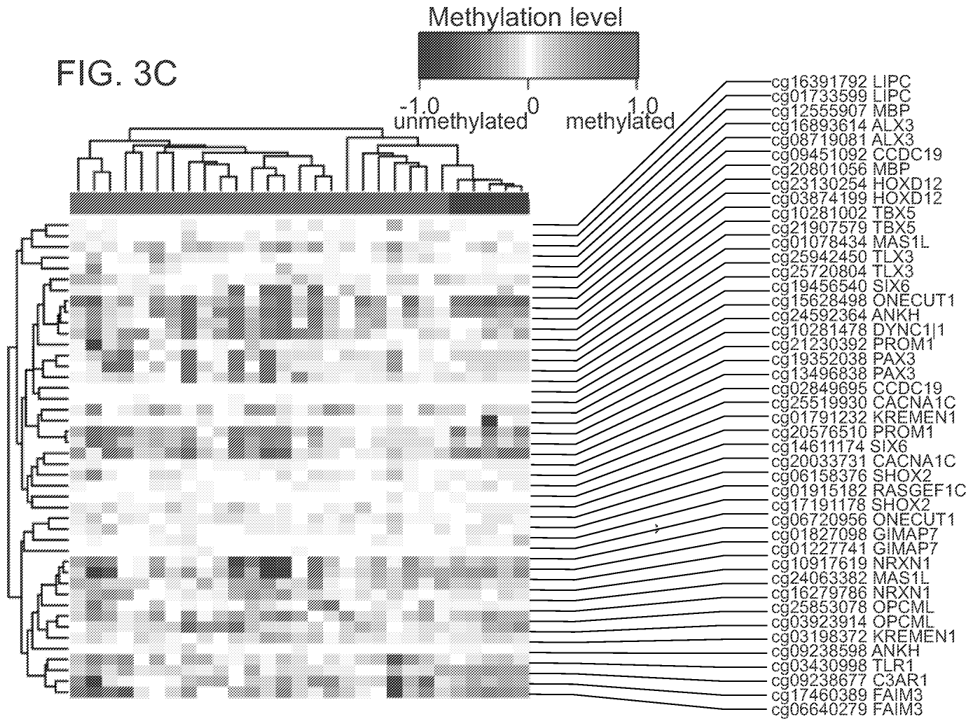

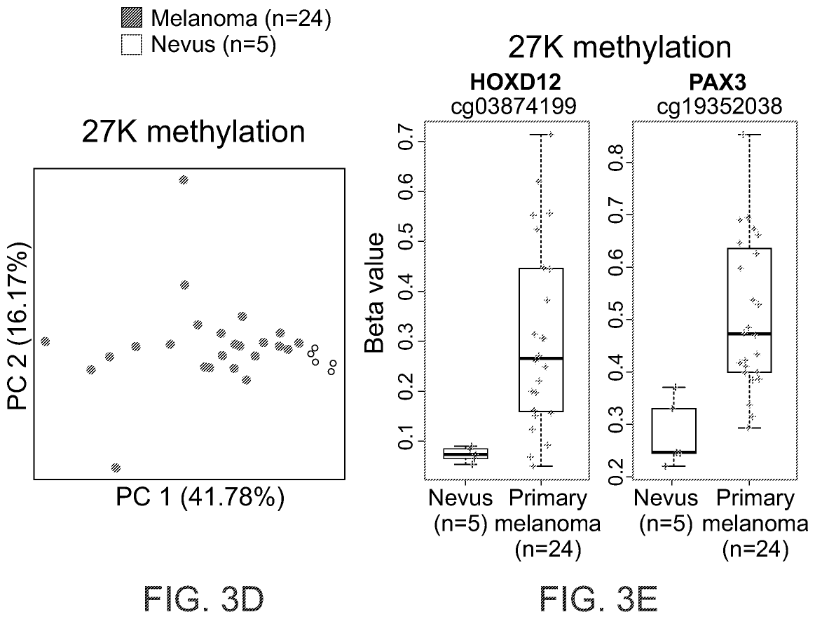

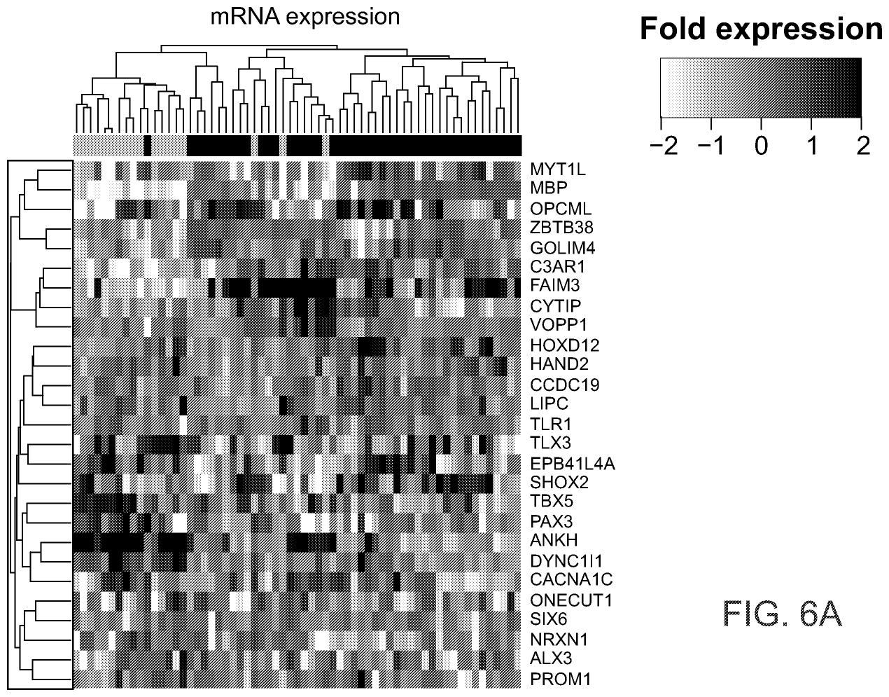

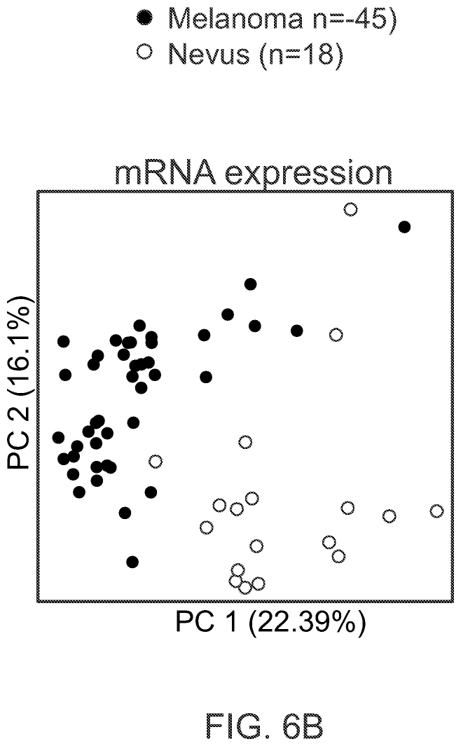

Validation of diagnostic genes in independent methylation or expression datasets Data from published datasets were used to confirm diagnostic methylation differences or to assess the biological relevance of differentially methylated genes by examining associated mRNA expression differences in melanomas versus nevi. As shown in the heatmap and associated waterfall plot in FIG. 3A, application of the 40-CpG diagnostic predictor to 105 primary melanomas in the TCGA 450K methylation dataset (TCGA, 2015) confirmed 103 of these as melanomas despite TCGA primary melanomas being larger and obtained as frozen specimens compared with UNC study samples. Moreover, 367 metastatic melanomas from TCGA showed a similar range of prediction scores as the TCGA primary melanomas (FIG. 3B). The heatmap in FIG. 3C and PCA plot in FIG. 3D use Illumina 27K methylation data from the study of Gao et al (2013) and illustrate that differential CpG methylation in the promoters of diagnostic signature genes, such as PAX3, HOXD12, TLX3 and TBX5 and GIMAP7, distinguished primary melanomas from nevi. FIG. 3E confirms the differential methylation between melanomas and nevi for two probes (cg03874199 in HOXD12; cg19352038 in PAX3) in our diagnostic signature. Differential mRNA expression of several diagnostic genes, including PAX3, TBX5, MBP, GOLIM4, and ANKH, also differentiated primary melanomas from benign nevi in the dataset of Talantov et al (2005) (FIG. 6A).

### 6.3. Discussion

This study identified a 40-CpG methylation signature that distinguished cutaneous primary invasive melanomas from benign nevi with a sensitivity of 96.6% and specificity of 100.0%, and was successfully implemented in >97% of FFPE samples. The diagnostic predictor was developed from a genome-wide methylation platform, optimally trained and then independently validated on diverse sets of melanoma and benign nevus specimens concordant for diagnoses among multiple expert dermatopathologists, which was crucial to achieving the highest accuracy in diagnostic signature discovery. Importantly, the 40-CpG diagnostic signature confirmed the malignant nature of nearly all 472 primary and metastatic melanomas in TCGA and was further validated in published methylation and gene expression datasets. Moreover, the diagnostic signature incorporated CpG probes exhibiting larger methylation differences between melanomas and nevi, maximizing the robustness of the predictor. Since the 40 CpG signature was developed using FFPE samples and requires small amounts of DNA, it can be potentially considered as a diagnostic assay for clinical use.

Melanocytic samples exhibited a broad spectrum of histolopathologic and clinical features as would be expected in routine dermatopathology practice. In particular, nevi included several diagnostically challenging specimens displaying potentially premalignant features, such as dysplasia and/or atypia, as well as less common subtypes such as Spitz nevi. Importantly, although melanoma patients are typically older than those being biopsied for benign nevi, as in this dataset, the diagnostic accuracy of the methylation signature was similarly very high among both younger and older patients.

Application of this diagnostic assay to melanocytic specimens of uncertain malignant potential placed many among histopathologically-confirmed nevi. However, others displaying less distinct patterns of differential methylation, including atypical Spitz tumors, fell in an intermediate zone, suggesting that some lesions may be in transition toward melanoma. Analysis of larger tumor tissue sets, including rare melanocytic subtypes together with long-term clinical follow-up could help to more clearly identify the earliest methylation events associated with melanoma genesis and potentially resolve the diagnostic status of these lesions. Alternatively, inclusion of other biomarkers with the methylation predictor could improve diagnostic accuracy for these borderline lesions; otherwise, such specimens may need to be treated clinically as melanomas.

The melanoma diagnostic signature is heavily enriched in genes coding for homeobox developmental transcription factors (ALX3, HOXD12, ONECUT1, PAX3, SHOX2, SIX6, TLX3) and other transcriptional regulators (TBX5, ZBTB38, MYT1L). PAX3, a marker of melanocytic cells, is a key regulator of melanocyte development and has putative roles in cell survival, migration, and differentiation (Medic and Ziman, 2009; Medic and Ziman, 2010; Dye et al, 2013). Altered methylation of PAX3 and several other diagnostic signature genes (HOXD12, OPCML, GIMAP7, FAIM3) has previously been reported in melanomas versus nevi (Conway et al, 2011; Gao et al, 2013; Furuta et al, 2004; Jin et al, 2015). PROM1 (CD133), a stem cell marker involved in maintaining stem cell pluripotency, is frequently expressed in melanomas (Zimmerer et al, 2016; Sharma et al, 2010). Gene ontology analysis revealed associations of several diagnostic genes with neural tissues/processes (e.g., OPCML, NRXN1, HAND2, MYT1L, MBP, TLX3), reflecting their common embryologic derivation with melanocytes from neural crest cells (Noisa and Raivio, 2014). FLJ22536, recently identified as CASC15, is a putative mediator of neural growth and differentiation and a tumor suppressor in neuroblastoma (Russell et al, 2015), and in melanoma is linked to disease progression and phenotype switching between proliferative and invasive states (Lessard et al, 2015). Other diagnostic genes lack well-defined roles in melanoma; however, a number exhibit aberrant expression (Makiyama et al, 2005; Jiang et al, 2008; Gao et al, 2015) and/or methylation (Lai et al, 2008; Semaan et al, 2016; Song et al, 2015; Kikuchi et al, 2013; Li et al, 2015; Yu et al, 2010; Zhao et al, 2013; Jones et al, 2013; Wimmer et al, 2002), function in apoptosis (Causeret et al, 2016; Baras et al, 2011; Baras et al, 2009) or differentiation (Zha et al, 2012), or are diagnostic (Semann et al, 2016; Song et al, 2015; Xing et al, 2015), prognostic (Dietrich et al, 2013; Zhou et al, 2014; Zheng et al, 2015; Galluzzi et al, 2013; Qiu et al, 2015) or predictive biomarkers (Tada et al, 2011) in other cancer types.

Given that ˜15% of melanocytic lesions are diagnostically ambiguous even among expert dermatopathologists, a molecular diagnostic test for melanoma that could be used in conjunction with histopathology, such as that described here, is urgently needed. In current clinical pathology practice, immunostains (e.g., Ki67, HMB45, p16) can aid pathologists' interpretation of melanocytic lesions, but single stains have low diagnostic accuracy (Uguen et al, 2015); combination staining may have higher accuracy but requires pathologist interpretation and lacks independent validation. Copy number analyses by comparative genomic hybridization (CGH) show that most melanomas, but few nevi, harbor numerous chromosomal changes (Bauer and Bastian, 2006; Bastian et al, 2000); however, CGH requires more tissue than is typically available from melanocytic samples. Fluorescence in situ hybridization detection of specific chromosomal changes is viewed directly on slides, using little tissue, but requires technical expertise for interpretation (Busam, 2013). All of these currently utilized tests suffer from unclear diagnostic accuracy across the broad spectrum of melanoma and nevus subtypes (Ivan and Prieto, 2010) and limited independent validation. The Myriad MyPath Melanoma mRNA expression-based test showed reasonably high diagnostic accuracy (sensitivity of 90%, specificity of 91%) for melanoma, but failed in 25% of FFPE samples (vs. <3% in this study) (Clarke et al, 2015). Needed is an approach that combines high accuracy across diverse melanocytic subtypes, technical robustness, and the ability to reliably screen early, small melanomas.

The advantages of a methylation-based diagnostic test include the stability of DNA methylation in FFPE samples and the ability to analyze methylation despite considerable DNA degradation. Our test was optimized in mostly smaller FFPE melanocytic samples and included some archival specimens more than 10 years old. Moreover, initiating unbiased diagnostic signature discovery from a whole-genome methylation platform allowed for optimal selection of loci performing critical functions in the neoplastic transition toward melanoma. Our diagnostic methylation signature showed high accuracy in the validation set comprised of varied melanoma and nevus types; however, additional studies are needed to fully validate the performance of the signature and optimize prediction score thresholds among larger numbers of samples, particularly rare melanocytic subtypes, especially in prospective studies with patient observation and/or follow-up.

### 6.4. Materials and Methods

Patients and tissues FFPE primary melanomas, benign nevi, and uncertain melanocytic samples were assembled from the pathology archives of the University of North Carolina (UNC) Hospitals or from the University of Rochester Medical Center based on original diagnoses abstracted from pathology reports and diagnosed between 2001 and 2012. The Institutional Review Boards at UNC and the University of Rochester approved the study. Melanomas were chosen to span AJCC tumor stages and included common and less common subtypes (e.g., Spitzoid, nevoid, and desmoplastic melanomas). Nevi were chosen to include intradermal melanocytic nevi including those with congenital pattern, compound melanocytic nevi with mild to severe dysplasia, Spitz and blue nevi, and other uncommon nevi (e.g. deep penetrating nevus, pigmented spindle cell nevus, and proliferative nodule in congenital pattern nevus). In addition, melanocytic lesions of uncertain malignant potential were selected. Age, sex, race, and anatomic site were abstracted from the medical chart. Pathologic review of all specimens was conducted independently by three expert dermatopathologists in order to assign diagnoses of melanoma or benign nevus or to identify uncertain melanocytic lesions. One pathologist conducted a centralized histopathological review for histologic pigment and adjacent solar elastosis of the melanocytic lesions; for nevus type of the nevi, and for histologic subtype, Breslow thickness, mitoses, ulceration, and tumor infiltrating lymphocytes of the melanomas. Details of the histopathology and interobserver review are provided in TABLE 1 and Supp. TABLE 51.

DNA preparation Melanocytic lesions were manually microdissected using H&E slides as guides, and DNA was prepared as described (Thomas et al, 2004).

Bisulfite treatment Sodium bisulfite modification of 250-300 ng DNA from each FFPE tissue was performed using the EZ DNA Methylation Lightning kit (Zymo Research, Orange, Calif.) according to the manufacturer's protocol.

HumanMethylation450 Beadchip analysis. Bisulfite-modified DNA (120 ng) was processed through the Illumina Infinium HD FFPE Restore protocol according to the manufacturer's instructions, and Illumina Infinium HumanMethylation450 BeadChip (450K) array analysis was performed in the Mammalian Genotyping Core at UNC. Details on methylation array analysis and data preprocessing are provided in the Supplemental Methods. The final dataset contained 383,229 probes and 203 samples (89 melanomas, 73 nevi, 41 diagnostically uncertain, 12 controls).

Statistical analyses To develop a diagnostic signature distinguishing melanomas from nevi, melanomas and nevi were each split into training (67%) and test (the remaining 33%) sets. Multiple predictive models based on different probe sets were tested for their ability to distinguish melanomas from benign nevi; these included accounting for effects of age and limiting probes to the most differentially methylated. For each probe set, Monte-Carlo cross validation was performed on training samples using the ElasticNet algorithm implemented in R package glmnet (Zou and Hastie, 2005) to select CpG subsets that best differentiate melanomas, and prediction scores were calculated for the final model. Heatmaps were generated to illustrate methylation at the diagnostic probe set, and principal component analysis (PCA) was performed to illustrate the segregation of melanomas and nevi. Full details of prediction model development and validation are provided in the Supplemental Methods.

Independent validation in published methylation datasets Illumina 450K methylation data for TCGA-SKCM (skin cutaneous melanomas; 105 primary and 367 metastatic) were downloaded from the Broad Institute Firehose web portal (http://firebrowse.org/) (version 2016012800). Beta values for each of the 40 CpG probes were converted to 0s if they were ‘NA’. The final prediction model was applied using the beta values to calculate a prediction score for each melanoma sample. A heatmap and waterfall plot, ordered left to right according to increasing prediction score, display beta values and corresponding prediction scores for each TCGA primary melanoma or UNC melanoma or nevus. Boxplots illustrate the range of predictions cores for TCGA primary and metastatic melanomas versus UNC samples. Using the Gao et al (2013) Illumina Infinium HumanMethylation27 (27K) methylation dataset in 24 melanomas and 5 nevi downloaded from Gene Expression Omnibus (GEO) (accession number GSE45266), methylation beta values at probes corresponding to diagnostic signature genes were median centered and used to generate a heatmap in R using Spearman rank correlation and average linkage clustering.

Dermatopathologic inter-observer review Pathologic review of all specimens was conducted independently by three expert dermatopathologists in order to assign diagnoses of melanoma or benign nevus or to identify uncertain melanocytic lesions. Five μm-thick tissue sections were cut from each tissue block containing melanoma, nevus, or uncertain melanocytic lesion and were mounted on uncoated glass slides. A hematoxylin and eosin (H&E)-stained slide of each tissue was initially reviewed by an expert dermatopathologist to assign diagnosis, classify histologic subtype, score standard histopathology features, and evaluate each specimen for adequacy of formalin-fixation, tissue size, percent melanocytic cells, and percent necrosis. This reviewer also encircled the melanocytic tissue areas on the H&E slides for use as guides in manual microdissection. Two additional expert dermatopathologists reviewed the same series of melanocytic samples using H&E-stained slides or high-resolution Aperio images and assigned diagnoses of melanoma, nevus, or uncertain. In the final assignment of diagnosis, melanocytic specimens were considered uncertain if there was inter-observer disagreement in the diagnosis of melanoma versus nevus between any of the 3 dermatopathology readers or the pathology report, or if any dermatopathogist or the pathology report described the specimen as having uncertain diagnosis. Based on the pathology report, 30 of the melanocytic lesions were uncertain; however, taking into account the subsequent dermatopathologist reviews, 7 additional nevi (based on the pathology report) and 4 additional melanomas (based on the pathology report) were reclassified as uncertain. One nevoid melanoma based on the pathology report had two expert reviews as a melanoma and one as a nevus, but was allowed to remain in the data as a melanoma as the patient had had visceral metastases and died of disease. Details of the histopathology and inter-observer pathology review for the melanocytic specimens that were successfully profiled using the 450K arrays are provided in TABLE 1 and Supp. TABLE S1.

Illumina Infinium HumanMethylation450 Beadchip analysis Sodium bisulfite modified DNA (100 ng) was processed through the Illumina Infinium HD FFPE Restore protocol according to the manufacturer's instructions. Genome-wide DNA methylation profiling was performed on Restore-treated DNA from melanocytic samples using the Illumina Infinium HumanMethylation450 BeadChip (450K) array in the Mammalian Genotyping Core at UNC. Samples were analyzed in three batches that included mixtures of melanomas, nevi, melanocytic lesions of uncertain diagnoses, positive (fully methylated) and negative (unmethylated) controls, and melanoma cell line controls (MCF7, VMM39, A375). BeadArrays were scanned and data assembled using the Illumina BeadStudio methylation module (v 3.2). Each CpG methylation data point is represented by fluorescent signals from the M (methylated; Cy5) and U (unmethylated; Cy3) alleles. Background intensity computed from a set of negative controls was subtracted from each data point. The methylation level of individual CpG sites was determined by calculating the β value, defined as the ratio of the fluorescent signal from the methylated allele to the sum of the fluorescent signals of both the methylated and unmethylated alleles. β values range from 0 (completely unmethylated) to 1.0 (fully methylated). Infinium HumanMethylation450 BeadChip data were imported into R (http://cran.r-project.org).

Methylation array data preprocessing and filtering Preprocessing of the Infinium HumanMethylation450 BeadChip methylation dataset (n=485,557 probes) was performed by removing probes (n=41,937) mapping to more than one location in the genome (Price et al, 2013), with any missing values or poor-performing probes with detection p-values>0.05 in over 20% of the samples, probes on the X and Y chromosomes, and additional probes overlapping a SNP (n=56; Illumina tech note link). Beta mixture quantile (BMIQ) normalization (Teschendorff et al, 2013) was then applied to the methylation β values for correction of bias due to the type I and type II probe sets. Three melanomas, one nevus, and one uncertain sample (of the 203 samples) failed array analysis due to inadequate DNA quantity and/or quality. The final dataset contained 383,229 probes and 203 samples (89 melanomas, 73 nevi, 41 diagnostically uncertain, plus 12 controls).

Statistical analyses To develop a diagnostic signature distinguishing melanomas from nevi, the sample set of melanomas and nevi was randomly split into a training set (67% of each sample class, balanced for age and sex) and an independent test set (the remaining 33%). Multiple predictive models based on different probe sets, including models accounting for patient age, were tested for their ability to distinguish melanomas from benign nevi, as described below. For each probe set, Monte-Carlo cross validation with 100 iterations was performed on training samples using the ElasticNet algorithm implemented in R package glmnet (Zou and Hastie, 2005) to obtain optimal regularization parameters (alpha and lambda) for automatic selection of a subset of CpG probes that best differentiate melanomas. In each iteration, ⅔ of the training set was randomly selected to build the elastic model and to predict on the rest of ⅓ of the training set. Based on the average AUC (Area Under the ROC curve) across 100 iterations, we determined the number of probes to be included in the final model. Finally, we calculated the prediction score using the beta value of selected CpG probes in the final model. Heatmaps depicting methylation levels at diagnostic probes in melanomas and nevi were generated in R using Euclidean distance and average linkage clustering. Columns were annotated with diagnostic category, sample set and age. Principal component analysis (PCA) was performed on the methylation matrix (centered to zero and scaled to unit variance one) to illustrate the segregation of melanomas and nevi.

Diagnostic models tested Multiple models based on different probe sets or their combinations were tested for their ability to distinguish melanomas from benign nevi. First, to allow for future validation using the new Illumina Infinium MethylationEPIC (850K) array, we limited CpG probes in all models to those that were on both the 450K and EPIC (850K) arrays (maximum n=358,049). Second, we further tested models that restricted CpG probes to those associated with specific ‘candidate genes’ (according to Illumina annotation) that were previously found to be differentially methylated between melanomas and nevi in our prior study (Conway et al, 2011) using the Illumina Cancer Panel I methylation array (maximum n=6,003). Within each of these probe sets, we imposed several additional levels of filtering. We assessed the effect of limiting probes to those exhibiting larger differential methylation between melanomas and nevi (with interquartile range (IQR)>0.2 β). Because melanoma patients are typically older than those biopsied for nevi (as in this study), we addressed the potential effect of age on probe selection by testing the inclusion of patient age in the model, the effect of removing probes significantly associated with age in linear regression analysis of logit transformed beta values (probes with p<0.01; n=271,892 or 4,324 probes associated with ‘candidate’ genes), or adjusting for age after exclusion of age-associated probes. Finally, we also tested only CpG probes with annotation indicating genomic location in one or more genes. In total, we tested 19 models in the training set.

Analysis of association of methylation with patient age A linear regression model on logit transformed beta values was employed to determine whether individual CpG probes, including those selected as part of the diagnostic signature defining differences between melanomas and nevi, were associated with patient age. The Benjamini-Hochberg false discovery rate (FDR) was used to control for multiple comparisons, and probes significantly associated with age were significant at p<0.01.

Gene ontology analysis The DAVID Bioinformatics Resources 6.7 Functional Annotation Tool (https://david.ncifcrf.gov/) was used to perform gene-GO term enrichment analysis to identify the most relevant GO terms associated with the 38 genes found to be diagnostic for melanomas versus nevi. Entrez gene IDs for each gene were compared to the human whole genome background. We performed functional annotation clustering with default settings.

mRNA expression associated with diagnostic genes in an independent dataset The Affymetrix Hu133A gene expression dataset from Talantov et al (2005) with 18 benign nevi and 45 primary melanomas was downloaded from GEO (accession number GSE3189). Expression levels were summarized to the gene level by selecting the probe set with highest standard deviation for each gene. Expression data for each gene were median-centered and clustered in R using Spearman rank correlation and average linkage. Principal component analysis was also performed to illustrate the segregation between melanomas and nevi.

TABLE 1
Clinical and histologic characteristics of cutaneous melanocytic nevi, primary melanomas, and
melanocytic proliferations of uncertain diagnosis that were evaluated for DNA methylation

| | | | | | | | | | | | Melanocytic | |
|---|---|---|---|---|---|---|---|---|---|---|---|---|
| | Training Set | | | | Validation Set | | | | | | Proliferation | |
| | | | Primary | | | | Primary | | Validation vs | | Uncertain | |
| | Nevi | | Melanomas | | Nevi | | Melanomas | | Training | | Diagnosis^{b} | |
| | (n = 48) | | (n = 60) | | (n = 25) | | (n = 29) | | Nevi | Melanomas | (n = 41) | |
| Characteristic | No | % | No | % | No | % | No | % | P^{a} | P^{a} | No | % |
| Laboratory processing of unstained FFPE tissue | | | | | | | | | | | | |
| University of North Carolina Pathology | 45 | 94 | 56 | 93 | 22 | 88 | 28 | 97 | .41 | 1.00 | 41 | 100 |
| University of Rochester Pathology Laboratories | 3 | 6 | 4 | 7 | 3 | 12 | 1 | 3 | | | — | — |
| Sex | | | | | | | | | | | | |
| Male | 23 | 48 | 38 | 63 | 12 | 48 | 19 | 66 | 1.00 | 1.00 | 16 | 39 |
| Female | 25 | 52 | 22 | 37 | 13 | 52 | 10 | 34 | | | 25 | 61 |
| Age at diagnosis of mole or primary melanoma | | | | | | | | | | | | |
| ≤50 yrs | 40 | 83 | 13 | 22 | 22 | 88 | 8 | 28 | .74 | .60 | 30 | 73 |
| >50 yrs | 8 | 17 | 47 | 78 | 3 | 12 | 21 | 72 | | | 11 | 27 |
| Race | | | | | | | | | | | | |
| Caucasian | 35 | 73 | 52 | 87 | 16 | 64 | 27 | 93 | .42 | .41 | 24 | 59 |
| Other | 4 | 8 | 1 | 2 | 1 | 4 | 1 | 3 | | | 7 | 17 |
| Unknown | 9 | 19 | 7 | 12 | 8 | 32 | 1 | 3 | | | 10 | 24 |
| Anatomic site of mole or primary melanoma | | | | | | | | | | | | |
| Head/neck | 1 | 23 | 20 | 33 | 8 | 32 | 9 | 31 | .46 | 1.00 | 4 | 10 |
| Trunk | 25 | 52 | 18 | 30 | 12 | 48 | 9 | 31 | | | 23 | 56 |
| Upper extremities | 5 | 10 | 11 | 18 | 4 | 1 | 6 | 21 | | | 3 | 7 |
| Lower extremities | 7 | 15 | 11 | 18 | 1 | 4 | 5 | 17 | | | 11 | 27 |
| Histologic subtype of primary melanoma | | | | | | | | | | | | |
| Superficial Spreading | — | — | 27 | 45 | — | — | 16 | 55 | — | .93 | — | — |
| Nodular | — | — | 9 | 15 | — | — | 4 | 14 | | | — | — |
| Lentigo maligna | — | — | 12 | 20 | — | — | 5 | 17 | | | — | — |
| Acral lentiginous | — | — | 5 | 8 | — | — | 1 | 3 | | | — | — |
| Other/unclassified^{c} | — | — | 7 | 12 | — | — | 3 | 10 | | | — | — |
| Melanocytic nevus type | | | | | | | | | | | | |
| Intradermal | 10 | 21 | — | — | 7 | 28 | — | — | .62 | — | — | — |
| Common acquired | 8 | 17 | — | — | 1 | 4 | — | — | | | — | — |
| Congenital pattern | 8 | 17 | — | — | 6 | 24 | — | — | | | — | — |
| Dysplastic | 9 | 19 | — | — | 5 | 20 | — | — | | | — | — |
| Spitz | 6 | 13 | — | — | 4 | 16 | — | — | | | — | — |
| Other^{d} | 7 | 15 | — | — | 2 | 8 | — | — | | | — | — |
| Breslow thickness of primary melanoma, mm | | | | | | | | | | | | |
| Median, range | — | — | 2.3 | 0.48- | — | — | 1.4 | 0.37- | — | .17 | — | — |
| 0.01 to 1.00 | — | — | 9 | 15 | — | — | 11 | 38 | — | .14 | — | — |
| 1.01 to 2.00 | — | — | 20 | 33 | — | — | 7 | 24 | — | | — | — |
| 2.01 to 4.00 | — | — | 13 | 22 | — | — | 4 | 14 | — | | — | — |
| >4.00 | — | — | 18 | 30 | — | — | 7 | 24 | — | | — | — |
| Ulceration of primary melanoma | | | | | | | | | | | | |
| Absent | — | — | 33 | 55 | — | — | 20 | 69 | — | .50 | — | — |
| Present | — | — | 26 | 43 | — | — | 9 | 31 | — | | — | — |
| Indeterminate | — | — | 1 | 2 | — | — | 0 | — | — | | — | — |
| Mitoses of primary melanoma | | | | | | | | | | | | |
| Absent | — | — | 9 | 15 | — | — | 8 | 28 | — | .25 | — | — |
| Present | — | — | 51 | 85 | — | — | 21 | 72 | — | | — | — |
| AJCC tumor stage at diagnosis | | | | | | | | | | | | |
| T1a | — | — | 8 | 13 | — | — | 6 | 21 | — | .36 | — | — |
| T1b/T2a | — | — | 14 | 23 | — | — | 11 | 38 | — | | — | — |
| T2b/T3a | — | — | 13 | 22 | — | — | 4 | 14 | — | | — | — |
| T3b/T4a | — | — | 12 | 20 | — | — | 2 | 7 | — | | — | — |
| T4b | — | — | 12 | 20 | — | — | 6 | 21 | — | | — | — |
| Indeterminate | — | — | 1 | 2 | — | — | 0 | | — | | — | — |
| Tumor infiltrating lymphocyte (TIL) grade of primary | | | | | | | | | | | | |
| Absent | — | — | 17 | 28 | — | — | 4 | 14 | — | .43 | — | — |
| Nonbrisk | — | — | 29 | 48 | — | — | 17 | 59 | — | | — | — |
| Brisk | — | — | 13 | 22 | — | — | 8 | 28 | — | | — | — |
| Indeterminate | — | — | 1 | 2 | — | — | 0 | — | — | | — | — |
| Pigment of the melanocytic lesion | | | | | | | | | | | | |
| Absent | 9 | 1 | 14 | 23 | 4 | 1 | 3 | 10 | .37 | .10 | 8 | 20 |
| Medium | 27 | 5 | 31 | 52 | 18 | 7 | 22 | 76 | | | 22 | 54 |
| Heavy | 12 | 2 | 15 | 25 | 3 | 1 | 4 | 14 | | | 11 | 27 |
| Solar Elastosis adjacent to the melanocytic | | | | | | | | | | | | |
| Absent | 27 | 5 | 14 | 23 | 17 | 6 | 8 | 28 | .79 | .30 | 35 | 85 |
| Mild to moderate | 4 | 8 | 26 | 43 | 2 | 8 | 16 | 55 | | | 4 | 10 |
| Severe | 2 | 4 | 14 | 23 | 1 | 4 | 5 | 17 | | | 1 | 2 |
| Indeterminate | 15 | 3 | 6 | 10 | 5 | 2 | 0 | — | | | 1 | 2 |

^{a}P-values were derived from the Fisher's exact test.
^{a}Melanocytic proliferations were considered uncertain if there was interobserver disagreement between any of 3 dermatopathology readers or the pathology report diagnosis of nevus vs. melanoma or one of the dermatopathogists or pathology report described the specimen as having uncertain diagnosis.
^{c}Other types of melanoma include nevoid (n = 2), desmoplastic (n = 1), spindle cell (n = 1), Spitzoid (n = 1), unclassified (n = 5).
^{d}Other includes cellular blue nevus (n = 2), combined intradermal or sclerotic blue nevus, not cellular (n = 1), combined nevus with compound congenital pattern and deep penetrating nevus (n = 2), pigmented spindle cell nevus (n = 2), and proliferative nodule in congenital pattern nevus (n = 2).

TABLE 2
40 CpG probes in the melanoma diagnostic classifier

| | | | | | Location | | | | | | |
|---|---|---|---|---|---|---|---|---|---|---|---|
| | | | | Location | relative | | | Methylation: | | | |
| | | | | relative | to CpG | | Regulatory | melanomas | Mean β | Mean | |
| CpG ID | Gene(s) | Gene name | Chr | to gene¹ | Island | Enhancer | Feature² | vs. nevi | melanomas | β nevi | p value³ |
| cg02936049 | ZBTB38 | Zinc Finger And | 3 | 5′UTR | — | Yes | | hyper | 0.6439 | 0.2438 | 1.79E−24 |
| | | BTB Domain | | | | | | | | | |
| | | Containing 38 | | | | | | | | | |
| cg19352038 | PAX3; | Paired Box 3; | 2 | TSS1500; | S_Shore | Yes | | hyper | 0.7275 | 0.2998 | 5.41E−24 |
| | CCDC140 | Coiled-Coil Domain | | 5′UTR | | | | | | | |
| | | Containing 140 | | | | | | | | | |
| cg16325502 | CCDC140 | Coiled-Coil Domain | 2 | 5′UTR | N_Shore | | | hyper | 0.6445 | 0.1971 | 1.46E−23 |
| | | Containing 140 | | | | | | | | | |
| cg05787556 | TLX3 | T-Cell Leukemia | 5 | TSS1500 | Island | Yes | | hyper | 0.5660 | 0.1849 | 2.97E−23 |
| | | Homeobox 3 | | | | | | | | | |
| cg12993163 | SHOX2 | Short Stature | 3 | Body | Island | | UCTS | hyper | 0.5156 | 0.1211 | 3.18E−23 |
| | | Homeobox 2 | | | | | | | | | |
| cg08697503 | CCDC140 | Coiled-Coil Domain | 2 | 5′UTR | N_Shore | | | hyper | 0.6263 | 0.2026 | 4.76E−23 |
| | | Containing 140 | | | | | | | | | |
| cg18077971 | PAX3; | Paired Box 3; | 2 | TSS1500; | S_Shore | Yes | | hyper | 0.6626 | 0.2259 | 1.44E−22 |
| | CCDC140 | Coiled-Coil Domain | | 5′UTR | | | | | | | |
| | | Containing 140 | | | | | | | | | |
| cg06215569 | ALX3 | ALX Homeobox 3 | 1 | Body | Island | Yes | | hyper | 0.6228 | 0.1524 | 9.07E−22 |
| cg16919569 | NBLA00301; | (HAND2-AS1) | 4 | Body; | Island | | | hyper | 0.6748 | 0.3470 | 1.74E−21 |
| | HAND2 | HAND2 Antisense | | TSS1500 | | | | | | | |
| | | RNA 1 (Head To | | | | | | | | | |
| | | Head); Heart And | | | | | | | | | |
| | | Neural Crest | | | | | | | | | |
| | | Derivatives | | | | | | | | | |
| | | Expressed 2 | | | | | | | | | |
| cg13164157 | PROM1 | Prominin 1 (CD133) | 4 | 5′UTR | Island | Yes | UCTS | hyper | 0.4445 | 0.0586 | 3.77E−21 |
| cg07230581 | OPCML | Opioid Binding | 11 | TSS1500 | — | | | hypo | 0.4214 | 0.7547 | 6.71E−21 |
| | | Protein/Cell | | | | | | | | | |
| | | Adhesion Molecule- | | | | | | | | | |
| | | Like | | | | | | | | | |
| cg00387964 | SORCS2 | Sortilin Related | 4 | Body | S_Shelf | | Uncl | hypo | 0.3893 | 0.8097 | 1.05E−20 |
| | | VPS10 Domain | | | | | | | | | |
| | | Containing | | | | | | | | | |
| | | Receptor 2 | | | | | | | | | |
| cg03315407 | ANKH | ANKH Inorganic | 5 | Body | — | Yes | Uncl | hypo | 0.2717 | 0.6184 | 2.04E−20 |
| | | Pyrophosphate | | | | | | | | | |
| | | Transport Regulator | | | | | | | | | |
| cg02744046 | LIPC | Lipase C, Hepatic | 15 | Body | — | | | hyper | 0.6004 | 0.2332 | 2.24E−20 |
| | | Type | | | | | | | | | |
| cg13019491 | SIX6 | SIX Homeobox 6 | 14 | Body | Island | | | hyper | 0.5128 | 0.1094 | 2.54E−20 |
| cg17918270 | MYT1L | Myelin | 2 | Body | — | | | hypo | 0.5044 | 0.8731 | 6.10E−20 |
| | | Transcription | | | | | | | | | |
| | | Factor 1 Like | | | | | | | | | |
| cg18689332 | TBX5 | T-Box 5 | 12 | Body | N_Shore | Yes | | hyper | 0.7265 | 0.3632 | 6.29E−20 |
| cg08337633 | VOPP1 | Vesicular, | 7 | Body | — | Yes | PA | hypo | 0.3099 | 0.6939 | 1.21E−19 |
| | | Overexpressed In | | | | | | | | | |
| | | Cancer, Prosurvival | | | | | | | | | |
| | | Protein 1 | | | | | | | | | |
| cg15849098 | GIMAP7 | GTPase, IMAP | 7 | TSS200 | — | | UCTS | hypo | 0.3606 | 0.6898 | 1.54E−19 |
| | | Family Member 7 | | | | | | | | | |
| cg26967305 | KREMEN1 | Kringle Containing | 22 | 3′UTR | — | | UCTS | hypo | 0.4830 | 0.7867 | 1.59E−19 |
| | | Transmembrane | | | | | | | | | |
| | | Protein 1 | | | | | | | | | |
| cg03874199 | HOXD12 | Homeobox D12 | 2 | TSS200 | Island | | | hyper | 0.5570 | 0.1707 | 3.22E−19 |
| cg10559416 | CYTIP | Cytohesin 1 | 2 | 1st Exon | — | | | hypo | 0.5015 | 0.8105 | 1.19E−18 |
| | | Interacting Protein | | | | | | | | | |
| cg14064356 | CCDC140 | Coiled-Coil Domain | 2 | 5′UTR | N_Shore | Yes | | hyper | 0.6245 | 0.2139 | 1.70E−18 |
| | | Containing 140 | | | | | | | | | |
| cg22322562 | NRXN1 | Neurexin 1 | 2 | Body | — | | | hyper | 0.6634 | 0.3041 | 3.79E−18 |
| cg02468320 | CACNA1C | Calcium Voltage- | 12 | Body | — | Yes | | hypo | 0.4413 | 0.7889 | 4.40E−18 |
| | | Gated Channel | | | | | | | | | |
| | | Subunit Alpha1 C | | | | | | | | | |
| cg04499514 | C3AR1 | Complement | 12 | TSS200 | — | | PA | hypo | 0.4203 | 0.7683 | 2.69E−17 |
| | | Component 3a | | | | | | | | | |
| | | Receptor 1 | | | | | | | | | |
| cg07569216 | ONECUT1 | One Cut Homeobox | 15 | Body | N_Shore | | | hyper | 0.6641 | 0.3067 | 3.29E−17 |
| | | 1 | | | | | | | | | |
| cg07637837 | MBP | Myelin Basic | 18 | 5′UTR | Island | | | hypo | 0.4298 | 0.7612 | 3.49E−17 |
| | | Protein | | | | | | | | | |
| cg08898055 | RASGEF1C | RasGEF Domain | 5 | 5′UTR | — | Yes | | hyper | 0.5153 | 0.1971 | 3.59E−17 |
| | | Family Member 1C | | | | | | | | | |
| cg09476130 | CCDC19 | (CFAP45) Cilia And | 1 | TSS200 | Island | Yes | | hyper | 0.6648 | 0.3535 | 4.52E−17 |
| | | Flagella Associated | | | | | | | | | |
| | | Protein 45 | | | | | | | | | |
| cg15158847 | FAIM3 | Fas Apoptotic | 1 | 5′UTR; | — | | Uncl | hypo | 0.4876 | 0.7843 | 4.65E−17 |
| | | Inhibitory Molecule | | 1stExon | | | | | | | |
| | | 3; (alias FCMR) Fc | | | | | | | | | |
| | | Fragment Of IgM | | | | | | | | | |
| | | Receptor | | | | | | | | | |
| cg18851100 | SHANK3 | SH3 And Multiple | 2 | Body | Island | | | hyper | 0.6274 | 0.3162 | 8.95E−17 |
| | | Ankyrin Repeat | | | | | | | | | |
| | | Domains 3 | | | | | | | | | |
| cg07553475 | FLJ22536 | CASC15; Cancer | 6 | TSS1500 | Island | | | hyper | 0.5285 | 0.1341 | 5.39E−16 |
| | | Susceptibility | | | | | | | | | |
| | | Candidate 15 | | | | | | | | | |
| | | (Non-Protein | | | | | | | | | |
| | | Coding) | | | | | | | | | |
| cg15536663 | EPB41L4A | Erythrocyte | 5 | Body | — | Yes | | hypo | 0.3481 | 0.6444 | 7.30E−16 |
| | | Membrane Protein | | | | | | | | | |
| | | Band 4.1 Like 4A | | | | | | | | | |
| cg06573459 | SGEF | (ARHGEF26) Rho | 3 | Body | S_Shore | | UCTS | hyper | 0.5123 | 0.1580 | 9.35E−16 |
| | | Guanine Nucleotide | | | | | | | | | |
| | | Exchange Factor 26 | | | | | | | | | |
| cg03653573 | C5orf56 | Chromosome 5 | 5 | Body | — | | PA | hypo | 0.3512 | 0.6451 | 2.12E−15 |
| | | Open Reading | | | | | | | | | |
| | | Frame 56 | | | | | | | | | |
| cg18098839 | GOLIM4 | Golgi Integral | 3 | Body | — | Yes | | hypo | 0.3378 | 0.6652 | 9.51E−15 |
| | | Membrane Protein 4 | | | | | | | | | |
| cg17889682 | DYNC1I1 | Dynein Cytoplasmic | 7 | 5′UTR | S_Shore | Yes | | hyper | 0.6412 | 0.3127 | 1.70E−14 |
| | | 1 Intermediate | | | | | | | | | |
| | | Chain 1 | | | | | | | | | |
| cg08757862 | TLR1 | Toll Like Receptor 1 | 4 | TSS1500 | — | | PA | hypo | 0.5317 | 0.8120 | 2.58E−14 |
| cg12423733 | MAS1L | MAS1 Proto- | 6 | 1stExon | — | | | hypo | 0.4461 | 0.7019 | 5.69E−12 |
| | | Oncogene Like, | | | | | | | | | |
| | | G Protein-Coupled | | | | | | | | | |
| | | Receptor | | | | | | | | | |

¹TSS; transcription start site, UTR; untranslated region.
²UCTS, unclassified cell type-specific; Uncl, unclassified; PA, promoter-associated.
³Wilcoxon p value for mean β in melanomas versus nevi.

SUPP. TABLE S1
Pathology report information, expert dermatopathologic review, final diagnostic category, 40-probe prediction call, and
prediction score of melanocytic lesions deemed uncertain

| | | | | | | | | 40 |
|---|---|---|---|---|---|---|---|---|
| | | Reviewer 1 | | | | Final | 40 Probe | Probe |
| | | (pathology | Reviewer | Reviewer | Reviewer | diagnostic | Prediction | Prediction |
| Sample | Pathology report description | report) | 2 | 3 | 4 | category | Score | Call |
| 1593 | Cellular blue nevus, atypical | Uncertain | Nevus | Nevus | Uncertain | Uncertain | −3.12773 | Nevus |
| 1332 | Nevus of the groin | Nevus | Uncertain | Nevus | Nevus | Uncertain | −2.74323 | Nevus |
| 1552 | Atypical compound dysplastic nevus/thin | Uncertain | Nevus | Nevus | Nevus | Uncertain | −2.69522 | Nevus |
| | invasive melanoma | | | | | | | |
| 1533 | Compound nevus with melanoma-in-situ | Uncertain | Nevus | Nevus | Nevus | Uncertain | −2.58955 | Nevus |
| 1190 | Melanoma | Melanoma | Melanoma | Nevus | Melanoma | Uncertain | −2.33431 | Nevus |
| 794 | Cellular blue nevus | Nevus | Nevus | Uncertain | Nevus | Uncertain | −2.33151 | Nevus |
| 1321 | Nevus | Nevus | Uncertain | Nevus | Nevus | Uncertain | −2.28870 | Nevus |
| 1548 | Atypical compound dysplastic nevus/thin nevoid | Uncertain | Nevus | Nevus | Nevus | Uncertain | −2.17733 | Nevus |
| | invasive melanoma | | | | | | | |
| 1556 | Atypical compound dysplastic nevus/thin | Uncertain | Nevus | Nevus | Nevus | Uncertain | −2.07719 | Nevus |
| | invasive melanoma | | | | | | | |
| 1205 | Viewed by multiple pathologists with differing | Uncertain | Uncertain | Melanoma | Melanoma | Uncertain | −1.45668 | Nevus |
| | opinions | | | | | | | |
| 1538 | Combined blue and intradermal nevus, atypical | Uncertain | Nevus | Nevus | Uncertain | Uncertain | −1.45415 | Nevus |
| 1580 | Nevus | Nevus | Uncertain | Nevus | Nevus | Uncertain | −1.39522 | Nevus |
| 1542 | Atypical compound dysplatic nevus/thin | Uncertain | Uncertain | Nevus | Uncertain | Uncertain | −1.26519 | Nevus |
| | invasive melanoma | | | | | | | |
| 1540 | Atypical compound dysplastic nevus/thin | Uncertain | Uncertain | Nevus | Nevus | Uncertain | −1.23147 | Nevus |
| | invasive melanoma | | | | | | | |
| 1553 | Spitz tumor, atypical | Uncertain | Uncertain | Nevus | Uncertain | Uncertain | −1.20256 | Nevus |
| 1292 | Atypical nevus/thin invasive melanoma | Uncertain | Melanoma | Melanoma | Melanoma | Uncertain | −1.19044 | Nevus |
| | (favored) | | | | | | | |
| 1274 | Melanoma | Melanoma | Nevus | Nevus | Melanoma | Uncertain | −1.17831 | Nevus |
| 1541 | Atypical melanocytic nevus/thin invasive | Uncertain | Melanoma | Nevus | Uncertain | Uncertain | −1.09320 | Nevus |
| | melanoma | | | | | | | |
| 1191 | Melanoma but difficult lesion due to conflicting | Uncertain | Uncertain | Nevus | Melanoma | Uncertain | −1.06850 | Nevus |
| | criteria | | | | | | | |
| 1531 | Spitz tumor, atypical | Uncertain | Melanoma | Uncertain | Melanoma | Uncertain | −1.00901 | Nevus |
| 771 | Spitz tumor, atypical/UMP | Uncertain | Uncertain | Uncertain | Uncertain | Uncertain | −0.93935 | Nevus |
| 1554 | Spitz tumor, atypical | Uncertain | Uncertain | Nevus | Uncertain | Uncertain | −0.70002 | Nevus |
| 1544 | Spitz tumor, atypical | Uncertain | Uncertain | Nevus | Uncertain | Uncertain | −0.69067 | Nevus |
| 1583 | Epitheliod blue nevus/pigmented epitheliod | Uncertain | Nevus | Nevus | Uncertain | Uncertain | −0.65540 | Nevus |
| | melanocytoma, atypical | | | | | | | |
| 1549 | Atypical compound genital melanocytic nevus/ | Uncertain | Melanoma | Nevus | Uncertain | Uncertain | −0.57115 | Nevus |
| | thin invasive melanoma | | | | | | | |
| 1320 | Nevus | Nevus | Uncertain | Nevus | Nevus | Uncertain | −0.56191 | Nevus |
| 1545 | Spitz tumor, desmplastic, atypical/Spitzoid | Uncertain | Uncertain | Nevus | Uncertain | Uncertain | −0.52197 | Nevus |
| | invasive melanoma | | | | | | | |
| 808 | Pigmented epithelioid melanocytoma, atypical | Uncertain | Nevus | Nevus | Uncertain | Uncertain | −0.49093 | Nevus |
| 1543 | Spitz tumor, atypical | Uncertain | Uncertain | Nevus | Uncertain | Uncertain | −0.44768 | Nevus |
| 748 | Polypoid inflamed Spitz tumor with several | Nevus | Uncertain | Uncertain | Nevus | Uncertain | −0.32519 | Nevus |
| | mitoses | | | | | | | |
| 1539 | Atypical compound pigmented spindle cell | Uncertain | Nevus | Nevus | Uncertain | Uncertain | −0.23936 | Nevus |
| | nevus/thin invasive melanoma | | | | | | | |
| 774 | Spitz tumor, sclerosing, atypical/UMP | Uncertain | Nevus | Nevus | Nevus | Uncertain | −0.21985 | Nevus |
| 1314 | Compound nevus with atypical features | Nevus | Uncertain | Nevus | Nevus | Uncertain | −0.21341 | Nevus |
| 1306 | Atypical nevus/thin invasive melanoma | Uncertain | Uncertain | Nevus | Nevus | Uncertain | −0.20317 | Nevus |
| 1547 | Compound dysplastic nevus, atypical/thin | Uncertain | Uncertain | Nevus | Uncertain | Uncertain | −0.16890 | Nevus |
| | invasive melanoma | | | | | | | |
| 1257 | Superficial spreading melanoma | Melanoma | Nevus | Nevus | Melanoma | Uncertain | −0.01628 | Nevus |
| 785 | Melanoma (favored)/Spitz tumor, atypical | Uncertain | Uncertain | Uncertain | Melanoma | Uncertain | 0.02616 | Melanoma |
| 1555 | Atypical compound dysplastic nevus/thin | Uncertain | Uncertain | Nevus | Nevus | Uncertain | 0.11857 | Melanoma |
| | invasive melanoma | | | | | | | |
| 1568 | Epitheliod blue nevus/pigmented epitheliod | Uncertain | Nevus | Nevus | Uncertain | Uncertain | 0.17649 | Melanoma |
| | melanocytoma, atypical | | | | | | | |
| 1551 | Spitz tumor, atypical | Uncertain | Uncertain | Uncertain | Uncertain | Uncertain | 0.23579 | Melanoma |
| 1188 | Melanoma UMP, uncertain malignant potential. | Melanoma | Nevus | Nevus | Melanoma | Uncertain | 0.62767 | Melanoma |

Abbreviations:
UMP, uncertain malignant potential.

SUPP. TABLE S2
Diagnostic accuracy of the 40 CpG signature in the validation set

| Characteristic | | AUC | Sensitivity | Specificity | NPV | PPV |
|---|---|---|---|---|---|---|
| All patients | | 0.996 | 96.6% | 100.0% | 96.2% | 100.0% |
| Patient or lesion | | | | | | |
| Age | <50 | 0.996 | 95.2% | 100.0% | 98.4% | 100.0% |
| | >50 | 1.000 | 100.0% | 100.0% | 100.0% | 100.0% |
| Sex | Male | 0.999 | 98.2% | 100.0% | 97.2% | 100.0% |
| | Female | 1.000 | 100.0% | 100.0% | 100.0% | 100.0% |
| Anatomic Site | Trunk | 1.000 | 100.0% | 100.0% | 100.0% | 100.0% |
| of Lesion | Head/neck/extremities | 0.999 | 98.4% | 100.0% | 97.3% | 100.0% |
| Lesion | Heavy/Medium | 0.999 | 98.6% | 100.0% | 98.4% | 100.0% |
| Pigmentation | Absent | 1.000 | 100.0% | 100.0% | 100.0% | 100.0% |
| Solar elastosis | Absent | 1.000 | 100.0% | 100.0% | 100.0% | 100.0% |
| in skin | Mild to Severe | 0.998 | 98.4% | 100.0% | 90.0% | 100.0% |
| Tissue or technical factor | | | | | | |
| Institutional | UNC-Chapel Hill | 0.999 | 98.8% | 100.0% | 98.5% | 100.0% |
| source | U Rochester | 1.000 | 100.0% | 100.0% | 100.0% | 100.0% |
| Illumina array* | 450K | 1.000 | 100.0% | 100.0% | 100.0% | 100.0% |
| | EPIC 850K | 1.000 | 100.0% | 100.0% | 100.0% | 100.0% |
| Presence of | Moderate/brisk | 0.998 | 98.7% | 100.0% | 96.7% | 100.0% |
| lymphocytes | Absent/minimal | 1.000 | 100.0% | 100.0% | 100.0% | 100.0% |
| % melanocytic | ≥50% | 1.000 | 98.5% | 100.0% | 98.3% | 100.0% |
| cells | <50% | 1.000 | 100.0% | 100.0% | 100.0% | 100.0% |

*Comparison restricted to 25 samples run on both platforms. PPV; positive predictive value. NPV; negative predictive value.

SUPPLEMENTARY TABLE S3
Top 50 functional annotation terms from DAVID GO analysis of 38 genes in the melanoma diagnostic signature

| | | | | | List | Pop | Pop | Fold | |
|---|---|---|---|---|---|---|---|---|---|
| Category | Term | Count | % | P value | Total | Hits | Total | Enrichment | Benjamini |
| INTERPRO | IPR012287: Homeodomain-related | 7 | 18.4 | 3.56E−06 | 31 | 238 | 16659 | 15.8 | 1.14E−04 |
| INTERPRO | IPR001356: Homeobox | 7 | 18.4 | 3.31E−06 | 31 | 235 | 16659 | 16.0 | 1.59E−04 |
| UP_SEQ_FEATURE | DNA-binding region: Homeobox | 7 | 18.4 | 1.14E−06 | 36 | 190 | 19113 | 19.6 | 1.88E−04 |
| INTERPRO | IPR017970: Homeobox, conserved site | 7 | 18.4 | 3.07E−06 | 31 | 232 | 16659 | 16.2 | 2.95E−04 |
| SMART | SM00389: HOX | 7 | 18.4 | 1.49E−05 | 23 | 235 | 9079 | 11.8 | 4.16E−04 |
| SP_PIR_KEYWORDS | Homeobox | 7 | 18.4 | 4.46E−06 | 36 | 242 | 19235 | 15.5 | 4.77E−04 |
| GOTERM_MF_FAT | GO: 0043565~sequence-specific DNA binding | 9 | 23.7 | 1.16E−05 | 26 | 607 | 12983 | 7.4 | 6.10E−04 |
| GOTERM_MF_FAT | GO: 0003700~transcription factor activity | 1 | 28.9 | 6.28E−06 | 26 | 975 | 12983 | 5.6 | 6.59E−04 |
| SP_PIR_KEYWORDS | developmental protein | 9 | 23.7 | 6.21E−05 | 36 | 779 | 19235 | 6.2 | 3.32E−03 |
| GOTERM_MF_FAT | GO: 0030528~transcription regulator activity | 11 | 28.9 | 2.79E−04 | 26 | 1512 | 12983 | 3.6 | 9.72E−03 |
| SP_PIR_KEYWORDS | dna-binding | 11 | 28.9 | 1.38E−03 | 36 | 1868 | 19235 | 3.1 | 4.80E−02 |
| GOTERM_MF_FAT | GO: 0003677~DNA binding | 1 | 28.9 | 8.10E−03 | 26 | 2331 | 12983 | 2.4 | 1.57E−01 |
| GOTERM_MF_FAT | GO: 0016563~transcription activator activity | 5 | 13.2 | 7.33E−03 | 26 | 410 | 12983 | 6.1 | 1.76E−01 |
| GOTERM_MF_FAT | GO: 0001653~peptide receptor activity | 3 | 7.9 | 2.01E−02 | 26 | 114 | 12983 | 13.1 | 2.99E−01 |
| GOTERM_MF_FAT | GO: 0008528~peptide receptor activity, G-protein | 3 | 7.9 | 2.01E−02 | 26 | 114 | 12983 | 13.1 | 2.99E−01 |
| | coupled | | | | | | | | |
| SP_PIR_KEYWORDS | Transcription | 9 | 23.7 | 2.96E−02 | 36 | 2071 | 19235 | 2.3 | 4.15E−01 |
| GOTERM_BP_FAT | GO: 0048598~embryonic morphogenesis | 5 | 13.2 | 3.95E−03 | 30 | 307 | 13528 | 7.3 | 4.28E−01 |
| SP_PIR_KEYWORDS | transcription regulation | 9 | 23.7 | 2.64E−02 | 36 | 2026 | 19235 | 2.4 | 4.35E−01 |
| SP_PIR_KEYWORDS | disease mutation | 8 | 21.1 | 2.25E−02 | 36 | 1591 | 19235 | 2.7 | 4.56E−01 |
| GOTERM_BP_FAT | GO: 0051252~regulation of RNA metabolic process | 11 | 28.9 | 3.23E−03 | 30 | 1813 | 13528 | 2.7 | 4.97E−01 |
| GOTERM_CC_FAT | GO: 0044459~plasma membrane part | 10 | 26.3 | 7.63E−03 | 23 | 2203 | 12782 | 2.5 | 5.28E−01 |
| GOTERM_BP_FAT | GO: 0030326~embryonic limb morphogenesis | 3 | 7.9 | 1.48E−02 | 30 | 87 | 13528 | 15.5 | 5.47E−01 |
| GOTERM_BP_FAT | GO: 0035113~embryonic appendage morphogenesis | 3 | 7.9 | 1.48E−02 | 30 | 87 | 13528 | 15.5 | 5.47E−01 |
| GOTERM_BP_FAT | GO: 0048736~appendage development | 3 | 7.9 | 2.04E−02 | 30 | 103 | 13528 | 13.1 | 5.48E−01 |
| GOTERM_BP_FAT | GO: 0060173~limb development | 3 | 7.9 | 2.04E−02 | 30 | 103 | 13528 | 13.1 | 5.48E−01 |
| GOTERM_BP_FAT | GO: 0002009~morphogenesis of an epithelium | 3 | 7.9 | 1.97E−02 | 30 | 101 | 13528 | 13.4 | 5.69E−01 |
| GOTERM_BP FAT | GO: 0060429~epithelium development | 4 | 10.5 | 1.24E−02 | 30 | 227 | 13528 | 7.9 | 5.85E−01 |
| GOTERM_MF_FAT | GO: 0042277~peptide binding | 3 | 7.9 | 5.77E−02 | 26 | 203 | 12983 | 7.4 | 5.90E−01 |
| GOTERM_BP_FAT | GO: 0045449~regulation of transcription | 12 | 31.6 | 1.47E−02 | 30 | 2601 | 13528 | 2.1 | 5.93E−01 |
| GOTERM_BP_FAT | GO: 0035108~limb morphogenesis | 3 | 7.9 | 1.89E−02 | 30 | 99 | 13528 | 13.7 | 5.94E−01 |
| GOTERM_BP_FAT | GO: 0035107~appendage morphogenesis | 3 | 7.9 | 1.89E−02 | 30 | 99 | 13528 | 13.7 | 5.94E−01 |
| GOTERM_BP_FAT | GO: 0035295~tube development | 4 | 10.5 | 1.14E−02 | 30 | 220 | 13528 | 8.2 | 6.20E−01 |
| GOTERM_BP_FAT | GO: 0001501 ~skeletal system development | 4 | 10.5 | 3.02E−02 | 30 | 319 | 13528 | 5.7 | 6.32E−01 |
| GOTERM_BP_FAT | GO: 0035239~tube morphogenesis | 3 | 7.9 | 3.01E−02 | 30 | 127 | 13528 | 10.7 | 6.60E−01 |
| GOTERM_BP_FAT | GO: 0050877~neurological system process | 7 | 18.4 | 4.03E−02 | 30 | 1210 | 13528 | 2.6 | 6.63E−01 |
| GOTERM_BP_FAT | GO: 0045165~cell fate commitment | 3 | 7.9 | 3.55E−02 | 30 | 139 | 13528 | 9.7 | 6.65E−01 |
| GOTERM_BP_FAT | GO: 0035136~forelimb morphogenesis | 2 | 5.3 | 4.61E−02 | 30 | 22 | 13528 | 41.0 | 6.71E−01 |
| GOTERM_CC_FAT | GO: 0044456~synapse part | 3 | 7.9 | 6.62E−02 | 23 | 246 | 12782 | 6.8 | 6.73E−01 |
| GOTERM_BP_FAT | GO: 0045944~positive regulation of transcription | 4 | 10.5 | 4.42E−02 | 30 | 371 | 13528 | 4.9 | 6.76E−01 |
| | from RNA polymerase II promoter | | | | | | | | |
| GOTERM_BP_FAT | GO: 0006350~transcription | 9 | 23.7 | 6.92E−02 | 30 | 2101 | 13528 | 1.9 | 6.76E−01 |
| GOTERM_BP_FAT | GO: 0010557~positive regulation of macromolecule | 5 | 13.2 | 4.93E−02 | 30 | 654 | 13528 | 3.4 | 6.76E−01 |
| | biosynthetic process | | | | | | | | |
| GOTERM_BP_FAT | GO: 0007507~heart development | 4 | 10.5 | 1.07E−02 | 30 | 215 | 13528 | 8.4 | 6.79E−01 |
| GOTERM_BP_FAT | GO: 0035115~embryonic forelimb morphogenesis | 2 | 5.3 | 4.00E−02 | 30 | 19 | 13528 | 47.5 | 6.84E−01 |
| GOTERM_BP_FAT | GO: 0014032~neural crest cell development | 2 | 5.3 | 6.84E−02 | 30 | 33 | 13528 | 27.3 | 6.85E−01 |
| GOTERM_BP_FAT | GO: 0014033~neural crest cell differentiation | 2 | 5.3 | 6.84E−02 | 30 | 33 | 13528 | 27.3 | 6.85E−01 |
| GOTERM_BP_FAT | GO: 0006355~regulation of transcription, | 11 | 28.9 | 2.74E−03 | 30 | 1773 | 13528 | 2.8 | 6.87E−01 |
| | DNA-dependent | | | | | | | | |
| GOTERM_BP_FAT | GO: 0009891~positive regulation of biosynthetic | 5 | 13.2 | 5.92E−02 | 30 | 695 | 13528 | 3.2 | 6.91E−01 |
| | process | | | | | | | | |
| GOTERM_BP_FAT | GO: 0031328~positive regulation of cellular | 5 | 13.2 | 5.67E−02 | 30 | 685 | 13528 | 3.3 | 6.92E−01 |
| | biosynthetic process | | | | | | | | |
| GOTERM_BP_FAT | GO: 0006357~regulation of transcription from RNA | 5 | 13.2 | 6.76E−02 | 30 | 727 | 13528 | 3.1 | 6.95E−01 |
| | polymerase II promoter | | | | | | | | |
| GOTERM_BP_FAT | GO: 0046620~regulation of organ growth | 2 | 5.3 | 6.24E−02 | 30 | 30 | 13528 | 30.1 | 6.95E−01 |

SUPP. TABLE S4
SEQ ID NO: 1-40 (These sequences are
associated with the 40 CpG signature)

| | Forward_ | Genome_ | | |
|---|---|---|---|---|
| CpG Name | Sequence | Build | CHR | MAPINFO |
| cg19352038 | TTTATAACTTGGTAA | 37 | 2 | 223164869 |
| | GTGCCAGCGAACTCG | | | |
| | CCTCCTTTACACCCC | | | |
| | CGAGTGCCAGCCCCG | | | |
| | [CG]CTCTGCACTGC | | | |
| | GCTTTATTCGCTCGA | | | |
| | GCCTATTCAGGGACT | | | |
| | GTCACTCCGGGGCCG | | | |
| | CGAG | | | |
| cg02936049 | AGACCACGAGCAAGT | 37 | 3 | 141102599 |
| | AAGCACGTTAATCAA | | | |
| | AGTGAAAGGCTCACC | | | |
| | CCTCACGTCTAGCTC | | | |
| | [CG]TCCTTCTCCAG | | | |
| | CCTGTGCCTGCCAGA | | | |
| | TTATTTCGGGTTCCT | | | |
| | CGTGTTTGACTCGTC | | | |
| | AAAG | | | |
| cg16325502 | GCCACTCTTTCTCTG | 37 | 2 | 223166435 |
| | TCTCCGAGTCTTGGG | | | |
| | CCTCCCCTTTATTTC | | | |
| | TTTCTGAAGTCTCTC | | | |
| | [CG]GAGCCCAAGCC | | | |
| | ACCCCACACCCAAAC | | | |
| | CCCGCAGCTGGATGG | | | |
| | GAGTCCAGGCCACTT | | | |
| | CCCT | | | |
| cg18077971 | TATTTATAACTTGGT | 3 | 2 | 223164867 |
| | AAGTGCCAGCGAACT | | | |
| | CGCCTCCTTTACACC | | | |
| | CCCGAGTGCCAGCCC | | | |
| | [CG]CGCTCTGCACT | | | |
| | GCGCTTTATTCGCTC | | | |
| | GAGCCTATTCAGGGA | | | |
| | CTGTCACTCCGGGGC | | | |
| | CGCG | | | |
| cg08697503 | TGGCTGTCCAGGCCT | 37 | 2 | 223166946 |
| | GAGTGGAGCGTGCCC | | | |
| | TTGTTAGCTTGAAAG | | | |
| | TTCTCCCTCGCAGCC | | | |
| | [CG]TTTGGATGCGT | | | |
| | GCGTCTACAGCCCAG | | | |
| | TCGCACTTTGGTGAC | | | |
| | CGGCCTGGGCTGTGA | | | |
| | AGCA | | | |
| cg12993163 | GACAGCCAGGTAATC | 37 | 3 | 157821407 |
| | TCCGTCCCGCCTGCC | | | |
| | CGACCGGGGTCGCAC | | | |
| | GAGCACAGGCGCCCA | | | |
| | [CG]CCATGTTGGCT | | | |
| | GCCCAAAGGGCTCGC | | | |
| | CGCCCAAGCCGGGCC | | | |
| | AGAAGGCAGGAGGCG | | | |
| | GAAA | | | |
| cg06215569 | ATTTCCCTTCCCCTT | 37 | 1 | 110611465 |
| | TTCTTGGTTGTCGCT | | | |
| | CGCTTTCTTTGGTTT | | | |
| | TCTTTCTCGGTATTT | | | |
| | [CG]TTGTCAAGGCC | | | |
| | ACCCTTGCCGTCGGA | | | |
| | TCCCGGGGTGCTGGG | | | |
| | TTTCTCCCGGCCGCT | | | |
| | CGTT | | | |
| cg05787556 | TTCGCTGGAAGAAAA | 37 | 5 | 170735186 |
| | TGATTCCGCTTGTCT | | | |
| | CCCCAAAGCTGCAGC | | | |
| | GGAAGGTGACTACTT | | | |
| | [CG]TGTGCGGTCCT | | | |
| | GTCCACGGTGCCCTG | | | |
| | GGCCGGGTAGACAGT | | | |
| | CACTGAGGCGCGAGC | | | |
| | AGAA | | | |
| cg14064356 | CATCTAGAGCTGAGT | 37 | 2 | 223165753 |
| | CTCATTTGTTTTTGA | | | |
| | GCCGGAGGCTTGGTC | | | |
| | TCCAAGCCCTCCCAG | | | |
| | [CG]TCCACCCGTCT | | | |
| | CTCTCCTGCCGGGAG | | | |
| | TTTTCTCTCCTAAGA | | | |
| | GCCGGCAGATGCTGG | | | |
| | AGGG | | | |
| cg13019491 | CCGTAGACTCCAGCA | 37 | 14 | 60977856 |
| | GCAGGTCCTGTCACA | | | |
| | GGGTTCCGGGCGGGC | | | |
| | ACTACGGGCGGAGGG | | | |
| | [CG]ACGGCACGCCA | | | |
| | GAGGTGCTGGGCGTC | | | |
| | GCCACCAGCCCGGCC | | | |
| | GCCAGTCTATCCAGC | | | |
| | AAGG | | | |
| cg16919569 | TAGCCCAAGGGAGAC | 37 | 4 | 174452835 |
| | CAAAGACTCCACCTT | | | |
| | GAGCATCGCCCTTTG | | | |
| | GAGGCGGGCAGAGTC | | | |
| | [CG]GCCGCAGGCCA | | | |
| | CAAAGCGATCCCCAC | | | |
| | CCGAAGGACTCCACA | | | |
| | AGGACAGTCCTTTCC | | | |
| | TTGC | | | |
| cg02744046 | TTGTAGCTGAGTGGG | 37 | 15 | 58782685 |
| | TGAAACGGCATCACC | | | |
| | AACATTTGGCCCTGC | | | |
| | TGCTCCACTGAGAGC | | | |
| | [CG]GCGCCGTTCGC | | | |
| | GGGATAATTATCCTG | | | |
| | TAGTCTTCTCACCTC | | | |
| | CGGAGAGAATGCAAG | | | |
| | GCGC | | | |
| cg18689332 | AGGGAGGAGAAAGGC | 37 | 12 | 114837666 |
| | GAAGGGAGGAGGTAA | | | |
| | CAGCAGGCGGGCAAC | | | |
| | TGTAGGTAACCTAAG | | | |
| | [CG]GAAAACAAACC | | | |
| | AGGACGCATGCGCCT | | | |
| | CTAGAGAACGGGTTT | | | |
| | TGAAGATGCTTCAAA | | | |
| | GGGA | | | |
| cg03874199 | GATGTAGGCGGTGCT | 37 | 2 | 176964456 |
| | GAAATGACCGGCTTT | | | |
| | GAAGAACCTGCAGGC | | | |
| | AAAGTTTCGTCCAAT | | | |
| | [CG]TCTGAGCCTGT | | | |
| | CCTCTTATTCCCGGT | | | |
| | TGTAACTAAATACTG | | | |
| | TTGCGAGCGCAGCCG | | | |
| | AAGC | | | |
| cg13164157 | CTGCTGAGGGGCCAG | 37 | 4 | 16085180 |
| | GGAGGCGGCGCAGAT | | | |
| | GGCTAGGGTAAGGGG | | | |
| | GGCGCAGAGCGAACC | | | |
| | [CG]TCCACTCCTCA | | | |
| | CTGTACACCCCCAGT | | | |
| | ACAGTGGAAGGAGTG | | | |
| | CGCTCAGCCCCGCGC | | | |
| | CTGG | | | |
| cg22322562 | CCGGCACCACTCAAA | 37 | 2 | 50201511 |
| | AAGTCTCAGCAGTCG | | | |
| | TTGCTTTTCAATTTG | | | |
| | CTCCCCTAACGAGAC | | | |
| | [CG]CATAGGTAAAC | | | |
| | AGACCTCCCTCTAAC | | | |
| | CCCCGACCGAAAAAA | | | |
| | AGGCTTATTTTCATG | | | |
| | CACG | | | |
| cg07553475 | GGTGGAGGATGAGGA | 37 | 6 | 21665800 |
| | GGCGGCCTGGGACCC | | | |
| | CGAGTCAGATCTTTG | | | |
| | GGGTGAGCACGAGGA | | | |
| | [CG]TGGTGTAGGGA | | | |
| | AGAGGACGAGTGAGC | | | |
| | AGCGCCTGGCTGTAG | | | |
| | GGTCAGAGGGCGCCT | | | |
| | GGTC | | | |
| cg08898055 | GGCGCGGGCCTGTTC | 37 | 5 | 179597395 |
| | TGTGAGGGAGAAAAC | | | |
| | AAGCGTCCTATTTAC | | | |
| | CACGAGAATGAATAT | | | |
| | [CG]GGCTCTGTGTG | | | |
| | AAAATCCCACTTGCT | | | |
| | CTGAGATGTGTGAAG | | | |
| | CCAGCAGGGCCAGGG | | | |
| | ACGC | | | |
| cg07569216 | AGCCGCGTGGAGGGA | 3 | 15 | 53075533 |
| | CGAAAAGATCAACCA | | | |
| | CCCGATCGACGAGGA | | | |
| | TAGGTTTGATCTTTT | | | |
| | [CG]ATTACCTCAGT | | | |
| | GTGCCAGTGTATATT | | | |
| | CCCGGCTGGGCCTAG | | | |
| | CGCCCTAAGAAACTT | | | |
| | CGGA | | | |
| cg06573459 | CCACAAGAGACTCCT | 37 | 3 | 153840654 |
| | CAAGGTGCGCAGCAT | | | |
| | GGTGGAGGGCCTAGG | | | |
| | AGGACCCCTGGGTCA | | | |
| | [CG]CAGGGGAGGAG | | | |
| | AGTGAGGTCGATAAC | | | |
| | GACGTGGATAGCCCA | | | |
| | GGGTCTCTGCGGAGA | | | |
| | GGCT | | | |
| cg09476130 | ACCTCCAGCGGGCAG | 37 | 1 | 159870086 |
| | TTGCCTTGTGCTGGT | | | |
| | GGCTTAGGAACCGGA | | | |
| | GCCCGTCGCTCCAAC | | | |
| | [CG]TTGCAGCTCCA | | | |
| | CGCTCCAGCCCAACC | | | |
| | GCGGCTCTGAAGGAT | | | |
| | TGACCCGCCCTGGCG | | | |
| | TGCC | | | |
| cg18851100 | GGAGTCGGGTCAAGG | 37 | 22 | 51158550 |
| | CTGGCCTCTGTGGGA | | | |
| | GGGGGTTGCCGGGGT | | | |
| | CCCCAGGAACCTCTC | | | |
| | [CG]AAGGCAGCACC | | | |
| | ACCCCCCGCCCAGCG | | | |
| | CCCTGGCTGGTCTCA | | | |
| | CCGGCCCTTCCGTCC | | | |
| | GCAG | | | |
| cg17889682 | TTGTTTTGGTAAACA | 37 | 7 | 95402733 |
| | CCTTCACGGCCGCCT | | | |
| | GGCTCTCCTTCCCCC | | | |
| | GCTCCCATTCGGAAT | | | |
| | [CG]CTCTGGCCTTA | | | |
| | TAAATCCGTGCGTCG | | | |
| | TCATCATAAGGGCAG | | | |
| | TGATCCTGGCAGCGC | | | |
| | TGAT | | | |
| cg12423733 | TGTGCTCCCTGGGAA | 37 | 6 | 29454623 |
| | GAAGGTTCTCCACAT | | | |
| | GCTGAGTAGAGTGTG | | | |
| | GTTGCTCCATTGGGT | | | |
| | [CG]ATGCCAGCTGC | | | |
| | CTTTTTGTTCCTCCC | | | |
| | CACCTCTGGCTTATC | | | |
| | TGCTAACGCCCGTTG | | | |
| | GAGA | | | |
| cg08757862 | TTTTCTACCACACAG | 37 | 4 | 38807382 |
| | CGAGCAAGGCCAACT | | | |
| | TCCCTAAACTAAGAA | | | |
| | TGCTGAGATTCTTTT | | | |
| | [CG]ACTTATAATGT | | | |
| | TCTGACTGTCTCTCT | | | |
| | CTGTTTCCCCTTACC | | | |
| | TCAGAATTTGTTTAA | | | |
| | TAGA | | | |
| cg03653573 | CCTGGCTGTGTGTGT | 37 | 5 | 131762326 |
| | GGCGTCCAGTGCGAG | | | |
| | TGGTAGCCAGACATC | | | |
| | ATGCCCACCTGCCCT | | | |
| | [CG]AGCTGCTTGCC | | | |
| | TGCAGCTGGCTCCTT | | | |
| | ACTCACAGATCTGCA | | | |
| | TCCATCCGGCGCTGG | | | |
| | GGAG | | | |
| cg18098839 | ACTAACCTTTTACAT | 37 | 3 | 167742700 |
| | AAACCAGATGTTTCT | | | |
| | TAAAATAGCCCAGTT | | | |
| | AAATCCACCCTTCCT | | | |
| | [CG]TGGCATCTGCT | | | |
| | TACCACCAAATGTTC | | | |
| | CTCCACTTCTGTATT | | | |
| | CTCTTGCTTTTGATT | | | |
| | ACTT | | | |
| cg15536663 | TTTCAGCACCCCACC | 37 | 5 | 111665548 |
| | CCCTCTTCAGTTGAA | | | |
| | GGTAGCAAGCCATTC | | | |
| | CCACAGTGGGTGGCC | | | |
| | [CG]CAGGGTTATCT | | | |
| | GCCACATCAAAAAGA | | | |
| | GAGGCTTTATGGTAC | | | |
| | TCTACCAAGCATCCT | | | |
| | TACA | | | |
| cg15158847 | CACGCTGCTTACTCA | 37 | 1 | 207095315 |
| | GGAACCCTTCACAAT | | | |
| | CTGGAACTGGAAAGA | | | |
| | GATTTCTAGCCCCCA | | | |
| | [CG]AGGAACAAAGC | | | |
| | TTGACGATGAGGAAA | | | |
| | TGACAACCTCCCTTG | | | |
| | TTTGCTAACTATTCT | | | |
| | CAGG | | | |
| cg04499514 | TCATGAAGTATGGCA | 37 | 12 | 8219020 |
| | AGAAAATTTGCTGAG | | | |
| | CTTTCTCTTCTTCTG | | | |
| | TTCTCTCTCTGTTTC | | | |
| | [CG]GCAATAAGTTA | | | |
| | AGTCTTATGCTCTAG | | | |
| | ACCACTATCTGACCT | | | |
| | CACAGGAAGAGTTTC | | | |
| | AAAG | | | |
| cg10559416 | CATCGTAAGGCTGCC | 37 | 2 | 158300485 |
| | GGTGAGTGTGGAGTA | | | |
| | AGAGCTATACGCTGG | | | |
| | CCCAGCGCAGAAGTC | | | |
| | [CG]CCAAATTGCCA | | | |
| | TTGCTGCTGTGTTGC | | | |
| | AGGAGCCTTTGTAAA | | | |
| | GACATTGTGAATAAA | | | |
| | GATC | | | |
| cg17918270 | AATCGACCTCAGTTC | 37 | 2 | 1983484 |
| | CGCAGGATGAAGGTG | | | |
| | ACCCTGAGCCGGCCT | | | |
| | CAGGATGCAGGGAAG | | | |
| | [CG]CGGACATACCT | | | |
| | CGAACCCCTTTGGAC | | | |
| | CGCGTGCGATGCCGC | | | |
| | TTCTCCTCGGTGTCC | | | |
| | ACCT | | | |
| cg02468320 | GTGTAATCTCCGTGG | 37 | 12 | 2404134 |
| | TCAGTGAAGCAATGC | | | |
| | TGTGCGCACAGTTCT | | | |
| | GTGTTGTGCCTCTCC | | | |
| | [CG]GGGAAGGGGTG | | | |
| | TATTTGGCCTGTGCC | | | |
| | CCACCCTAGCCCTCC | | | |
| | TTCGTCTTCCCTCTT | | | |
| | TCAC | | | |
| cg07637837 | CAAAAACCCGTAGAA | 37 | 18 | 74824154 |
| | TGAACACCGTGCACA | | | |
| | CGCACACACACACAC | | | |
| | ACACACACGTGCGCG | | | |
| | [CG]CGGCAAAAAGA | | | |
| | AACAGCTCATTTCGG | | | |
| | AGCTGAGGACAAGGC | | | |
| | GTGGGAAGAAGACGC | | | |
| | GTTT | | | |
| cg15849098 | TCTAGGCAATATTTG | 37 | 7 | 150211761 |
| | GGTCATTTAATAAGG | | | |
| | CTCTTTTGCATCCAT | | | |
| | CACTATAACCTGAAG | | | |
| | [CG]AAAAATGTAGC | | | |
| | TTTGGAAATGGTGTT | | | |
| | TATAGCAGGCCCATG | | | |
| | GGCAAAACGTTTCAA | | | |
| | CCGG | | | |
| cg26967305 | CCTCAAAAGGCCCCA | 37 | 22 | 29563734 |
| | GGCCTACTGTGGTTT | | | |
| | TTTCTGAGAGGCTCC | | | |
| | CAGAACCAAGTGGCA | | | |
| | [CG]TTGGTTTCCTG | | | |
| | TGCGTCTGTGTCTTT | | | |
| | GTGCCTGTATCTCGC | | | |
| | TGGGGGACTTCACAG | | | |
| | GAAG | | | |
| cg08337633 | AGGTCCTGTCATGGT | 37 | 7 | 55602109 |
| | CACCTGTGGCTTGGG | | | |
| | CCAATTCTCACTTCC | | | |
| | CCTGAAGGGCAGCTG | | | |
| | [CG]TGTAGGGAGCG | | | |
| | GGGGCTGCCCAAAGT | | | |
| | TTCACTCTGACTGGA | | | |
| | GGTAAACTTAACATC | | | |
| | ATTT | | | |
| cg07230581 | TTTTCTGCTTCTCCT | 37 | 11 | 133403211 |
| | TTTCAGGGTCCAGTC | | | |
| | TGTCCCTTCCTCTGG | | | |
| | AATGGCAGTTTACAC | | | |
| | [CG]GCAGTTTACAC | | | |
| | AGGATGGCAGTATCT | | | |
| | CTGGGTGTAAGAAAC | | | |
| | CTCAGAACTTTCCCC | | | |
| | TGCT | | | |
| cg00387964 | ACAGATAATTCAAGT | 37 | 4 | 7651935 |
| | GCAGGTCTGACAGGG | | | |
| | GGTGACCCTGGGTGA | | | |
| | ATCACTCAATTGCTG | | | |
| | [CG]AGCCTCCATCT | | | |
| | TCCGTCTGCACGAGG | | | |
| | TATTGTTAGAAGCAT | | | |
| | CTACTTCCTGGCGAT | | | |
| | GTTG | | | |
| cg03315407 | CACTAGTGCCTGTTT | 37 | 5 | 14810180 |
| | TCCTGACTCTGACTT | | | |
| | CCTGGGTCTCGGCAC | | | |
| | CACAGATAGCTTCTG | | | |
| | [CG]TTTCTCTACAG | | | |
| | GAGGGAAGAAGCAAT | | | |
| | TTCCAATTCTGAGCT | | | |
| | TCATGAGGGAGGAGA | | | |
| | ATAA | | | |

Supp. TABLE S5 (SEQ ID NO: SEQ ID NO: 41-80)(Sequences associated with 40 CpG signature)

| | | UCSC_ | | UCSC_ | UCSC_CpG_ | Relation_ | | | | mean ß |
|---|---|---|---|---|---|---|---|---|---|---|
| | | RefGene_ | UCSC_ | RefGene_ | Islands | to_UCSC_ | | t. | mean ß | in |
| CpG Name | SourceSeq | Name | RefGene_Accession | Group | Name | CpG_Island | t.pvalue | statistics | in nevi | melanoma |
| cg19352038 | GAGTGACAGTCCCTGAA | PAX3/ | NM_013942; NM_000438; | TSS1500; | chr2: | S_Shore | 8.08E-42 | -18.96571 | 0.29981 | 0.72751 |
| | TAGGCTCGAGCGAATAA | CCDC140 | NM_181460; NM_181457; | 5′UTR | 223162946- | | | | | |
| | AGCGCAGTGCAGAGCG | | NM_181458; NM_153038; | | 223163912 | | | | | |
| | | | NM_181461; | | | | | | | |
| | | | NM_001127366; | | | | | | | |
| | | | NM_181459 | | | | | | | |
| cg02936049 | CGGAGCTAGACGTGAG | ZBTB38 | NM_001080412 | | | | 1.54E-41 | -18.52521 | 0.24379 | 0.64387 |
| | GGGTGAGCCTTTCACTT | | | | | | | | | |
| | TGATTAACGTGCTTACT | | | | | | | | | |
| cg16325502 | CGGAGCCCAAGCCACCC | CCDC140 | NM_ 153038 | 5′UTR | chr2: | N_Shore | 4.11E-39 | -18.05654 | 0.19713 | 0.64446 |
| | CACACCCAAACCCCGCA | | | | 223167205- | | | | | |
| | GCTGGATGGGAGTCCA | | | | 223167560 | | | | | |
| cg18077971 | GTGACAGTCCCTGAATA | PAX3/ | NM_013942;NM_000438; | TSS1500; | chr2: | S_Shore | 3.27E-38 | -18.02626 | 0.22592 | 0.66260 |
| | GGCTCGAGCGAATAAAG | CCDC140 | NM_181460; **NM_ | 5′UTR | 223162946- | | | | | |
| | CGCAGTGCAGAGCGCG | | 181457; NM_181458; | | 223163912 | | | | | |
| | | | NM_153038; NM_181461; | | | | | | | |
| | | | NM_001127366; | | | | | | | |
| | | | NM_181459 | | | | | | | |
| cg08697503 | CGGGCTGCGAGGGAGA | CCDC140 | NM 153038 | 5′UTR | chr2: | N Shore | 7.26E-38 | -17.63999 | 0.20261 | 0.62629 |
| | ACTTTCAAGCTAACAAG | | | | 223167205- | | | | | |
| | GGCACGCTCCACTCAGG | | | | 223167560 | | | | | |
| cg12993163 | CGCCATGTTGGCTGCCC | SHOX2 | NM_001163678; | Body | chr3: | Island | 8.36E-33 | 1-7.48068 | 0.12107 | 0.51557 |
| | AAAGGGCTCGCCGCCCA | | NM_006884; NM_003030 | | 157821232- | | | | | |
| | AGCCGGGCCAGAAGGC | | | | 157821604 | | | | | |
| cg06215569 | CTTTTCTTGGTTGTCGCT | ALX3 | NM_006492 | Body | chr1: | Island | 1.01E-32 | -15.89389 | 0.15243 | 0.62284 |
| | CGCTTTCTTTGGTTTTCT | | | | 110610265- | | | | | |
| | TTCTCGGTATTTCG | | | | 110613303 | | | | | |
| cg05787556 | AAATGATTCCGCTTGTCT | TLX3 | NM_021025 | TSS1500 | chr5: | Island | 8.04E-33 | -15.88562 | 0.18489 | 0.56603 |
| | CCCCAAAGCTGCAGCGG | | | | 170735169- | | | | | |
| | AAGGTGACTACTTCG | | | | 170739863 | | | | | |
| cg14064356 | CGCTGGGAGGGCTTGG | CCDC140 | NM_153038 | 5′UTR | chr2: | N_Shore | 1.78E-30 | -15.72046 | 0.21395 | 0.62450 |
| | AGACCAAGCCTCCGGCT | | | | 223167205- | | | | | |
| | CAAAAACAAATGAGACT | | | | 223167560 | | | | | |
| cg13019491 | CGCCCTCCGCCCGTAGT | SIX6 | NM_007374 | Body | chr14: | Island | 1.14E-27 | -15.52520 | 0.10943 | 0.51279 |
| | GCCCGCCCGGAACCCTG | | | | 60975732- | | | | | |
| | TGACAGGACCTGCTGC | | | | 60978180 | | | | | |
| cg16919569 | CGGCCGCAGGCCACAAA | NBLA00 | NR_003679; NM_021973 | Body; | chr4: | Island | 1.01E-31 | -14.79259 | 0.34705 | 0.67477 |
| | GCGATCCCCACCCGAAG | 301/ | | TSS1500 | 174451828- | | | | | |
| | GACTCCACAAGGACAG | HAND2 | | | 174452962 | | | | | |
| cg02744046 | GGGTGAAACGGCATCAC | LIPC | NM_000236 | Body | | | 5.63E-28 | -14.06229 | 0.23320 | 0.60040 |
| | CAACATTTGGCCCTGCT | | | | | | | | | |
| | GCTCCACTGAGAGCCG | | | | | | | | | |
| cg18689332 | ATCTTCAAAACCCGTTCT | TBX5 | NM_000192; NM_181486; | Body | chr12: | N Shore | 4.76E-28 | -13.51771 | 0.36317 | 0.72652 |
| | CTAGAGGCGCATGCGTC | | NM_080717; NM_080718 | | 114838312- | | | | | |
| | CTGGTTTGTTTTCCG | | | | 114838889 | | | | | |
| cg03874199 | GCTGAAATGACCGGCTT | HOXD12 | NM_021193 | TSS200 | chr2: | Island | 1.14E-24 | -13.11382 | 0.17066 | 0.55695 |
| | TGAAGAACCTGCAGGCA | | | | 176964062- | | | | | |
| | AAGTTTCGTCCAATCG | | | | 176965509 | | | | | |
| cg13164157 | CTGAGCGCACTCCTTCC | PROM1 | NM_001145848; | 5′UTR | chr4: | Island | 2.48E-22 | -12.75586 | 0.05858 | 0.44449 |
| | ACTGTACTGGGGGTGTA | | NM_001145847 | | 16084195- | | | | | |
| | CAGTGAGGAGTGGACG | | | | 16085735 | | | | | |
| cg22322562 | CGGTCTCGTTAGGGGAG | NRXN1 | NM_004801; | Body | | | 2.33E-24 | -12.28986 | 0.30412 | 0.66336 |
| | CAAATTGAAAAGCAACG | | NM_001135659; | | | | | | | |
| | ACTGCTGAGACTTTTT | | NM_138735 | | | | | | | |
| cg07553475 | CGTCCTCGTGCTCACCCC | FLJ22536 | NR_015410 | TSS1500 | chr6: | Island | 1.78E-21 | -12.28976 | 0.13410 | 0.52848 |
| | AAAGATCTGACTCGGGG | | | | 21665715- | | | | | |
| | TCCCAGGCCGCCTCC | | | | 21666031 | | | | | |
| cg08898055 | CGGGCTCTGTGTGAAAA | RASGEF1C | NM_175062 | 5′UTR | | | 4.69E-23 | -11.94232 | 0.19711 | 0.51529 |
| | TCCCACTTGCTCTGAGAT | | | | | | | | | |
| | GTGTGAAGCCAGCAG | | | | | | | | | |
| cg07569216 | CGATTACCTCAGTGTGC | ONECUT1 | NM_004498 | Body | chr15: | N_Shore | 1.55E-23 | -11.83081 | 0.30665 | 0.66411 |
| | CAGTGTATATTCCCGGC | | | | 53076187- | | | | | |
| | TGGGCCTAGCGCCCTA | | | | 53077926 | | | | | |
| cg06573459 | CCTCAAGGTGCGCAGCA | SGEF | NM_015595 | Body | chr3: | S_Shore | 9.41E-22 | -11.51727 | 0.15804 | 0.51230 |
| | TGGTGGAGGGCCTAGG | | | | 153838787- | | | | | |
| | AGGACCCCTGGGTCACG | | | | 153840380 | | | | | |
| cg09476130 | CGGGTCAATCCTTCAGA | CCDC19 | NM_012337 | TSS200 | chr1: | Island | 2.22E-21 | -11.02383 | 0.35350 | 0.66481 |
| | GCCGCGGTTGGGCTGG | | | | 159869901- | | | | | |
| | AGCGTGGAGCTGCAAC | | | | 159870143 | | | | | |
| | G | | | | | | | | | |
| cg18851100 | GGGCCGGTGAGACCAG | SHANK3 | NM_001080420 | Body | chr22: | Island | 2.36E-21 | -11.01549 | 0.31620 | 0.62738 |
| | CCAGGGCGCTGGGCGG | | | | 51158386- | | | | | |
| | GGGGTGGTGCTGCCTTC | | | | 51160060 | | | | | |
| | G | | | | | | | | | |
| cg17889682 | CGCTCTGGCCTTATAAAT | DYNC111 | NM_004411; | 5′UTR | chr7: | S_Shore | 4.20E-20 | -10.61031 | 0.31268 | 0.64122 |
| | CCGTGCGTCGTCATCAT | | NM_001135556; | | 95401691- | | | | | |
| | AAGGGCAGTGATCCT | | NM_001135557 | | 95402432 | | | | | |
| cg12423733 | CGACCCAATGGAGCAAC | MAS1L | NM_052967 | 1stExon | | | 6.53E-14 | 8.22460 | 0.70187 | 0.44611 |
| | CACACTCTACTCAGCAT | | | | | | | | | |
| | GTGGAGAACCTTCTTC | | | | | | | | | |
| cg08757862 | CAGCGAGCAAGGCCAAC | TLR1 | NM_003263 | TSS1500 | | | 1.34E-14 | 8.55054 | 0.81200 | 0.53167 |
| | TTCCCTAAACTAAGAAT | | | | | | | | | |
| | GCTGAGATTCTTTTCG | | | | | | | | | |
| cg03653573 | GGATGGATGCAGATCTG | C5orf56 | NM_001013717 | Body | | | 1.76E-18 | 9.96608 | 0.64508 | 0.35122 |
| | TGAGTAAGGAGCCAGCT | | | | | | | | | |
| | GCAGGCAAGCAGCTCG | | | | | | | | | |
| cg18098839 | CATAAACCAGATGTTTCT | GOLIM4 | NM_014498 | Body | | | 1.62E-18 | 9.97188 | 0.66523 | 0.33785 |
| | TAAAATAGCCCAGTTAA | | | | | | | | | |
| | ATCCACCCTTCCTCG | | | | | | | | | |
| cg15536663 | TGGTAGAGTACCATAAA | EPB41L4A | NM_022140 | Body | | | 3.44E-20 | 10.58951 | 0.64437 | 0.34814 |
| | GCCTCTCTTTTTGATGTG | | | | | | | | | |
| | GCAGATAACCCTGCG | | | | | | | | | |
| cg15158847 | TAGCAAACAAGGGAGG | FAIM3 | NM_001142472; | 5′UTR; | | | 1.91E-21 | 11.07534 | 0.78427 | 0.48757 |
| | TTGTCATTTCCTCATCGT | | NM_005449; | 1stExon | | | | | | |
| | CAAGCTTTGTTCCTCG | | NM_001142473; | | | | | | | |
| | | | NM_005449; | | | | | | | |
| | | | NM_001142473 | | | | | | | |
| cg04499514 | GCAAGAAAATTTGCTGA | C3AR1 | NM_004054 | TSS200 | | | 1.58E-21 | 11.07884 | 0.76832 | 0.42028 |
| | GCTTTCTCTTCTTCTGT | | | | | | | | | |
| | TCTCTCTCTGTTTCCG | | | | | | | | | |
| cg10559416 | CGCCAAATTGCCATTGC | CYTIP | NM 004288 | 1stExon | | | 3.70E-23 | 11.87553 | 0.81052 | 0.50154 |
| | TGCTGTGTTGCAGGAGC | | | | | | | | | |
| | CTTTGTAAAGACATTG | | | | | | | | | |
| cg17918270 | CGCGGACATACCTCGAA | MYT1L | NM_015025 | Body | | | 3.36E-21 | 12.08364 | 0.87310 | 0.50436 |
| | CCCCTTTGGACCGCGTG | | | | | | | | | |
| | CGATGCCGCTTCTCCT | | | | | | | | | |
| cg02468320 | TGGTCAGTGAAGCAATG | CACNA1C | NM_001129844; | Body | | | 6.65E-24 | 12.10212 | 0.78885 | 0.44127 |
| | CTGTGCGCACAGTTCTG | | NM_001129827; | | | | | | | |
| | TGTTGTGCCTCTCCCG | | NM_001129839; | | | | | | | |
| | | | NM_001129834; | | | | | | | |
| | | | NM_001129841; | | | | | | | |
| | | | NM_000719; | | | | | | | |
| | | | NM_001129830; | | | | | | | |
| | | | NM_001167625; | | | | | | | |
| | | | NM_001129843; | | | | | | | |
| | | | NM_001167624; | | | | | | | |
| | | | NM_001129835; | | | | | | | |
| | | | NM_001129837; | | | | | | | |
| | | | NM_001167623; | | | | | | | |
| | | | NM_001129840; | | | | | | | |
| | | | NM_199460; | | | | | | | |
| | | | NM_001129833; | | | | | | | |
| | | | NM_001129832; | | | | | | | |
| | | | NM_001129829; | | | | | | | |
| | | | NM_001129846; | | | | | | | |
| | | | NM_001129836; | | | | | | | |
| | | | NM_001129838; | | | | | | | |
| | | | NM_001129831; | | | | | | | |
| | | | NM_001129842 | | | | | | | |
| cg07637837 | TTCCCACGCCTTGTCCTC | MBP | NM_001025101; | 5′UTR | chr18: | Island | 1.16E-23 | 12.25545 | 0.76116 | 0.42981 |
| | AGCTCCGAAATGAGCTG | | NM_001025100 | | 74824149- | | | | | |
| | TTTCTTTTTGCCGCG | | | | 74824414 | | | | | |
| cg15849098 | TTTTGCCCATGGGCCTG | GIMAP7 | NM_153236 | TSS200 | | | 5.08E-26 | 12.72380 | 0.68982 | 0.36060 |
| | CTATAAACACCATTTCCA | | | | | | | | | |
| | AAGCTACATTTTTCG | | | | | | | | | |
| cg26967305 | CGTGCCACTTGGTTCTG | KREMEN1 | NM_032045 | 3′UTR | | | 2.11E-25 | 12.75344 | 0.78673 | 0.48299 |
| | GGAGCCTCTCAGAAAAA | | | | | | | | | |
| | ACCACAGTAGGCCTGG | | | | | | | | | |
| cg08337633 | CGCAGCTGCCCTTCAGG | VOPP1 | NM_030796 | Body | | | 1.26E-27 | 13.29063 | 0.69393 | 0.30995 |
| | GGAAGTGAGAATTGGC | | | | | | | | | |
| | CCAAGCCACAGGTGACC | | | | | | | | | |
| cg07230581 | CGGCAGTTTACACAGGA | OPCML | NM_001012393 | TSS1500 | | | 1.97E-27 | 13.43349 | 0.75470 | 0.42139 |
| | TGGCAGTATCTCTGGGT | | | | | | | | | |
| | GTAAGAAACCTCAGAA | | | | | | | | | |
| cg00387964 | CGCAGCAATTGAGTGAT | SORCS2 | NM_020777 | Body | chr4: | S_Shelf | 1.06E-28 | 13.76804 | 0.80972 | 0.38929 |
| | TCACCCAGGGTCACCCC | | | | 7647755- | | | | | |
| | CTGTCAGACCTGCACT | | | | 7647960 | | | | | |
| cg03315407 | CTCATGAAGCTCAGAAT | ANKH | NM_054027 | Body | | | 4.51E-29 | 13.93857 | 0.61837 | 0.27165 |
| | TGGAAATTGCTTCTTCCC | | | | | | | | | |
| | TCCTGTAGAGAAACG | | | | | | | | | |

SUPP. TABLE S6 (SEQ ID NO: 81-160) (Forward and reverse primers for the 40 CpG signature)

| UCSC_ | | | | |
|---|---|---|---|---|
| RefGene_Name | CpG ID | Chr | Forward primer _F1 | Reverse primer_R1 |
| ALX3 | cg06215569 | 1 | GGTAAGGGTGGTTTTGATAA | TATTTAATCTACTCCCTCCCTCTTTATCC |
| | | | | TTT |
| ANKH | cg03315407 | 5 | TAAGAGTGAAATTTTGTTTTAAAAA | TCCTAACTCTAACTTCCTAAATCTC |
| C3AR1 | cg04499514 | 12 | GAGGTTAGATAGTGGTTTAGAGTATAAGAT | AACTTCAACACCTAAATATCTCCAC |
| C5orf56 | cg03653573 | 5 | TGGGGTTTTTTGTTATTTTGGTTGT | AACCCACAAACCACTTCCTCTACTC |
| CACNA1C | cg02468320 | 12 | GTAGTAGGTGGGGTAGGGTGGTTTT | AAAAAAAACTAAAATAAAACACAAACCAAA |
| CCDC140 | cg16325502 | 2 | AAATTTGGTATTAGGATTTTTTGGTTTAT | CAACAAAATTAACTAAAACTACCTAACCTA |
| CCDC140 | cg08697503 | 2 | GAAATTTTTGTGTTAGTGTTTTTAAGTG | AAAAAAATAACTTCTATTATCCCCTCTAC |
| CCDC140 | cg14064356 | 2 | GTTAGTTTATGAATTTATGGGTATTTAAGA | TTAAAAAACATCTAAAACTAAATCTCATTT |
| CCDC19 | cg09476130 | 1 | AAGTTTGGTTAAAGTTAAAGTGGAGAGTAG | TTACCTTAACAACCACTAACCC |
| CYTIP | cg10559416 | 2 | GGTTATTTAATTATGTGTTAGTTGGAGTGA | ACTCTACCCTCAAAAAAATATAAACACTCT |
| DYNC111 | cg17889682 | 7 | TTGTAGAGGGAGGGGAAAGGATGTT | CTACCAAAATCACTACCCTTATAATAAC |
| EPB41L4A | cg15536663 | 5 | TTAGTTTGTGGTGTAGTTGGGTAGATATTA | AACAAACCATTCCCACAATAAATAAC |
| FAIM3 | cg15158847 | 1 | AAGATAAAGGAATATTAGGTTTGGT | CCATCCAAAAACCCCTAAAAA |
| FLJ22536 | cg07553475 | 6 | TTAATTTTGGAGGTTGAGAATGATTGGAAG | AAAACAAAACTCTCAAAATAACCAAAC |
| GIMAP7 | cg15849098 | 7 | TGTTTTGTTTTTTAGGTAATATTTGGGTTA | AAAACCACACACACAAAAATATTTATCTTT |
| GOLIM4 | cg18098839 | 3 | GAAGTGGAGGAATATTTGGTGGTA | AAAAAATAATAAATAATCATTCCCAATACA |
| HOXD12 | cg03874199 | 2 | TAATTTGATTTGGTTTTGTTGGTAGTT | CTCACACATCTCCAACAAAAAAC |
| KREMEN1 | cg26967305 | 22 | ATTATGGTTTAATTTTAAGAGGGATT | CTACTTCCTATAAAATCCCCCAAC |
| LIPC | cg02744046 | 15 | TTGGTTTTGTTGTTTTATTGAGAGT | AAAAACATTTCTCCATATTTCATTATTATA |
| MAS1L | cg12423733 | 6 | AAGGAATTTTTTAGAGTGATTTTTTAA | CTATATATACAATTCCCCAACTCAAATATA |
| MBP | cg07637837 | 18 | AATTAGGATATGTTCGATTTTTCGT | CCAAAAATATAATTATAACACTCTATATTCG |
| | | | | A |
| MYT1L | cg17918270 | 2 | TGTAGGGTTGGTTTGTATAGGTAGG | TTCCACAAAAAAATTACCAAAAAAATA |
| NBLA00301/ | cg16919569 | 4 | TTTAAGATTTTAGGGTTAGTGGAGGGTAGA | CCCAAAAAAAACCAAAAACTCCACCTTAA |
| HAND2 | | | | |
| NRXN1 | cg22322562 | 2 | TGTGTTTAGTAATTATATTGGATTTGAATG | AAACTATTAATACACAACCCAACCC |
| ONECUT1 | cg07569216 | 15 | TTTTTGGGAAGTTATAGTAAGAAAATAAAA | TACACTCAAATACTCACACAAAACC |
| OPCML | cg07230581 | 11 | AGGAATTTAAGTTATTTGAGGTTTT | CTATCCCTTCCTCTAAAATAACAAT |
| PAX3/CCDC140 | cg19352038 | 2 | TTTTTGATTGATTAAGGTTTTGAATAT | AACTAAAATATCCCCAACAAAATATAAC |
| PAX3/CCDC140 | cg18077971 | 2 | TTTTTATTTTAAAGGGAAAAATTTGTT | CCTTAAAAAAAATACCATTATACATAACCT |
| PROM1 | cg13164157 | 4 | GGTGAGTAGTTGGGTTTTTATTT | CAATTCCTCTAACCCCCAAC |
| RASGEF1C | cg08898055 | 5 | AGGGTAGTGAGTTTGGTTAGGG | AACCAAAAAACAACTACAAAAAAAA |
| SGEF | cg06573459 | 3 | AATAATTAAAATGTGGAGTTTTATAAGAGA | TCAAAACCACTAACCACTACCCTAC |
| SHANK3 | cg18851100 | 22 | GGTTAAGGTTGGTTTTTGTGGGAGG | AAAAAAAACAAAAAACCCAAAACCC |
| SHOX2 | cg12993163 | 3 | TTGTATGAAAAGGTTTTGAGTAATTAATA | TCCCCTAAACAACCAAATAATCTCC |
| | | | GAA | |
| SIX6 | cg13019491 | 14 | TGGTGGGGTATAATAGTAGGGATT | CATCCTAAATAAACAACTCAAATATC |
| SORCS2 | cg00387964 | 4 | GGAAATTGTTGTGGTTAAATGATTTATTTT | TTTACCTCTCAAAATACCCCCACA |
| TBX5 | cg18689332 | 12 | GATATTTTAGTAACGCGAGGATCGGC | CGTAAAAACGAAAACTAACCCCGTT |
| TLR1 | cg08757862 | 4 | AGGGGAAATAGAGAGAGATAGTTAGAATAT | AACCTACATAATATCCAATCAAAACC |
| TLX3 | cg05787556 | 5 | GGAAGAATTTAGGTTAGGGGTGCGA | TATCTACCCGACCCAAAACACCGTA |
| VOPP1 | cg08337633 | 7 | TTAGAGTGAAATTTTGGGTAGTTTT | ACTATCAAAAAATAATCATCTCTTACTTCA |
| ZBTB38 | cg02936049 | 3 | TTTTGGGATTTAGTGTTTTGATTTT | CATTTTAACCTATTTCTACCACTTTAAC |

SUPP. TABLE S7 (SEQ ID NO: 161-219) (Sequences associated with 59 CpG signature)

| | | Genome_ | | |
|---|---|---|---|---|
| CpG Name | Forward_Sequence | Build | CHR | MAPINFO |
| cg00295418 | CTTTCTTTGCCTGCTGGGGTGATTTTGGATGCAAACCTTCCGTGTTAACGCTCTTTCAGA[CG]TGCTGTTGAAAGAGTCCAAGTGGAC | 37 | 8 | 2021420 |
| | GAAGATGTTCTTTGGAGAAGGCCAGGCCTCCCTGT | | | |
| cg00387964 | ACAGATAATTCAAGTGCAGGTCTGACAGGGGGTGACCCTGGGTGAATCACTCAATTGCTG[CG]AGCCTCCATCTTCCGTCTGCACGA | 37 | 4 | 7651935 |
| | GGTATTGTTAGAAGCATCTACTTCCTGGCGATGTTG | | | |
| cg00916635 | GAGCAAGAAAAATAGTCTATAAGTAGGTTGAGGGAGGGCATGTCAAATGTGTGTCATGGC[CG]AGACACCTCCAAAAAGCCACTGC | 37 | 1 | 114414312 |
| | TGCCGCCTGAGCAGAGCATGCTGAAGGCTGTGGTTTA | | | |
| cg01725872 | TCTCTGGGACTCAGTTTCCCCAACGGTGAATGAAGGGGTGAATCTGGAAGGCTGACATTC[CG]TACTCAGCAATGCTGTCACCCCCT | 37 | 22 | 45635600 |
| | CAGAAATCCCCAGCCTAGCCTGGGGGTGGGGTGGGG | | | |
| cg01975505 | CCCTGACCCCAGCCCCCGCAAGGCCCCTTCTGTGTACACTTGACTGAATTTTATGGGCCT[CG]TGGATATGCATGGGCTTATCTCCAG | 37 | 19 | 48497828 |
| | ACATCATAAATTAACCACAGCTGATCTCATCCAGA | | | |
| cg02192204 | GATGCAGTTCAGGAGCTGGGGGCGGGCGGCGCAGCCTTCAGCACCTCAGAGGGACCGGGA[CG]CACCAGACACTCCCCCAGCCCA | 37 | 16 | 88947943 |
| | CTCGGCCAGAGCCCCGGACAGGATGGGTGCCCGACCAG | | | |
| cg02468320 | GTGTAATCTCCGTGGTCAGTGAAGCAATGCTGTGCGCACAGTTCTGTGTTGTGCCTCTCC[CG]GGGAAGGGGTGTATTTGGCCTGTG | 37 | 12 | 2404134 |
| | CCCCACCCTAGCCCTCCTTCGTCTTCCCTCTTTCAC | | | |
| cg02585849 | TTCCATTTCTTTTCTTTTCTCCTCTCTCTCTCTCTTTCTCTCTGTCTCTCCTCCACTCCC[CG]ACCCCCAACTAGGATGCATCCTGTAAAGC | 37 | 12 | 126014887 |
| | TCCATCTGGTTTGGGGGTGGGAAGTGGGTTT | | | |
| cg02936049 | AGACCACGAGCAAGTAAGCACGTTAATCAAAGTGAAAGGCTCACCCCTCACGTCTAGCTC[CG]TCCTTCTCCAGCCTGTGCCTGCCAG | 37 | 3 | 141102599 |
| | ATTATTTCGGGTTCCTCGTGTTTGACTCGTCAAAG | | | |
| cg03315407 | CACTAGTGCCTGTTTTCCTGACTCTGACTTCCTGGGTCTCGGCACCACAGATAGCTTCTG[CG]TTTCTCTACAGGAGGGAAGAAGCAA | 37 | 5 | 14810180 |
| | TTTCCAATTCTGAGCTTCATGAGGGAGGAGAATAA | | | |
| cg03874199 | GATGTAGGCGGTGCTGAAATGACCGGCTTTGAAGAACCTGCAGGCAAAGTTTCGTCCAAT[CG]TCTGAGCCTGTCCTCTTATTCCCG | 37 | 2 | 176964456 |
| | GTTGTAACTAAATACTGTTGCGAGCGCAGCCGAAGC | | | |
| cg04131969 | GGCCCAATTCCCACTCCCCCAAACACACACAAGTACACACTGACTAAGGCACAGCTAGGG[CG]GGGGCGGGCAGAAGGCCCCTTGG | 37 | 2 | 33951647 |
| | GAGGACGTGGCGCCACAGCTGCAATGGGTGTGGGGGT | | | |
| cg04499514 | TCATGAAGTATGGCAAGAAAATTTGCTGAGCTTTCTCTTCTTCTGTTCTCTCTCTGTTTC[CG]GCAATAAGTTAAGTCTTATGCTCTAGA | 37 | 12 | 8219020 |
| | CCACTATCTGACCTCACAGGAAGAGTTTCAAAG | | | |
| cg05208607 | GGCTTGACTTCTCCCACGCCCCATAGACCCGGCACCGTGTAATAACTGGGCCCGTGTCCT[CG]CCTGAAAACTGGGGGTCACACGGC | 37 | 16 | 84520021 |
| | CTGTCCTGAAGAACTCTGATGTGATAAACACCATAG | | | |
| cg05594873 | TCATGGGAGAGGTATAGGTCCATAAGAAACAAAGTCATCTTACCAACTGGATATCTGCTC[CG]AGCTGCTGCTGCTTCCTCTTCCTTT | 37 | 3 | 79814509 |
| | TTTTGGGGGGTGGGGGCCGATTTTGGAAGGGCAAA | | | |
| cg05787556 | TTCGCTGGAAGAAAATGATTCCGCTTGTCTCCCCAAAGCTGCAGCGGAAGGTGACTACTT[CG]TGTGCGGTCCTGTCCACGGTGCCC | 37 | 5 | 170735186 |
| | TGGGCCGGGTAGACAGTCACTGAGGCGCGAGCAGAA | | | |
| cg06215569 | ATTTCCCTTCCCCTTTTCTTGGTTGTCGCTCGCTTTCTTTGGTTTTCTTTCTCGGTATTT[CG]TTGTCAAGGCCACCCTTGCCGTCGGATC | 37 | 1 | 110611465 |
| | CCGGGGTGCTGGGTTTCTCCCGGCCGCTCGTT | | | |
| cg07637837 | CAAAAACCCGTAGAATGAACACCGTGCACACGCACACACACACACACACACACGTGCGCG[CG]CGGCAAAAAGAAACAGCTCATTT | 37 | 18 | 74824154 |
| | CGGAGCTGAGGACAAGGCGTGGGAAGAAGACGCGTTT | | | |
| cg07817686 | TTCCCTTAGCTCGCCAGACCCTGGCTCGTGAATTATTTATGACCCGGCTTCTGGGACCAC[CG]CGACGGCTTTCGGAGAGCCCGCCTC | 37 | 4 | 16085401 |
| | CCACTGCCGGCCGCGGAGGGGCTCAGGCGGCGCTG | | | |
| cg08258526 | AGGTGTCCGTCAGCTTCTGCAGTTTCTTGCGGTTCATGTCTGGCGCTCAGAGAATCGGGC[CG]CGGCGGGGGTCTCTGGGCGCGCG | 37 | 8 | 49647734 |
| | GCTACCGAGACCCTCGCGGGACCCCCGCGAGCCCTGG | | | |
| cg08331829 | CCTTTCTCTCAGCACTCAACCTAAAGTGTTTTCCCTCTCCCTCCCCATTATTCTCTAGCA[CG]TGATTCCTTCAAAGCCCTACCTTTGTGA | 37 | 20 | 10412596 |
| | TGAATGCTGTGATGTGCCACCCTGACCCCCCT | | | |
| cg08337633 | AGGTCCTGTCATGGTCACCTGTGGCTTGGGCCAATTCTCACTTCCCCTGAAGGGCAGCTG[CG]TGTAGGGAGCGGGGGCTGCCCAA | 37 | 7 | 55602109 |
| | AGTTTCACTCTGACTGGAGGTAAACTTAACATCATTT | | | |
| cg08657228 | CGTGGGCCTTGGGTTGTGTGGGATAATCCTGTCTTATTCCTATTGTTCCAATGTTCCATC[CG]GCTACTGCTGCCTCTAACAAAACTAA | 37 | 20 | 170641 |
| | CCCAGTTTGGAAGATAAATTAAGTCATTAGTCGA | | | |
| cg08697503 | TGGCTGTCCAGGCCTGAGTGGAGCGTGCCCTTGTTAGCTTGAAAGTTCTCCCTCGCAGCC[CG]TTTGGATGCGTGCGTCTACAGCCC | 37 | 2 | 223166946 |
| | AGTCGCACTTTGGTGACCGGCCTGGGCTGTGAAGCA | | | |
| cg08757862 | TTTTCTACCACACAGCGAGCAAGGCCAACTTCCCTAAACTAAGAATGCTGAGATTCTTTT[CG]ACTTATAATGTTCTGACTGTCTCTCT | 37 | 4 | 38807382 |
| | CTGTTTCCCCTTACCTCAGAATTTGTTTAATAGA | | | |
| cg08898055 | GGCGCGGGCCTGTTCTGTGAGGGAGAAAACAAGCGTCCTATTTACCACGAGAATGAATAT[CG]GGCTCTGTGTGAAAATCCCACTT | 37 | 5 | 179597395 |
| | GCTCTGAGATGTGTGAAGCCAGCAGGGCCAGGGACGC | | | |
| cg09120722 | CCTCCCTTCCTCCCGCTCATCCTGGCACCCACTGATCTTTCCACACGGCCTCCACAGTTT[CG]CCTTTTCACCATGCCCAGTTGATATCA | 37 | 4 | 186549051 |
| | TGCAGAATGTGGCCTCTCAGGTTGGCTTCCTGC | | | |
| cg09785377 | ACCATAATTTTTTTAAAAATTGAGGATGATCACAGCATCCTAGGAGCTTAGAGGTTACCA[CG]GTGACCAGAGCCAACATTGGCCAA | 37 | 15 | 60644157 |
| | GTTTGTCGTGGAACAGCCATACCACCTGTCCTGAAT | | | |
| cg09935388 | CAGGCTGGCCACTTCAGTGAGAGGCTGTACCCGGAGTTTCGTTCGGAGGGGTTCGGGGCG[CG]CCCAATCCTTGTCTGGCCACTTG | 37 | 1 | 92947588 |
| | ACGCCCTGGCAGGAAGAATCCTCCCGCCGCCGGCTCC | | | |
| cg10119160 | GGTTAATGTAATACAGGAGTGTAAGAATTTGTTCTTTCCACTAAAGAGAAAACAAGGCCA[CG]CATTCCCAATTATATGCTCACAAAG | 37 | 2 | 216859796 |
| | GGCAGCCAGGGAGTGCAGTTCTTGGCAGCAGCAGG | | | |
| cg11033617 | AATCATCAACTGTTCCCTGGCCCTGTTCTGTGCTGCATCCTAAGCCAAATGTCACAATCT[CG]AGATGGATTAGGGGATGCGGAGGA | 37 | 5 | 179562118 |
| | GGGAGAGGAGGCCATGAGGAGCAGAACAGAGGAGGG | | | |
| cg11523712 | GGCCTGTTTTCACATCATTCTGATCATCTCTGTCCTTCGTCTTCATTTTGCTGTGCAACT[CG]GGGAGCCGAGGAGAGGTGGCAAAAA | 37 | 2 | 176957055 |
| | CAGCGGTTGCCGAGACAAGGCGCAGGCCTTGGCGC | | | |
| cg12072972 | ACATATCATCCTTGGACACCAGGCAGTAGAAGTCTGTGCGGGCACTGTAGTTTCGCGAGC[CG]AGATCCGAGACGTCCACTTCGCTG | 37 | 11 | 405958 |
| | CTCCGGCTCTCTCCCAGCGAGACCCCACTGGTGTGC | | | |
| cg12515659 | ATAAAACAGATAAGGAGAAGGCTGTATCTAGGCTGAATGGCTGGCCAATGTTTTCCTCTC[CG]TCAGTATAAATAAAATGGATGGAA | 37 | 5 | 16614241 |
| | GAAAACACCCCTGGATACTATCAAATATGCCTTTCA | | | |
| cg12983971 | GCAGGGGAAGAGGGGAGACCTGGCACGTGCGGGAGGCCTGGAGAAGAGACAAGGAAACAG[CG]TGAGTATCGTAGGCACATAA | 37 | 22 | 45634621 |
| | AGACCTGGCTGGGGATATTCAGGGAGTGGAGGGCAGGCTC | | | |
| cg12993163 | GACAGCCAGGTAATCTCCGTCCCGCCTGCCCGACCGGGGTCGCACGAGCACAGGCGCCCA[CG]CCATGTTGGCTGCCCAAAGGGCT | 37 | 3 | 157821407 |
| | CGCCGCCCAAGCCGGGCCAGAAGGCAGGAGGCGGAAA | | | |
| cg13019491 | CCGTAGACTCCAGCAGCAGGTCCTGTCACAGGGTTCCGGGCGGGCACTACGGGCGGAGGG[CG]ACGGCACGCCAGAGGTGCTGG | 37 | 14 | 60977856 |
| | GCGTCGCCACCAGCCCGGCCGCCAGTCTATCCAGCAAGG | | | |
| cg13164157 | CTGCTGAGGGGCCAGGGAGGCGGCGCAGATGGCTAGGGTAAGGGGGGCGCAGAGCGAACC[CG]TCCACTCCTCACTGTACACCCC | 37 | 4 | 16085180 |
| | CAGTACAGTGGAAGGAGTGCGCTCAGCCCCGCGCCTGG | | | |
| cg13782322 | ATGCCGTCTGGCTTTGGCCATGAGACCCTCGTGTGACCAGGTGCGTGCCTAAGTTAGAAT[CG]CCCAGGCTAAGTTCGTGAACCCCC | 37 | 15 | 90756121 |
| | TGGATGAGGGAGGCCCGACTCCCGCAAGGAGCCCTG | | | |
| cg14064356 | CATCTAGAGCTGAGTCTCATTTGTTTTTGAGCCGGAGGCTTGGTCTCCAAGCCCTCCCAG[CG]TCCACCCGTCTCTCTCCTGCCGGGA | 37 | 2 | 223165753 |
| | GTTTTCTCTCCTAAGAGCCGGCAGATGCTGGAGGG | | | |
| cg14405813 | CCATCTTCTCATGGGAGTTTCAGTTTTGTTTCTTGTGACCTCTATCTCCACTGCACTGTT[CG]GCACCACCCCAAACACACCCCCAGGCT | 37 | 7 | 139414573 |
| | GGTTCCACAGAGCAGGGTCTTGCTTTCCATCCC | | | |
| cg15158847 | CACGCTGCTTACTCAGGAACCCTTCACAATCTGGAACTGGAAAGAGATTTCTAGCCCCCA[CG]AGGAACAAAGCTTGACGATGAGGA | 37 | 1 | 207095315 |
| | AATGACAACCTCCCTTGTTTGCTAACTATTCTCAGG | | | |
| cg15536663 | TTTCAGCACCCCACCCCCTCTTCAGTTGAAGGTAGCAAGCCATTCCCACAGTGGGTGGCC[CG]CAGGGTTATCTGCCACATCAAAAAG | 37 | 5 | 111665548 |
| | AGAGGCTTTATGGTACTCTACCAAGCATCCTTACA | | | |
| cg16113793 | TGACACAGGAAGTGGGCGCATAGCACTTGCTGTGGAAATCTGTGCCTGCCCCCCTGCCTA[CG]CTGGTGACTCTTGTCAGGTAGGAA | 37 | 18 | 21451607 |
| | GTTTTCCCATCCGCAACATTTCCCTAGGGAGCTCCT | | | |
| cg16325502 | GCCACTCTTTCTCTGTCTCCGAGTCTTGGGCCTCCCCTTTATTTCTTTCTGAAGTCTCTC[CG]GAGCCCAAGCCACCCCACACCCAAACC | 37 | 2 | 223166435 |
| | CCGCAGCTGGATGGGAGTCCAGGCCACTTCCCT | | | |
| cg18077971 | TATTTATAACTTGGTAAGTGCCAGCGAACTCGCCTCCTTTACACCCCCGAGTGCCAGCCC[CG]CGCTCTGCACTGCGCTTTATTCGCTC | 37 | 2 | 223164867 |
| | GAGCCTATTCAGGGACTGTCACTCCGGGGCCGCG | | | |
| cg18098839 | ACTAACCTTTTACATAAACCAGATGTTTCTTAAAATAGCCCAGTTAAATCCACCCTTCCT[CG]TGGCATCTGCTTACCACCAAATGTTCC | 37 | 3 | 167742700 |
| | TCCACTTCTGTATTCTCTTGCTTTTGATTACTT | | | |
| cg18689332 | AGGGAGGAGAAAGGCGAAGGGAGGAGGTAACAGCAGGCGGGCAACTGTAGGTAACCTAAG[CG]GAAAACAAACCAGGACGCAT | 37 | 12 | 114837666 |
| | GCGCCTCTAGAGAACGGGTTTTGAAGATGCTTCAAAGGGA | | | |
| cg18694313 | GAGAGGCTGCTCTGTGAGGAGCAGTGTTTTCCACAGCTGCTCTGAGAATTCAGTAGAAAT[CG]AGCCATTTGTTACTCAGAAGTCTA | 37 | 13 | 33224942 |
| | GCTGGATGGCAAGTGGAAATCGTTGCAGCATAAAGG | | | |
| cg19352038 | TTTATAACTTGGTAAGTGCCAGCGAACTCGCCTCCTTTACACCCCCGAGTGCCAGCCCCG[CG]CTCTGCACTGCGCTTTATTCGCTCGA | 37 | 2 | 223164869 |
| | GCCTATTCAGGGACTGTCACTCCGGGGCCGCGAG | | | |
| cg21966754 | ACATTCAGGAAACATGTTGATGTTGCTGATTGCAACATGCTCCTTACACACACCAGTGTT[CG]AGCACTTGACTCACAGGAAATGCTC | | 19 | 54584816 |
| | CTCTGTCTCAGGCAGATTTCAGGCATCAAACAGGT | | | |
| cg22322562 | CCGGCACCACTCAAAAAGTCTCAGCAGTCGTTGCTTTTCAATTTGCTCCCCTAACGAGAC[CG]CATAGGTAAACAGACCTCCCTCTAA | 37 | 2 | 50201511 |
| | CCCCCGACCGAAAAAAAGGCTTATTTTCATGCACG | | | |
| cg23350716 | TATTCACCCAGGATATGTTATTGTGTGCTTAGCCTTGGACTTTGTTGCTTGTGTGTTGGA[CG]CCTAATAGTCTTGACAGCTAAATAG | 37 | 1 | 147956744 |
| | GCTTTTGTAAGCGAGAGTGGTAAAGTCCAACATGT | | | |
| cg24107163 | AACCCTTCACAATCTGGAACTGGAAAGAGATTTCTAGCCCCCACGAGGAACAAAGCTTGA[CG]ATGAGGAAATGACAACCTCCCTTG | 37 | 1 | 207095332 |
| | TTTGCTAACTATTCTCAGGCTAAAAAGAGAGTGCTG | | | |
| cg24874003 | CTGTGGCCTCGCACAGGCGGACAGACGGGCAGCAGGGATCTCCAACCGGGCCCCGGAGCA[CG]AACCACTACGCAATCGTCACAG | 37 | 19 | 2602614 |
| | CATGTGCTACCTTCCGTGGTGTTCTGGGAATCTAAATC | | | |
| cg25790133 | CATAACTGGTCTATTCTGTTCTCTTTTTAAAACAGAGCCAAGATTTTCTTCTTCACTCCT[CG]CTTGGTGGCTCCCAGCCAGAGGCCGG | 37 | 4 | 2627014 |
| | CAGTGGTGGGAGGCTCGCTCTGGGTGCACAGACG | | | |
| cg25975621 | TGGGACGCCTGGCCAAATGCGGGGCGGTCTCTCGCGGGCCATTGGCTTGGGCCACCGTTC[CG]AGTCAGCTCCTAGGATTTCCCCAG | 37 | 1 | 217311177 |
| | GCTTTGCGGCCCCTTTGTGGGTCTAGGCCAGCGCCT | | | |
| cg26579713 | ACTGAGTCATGAGACCAACTCAGAGACCACAGATGCAAATGAGCCTTGTGGTTTCTGGTA[CG]CTGCATACACATCTGGACCTACCC | | 15 | 65701865 |
| | AGGAAGCCACAAGAGGGACCAAGTCCAAGGTCTAGC | | | |
| cg26820259 | GTCCCACTGCCCATTCTGATTCACATCCCCCAATTCCTGATCATGTTTGTCTACGTCTGG[CG]ATACCTGACCAGAAGACGTCCTTATC | 37 | 6 | 51953096 |
| | TTCTATCCTCTCCCTTTCCTTTGAGGGAGAAGTC | | | |

SUPP. TABLE S8 (SEQ ID NO: 220-278)(Sequences associated with the 59 CpG signature)

| | | | | | | Re- | | | | | |
|---|---|---|---|---|---|---|---|---|---|---|---|
| | | | | | | lation_ | | | | | |
| | | | | | UCSC_ | to_ | | | | | |
| | | UCSC_ | | UCSC_ | CpG_ | UCSC_ | | | | Mean ß | Mean ß |
| CpG | | RefGene_ | UCSC_ | RefGene_ | Islands_ | CPG_ | t-test p | t-test q | t. | in | in |
| Name | SourceSeq | Name | RefGene_Accession | Group | Name | Island | value | value | statistics | nevi | melanoma |
| cg00295418 | GCTGGGGTGATTTTGGA | MYOM2 | NM_003970 | Body | | | 3.48E-02 | 5.90E-01 | 2.13104 | 0.57969 | 0.47685 |
| | TGCAAACCTTCCGTGTT | | | | | | | | | | |
| | AACGCTCTTTCAGACG | | | | | | | | | | |
| cg00387964 | CGCAGCAATTGAGTGAT | SORCS2 | NM_020777 | Body | chr4: | S_Shelf | 1.06E-28 | 3.29E-06 | 13.76804 | 0.80972 | 0.38929 |
| | TCACCCAGGGTCACCCC | | | | 7647755- | | | | | | |
| | CTGTCAGACCTGCACT | | | | 7647960 | | | | | | |
| cg00916635 | TTCAGCATGCTCTGCTC | PTPN22 | NM_012411; NM_012411; | 5′UTR; | | | 5.13E-18 | 5.12E-04 | 9.80750 | 0.78035 | 0.48516 |
| | AGGCGGCAGCAGTGGC | | NM_015967; NM_015967 | 1stExon | | | | | | | |
| | TTTTTGGAGGTGTCTCG | | | | | | | | | | |
| cg01725872 | CCCAGGCTAGGCTGGG | C22orf9 | NM_001009880 | Body | chr22: | N_Shore | 9.73E-24 | 2.78E-05 | -11.97531 | 0.31778 | 0.60066 |
| | GATTTCTGAGGGGGTG | | | | 45636070- | | | | | | |
| | ACAGCATTGCTGAGTAC | | | | 45636606 | | | | | | |
| | G | | | | | | | | | | |
| cg01975505 | CGAGGCCCATAAAATTC | ELSPBP1 | NM_022142 | TSS200 | | | 3.52E-15 | 6.24E-03 | 9.11901 | 0.43038 | 0.17456 |
| | AGTCAAGTGTACACAGA | | | | | | | | | | |
| | AGGGGCCTTGCGGGGG | | | | | | | | | | |
| cg02192204 | CGCACCAGACACTCCCC | CBFA2T3 | NM_175931; NM_005187 | Body | chr16: | Island | 1.61E-15 | 6.97E-03 | -8.86408 | 0.40689 | 0.65423 |
| | CAGCCCACTCGGCCAGA | | | | 88947776- | | | | | | |
| | GCCCCGGACAGGATGG | | | | 88948015 | | | | | | |
| cg02468320 | TGGTCAGTGAAGCAATG | CACNA1C | NM_001129844; NM_001129827; | Body | | | 6.65E-24 | 8.85E-08 | 12.10212 | 0.78885 | 0.44127 |
| | CTGTGCGCACAGTTCTG | | NM_001129839; NM_001129834; | | | | | | | | |
| | TGTTGTGCCTCTCCCG | | NM_001129841; NM_000719; | | | | | | | | |
| | | | NM_001129830; NM_001167625; | | | | | | | | |
| | | | NM_001129843; NM_001167624; | | | | | | | | |
| | | | NM_001129835; NM_001129837; | | | | | | | | |
| | | | NM_001167623; NM_001129840; | | | | | | | | |
| | | | NM_199460; NM_001129833; | | | | | | | | |
| | | | NM_001129832; NM_001129829; | | | | | | | | |
| | | | NM_001129846; NM_001129836; | | | | | | | | |
| | | | NM_001129838; NM_001129831; | | | | | | | | |
| | | | NM_001129842 | | | | | | | | |
| cg02585849 | CACCCCCAAACCAGATG | TMEM132B | NM_052907 | Body | chr12: | N_Shelf | 2.67E-16 | 1.37E-02 | 9.15540 | 0.68691 | 0.43451 |
| | GAGCTTTACAGGATGCA | | | | 126018100- | | | | | | |
| | TCCTAGTTGGGGGTCG | | | | 126018365 | | | | | | |
| cg02936049 | CGGAGCTAGACGTGAG | ZBTB38 | NM_001080412 | 5′UTR | | | 1.54E-41 | 2.08E-08 | -18.52521 | 0.24379 | 0.64387 |
| | GGGTGAGCCTTTCACTT | | | | | | | | | | |
| | TGATTAACGTGCTTACT | | | | | | | | | | |
| cg03315407 | CTCATGAAGCTCAGAAT | ANKH | NM_054027 | Body | | | 4.51E-29 | 7.83E-05 | 13.93857 | 0.61837 | 0.27165 |
| | TGGAAATTGCTTCTTCCC | | | | | | | | | | |
| | TCCTGTAGAGAAACG | | | | | | | | | | |
| cg03874199 | GCTGAAATGACCGGCTT | HOXD12 | NM_021193 | TSS200 | chr2: | Island | 1.14E-24 | 2.02E-06 | -13.11382 | 0.17066 | 0.55695 |
| | TGAAGAACCTGCAGGC | | | | 176964062- | | | | | | |
| | AAAGTTTCGTCCAATCG | | | | 176965509 | | | | | | |
| cg04131969 | ACTCCCCCAAACACACA | MYADML | NR_003143 | Body | chr2: | N_Shore | 5.74E-01 | 8.99E-01 | -0.56332 | 0.51820 | 0.54598 |
| | CAAGTACACACTGACTA | | | | 33952422- | | | | | | |
| | AGGCACAGCTAGGGCG | | | | 33952684 | | | | | | |
| cg04499514 | GCAAGAAAATTTGCTGA | C3AR1 | NM_004054 | TSS200 | | | 1.58E- | 2.68E-04 | 11.07884 | 0.76832 | 0.42028 |
| | GCTTTCTCTTCTTCTGT | | | | | | 21 | | | | |
| | TCTCTCTCTGTTTCCG | | | | | | | | | | |
| cg05208607 | CGCCTGAAAACTGGGG | KIAA1609 | NM_020947 | Body | | | 1.84E-01 | 9.95E-01 | 1.33321 | 0.75199 | 0.68854 |
| | GTCACACGGCCTGTCCT | | | | | | | | | | |
| | GAAGAACTCTGATGTGA | | | | | | | | | | |
| cg05594873 | CGGAGCAGATATCCAGT | ROBO1 | NM_002941 | 5′UTR | chr3: | N_Shore | 6.73E-29 | 4.26E-05 | 13.79670 | 0.71513 | 0.36229 |
| | TGGTAAGATGACTTTGT | | | | 79815638- | | | | | | |
| | TTCTTATGGACCTATA | | | | 79815900 | | | | | | |
| cg05787556 | AAATGATTCCGCTTGTC | TLX3 | NM_021025 | TSS1500 | chr5: | Island | 8.04E-33 | 1.41E-07 | -15.88562 | 0.18489 | 0.56603 |
| | TCCCCAAAGCTGCAGCG | | | | 170735169- | | | | | | |
| | GAAGGTGACTACTTCG | | | | 170739863 | | | | | | |
| cg06215569 | CTTTTCTTGGTTGTCGCT | ALX3 | NM_006492 | Body | chr1: | Island | 1.01E-32 | 2.94E-08 | -15.89389 | 0.15243 | 0.62284 |
| | CGCTTTCTTTGGTTTTCT | | | | 110610265- | | | | | | |
| | TTCTCGGTATTTCG | | | | 110613303 | | | | | | |
| cg07637837 | TTCCCACGCCTTGTCCTC | MBP | NM_001025101; | 5′UTR | chr18: | Island | 1.16E-23 | 1.93E-06 | 12.25545 | 0.76116 | 0.42981 |
| | AGCTCCGAAATGAGCTG | | NM_001025100 | | 74824149- | | | | | | |
| | TTTCTTTTTGCCGCG | | | | 74824414 | | | | | | |
| cg07817686 | GCCAGACCCTGGCTCGT | PROM1 | NM_001145848; NM_001145847; | 5′UTR; | chr4: | Island | 4.83E-30 | 2.09E-05 | -15.84709 | 0.14450 | 0.43897 |
| | GAATTATTTATGACCCG | | NM_001145847 | 1stExon | 16084195- | | | | | | |
| | GCTTCTGGGACCACCG | | | | 16085735 | | | | | | |
| cg08258526 | GCTTCTGCAGTTTCTTGC | EFCAB1 | NM_001142857; NM_024593; | 5′UTR; | chr8: | Island | 1.07E-23 | 4.85E-05 | -11.93812 | 0.20693 | 0.56413 |
| | GGTTCATGTCTGGCGCT | | NM_001142857; NM_024593; | 1stExon; | 49647702- | | | | | | |
| | CAGAGAATCGGGCCG | | NR_024605 | Body | 49647988 | | | | | | |
| cg08331829 | GTGGCACATCACAGCAT | MKKS | NM_170784;NM_018848 | 5′UTR; | chr20: | N_Shore | 2.52E-01 | 9.48E-01 | 1.14944 | 0.49343 | 0.43940 |
| | TCATCACAAAGGTAGGG | | | TSS200 | 10414276- | | | | | | |
| | CTTTGAAGGAATCACG | | | | 10414993 | | | | | | |
| cg08337633 | CGCAGCTGCCCTTCAGG | VOPP1 | NM_030796 | Body | | | 1.26E-27 | 4.56E-07 | 13.29063 | 0.69393 | 0.30995 |
| | GGAAGTGAGAATTGGC | | | | | | | | | | |
| | CCAAGCCACAGGTGACC | | | | | | | | | | |
| cg08657228 | GTTGTGTGGGATAATCC | DEFB128 | NM_001037732 | TSS1500 | | | 7.68E-01 | 6.97E-01 | -0.29569 | 0.39371 | 0.40575 |
| | TGTCTTATTCCTATTGTT | | | | | | | | | | |
| | CCAATGTTCCATCCG | | | | | | | | | | |
| cg08697503 | CGGGCTGCGAGGGAGA | CCDC140 | NM_153038 | 5′UTR | chr2: | N_Shore | 7.26E-38 | 3.16E-08 | -17.63999 | 0.20261 | 0.62629 |
| | ACTTTCAAGCTAACAAG | | | | 223167205- | | | | | | |
| | GGCACGCTCCACTCAGG | | | | 223167560 | | | | | | |
| cg08757862 | CAGCGAGCAAGGCCAA | TLR1 | NM_003263 | TSS1500 | | | 1.34E-14 | 2.59E-04 | 8.55054 | 0.81200 | 0.53167 |
| | CTTCCCTAAACTAAGAA | | | | | | | | | | |
| | TGCTGAGATTCTTTTCG | | | | | | | | | | |
| cg08898055 | CGGGCTCTGTGTGAAAA | RASGEF1C | NM_175062 | 5′UTR | | | 4.69E-23 | 6.46E-06 | -11.94232 | 0.19711 | 0.51529 |
| | TCCCACTTGCTCTGAGA | | | | | | | | | | |
| | TGTGTGAAGCCAGCAG | | | | | | | | | | |
| cg09120722 | CGCCTTTTCACCATGCCC | SORBS2 | NM_001145670; NM_001145673; | Body | chr4: | S_Shelf | 2.78E-01 | 8.32E-01 | 1.08809 | 0.63925 | 0.59305 |
| | AGTTGATATCATGCAGA | | NM_001145671; NM_001145675; | | 186544754- | | | | | | |
| | ATGTGGCCTCTCAGG | | NM_003603; NM_021069; | | 186545503 | | | | | | |
| | | | NM_001145672; | | | | | | | | |
| | | | NM_001145674 | | | | | | | | |
| cg09785377 | CGGTGACCAGAGCCAA | ANXA2 | NM_001136015;NM_0010028 | Body | | | 1.17E-01 | 1.00E+00 | 1.57826 | 0.71083 | 0.63941 |
| | CATTGGCCAAGTTTGTC | | 58;NM_004039;NM_0010028 | | | | | | | | |
| | GTGGAACAGCCATACCA | | 57 | | | | | | | | |
| cg09935388 | GGGAGGATTCTTCCTGC | GFI1 | NM_001127215;NM_0011272 | Body | chr1:929 | Island | 1.69E-33 | 3.27E-06 | -15.51734 | 0.28349 | 0.70576 |
| | CAGGGCGTCAAGTGGC | | 16;NM_005263 | | 45907- | | | | | | |
| | CAGACAAGGATTGGGC | | | | 9295260 | | | | | | |
| | G | | | | 9 | | | | | | |
| cg10119160 | CGCATTCCCAATTATAT | MREG | NM 018000 | Body | | | 4.57E-11 | 2.24E-02 | -7.09708 | 0.39643 | 0.60369 |
| | GCTCACAAAGGGCAGC | | | | | | | | | | |
| | CAGGGAGTGCAGTTCTT | | | | | | | | | | |
| cg11033617 | TTCCCTGGCCCTGTTCTG | RASGEF1C | NM_175062 | Body | chr5: | N_Shore | 5.68E-18 | 1.79E-04 | 9.79545 | 0.55980 | 0.28672 |
| | TGCTGCATCCTAAGCCA | | | | 179563199- | | | | | | |
| | AATGTCACAATCTCG | | | | 179563779 | | | | | | |
| cg11523712 | CATCATTCTGATCATCTC | HOXD13 | NM_000523 | TSS1500 | chr2: | Island | 5.07E-23 | 2.75E-04 | -12.03726 | 0.12688 | 0.48866 |
| | TGTCCTTCGTCTTCATTT | | | | 176957054- | | | | | | |
| | TGCTGTGCAACTCG | | | | 176958279 | | | | | | |
| cg12072972 | CGGCTCGCGAAACTACA | SIGIRR | NM_001135054; NM_021805; | Body | chr11: | N_Shore | 1.19E-24 | 8.57E-05 | -12.28158 | 0.32638 | 0.66064 |
| | GTGCCCGCACAGACTTC | | NM_001135053 | | 406491- | | | | | | |
| | TACTGCCTGGTGTCCA | | | | 407871 | | | | | | |
| cg12515659 | CGGAGAGGAAAACATT | FAM134B | NM_001034850 | Body | chr5: | N_Shelf | 6.49E-01 | 2.07E-01 | -0.45607 | 0.42453 | 0.44462 |
| | GGCCAGCCATTCAGCCT | | | | 16616509- | | | | | | |
| | AGATACAGCCTTCTCCT | | | | 16617428 | | | | | | |
| cg12983971 | CACTCCCTGAATATCCCC | C22orf9 | NM_001009880 | Body | chr22: | N_Shore | 3.24E-23 | 1.57E-04 | -11.69144 | 0.37890 | 0.67652 |
| | AGCCAGGTCTTTATGTG | | | | 45636070- | | | | | | |
| | CCTACGATACTCACG | | | | 45636606 | | | | | | |
| cg12993163 | CGCCATGTTGGCTGCCC | SHOX2 | NM_001163678; NM_006884; | Body | chr3: | Island | 8.36E-33 | 1.46E-07 | -17.48068 | 0.12107 | 0.51557 |
| | AAAGGGCTCGCCGCCCA | | NM_003030 | | 157821232- | | | | | | |
| | AGCCGGGCCAGAAGGC | | | | 157821604 | | | | | | |
| cg13019491 | CGCCCTCCGCCCGTAGT | SIX6 | NM_007374 | Body | chr14: | Island | 1.14E-27 | 8.36E-08 | -15.52520 | 0.10943 | 0.51279 |
| | GCCCGCCCGGAACCCTG | | | | 60975732- | | | | | | |
| | TGACAGGACCTGCTGC | | | | 60978180 | | | | | | |
| cg13164157 | CTGAGCGCACTCCTTCC | PROM1 | NM_001145848; | 5′UTR | chr4: | Island | 2.48E-22 | 6.40E-06 | -12.75586 | 0.05858 | 0.44449 |
| | ACTGTACTGGGGGTGTA | | NM_001145847 | | 16084195- | | | | | | |
| | CAGTGAGGAGTGGACG | | | | 16085735 | | | | | | |
| cg13782322 | TTTGGCCATGAGACCCT | SEMA4B | NM_020210; NM_198925 | Body | | | 1.13E-21 | 8.96E-05 | -11.13136 | 0.38997 | 0.70568 |
| | CGTGTGACCAGGTGCGT | | | | | | | | | | |
| | GCCTAAGTTAGAATCG | | | | | | | | | | |
| cg14064356 | CGCTGGGAGGGCTTGG | CCDC140 | NM_153038 | 5′UTR | chr2: | N_Shore | 1.78E-30 | 2.41E-07 | -15.72046 | 0.21395 | 0.62450 |
| | AGACCAAGCCTCCGGCT | | | | 223167205- | | | | | | |
| | CAAAAACAAATGAGACT | | | | 223167560 | | | | | | |
| cg14405813 | GGGAGTTTCAGTTTTGT | HIPK2 | NM_001113239;NM_022740 | Body | chr7: | N_Shore | 8.45E-18 | 4.22E-03 | -9.71093 | 0.43602 | 0.69349 |
| | TTCTTGTGACCTCTATCT | | | | 139416286- | | | | | | |
| | CCACTGCACTGTTCG | | | | 139416522 | | | | | | |
| cg15158847 | TAGCAAACAAGGGAGG | FAIM3 | NM_001142472; NM_005449; | 5′UTR; | | | 1.91E-21 | 2.14E-03 | 11.07534 | 0.78427 | 0.48757 |
| | TTGTCATTTCCTCATCGT | | NM_001142473; NM_005449; | 1stExon | | | | | | | |
| | CAAGCTTTGTTCCTCG | | NM_001142473 | | | | | | | | |
| cg15536663 | TGGTAGAGTACCATAAA | EPB41L4A | NM_022140 | Body | | | 3.44E-20 | 7.87E-05 | 10.58951 | 0.64437 | 0.34814 |
| | GCCTCTCTTTTTGATGTG | | | | | | | | | | |
| | GCAGATAACCCTGCG | | | | | | | | | | |
| cg16113793 | CGCTGGTGACTCTTGTC | LAMA3 | NM_000227; NM_001127718; | TSS1500; | | | 4.04E-18 | 4.70E-03 | 9.83143 | 0.74045 | 0.47274 |
| | AGGTAGGAAGTTTTCCC | | NM_198129; NM_001127717 | Body | | | | | | | |
| | ATCCGCAACATTTCCC | | | | | | | | | | |
| cg16325502 | CGGAGCCCAAGCCACCC | CCDC140 | NM_153038 | 5′UTR | chr2: | N_Shore | 4.11E-39 | 1.07E-08 | -18.05654 | 0.19713 | 0.64446 |
| | CACACCCAAACCCCGCA | | | | 223167205- | | | | | | |
| | GCTGGATGGGAGTCCA | | | | 223167560 | | | | | | |
| cg18077971 | GTGACAGTCCCTGAATA | PAX3/ | NM_013942; NM_000438; | TSS1500; | chr2: | S_Shore | 3.27E-38 | 8.19E-08 | -18.02626 | 0.22592 | 0.66260 |
| | GGCTCGAGCGAATAAA | CCDC140 | NM_181460; NM_181457; | 5′ | 223162946- | | | | | | |
| | GCGCAGTGCAGAGCGC | | NM_181458; NM_153038; | UTR | 223163912 | | | | | | |
| | G | | NM_181461; NM_001127366; | | | | | | | | |
| | | | NM_181459 | | | | | | | | |
| cg18098839 | CATAAACCAGATGTTTC | GOLIM4 | NM 014498 | Body | | | 1.62E-18 | 6.93E-06 | 9.97188 | 0.66523 | 0.33785 |
| | TTAAAATAGCCCAGTTA | | | | | | | | | | |
| | AATCCACCCTTCCTCG | | | | | | | | | | |
| cg18689332 | ATCTTCAAAACCCGTTCT | TBX5 | NM_000192;NM_181486;NM | Body | chr12: | N_Shore | 4.76E-28 | 1.46E-07 | -13.51771 | 0.36317 | 0.72652 |
| | CTAGAGGCGCATGCGTC | | 080717;NM_080718 | | 114838312- | | | | | | |
| | CTGGTTTGTTTTCCG | | | | 114838889 | | | | | | |
| cg18694313 | CGAGCCATTTGTTACTC | PDS5B | NM 015032 | Body | | | 8.49E-20 | 4.55E-04 | 10.45887 | 0.53610 | 0.31837 |
| | AGAAGTCTAGCTGGATG | | | | | | | | | | |
| | GCAAGTGGAAATCGTT | | | | | | | | | | |
| cg19352038 | GAGTGACAGTCCCTGAA | PAX3/ | NM_013942; NM_000438; | TSS1500; | chr2: | S_Shore | 8.08E-42 | 2.22E-08 | -18.96571 | 0.29981 | 0.72751 |
| | TAGGCTCGAGCGAATAA | CCDC140 | NM_181460; NM_181457; | 5′ | 223162946- | | | | | | |
| | AGCGCAGTGCAGAGCG | | NM_181458; NM_153038; | UTR | 223163912 | | | | | | |
| | | | NM_181461; NM_001127366; | | | | | | | | |
| | | | NM_181459 | | | | | | | | |
| cg21966754 | CATGTTGATGTTGCTGA | TARM1 | NM_001135686 | TSS20 | | | 6.62E-22 | 1.39E-03 | 11.22237 | 0.69724 | 0.38804 |
| | TTGCAACATGCTCCTTAC | | | 0 | | | | | | | |
| | ACACACCAGTGTTCG | | | | | | | | | | |
| cg22322562 | CGGTCTCGTTAGGGGA | NRXN1 | NM_004801;NM_001135659; | Body | | | 2.33E-24 | 2.66E-08 | -12.28986 | 0.30412 | 0.66336 |
| | GCAAATTGAAAAGCAAC | | NM 138735 | | | | | | | | |
| | GACTGCTGAGACTTTTT | | | | | | | | | | |
| cg23350716 | CGCCTAATAGTCTTGAC | PPIAL4B/ | NM_001143883;NM_178230 | TSS15 | | | 5.35E-04 | 3.03E-01 | 3.53509 | 0.74649 | 0.60626 |
| | AGCTAAATAGGCTTTTG | PPIAL4A | | 00 | | | | | | | |
| | TAAGCGAGAGTGGTAA | | | | | | | | | | |
| cg24107163 | TTTAGCCTGAGAATAGT | FAIM3 | NM_001142472; NM_005449; | 5′UTR; | | | 3.40E-18 | 6.66E-03 | 9.91235 | 0.78582 | 0.51156 |
| | TAGCAAACAAGGGAGG | | NM_001142473; NM_005449; | 1stExon; | | | | | | | |
| | TTGTCATTTCCTCATCG | | NM_001142473 | 5′ | | | | | | | |
| | | | | UTR | | | | | | | |
| cg24874003 | CAGAACACCACGGAAG | GNG7 | NM_052847 | 5′UTR | | | 3.01E-23 | 2.58E-04 | -11.77389 | 0.29510 | 0.64370 |
| | GTAGCACATGCTGTGAC | | | | | | | | | | |
| | GATTGCGTAGTGGTTCG | | | | | | | | | | |
| cg25790133 | ATTCTGTTCTCTTTTTAA | FAM193A | NM_003704 | TSS200 | | | 5.79E-26 | 7.50E-05 | -14.58600 | 0.48266 | 0.87198 |
| | AACAGAGCCAAGATTTT | | | | | | | | | | |
| | CTTCTTCACTCCTCG | | | | | | | | | | |
| cg25975621 | AGACCCACAAAGGGGC | ESRRG | NM_001134285 | TSS200 | chr1: | Island | 7.24E-20 | 1.90E-05 | -10.75327 | 0.16622 | 0.51064 |
| | CGCAAAGCCTGGGGAA | | | | 217310749- | | | | | | |
| | ATCCTAGGAGCTGACTC | | | | 217311178 | | | | | | |
| | G | | | | | | | | | | |
| cg26579713 | CGCTGCATACACATCTG | IGDCC4 | NM_020962 | Body | | | 3.14E-17 | 3.13E-03 | 9.51120 | 0.62955 | 0.34350 |
| | GACCTACCCAGGAAGCC | | | | | | | | | | |
| | ACAAGAGGGACCAAGT | | | | | | | | | | |
| cg26820259 | CGCCAGACGTAGACAA | PKHD1 | NM_138694; NM_170724 | TSS1500 | | | 3.91E-01 | 8.71E-01 | 0.86108 | 0.55941 | 0.51564 |
| | ACATGATCAGGAATTGG | | | | | | | | | | |
| | GGGATGTGAATCAGAA | | | | | | | | | | |
| | T | | | | | | | | | | |

SUPP. TABLE S9 (Probes and primers for the 59 CpG Signature) (SEQ ID NO: 279-396)
59 CpG Signature

| UCSC_ | | | |
|---|---|---|---|
| RefGene_ | | | |
| Name | cg_ID | Forward Primer_BS | Reverse Primer_BS |
| ALX3 | cg06215569 | GGTAAGGGTGGTTTTGATAA | TATTTAATCTACTCCCTCCCTCTTTATCC |
| | | | TTT |
| ANKH | cg03315407 | TAAGAGTGAAATTTTGTTTTAAAAA | TCCTAACTCTAACTTCCTAAATCTC |
| ANXA2 | cg09785377 | TATTTAGGGGAGATAGAGATGGTTTAAA | AAAAAAACAACAAAAAATTATCAAATC |
| | | TATG | C |
| C22orf9 | cg01725872 | TATTATTGATGGATTATGTTTAGATTGAA | TTATTCTCTAAAACTCAATTTCCCC |
| | | G | |
| C22orf9 | cg12983971 | AGAGAGAGAGAGAGAGTTTGTTTAGGG | CAAAAACTAAATTCAACAACAAAAAAA |
| | | ATA | A |
| C3AR1 | cg04499514 | GAGGTTAGATAGTGGTTTAGAGTATAAG | AACTTCAACACCTAAATATCTCCAC |
| | | AT | |
| CACNA1C | cg02468320 | GTAGTAGGTGGGGTAGGGTGGTTTT | AAAAAAAACTAAAATAAAACACAAACC |
| | | | AAA |
| CBFA2T3 | cg02192204 | TGTAAGTTATGGAGGTGGGTTTTTTTT | CCACCATATCCATAATACAATTCAAAAA |
| | | | CT |
| CCDC140 | cg08697503 | GAAATTTTTGTGTTAGTGTTTTTAAGTG | AAAAAAATAACTTCTATTATCCCCTCTA |
| | | | C |
| CCDC140 | cg14064356 | GTTAGTTTATGAATTTATGGGTATTTAAG | TTAAAAAACATCTAAAACTAAATCTCAT |
| | | A | TT |
| CCDC140 | cg16325502 | AAATTTGGTATTAGGATTTTTTGGTTTAT | CAACAAAATTAACTAAAACTACCTAAC |
| | | | CTA |
| DEFB128 | cg08657228 | TTTTTTAAATTGGGTTAGTTTTGTTAGAG | AAAAAAAATTCCAAATCCACCCCC |
| | | G | |
| EFCAB1 | cg08258526 | GCGTACGAGATTAGTAATTGAGATTTTA | AACACAACAACGAACCTTAACACGA |
| | | TC | |
| ELSPBP1 | cg01975505 | TTTTTTATAGTTGAATTTTTGGGAA | AATTAAATAACTTTAACCCCAACCTATC |
| | | | TA |
| EPB41L4A | cg15536663 | TTAGTTTGTGGTGTAGTTGGGTAGATAT | AACAAACCATTCCCACAATAAATAAC |
| | | TA | |
| ESRRG | cg25975621 | TTGGATTTTGGAGGAGGGATGC | AACGCCTAACCAAATACGAAACGAT |
| FAIM3 | cg15158847 | AAGATAAAGGAATATTAGGTTTGGT | CCATCCAAAAACCCCTAAAAA |
| FAIM3 | cg24107163 | TAGATGTTTTTTGAATAGGGTGATTTTTT | CCATTATCCCTTCTAAAATACAAAATCC |
| | | T | AT |
| FAM134B | cg12515659 | GTATATTTGATAGTATTTAGGGGTGTTTT | AAACTTAAAAAACAAAACAATCATTTTT |
| | | T | AT |
| FAM193A | cg25790133 | GGGGGAAGGAATGAGTAGATTAGT | AACCACTTCAATAAAAAACTATACCC |
| GFI1 | cg09935388 | GGGGGAAGGAATGAGTAGATTAGT | AATTCAAACTAACCACTTCAATAAAAA |
| | | | ACT |
| GNG7 | cg24874003 | TTGTTTATAAAAGGTAATTTTGATTGAAG | ACAACAAATTCTACCAACTCCTCCC |
| | | G | |
| GOLIM4 | cg18098839 | GAAGTGGAGGAATATTTGGTGGTA | AAAAAATAATAAATAATCATTCCCAAT |
| | | | ACA |
| HIPK2 | cg14405813 | GTTTTGTGGAATTAGTTTGGGGGT | TTCCAACCTTCTCTCTATAACCTTAAAA |
| | | | AA |
| HOXD12 | cg03874199 | TAATTTGATTTGGTTTTGTTGGTAGTT | CTCACACATCTCCAACAAAAAAC |
| HOXD13 | cg11523712 | TTGAGGGATTTAGTAATAGGATAAAAA | ACCCATCCCAAACCCTATCTAC |
| IGDCC4 | cg26579713 | TTGGGTAGGTTTAGATGTGTATGTAG | AACAAAAAATTAAATCCAAAAAAAA |
| KIAA1609 | cg05208607 | TTTTATTTTTTTTAAGTGTTTTTTTAGA | AACATCTATAACTTAACTTCTCCCAC |
| LAMA3 | cg16113793 | GAGGTGGGGTTAGAGGAAGTTTTTGAT | CCCCAAACCATCCCACAATACTAAA |
| | | ATA | |
| MBP | cg07637837 | AATTAGGATATGTTCGATTTTTCGT | CCAAAAATATAATTATAACACTCTATAT |
| | | | TCGA |
| MKKS | cg08331829 | TTTGGAAAGGGTTTAATTTTAATTTTTTTT | ACCTTTCTCTCAACACTCAACCTAAAAT |
| | | | AT |
| MREG | cg10119160 | TGATGGGTAATGTTGAAGGTAAGTT | AAAAAAAATAACTCTATTCTCACCAAC |
| | | | AAA |
| MYADML | cg04131969 | TTTTTTTTGTTTTTAAGTATTTTTAG | AAAAAAATACACAACACACCTTCC |
| MYOM2 | cg00295418 | GTGTAGTTGTTGGGATTTTATTAGGTTG | ACAAAAAAACCTAACCTTCTCCAAAAA |
| | | AG | |
| NRXN1 | cg22322562 | TGTGTTTAGTAATTATATTGGATTTGAAT | AAACTATTAATACACAACCCAACCC |
| | | G | |
| PAX3/ | cg18077971 | TTTTTATTTTAAAGGGAAAAATTTGTT | CCTTAAAAAAAATACCATTATACATAAC |
| CCDC140 | | | CT |
| PAX3; | cg19352038 | TTTTTGATTGATTAAGGTTTTGAATAT | AACTAAAATATCCCCAACAAAATATAA |
| CCDC140 | | | c |
| PDS5B | cg18694313 | AGGTTGTTTTGTGAGGAGTAGTGTTTTTT | AAAAAAATAACCATCCAACATCCACTA |
| | | A | AAT |
| PKHD1 | cg26820259 | AAGGTGGAGATTTAGGGTTATTAAATTT | ATCAACAACACCTTCTTACTTAATCCAT |
| | | TA | AT |
| PPIAL4B | cg23350716 | TAGTAATATTGGTGGTAGTAGTAGAGAA | AAAATAAAAATAAATTCCATTTACAA |
| | | TA | |
| PROM1 | cg07817686 | TATGTTTAAGGAATTTTTTTTATTA | ATAAAAACCCAACTACTCACC |
| PROM1 | cg13164157 | GGTGAGTAGTTGGGTTTTTATTT | CAATTCCTCTAACCCCCAAC |
| PTPN22 | cg00916635 | GTGAGATGATGGTTGTGTTATGTGATTA | CATCCAAAAACTTCTACAAAATTTCTCT |
| | | TA | TT |
| RASGEF1C | cg08898055 | AGGGTAGTGAGTTTGGTTAGGG | AACCAAAAAACAACTACAAAAAAAA |
| RASGEF1C | cg11033617 | TTGTTTTGTTTTTTATGGTTTTTTTT | AAATAACCAAATATCTTCCCAACC |
| ROBO1 | cg05594873 | AAGGTAATTTGTAAGTATGTATTATGTTG | AAAAACTAAAAAAAATCCAAAAACC |
| | | A | |
| SEMA4B | cg13782322 | TTGTAGTTATTTTGGAGGTGATTTAAGTA | AAACTAACAAAAAAACAAAACTCCTTA |
| | | T | C |
| SHOX2 | cg12993163 | TTGTATGAAAAGGTTTTGAGTAATTAAT | TCCCCTAAACAACCAAATAATCTCC |
| | | AGAA | |
| SIGIRR | cg12072972 | TAAGGTTGTGGGTGGTTATTTTAGG | CAAAAATTATAATCCTATTAAACAAAA |
| | | | AAA |
| SIX6 | cg13019491 | TGGTGGGGTATAATAGTAGGGATT | CATCCTAAATAAACAACTCAAATATC |
| SORBS2 | cg09120722 | TGGAGGAGTTTTAAAAGTGTATTAT | AATATATATCCATCATTAATATATCAAT |
| | | | CA |
| SORCS2 | cg00387964 | GGAAATTGTTGTGGTTAAATGATTTATTT | TTTACCTCTCAAAATACCCCCACA |
| | | T | |
| TARM1 | cg21966754 | TGTTTGATGTTTGAAATTTGTTTGAGATA | TAACTCCTTAACCCTCCCAAAATCC |
| | | G | |
| TBX5 | cg18689332 | GATATTTTAGTAACGCGAGGATCGGC | CGTAAAAACGAAAACTAACCCCGTT |
| TLR1 | cg08757862 | AGGGGAAATAGAGAGAGATAGTTAGAA | AACCTACATAATATCCAATCAAAACC |
| | | TAT | |
| TLX3 | cg05787556 | GGAAGAATTTAGGTTAGGGGTGCGA | TATCTACCCGACCCAAAACACCGTA |
| TMEM132B | cg02585849 | GAATATTTAGGTTGTTTTTATTTTTTTT | AACATCATTTTTCCTACCTAACATAAC |
| VOPP1 | cg08337633 | TTAGAGTGAAATTTTGGGTAGTTTT | ACTATCAAAAAATAATCATCTCTTACTT |
| | | | CA |
| ZBTB38 | cg02936049 | TTTTGGGATTTAGTGTTTTGATTTT | CATTTTAACCTATTTCTACCACTTTAAC |

SUPP. TABLE S10 (Forward and reverse primers for the 40 CpG Signature) (SEQ ID NO: 397-476)
40 Cpg diagnostic Panel_Bisulfite Sequenceing Primers:

| UCSC_ | | | | |
|---|---|---|---|---|
| RefGene_ | | | | |
| Name | CpG ID | Chr | Forward primer F1 | Reverse primer _R1 |
| ALX3 | cg06215569 | 1 | GGTAAGGGTGGTTTTGATAA | TATTTAATCTACTCCCTCCCTCTTTATCCTTT |
| ANKH | cg03315407 | 5 | AGTTTGGGTGATAAGAGTGAAATTTTGTTTTAAA | TCCTAACTCTAACTTCCTAAATCTC |
| C3AR1 | cg04499514 | 12 | GAGGTTAGATAGTGGTTTAGAGTATAAGAT | AACTTCAACACCTAAATATCTCCAC |
| C5orf56 | cg03653573 | 5 | TGGGGTTTTTTGTTATTTTGGTTGT | AACCCACAAACCACTTCCTCTACTC |
| CACNA1C | cg02468320 | 12 | GTAGTAGGTGGGGTAGGGTGGTTTT | AAAAAAAACTAAAATAAAACACAAACCAAA |
| CCDC140 | cg08697503 | 2 | ATGAAATTTTTGTGTTAGTGTTTTTAAGTG | AAAAAAATAACTTCTATTATCCCCTCTAC |
| CCDC140 | cg14064356 | 2 | GTTAGTTTATGAATTTATGGGTATTTAAGA | TTAAAAAACATCTAAAACTAAATCTCATTT |
| CCDC140 | cg16325502 | 2 | AAATTTGGTATTAGGATTTTTTGGTTTAT | CAACAAAATTAACTAAAACTACCTAACCTA |
| CCDC19 | cg09476130 | 1 | AAGTTTGGTTAAAGTTAAAGTGGAGAGTAG | TTACCTTAACAACCACTAACCC |
| CYTIP | cg10559416 | 2 | GGTTATTTAATTATGTGTTAGTTGGAGTGA | ACTCTACCCTCAAAAAAATATAAACACTCT |
| DYNC111 | cg17889682 | 7 | TTGTAGAGGGAGGGGAAAGGATGTT | CTACCAAAATCACTACCCTTATAATAAC |
| EPB41L4A | cg15536663 | 5 | TTAGTTTGTGGTGTAGTTGGGTAGATATTA | AACAAACCATTCCCACAATAAATAAC |
| FAIM3 | cg15158847 | 1 | AAGATAAAGGAATATTAGGTTTGGT | CCATCCAAAAACCCCTAAAAA |
| FLJ22536 | cg07553475 | 6 | TTAATTTTGGAGGTTGAGAATGATTGGAAG | AAAACAAAACTCTCAAAATAACCAAAC |
| GIMAP7 | cg15849098 | 7 | TGTTTTGTTTTTTAGGTAATATTTGGGTTA | AAAACCACACACACAAAAATATTTATCTTT |
| GOLIM4 | cg18098839 | 3 | GAAGTGGAGGAATATTTGGTGGTA | AAAAAATAATAAATAATCATTCCCAATACA |
| HOXD12 | cg03874199 | 2 | TAATTTGATTTGGTTTTGTTGGTAGTT | CTCACACATCTCCAACAAAAAAC |
| KREMEN1 | cg26967305 | 22 | ATTATGGTTTAATTTTAAGAGGGATT | CTACTTCCTATAAAATCCCCCAAC |
| LIPC | cg02744046 | 15 | TTGGTTTTGTTGTTTTATTGAGAGT | AAAAACATTTCTCCATATTTCATTATTATA |
| MAS1L | cg12423733 | 6 | AAGGAATTTTTTAGAGTGATTTTTTAA | CTATATATACAATTCCCCAACTCAAATATA |
| MBP | cg07637837 | 18 | AATTAGGATATGTTCGATTTTTCGT | CCAAAAATATAATTATAACACTCTATATTCGA |
| MYT1L | cg17918270 | 2 | TGTAGGGTTGGTTTGTATAGGTAGG | TTCCACAAAAAAATTACCAAAAAAATA |
| NBLA00301; | cg16919569 | 4 | TTTAAGATTTTAGGGTTAGTGGAGGGTAGA | CCCAAAAAAAACCAAAAACTCCACCTTAA |
| HAND2 | | | | |
| NRXN1 | cg22322562 | 2 | TGTGTTTAGTAATTATATTGGATTTGAATG | AAACTATTAATACACAACCCAACCC |
| ONECUT1 | cg07569216 | 15 | TTTTTGGGAAGTTATAGTAAGAAAATAAAA | TACACTCAAATACTCACACAAAACC |
| OPCML | cg07230581 | 11 | AGGAATTTAAGTTATTTGAGGTTTT | CTATCCCTTCCTCTAAAATAACAAT |
| PAX3-Cg193 | cg19352038 | 2 | ATTTTTTTGATTGATTAAGGTTTTGAATAT | AACTAAAATATCCCCAACAAAATATAAC |
| PROM1 | cg13164157 | 4 | GGTGAGTAGTTGGGTTTTTATTT | CAATTCCTCTAACCCCCAAC |
| RASGEF1C | cg08898055 | 5 | AGGGTAGTGAGTTTGGTTAGGG | AACCAAAAAACAACTACAAAAAAAA |
| SGEF | cg06573459 | 3 | AATAATTAAAATGTGGAGTTTTATAAGAGA | TCAAAACCACTAACCACTACCCTAC |
| SHANK3 | cg18851100 | 22 | GGTTAAGGTTGGTTTTTGTGGGAGG | AAAAAAAACAAAAAACCCAAAACCC |
| SHOX2 | cg12993163 | 3 | TTGTATGAAAAGGTTTTGAGTAATTAATAGAA | TCCCCTAAACAACCAAATAATCTCC |
| SIX6 | cg13019491 | 14 | TGGTGGGGTATAATAGTAGGGATT | CATCCTAAATAAACAACTCAAATATC |
| SORCS2 | cg00387964 | 4 | GAAATTGTTGTGGTTAAATGATTTATTTT | TTTACCTCTCAAAATACCCCCACA |
| TBX5 | cg18689332 | 12 | GATATTTTAGTAACGCGAGGATCGGC | CGTAAAAACGAAAACTAACCCCGTT |
| TLR1 | cg08757862 | 4 | AGGGGAAATAGAGAGAGATAGTTAGAATAT | AACCTACATAATATCCAATCAAAACC |
| TLX3 | cg05787556 | 5 | GGAAGAATTTAGGTTAGGGGTGCGA | TATCTACCCGACCCAAAACACCGTA |
| VOPP1 | cg08337633 | 7 | AGTTAGAGTGAAATTTTGGGTAGTTT | ACTATCAAAAAATAATCATCTCTTACTTCA |
| ZBTB38 | cg02936049 | 3 | TTTTGGGATTTAGTGTTTTGATTTT | CATTTTAACCTATTTCTACCACTTTAAC |

### 6.5. References for Section 6.1-6.4

Alexandrescu D T, Kauffman C L, Jatkoe T A, et al. (2010) Melanoma-specific marker expression in skin biopsy tissues as a tool to facilitate melanoma diagnosis. J Invest Dermatol 130:1887-92.
Arai E, Kanai Y. (2010) DNA methylation profiles in precancerous tissue and cancers: carcinogenetic risk estimation and prognostication based on DNA methylation status. Epigenomics 2:467-81.
Baras A S, Solomon A, Davidson R, et al. (2011) Loss of VOPP1 overexpression in squamous carcinoma cells induces apoptosis through oxidative cellular injury. Lab Invest 91:1170-80.
Baras A, Yu Y, Filtz M, et al. (2009) Combined genomic and gene expression microarray profiling identifies ECOP as an upregulated gene in squamous cell carcinomas independent of DNA amplification. Oncogene 28:2919-24.
Bastian B C, LeBoit P E, Pinkel D. (2000) Mutations and copy number increase of HRAS in Spitz nevi with distinctive histopathological features. Am J Pathol 157:967-72.
Bastian B C, Olshen A B, LeBoit P E, et al. (2003) Classifying melanocytic tumors based on DNA copy number changes. Am J Pathol 163:1765-70.
Bauer J, Bastian B C. (2006) Distinguishing melanocytic nevi from melanoma by DNA copy number changes: comparative genomic hybridization as a research and diagnostic tool. Dermatol Ther 19:40-9.
Bibikova, M., Lin, Z., Zhou, L., Chudin, E., Garcia, E. W., Wu, B., Doucet, D., Thomas, N.J., Wang, Y., Vollmer, E., et al (2006). High-throughput DNA methylation profiling using universal bead arrays. Genome Res. 16, 383-393.
Brochez L, Verhaeghe E, Grosshans E, et al. (2002) Inter-observer variation in the histopathological diagnosis of clinically suspicious pigmented skin lesions. J Pathol 196:459-66.
Busam K J. (2013) Molecular pathology of melanocytic tumors. Semin Diagn Pathol 30:362-74.
Causeret F, Sumia I, Pierani A. (2016) Kremen1 and Dickkopf1 control cell survival in a Wnt-independent manner. Cell Death Differ 23:323-32.
Chapman P B, Hauschild A, Robert C, et al. (2011) Improved survival with vemurafenib in melanoma with BRAF V600E mutation. N Engl J Med 364:2507-16.
Clarke L E, Warf M B, Flake D D 2nd, et al. (2015) Clinical validation of a gene expression signature that differentiates benign nevi from malignant melanoma. J Cutan Pathol 42:244-252.
Conway K, Edmiston S N, Khondker Z S, et al. (2011) DNA-methylation profiling distinguishes malignant melanomas from benign nevi. Pigment Cell Melanoma Res 24:352-60.
Dietrich D, Jung M, Puetzer S, et al. (2013) Diagnostic and prognostic value of SHOX2 and SEPT9 DNA methylation and cytology in benign, paramalignant and malignant pleural effusions. PLoS One 8:e84225.
Dye D E, Medic S, Ziman M, et al. (2013) Melanoma biomolecules: independently identified but functionally intertwined. Front Oncol 3:252.
Farmer E R, Gonin R, Hanna M P. (1996) Discordance in the histopathologic diagnosis of melanoma and melanocytic nevi between expert pathologists. Hum Pathol 27:528-31.
Fung M A. (2003) Terminology and management of dysplastic nevi: responses from 145 dermatologists. Arch Dermatol 139:1374-5.
Furuta J, Umebayashi Y, Miyamoto K, et al. (2004) Promoter methylation profiling of 30 genes in human malignant melanoma. Cancer Sci 95:962-8.
Galluzzi L, Goubar A, Olaussen K A, et al. (2013) Prognostic value of LIPC in non-small cell lung carcinoma. Cell Cycle 12:647-54.
Gao C, Pang M, Zhou Z, et al. (2015) Epidermal growth factor receptor-coamplified and overexpressed protein (VOPP1) is a putative oncogene in gastric cancer. Clin Exp Med 15:469-75.
Gao L, Smit M A, van den Oord J J, et al. (2013) Genome-wide promoter methylation analysis identifies epigenetic silencing of MAPK13 in primary cutaneous melanoma. Pigment Cell Melanoma Res 26:542-54.
Gerami P, Busam K, Cochran A, et al. (2014) Histomorphologic assessment and interobserver diagnostic reproducibility of atypical spitzoid melanocytic neoplasms with long-term follow-up. Am J Surg Pathol 38:934-40.
Gerami P, Jewell S S, Morrison L E, et al. (2009) Fluorescence in situ hybridization (FISH) as an ancillary diagnostic tool in the diagnosis of melanoma. Am J Surg Pathol 33:1146-56.
Hamid O, Robert C, Daud A, et al. (2013) Safety and tumor responses with lambrolizumab (anti-PD-1) in melanoma. N Engl J Med 369:134-44.
Haqq C, Nosrati M, Sudilovsky D, et al. (2005) The gene expression signatures of melanoma progression. Proc Natl Acad Sci USA 102:6092-7.
Hauschild A, Grob J J, Demidov L V, et al. (2012) Dabrafenib in BRAF-mutated metastatic melanoma: a multicentre, open-label, phase 3 randomised controlled trial. Lancet 380:358-65.
Herman J G, Baylin S B. (2003) Gene silencing in cancer in association with promoter hypermethylation. N Engl J Med 349:2042-54.
Hodi F S, O'Day S J, McDermott D F, et al. (2010) Improved survival with ipilimumab in patients with metastatic melanoma. N Engl J Med 363:711-23.
Hoon D S, Spugnardi M, Kuo C, et al. (2004) Profiling epigenetic inactivation of tumor suppressor genes in tumors and plasma from cutaneous melanoma patients. Oncogene 23:4014-22.
Ivan D, Prieto V G. (2010) Use of immunohistochemistry in the diagnosis of melanocytic lesions: applications and pitfalls. Future Oncol 6:1163-75.
Jiang X, Zhang W, Kayed H, et al. (2008) Loss of ONECUT1 expression in human pancreatic cancer cells. Oncol Rep 19:157-63.
Jin S G, Xiong W, Wu X, et al. (2015) The DNA methylation landscape of human melanoma. Genomics 106:322-30.
Jones P A. (2012) Functions of DNA methylation: islands, start sites, gene bodies and beyond. Nat Rev Genet 13:484-92.
Jones A, Teschendorff A E, Li Q, et al. (2013) Role of DNA methylation and epigenetic silencing of HAND2 in endometrial cancer development. PLoS Med 10:e1001551.
Kikuchi Y, Tsuji E, Yagi K, et al. (2013) Aberrantly methylated genes in human papillary thyroid cancer and their association with BRAF/RAS mutation. Front Genet 4:271.
Koh S S, Opel M L, Wei J P, et al. (2008) Molecular classification of melanomas and nevi using gene expression microarray signatures and formalin-fixed and paraffin-embedded tissue. Mod Pathol 22:538-46.
Lai H C, Lin Y W, Huang T H, et al. (2008) Identification of novel DNA methylation markers in cervical cancer. Int J Cancer 123:161-7.
Lessard L, Liu M, Marzese D M, et al. (2015) The CASC15 Long Intergenic Noncoding RNA Locus Is Involved in Melanoma Progression and Phenotype Switching. J Invest Dermatol 135:2464-74.
Li C, Tang L, Zhao L, et al. (2015) OPCML is frequently methylated in human colorectal cancer and its restored expression reverses EMT via downregulation of smad signaling. Am J Cancer Res 5:1635-48.
Makiyama K, Hamada J, Takada M, et al. (2005) Aberrant expression of HOX genes in human invasive breast carcinoma. Oncol Rep 13:673-9.
Medic S, Ziman M. (2009) PAX3 across the spectrum: from melanoblast to melanoma. Crit Rev Biochem Mol Biol 44:85-97.
Medic S, Ziman M. (2010) PAX3 expression in normal skin melanocytes and melanocytic lesions (naevi and melanomas). PLoS One 5(4):e9977.
Noisa P, Raivio T. (2014) Neural crest cells: from developmental biology to clinical interventions. Birth Defects Res C Embryo Today 102:263-74.
North J P, Garrido M C, Kolaitis N A, et al. (2014) Fluorescence in situ hybridization as an ancillary tool in the diagnosis of ambiguous melanocytic neoplasms: a review of 804 cases. Am J Surg Pathol 38:824-31.
Plass C. (2002) Cancer epigenomics. Hum Mol Genet 11:2479-88.
Price M E, Cotton A M, Lam L L, et al. (2013) Additional annotation enhances potential for biologically-relevant analysis of the Illumina Infinium HumanMethylation450 BeadChip array. Epigenetics Chromatin. 6:4.
Qiu Z X, Zhao S, Mo X M, et al. (2015) Overexpression of PROM1 (CD133) confers poor prognosis in non-small cell lung cancer. Int J Clin Exp Pathol 8:6589-95.
Robert C, Long G V, Brady B, et al. (2015) Nivolumab in previously untreated melanoma without BRAF mutation. N Engl J Med 372:320-30.
Russell M R, Penikis A, Oldridge D A, et al. (2015) CASC15-S is a tumor suppressor lncRNA at the 6p22 neuroblastoma susceptibility locus. Cancer Res 75:3155-66.
Semaan A, van Ellen A, Meller S, et al. (2016) SEPT9 and SHOX2 DNA methylation status and its utility in the diagnosis of colonic adenomas and colorectal adenocarcinomas. Clin Epigenetics 8:100.
Shain A H, Yeh I, Kovalyshyn I, et al. (2015) The genetic evolution of melanoma from precursor lesions. N Engl J Med 373:1926-36.
Sharma B K, Manglik V, Elias E G. (2010) Immuno-expression of human melanoma stem cell markers in tissues at different stages of the disease. J Surg Res 163:e11-5.
Shoo B A, Sagebiel R W, Kashani-Sabet M. (2010) Discordance in the histopathologic diagnosis of melanoma at a melanoma referral center. J Am Acad Dermatol 62:751-6.
Siegel R, Ma J, Zou Z, et al. (2014) Cancer statistics, 2014. CA Cancer J Clin 64:9-29.
Song L, Yu H, Li Y. (2015) Diagnosis of lung cancer by SHOX2 gene methylation assay. Mol Diagn Ther 19:159-67.
Strauss R M, Elliott F, Affleck P, et al. A retrospective study addressed to understanding what predicts severe histological dysplasia/early melanoma in excised atypical melanocytic lesions. (2007) Br J Dermatol 157:758-64.
Tada Y, Yokomizo A, Shiota M, et al. (2011) Aberrant DNA methylation of T-cell leukemia, homeobox 3 modulates cisplatin sensitivity in bladder cancer. Int J Oncol 39:727-33.
Talantov D, Mazumder A, Yu J X, et al. (2005) Novel genes associated with malignant melanoma but not benign melanocytic lesions. Clin Cancer Res 11:7234-42.
Tanemura A, Terando A M, Sim M S, et al. (2009) CpG island methylator phenotype predicts progression of malignant melanoma. Clin Cancer Res 15:1801-1807.
Teschendorff A E, Marabita F, Lechner M, et al. (2013) A beta mixture quantile normalization method for correcting probe design bias in Illumina Infinium 450k DNA methylation data. Bioinformatics 29:189-196.
The Cancer Genome Atlas Network. (2015) Genomic classification of cutaneous melanoma. Cell 161:1681-1696.
Thomas N E, Alexander A, Edmiston S N, et al. (2004) Tandem BRAF mutations in primary invasive melanomas. J Invest Dermatol 122:1245-50.
Thomas N E, Slater N A, Edmiston S N, et al. (2014) DNA methylation profiles in primary cutaneous melanomas are associated with clinically significant pathologic features. Pigment Cell Melanoma Res 27:1097-105.
Uguen A, Talagas M, Costa S, et al. (2015) A p16-Ki-67-HMB45 immunohistochemistry scoring system as an ancillary diagnostic tool in the diagnosis of melanoma. Diagn Pathol 10:195.
Veenhuizen K C, De Wit P E, Mooi W J, et al. (1997) Quality assessment by expert opinion in melanoma pathology: experience of the pathology panel of the Dutch Melanoma Working Party. J Pathol 182:266-72.
Wimmer K, Zhu Xx X X, Rouillard J M, et al. (2002) Combined restriction landmark genomic scanning and virtual genome scans identify a novel human homeobox gene, ALX3, that is hypermethylated in neuroblastoma. Genes Chromosomes Cancer 33:285-94.
Xing B L, Li T, Tang Z H, et al. (2015) Cumulative methylation alternations of gene promoters and protein markers for diagnosis of epithelial ovarian cancer. Genet Mol Res 14:4532-40.
Yu J, Ma X, Cheung K F, et al. (2010) Epigenetic inactivation of T-box transcription factor 5, a novel tumor suppressor gene, is associated with colon cancer. Oncogene 29:6464-74.
Zha Y, Ding E, Yang L, et al. (2012) Functional dissection of HOXD cluster genes in regulation of neuroblastoma cell proliferation and differentiation. PLoS One 7:e40728.
Zhao Y, Zhou H, Ma K, et al. (2013) Abnormal methylation of seven genes and their associations with clinical characteristics in early stage non-small cell lung cancer. Oncol Lett 5:1211-1218.
Zheng Y, Li Y F, Wang W, et al. (2015) High expression level of T-box transcription factor 5 predicts unfavorable survival in stage I and II gastric adenocarcinoma. Oncol Lett 10:2021-2026.
Zimmerer R M, Matthiesen P, Kreher F, et al. (2016) Putative CD133+ melanoma cancer stem cells induce initial angiogenesis in vivo. Microvasc Res 104:46-54.
Zou H, Hastie T. (2005) Regularization and variable selection via the Elastic Net. Journal of the Royal Statistical Society: Series B (Statistical Methodology). 67:301-320.
Zhou F, Tao G, Chen X, et al. (2014) Methylation of OPCML promoter in ovarian cancer tissues predicts poor patient survival. Clin Chem Lab Med 52:735-42.

### 6.6. Identification of a Robust Classifier for Cutaneous Melanoma

### Abstract

Early diagnosis improves melanoma survival, yet the histopathological diagnosis of cutaneous primary melanoma can be challenging even for expert dermatopathologists. Analysis of epigenetic alterations, such as DNA methylation, that occur in melanoma can aid in its early diagnosis. Using a genome-wide methylation screen, we assessed CpG methylation in a diverse set of 89 primary invasive melanomas, 73 nevi, and 41 melanocytic proliferations of uncertain malignant potential, classified based on interobserver review by dermatopathologists. Melanomas and nevi were split into training and validation sets. Predictive modeling in the training set using ElasticNet identified a 40-CpG classifier distinguishing 60 melanomas from 48 nevi. High diagnostic accuracy (area under the receiver operator characteristics (ROC) curve (AUC)=0.996, sensitivity=96.6%, and specificity=100.0%) was independently confirmed in the validation set (29 melanomas, 25 nevi) and other published sample sets. The 40-CpG melanoma classifier included homeobox transcription factors and genes with roles in stem cell pluripotency or the nervous system. Application of the 40-CpG melanoma classifier to the diagnostically uncertain samples assigned melanoma or nevus status, potentially offering a diagnostic tool to assist dermatopathologists. In summary, the robust, accurate 40-CpG melanoma classifier offers a promising assay for improving primary melanoma diagnosis.

### 6.7. Introduction Section 6.6-6.11

Cutaneous melanoma is an aggressive malignancy with the potential to metastasize early, and there is a pronounced survival difference between localized and metastatic disease (Landow et al., 2017; Shaikh et al., 2016; Siegel et al., 2018; Whiteman et al., 2015). Despite newly available targeted and immunomodulatory agents for the treatment of melanoma (Andtbacka et al., 2015; Clarke et al., 2018; Hodi et al., 2016; Long et al., 2017; Ribas et al., 2016; Ribas et al., 2015; Robert et al., 2015; Schachter et al., 2017), the durability of the response is not yet known and systemic therapies lead to cures in a relatively small number of patients. Therefore, early detection is crucial for favorable outcomes, but early definitive diagnosis can be difficult due to the overlap in clinical and histopathological appearances of melanomas and highly prevalent melanocytic nevi (moles) (Strauss et al., 2007). Histopathological review is the ‘gold standard’ for melanoma diagnosis; however, numerous studies have reported interobserver discordance in the diagnosis of melanocytic lesions even by expert dermatopathologists (Brochez et al., 2002; Elmore et al., 2017; Shoo et al., 2010; Veenhuizen et al., 1997). In one study (Farmer et al., 1996), review of 40 benign and malignant melanocytic lesions by eight dermatopathologists produced discordant diagnoses in 38% of cases. Moreover, certain nevus subtypes, especially dysplastic nevi, Spitz nevi, and atypical blue nevi can be difficult to distinguish from melanoma (Brochez et al., 2002; Gerami et al., 2014). The difficulty in accurately diagnosing melanoma presents a quandary for clinicians, who biopsy and often re-excise with margins large numbers of dysplastic nevi in the population (Fung, 2003), due in part to lack of confidence in the histopathological diagnosis. A critical need exists for improving diagnostic methods to avoid under- and over-treatment of melanocytic lesions. However, the small size of melanocytic lesions and early melanomas, which are typically submitted in their entirety in formalin to the pathologist for diagnosis, present particular challenges as any new diagnostic test needs to perform reliably on small formalin-fixed paraffin-embedded (FFPE) samples.

Prior studies have shown that melanomas differ from nevi at the molecular level, exhibiting variations in mRNA expression (Alexandrescu et al., 2010; Clarke et al., 2015; Haqq et al., 2005; Koh et al., 2009; Talantov et al., 2005), gene copy number (Bastian et al., 2000; Bastian et al., 2003; Bauer and Bastian, 2006; Gerami et al., 2009; North et al., 2014; Shain et al., 2015), protein expression (Busam, 2013; Ivan and Prieto, 2010; Uguen et al., 2015), and DNA methylation (Conway et al., 2011; Gao et al., 2013; Gao et al., 2014), indicating that certain molecular biomarkers could provide valuable tools for melanoma diagnosis, alone or with histopathology. However, due to the practical limitations of typically small FFPE samples and technical challenges or labor intensity in the performance and implementation of some assays, few molecular differences have been translated to the clinic for melanoma diagnosis.

DNA methylation is a relatively stable epigenetic modification to the DNA that does not alter the nucleotide sequence but is associated with variation in gene expression (Plass, 2002). Changes in methylation at CpG dinucleotides in the upstream regulatory regions of genes are often among the earliest events observed during neoplastic progression of precancerous lesions (Arai and Kanai, 2010), and hypermethylation of CpG islands in tumor suppressor gene promoters is a common mechanism of gene silencing in human cancer (Herman and Baylin, 2003). Aberrant DNA methylation occurs widely in melanomas (Furuta et al., 2004; Hoon et al., 2004), and we (Conway et al., 2011) and others (Gao et al., 2013; Gao et al., 2014) have reported differences in DNA methylation between primary melanomas and nevi, supporting the use of epigenetic biomarkers for early melanoma diagnosis.

Our initial study using a methylation array that targeted cancer-related genes provided proof-of-principle that DNA methylation differences could distinguish invasive primary melanomas from benign nevi in small FFPE samples (Conway et al., 2011; Thomas et al., 2014). In the present study, we extend this work by identifying and independently validating a highly accurate 40-CpG melanoma classifier that distinguishes primary melanomas from a broad histopathologic spectrum of nevi within a set of melanocytic samples reviewed by a panel of expert dermatopathologists. These findings could translate to a robust melanoma diagnostic test ideal for use in FFPE melanocytic samples.

### 6.8. Results

Patient and Sample Characteristics

Illumina Infinium HumanMethylation450 BeadChip (450K) analysis was successfully performed on 97% of samples tested. The clinicopathologic characteristics of the sample set are included in Table 1. The sample set of FFPE tissues included 89 cutaneous primary invasive melanomas, 73 nevi, and 41 melanocytic proliferations of uncertain malignant potential (‘uncertain’ samples). All melanomas and nevi were classified based on complete consensus between the original pathology report and three dermatopathology reviewers (four interpretations), although we did not exclude a lesion as melanoma if the majority of dermatopathologists interpreted the lesion as melanoma and visceral metastases and/or death from melanoma provided unequivocal evidence of the malignancy of the lesion. The diagnostically uncertain samples lacked complete consensus between the four interpretations or were called uncertain by any dermatopathologist or the pathology report.

The melanomas had median Breslow thickness of 1.85 millimeters (mm) (range of 0.37-17.00 mm) and were balanced for 7th Edition American Joint Committee on Cancer (AJCC) tumor stages (Balch et al., 2009), and both sample classes were comprised of common and less common histopathological subtypes. The 73 nevi included intradermal, common acquired, dysplastic, Spitz, and blue nevi. The 203 specimens (89 melanomas, 73 nevi, and 41 uncertain samples) were from 202 different patients; one patient had two synchronous primary melanomas, both of which were included in the study. Melanoma patients were more frequently older than nevus patients (P<0.001). Melanomas and nevi (excluding uncertain samples) were randomly divided into training (67% of samples; 60 melanomas and 48 nevi) and validation (33%; 29 melanomas and 25 nevi) sets (Table 1); these did not differ significantly in patient age, sex or other clinical or histopathological characteristics.

Development of a 40-CpG Melanoma Classifier and Validation in an Independent Test Set

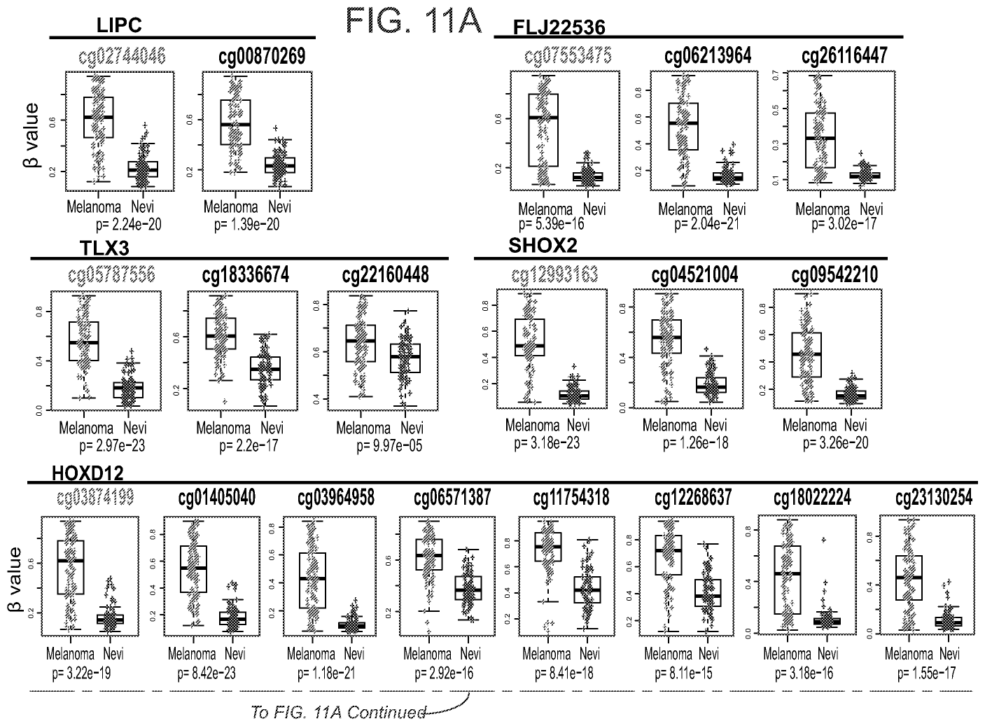

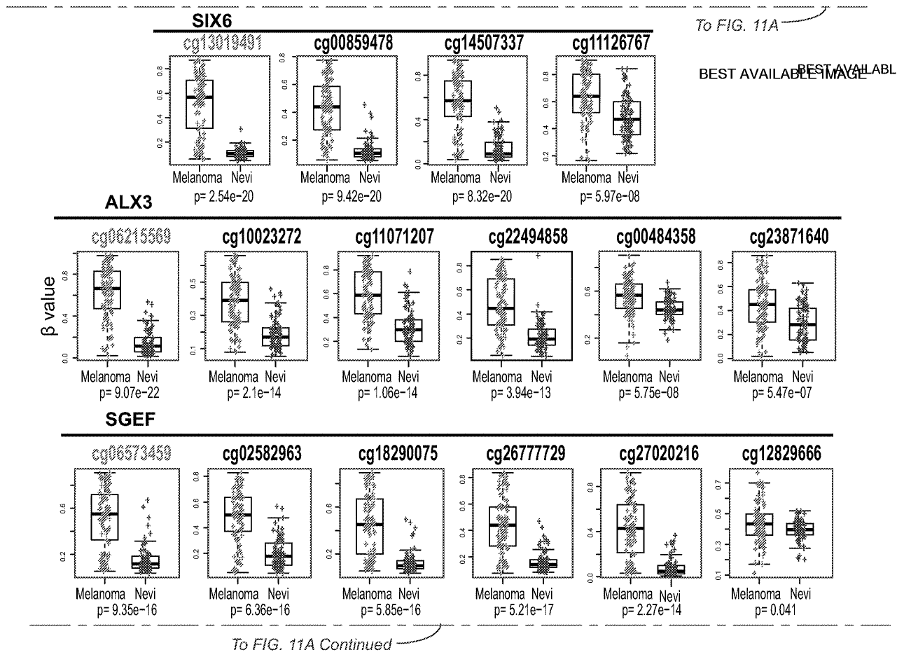

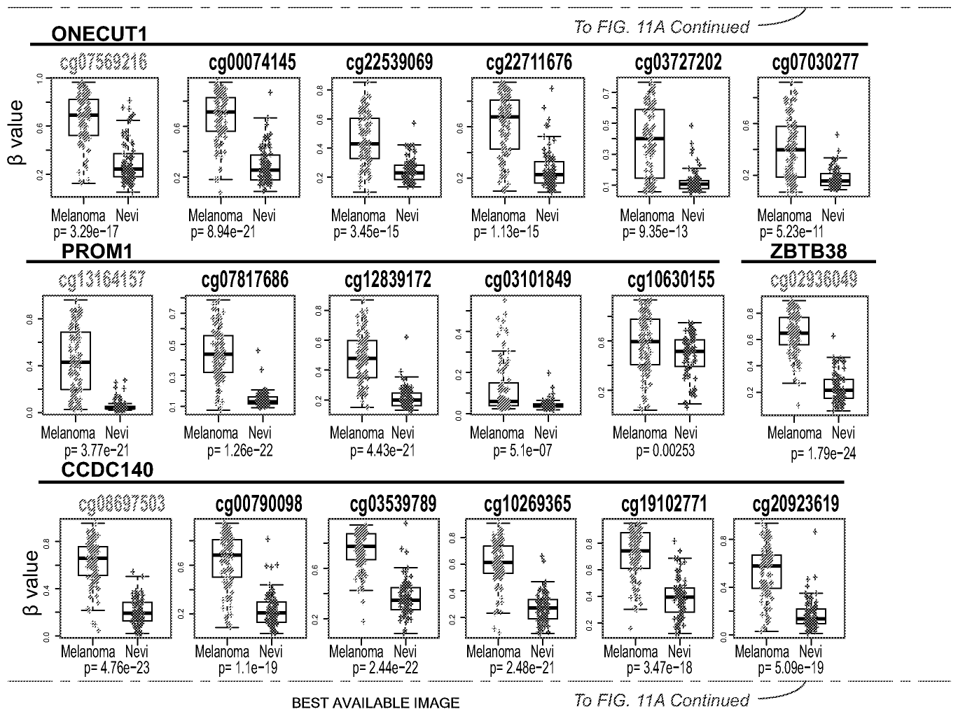

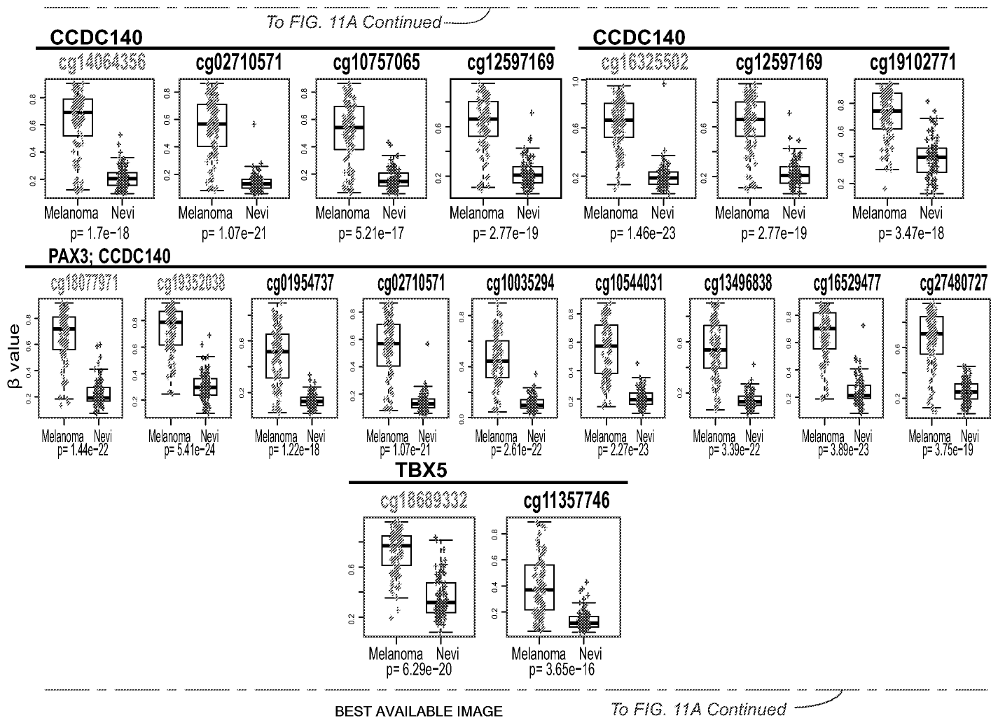

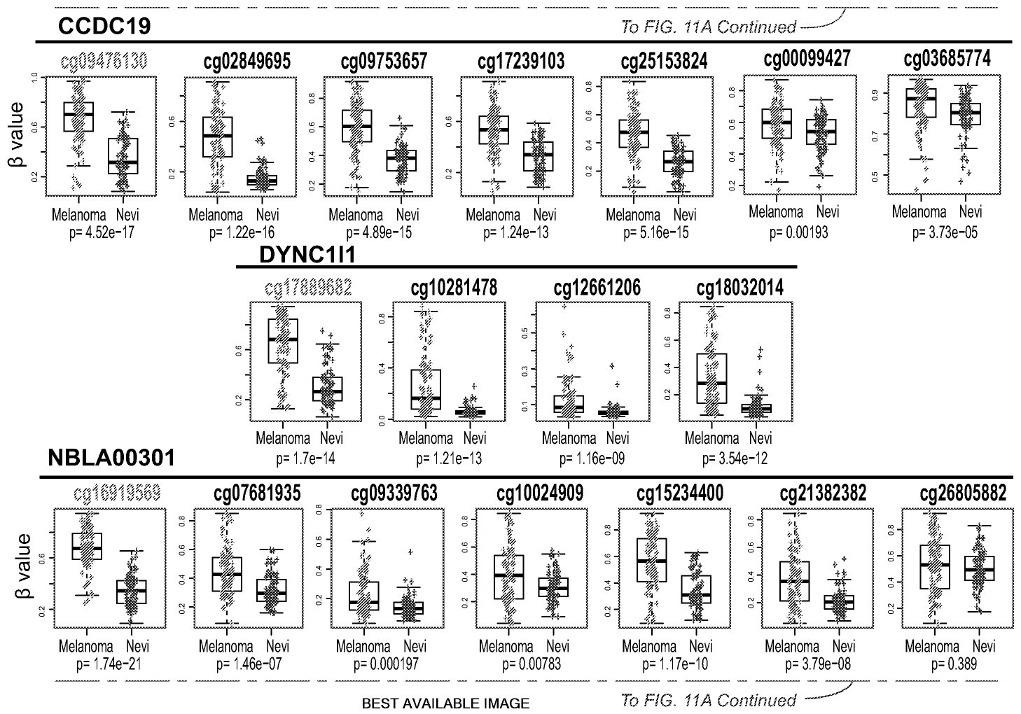

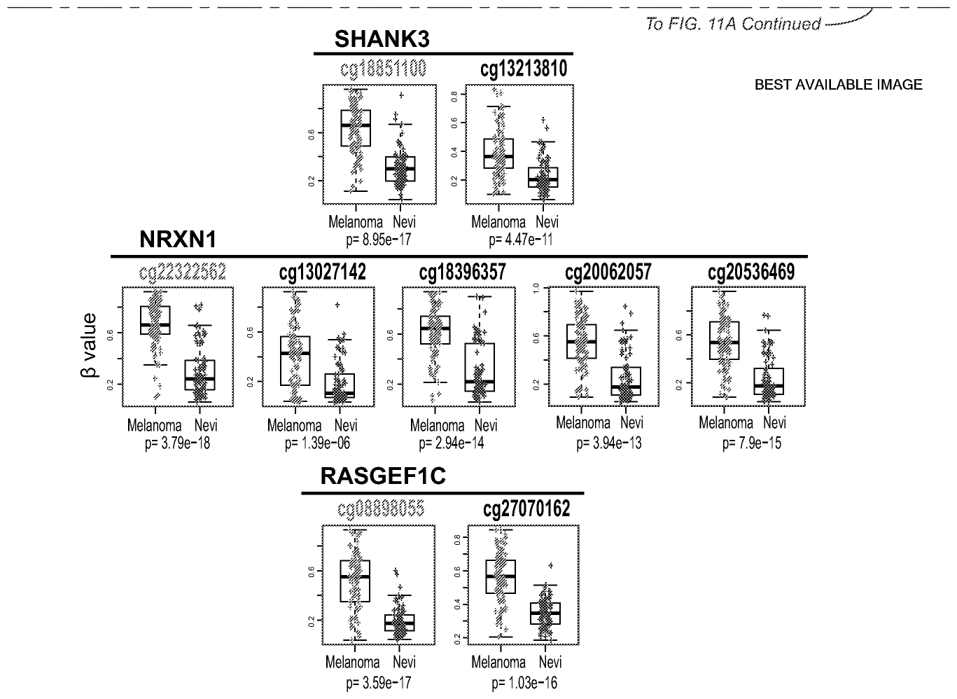

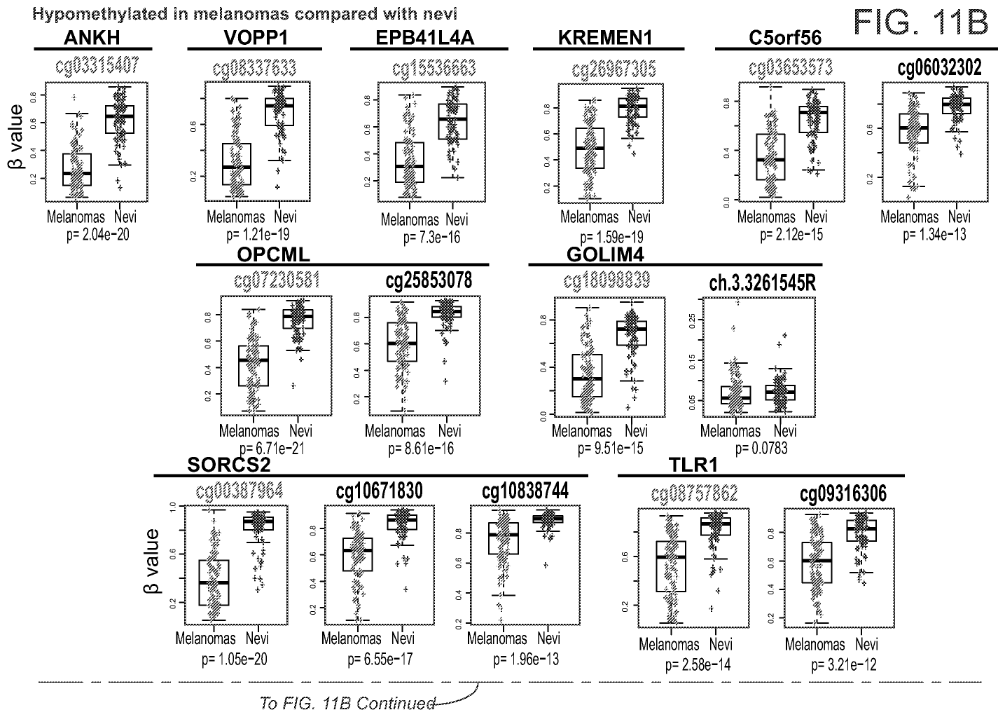

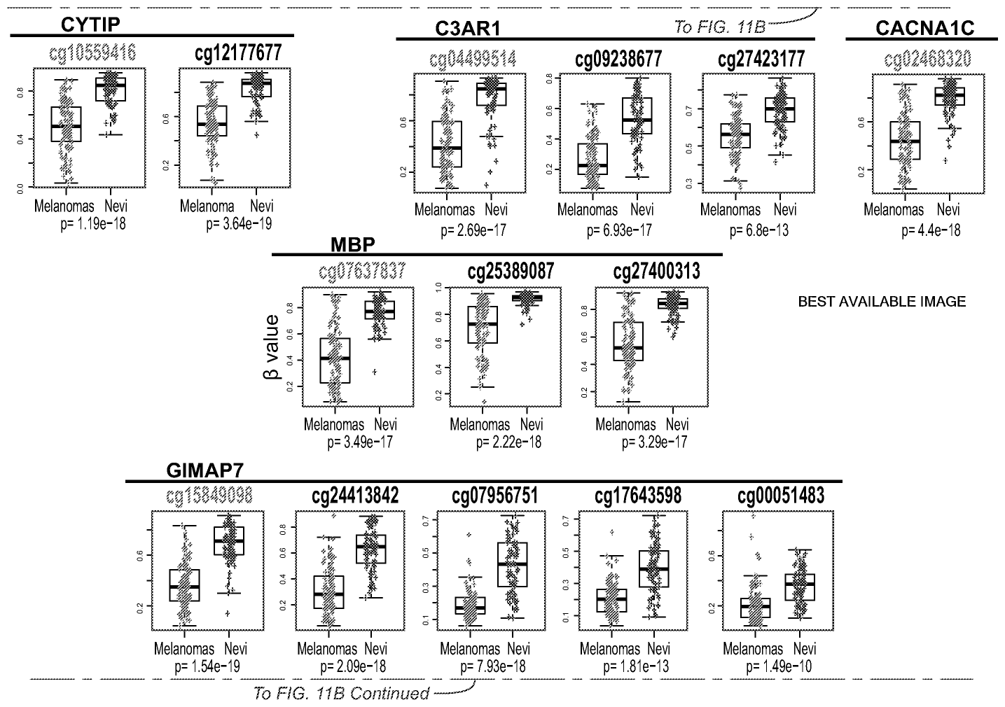

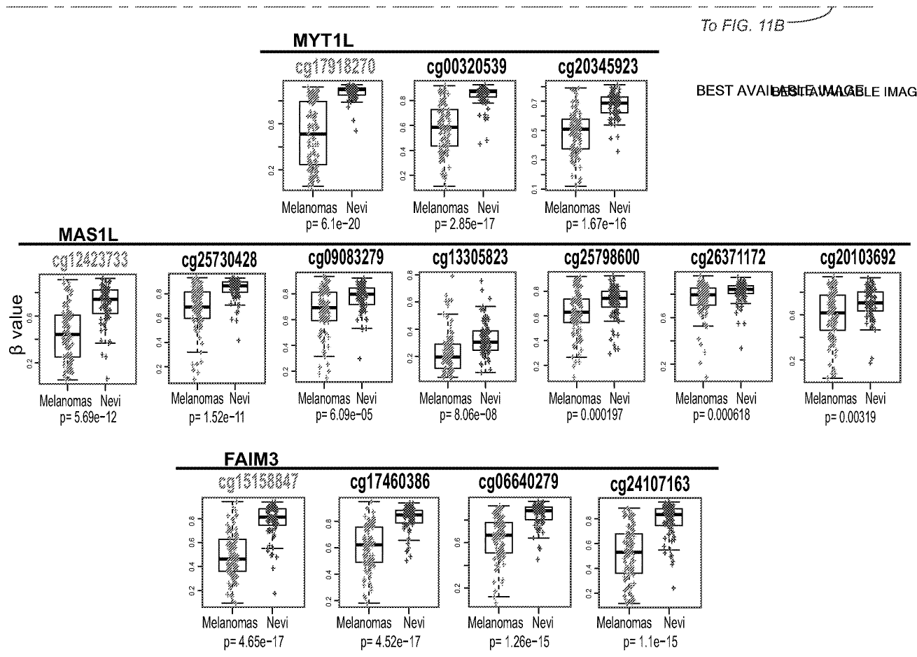

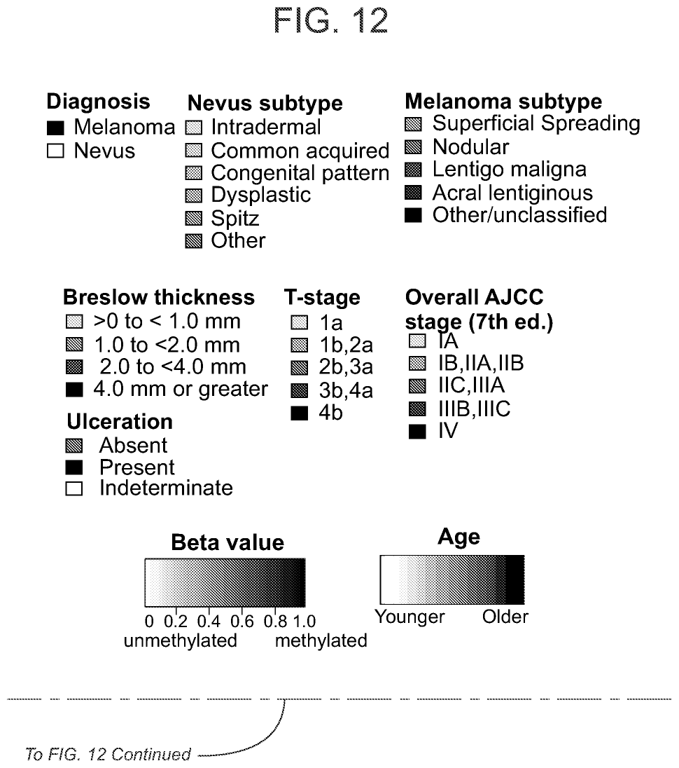

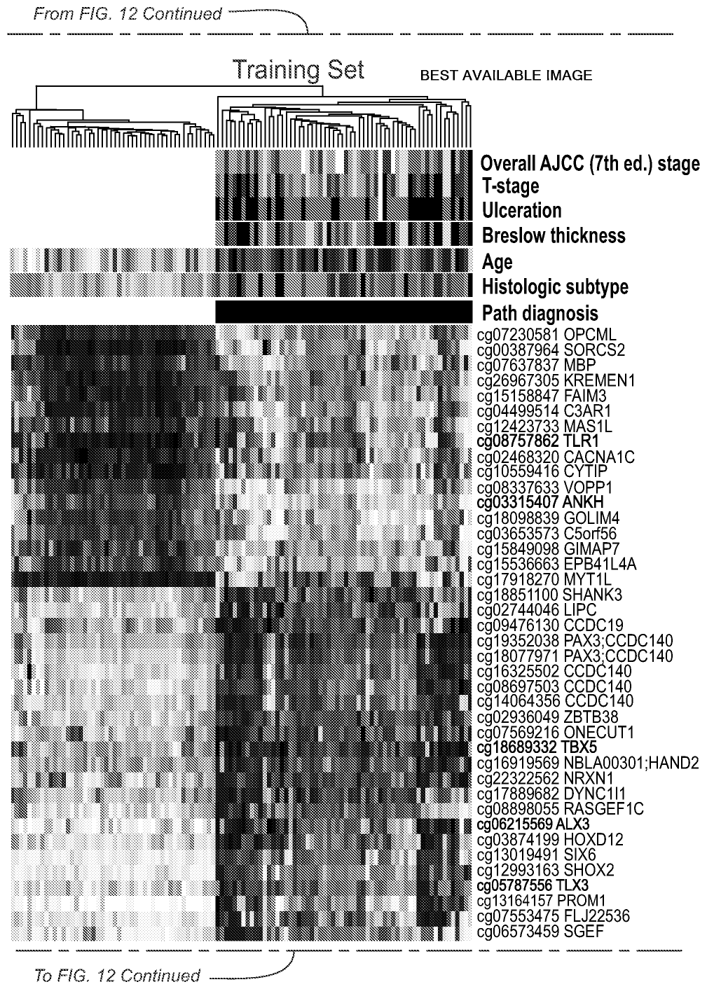

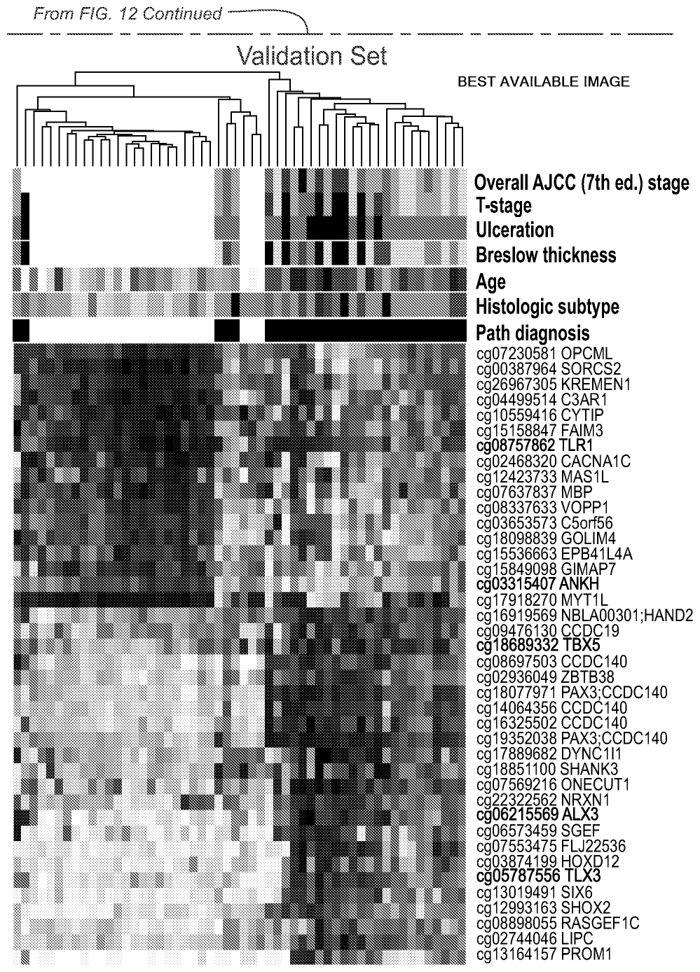

Monte-Carlo cross validation via ElasticNet was used to develop and compare the diagnostic accuracy of CpG classifiers derived from multiple Infinium HumanMethylation450 (450K) probe sets in the training set. Inclusion of all CpG probes provided slightly better diagnostic accuracy than a limited set of probes associated with candidate genes identified from our prior study (Conway et al., 2011) (FIG. 4A-4C). When accounting for age differences in the models by either removing age-associated probes or adjusting for age, or both, each method resulted in a prediction model with inferior diagnostic discrimination; however, this could be overcome by increasing the number of features in the age-adjusted models. Restricting the models to probes showing larger methylation differences (β interquartile range [IQR]>0.2) between melanomas and nevi (FIG. 4A-4B) and/or to probes with Illumina gene annotation (FIG. 4D) produced results very comparable to the more complete probe sets. Based on comparative performance of the models, we identified a 40-CpG melanoma classifier associated with 38 genes for further characterization derived from the probe set filtered for IQR>0.2 β and with gene annotation (n=41,448 probes; FIG. 4D). CpGs contributing to the 40-CpG melanoma classifier were hypermethylated (n=23) or hypomethylated (n=17) in melanomas relative to nevi. The majority of classifier CpGs were located in the upstream regulatory regions of genes (TSS200, TSS1500, 5′UTR), including one-third in enhancer regions (Table 2). Neighboring CpGs around the classifier probes were also similarly differentially methylated in melanomas (FIG. 11A-11B).

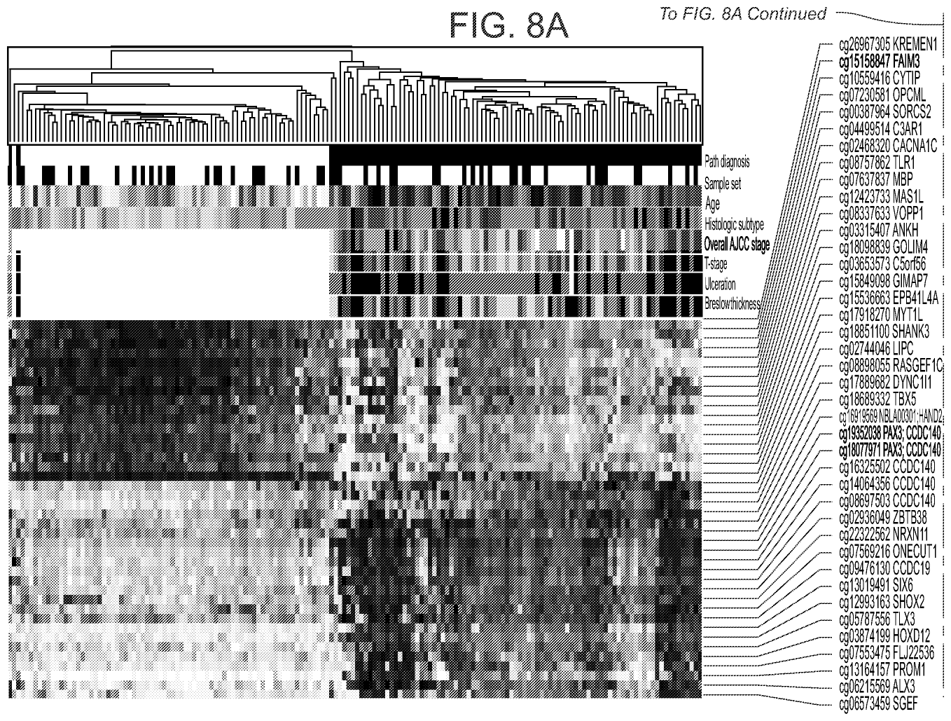

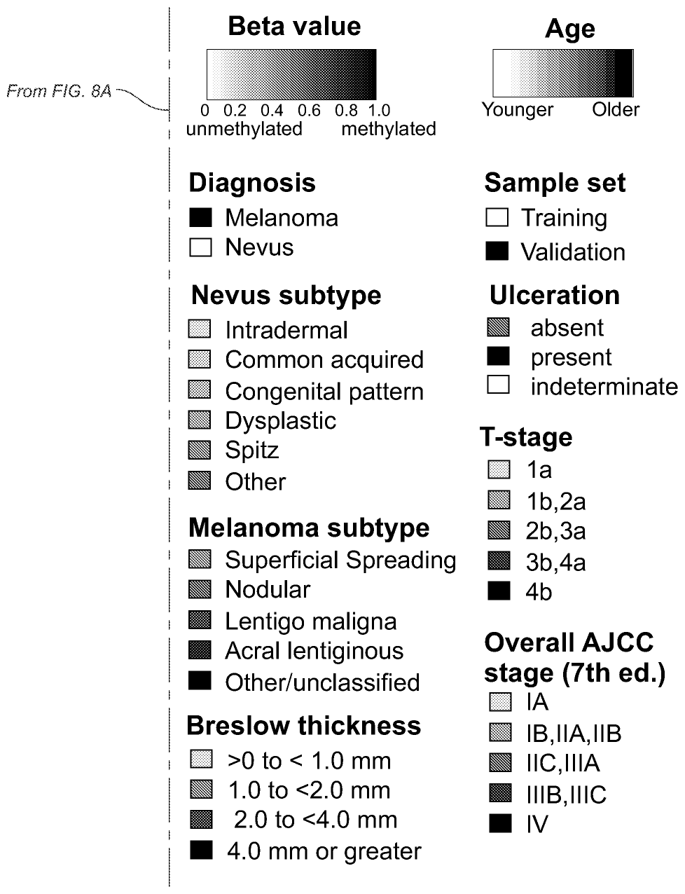

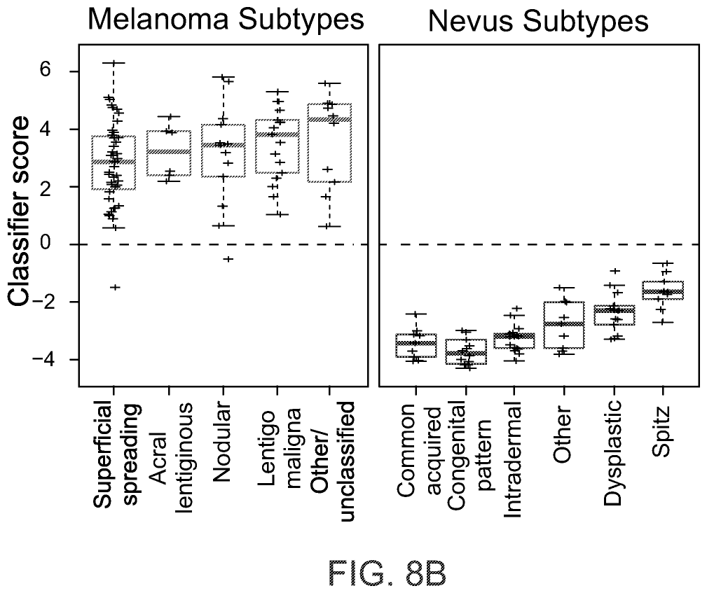

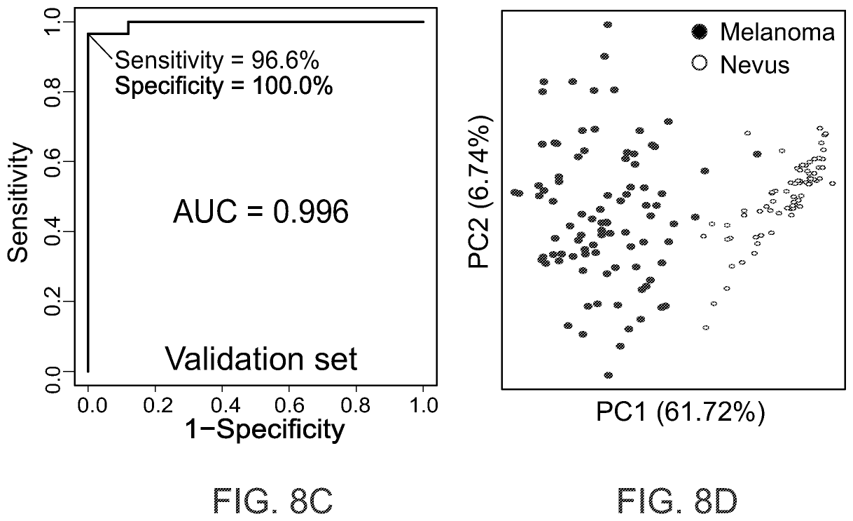

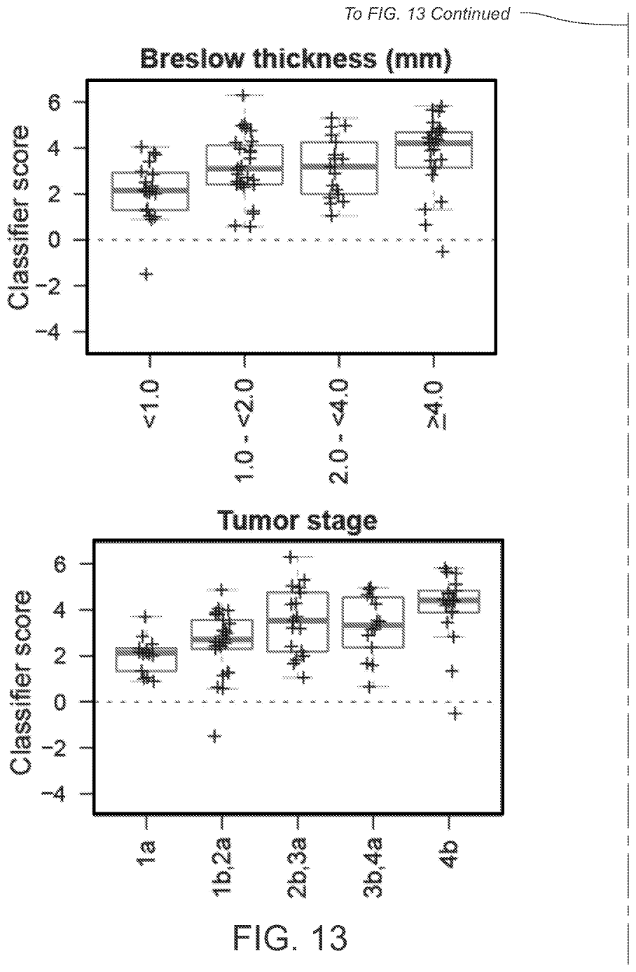

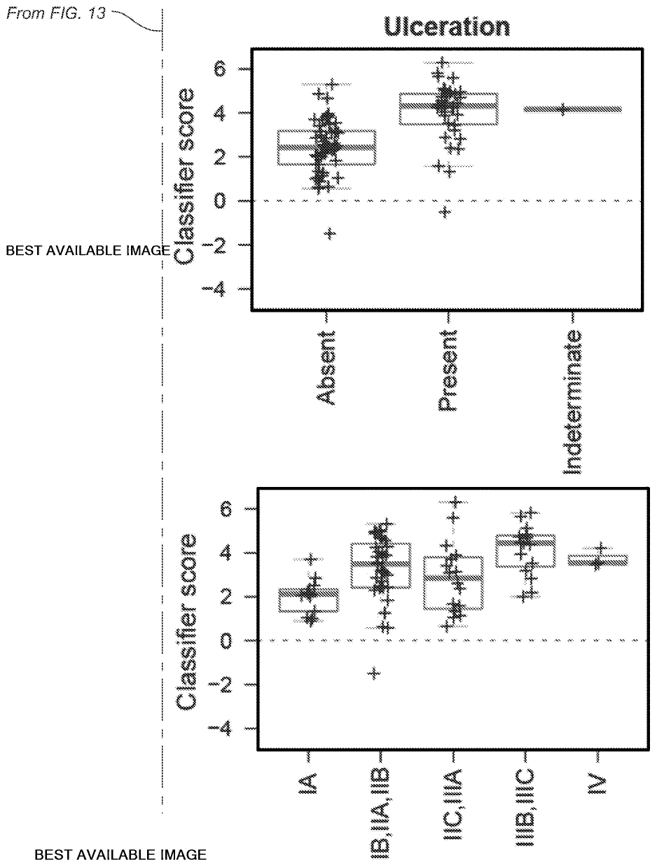

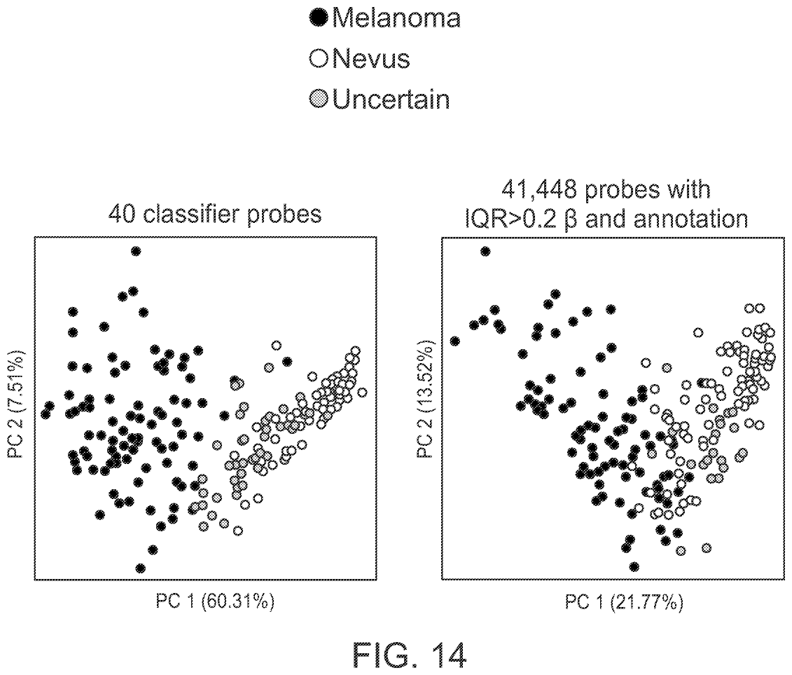

The heatmap in FIG. 8A illustrates the differential methylation at the 40-CpG melanoma classifier probes in primary melanomas and nevi with diagnostic consensus in the training and validation sets. Separate heatmaps for the training and validation sets are also provided in FIG. 13. As shown in FIG. 8B, the 40 CpG diagnostic classifier distinguishes all histological subtypes of nevi, including dysplastic and Spitz nevi, from melanomas. Moreover, early T1a melanomas or thin melanomas with Breslow thickness<1.0 mm were distinguished from nevi (FIG. 14). The diagnostic accuracy of the classifier for melanoma in the independent validation set was high (AUC=0.996), with a sensitivity of 96.6%, specificity of 100%, positive predictive value (PPV) of 100.0%, and negative predictive value (NPV) of 96.2% (FIG. 8C). Principle components analysis (PCA) confirmed the segregation of melanomas from nevi based on the 40-CpG melanoma classifier (FIG. 8D).

Despite the age difference between melanoma and nevus patients and age-associated CpGs being retained in the model, the 40-CpG melanoma classifier performed similarly in differentiating melanomas from nevi among both younger (≤50 years; AUC=0.996) and older (>50 years; AUC=1.00) patients (FIG. 5). The classifier was also accurate irrespective of patient sex, tissue source, anatomic site, pigmentation, purity of the lesion, or degree of solar elastosis in adjacent skin (Supplementary Table 51). Compared with the dermatopathologist consensus, 2 of 89 samples (2.2%) were molecularly reclassified by the 40-CpG classifier; both were melanomas identified as nevi. One was a thin superficial spreading melanoma (Breslow thickness=0.54 mm); the patient was alive with no evidence of disease (ANED) 15 months after diagnosis. The other was a nodular melanoma (Breslow thickness=6.86 mm) from a 5-year old child who was ANED 33 months after diagnosis.

DAVID gene ontology analysis indicated that the 40-CpG melanoma classifier was enriched in homeobox genes that play roles in embryonic development and differentiation (e.g., PAX3, TLX3, SHOX2, ALX3, SIX6, HOXD12, ONECUT1), other transcriptional regulatory genes (HAND2, TBX5, ZBTB38), and genes involved in neurological processes (NRXN1, SHANK3, HAND2, MBP, OPCML, SORCS2) (Supplementary Table S2).

Validation of the Classifier CpGs/Genes in Independent Datasets

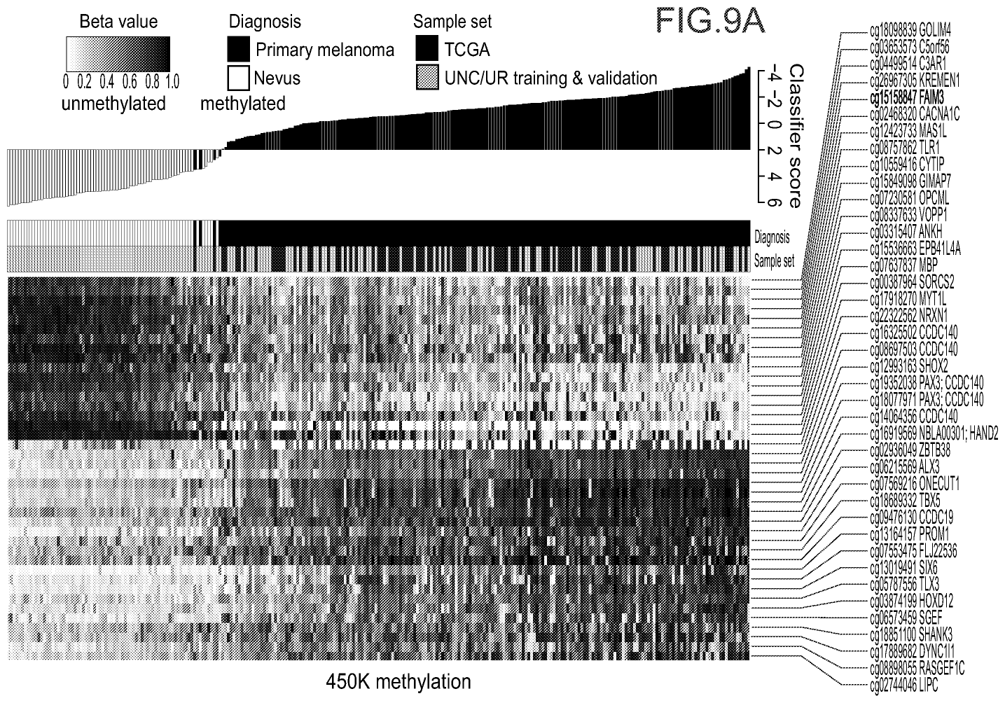

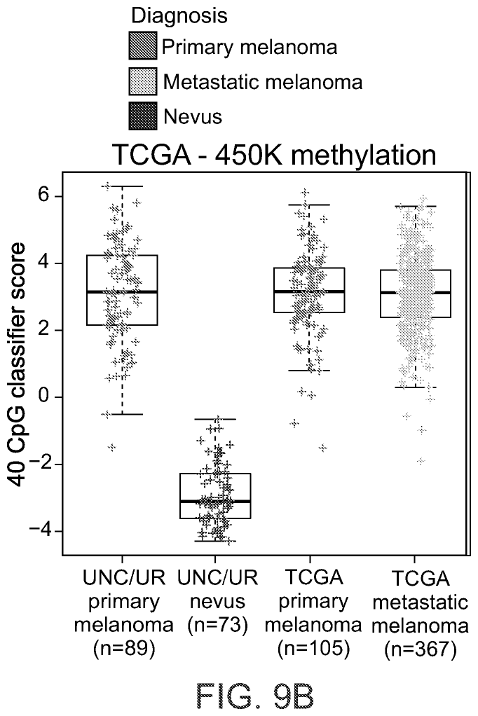

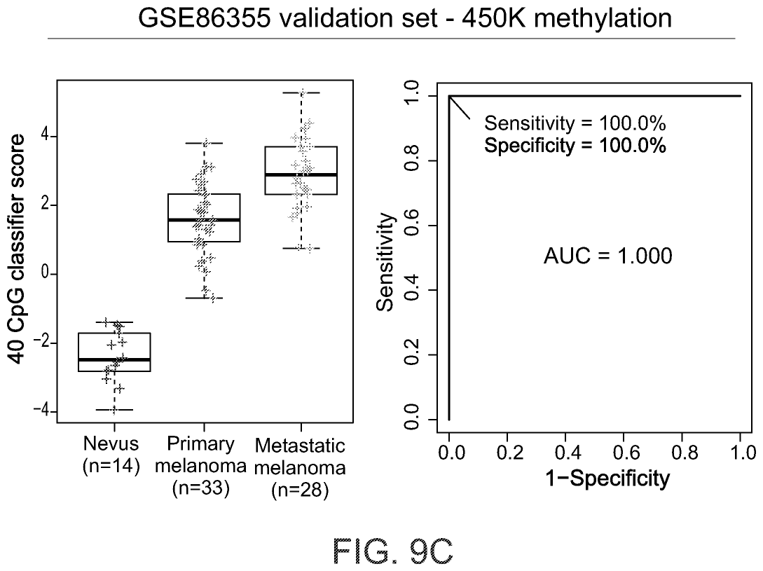

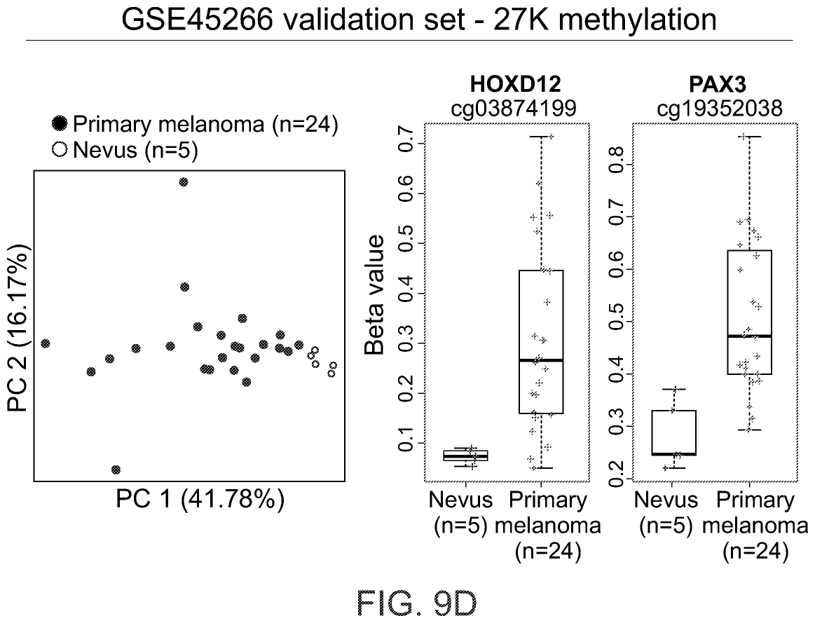

Data from published studies were used to confirm diagnostic methylation differences or to assess the biological relevance of differentially methylated genes by examining associated mRNA expression differences in melanomas versus nevi. As shown in the heatmap and associated waterfall plot in FIG. 9A, application of the 40-CpG melanoma classifier to 105 primary melanomas in The Cancer Genome Atlas (TCGA) 450K methylation dataset (TCGA, 2015) confirmed 103 of these as melanomas despite TCGA primary melanomas being generally of higher tumor stage and obtained as frozen samples compared with UNC/UR study samples. Moreover, 367 metastatic melanomas from TCGA showed a similar range of classifier scores as the TCGA primary melanomas (FIG. 9B). Using 450K methylation data from the study of Wouters et al. (Wouters et al., 2017), primary and metastatic melanomas were accurately distinguished from nevi with AUC of 1.000 (FIG. 9C-9D). Using 27K methylation data from the study of Gao and colleagues (Gao et al., 2013), PCA of methylation at 44 CpGs associated with genes in the 40-CpG diagnostic classifier distinguished primary melanomas from nevi (FIG. 9E); only two of these probes directly overlapped probes in the 40-CpG classifier (cg03874199 in HOXD12; cg19352038 in PAX3) and these exhibited large differences in methylation between melanomas and nevi (FIG. 9F). Differential mRNA expression of several diagnostic genes, including PAX3, TBX5, MBP, GOLIM4, and ANKH, also differentiated primary melanomas from nevi in the dataset of Talantov et al (Talantov et al., 2005).

40-CpG Melanoma Classifier Calls in Uncertain Samples

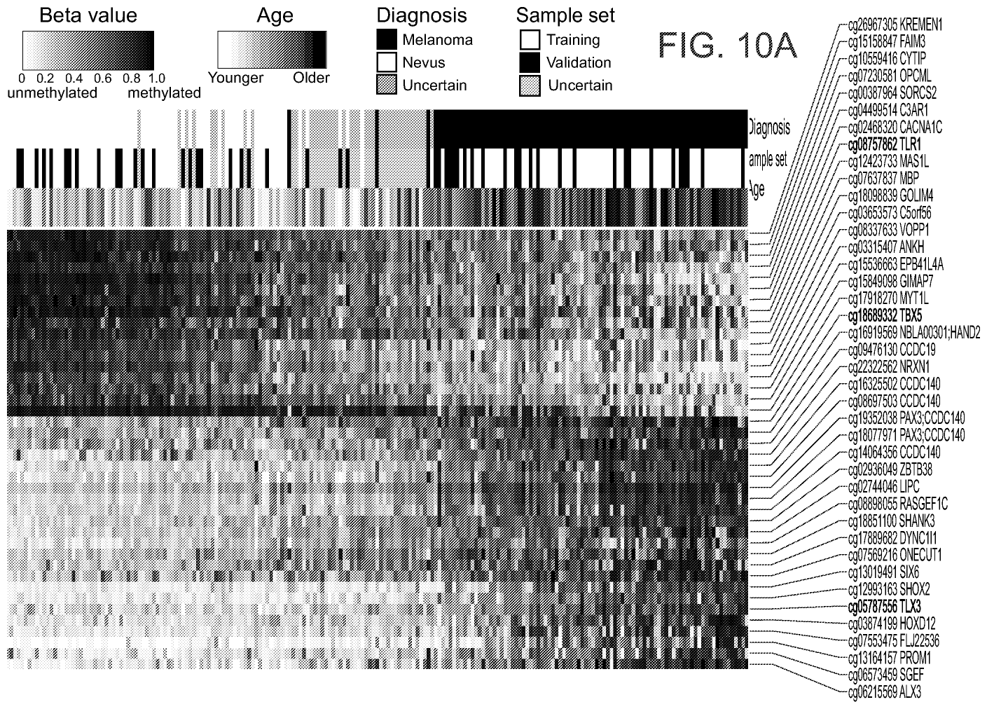

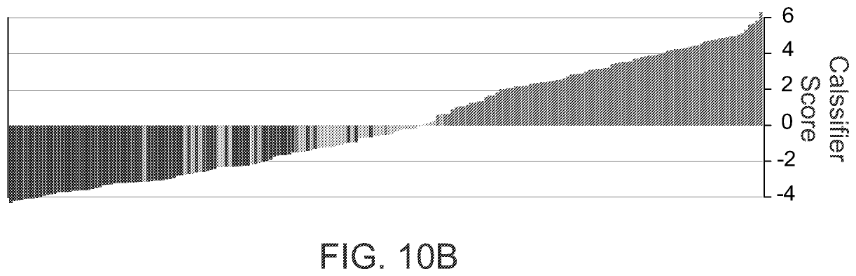

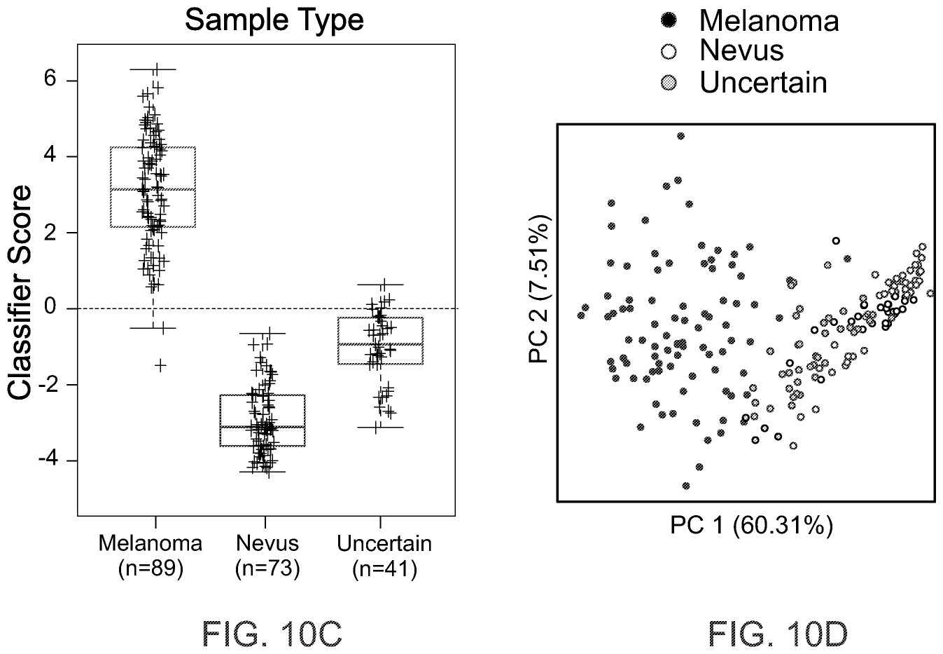

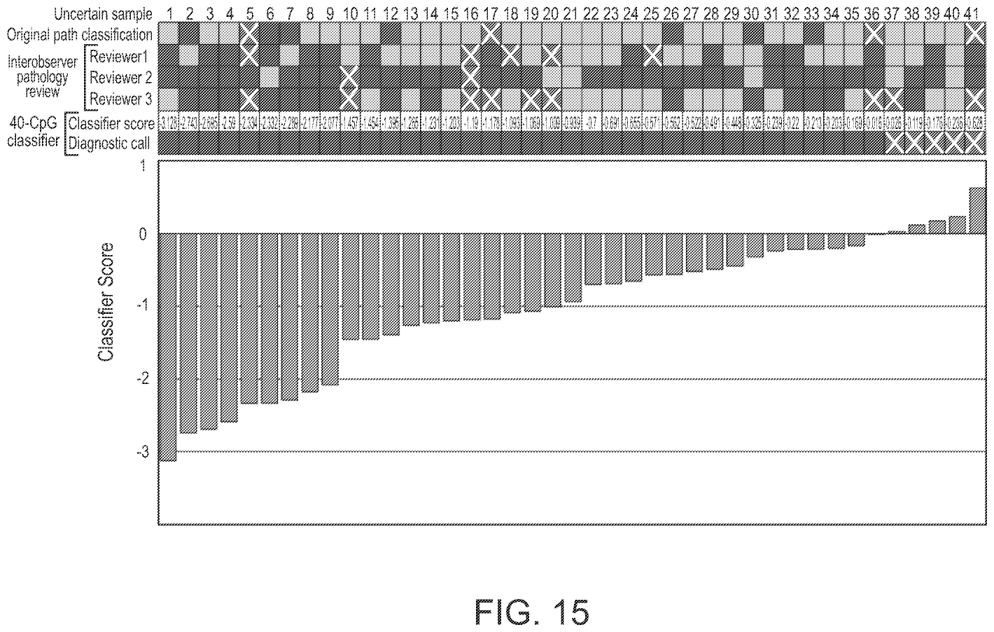

The 40-CpG classifier may be most clinically useful as an aid in the diagnosis of ambiguous melanocytic samples lacking agreement between dermatopathologists. Therefore, it was of interest to apply the 40-CpG melanoma classifier to the 41 diagnostically uncertain samples. The supervised heatmap in FIG. 10A illustrates methylation levels at the 40 diagnostic CpGs in uncertain samples along with the melanomas and nevi having diagnostic consensus, ordered from lowest (negative for nevi) to highest classifier score (positive for melanoma). In total, 36 uncertain samples were called nevus and 5 were called melanoma by the classifier, as shown in the waterfall plot (FIG. 10B). These results, together with the boxplots in FIG. 10C summarizing classifier scores for the three diagnostic categories, show that the uncertain samples reside mainly among the nevi or between the nevi and primary invasive melanomas. This is further confirmed by PCA based on either the 40 classifier CpGs (FIG. 10D) or the larger probe set (n=41,448) from which the classifier was derived (FIG. 14). The placement by the classifier of many diagnostically uncertain samples among the nevi is generally consistent with the pathology reviews in which 30 of 41 were called either nevus or uncertain by all the dermatopathology reviewers, while only 11 were called melanoma by any dermatopathology reviewer (Supplementary Table S8) and FIG. 15.

### 6.9. Discussion

This study identified a 40-CpG melanoma classifier that distinguished cutaneous primary invasive melanomas, including thin melanomas, from nevi with a sensitivity of 96.6% and specificity of 100.0% in the validation set. Methylation analysis was successfully performed on >97% of FFPE samples. The classifier is comprised of a combination of CpGs exhibiting hypermethylation (n=23) or hypomethylation (n=17) in melanomas relative to nevi. Although melanoma patients are typically older than those being biopsied for nevi, as in this dataset, the diagnostic accuracy of the classifier was similarly very high among both younger and older patients. Importantly, the classifier confirmed as melanoma nearly all 472 primary and metastatic melanomas in TCGA and was further independently validated in published methylation and gene expression datasets. Application of the classifier to uncertain samples predicted many to be nevi and a few to be melanomas. Thus, we believe the identification of a diagnostically uncertain melanocytic specimen as melanoma by the classifier increases the probability that it is a melanoma. As expected, some classifier scores for uncertain samples fell near the interface of melanoma and nevus, suggesting they may be in transition toward melanoma, and future work will focus on the characterization of such samples.

The 40 classifier CpGs for melanoma are associated with 38 genes heavily enriched for homeobox developmental transcription factors (ALX3, HOXD12, ONECUT1, PAX3, SHOX2, SIX6, TLX3) and other transcriptional regulators (TBX5, ZBTB38, MYT1L). PAX3, a marker of melanocytic cells, is a key regulator of melanocyte development and has putative roles in cell survival, migration, and differentiation (Dye et al., 2013; Medic and Ziman, 2009; 2010). Altered methylation of PAX3 and several other melanoma classifier genes (HOXD12, OPCML, GIMAP7, FAIM3) has previously been reported in melanomas versus nevi (Conway et al., 2011; Furuta et al., 2004; Gao et al., 2013; Jin et al., 2015). PROM1 (CD133), a stem cell marker involved in maintaining stem cell pluripotency, is frequently expressed in melanomas (Sharma et al., 2010; Zimmerer et al., 2016). Gene ontology analysis revealed associations of several diagnostic genes with neural tissues/processes (e.g., OPCML, NRXN1, HAND2, MYT1L, MBP, TLX3), reflecting their common embryologic derivation with melanocytes from neural crest cells (Noisa and Raivio, 2014). FLJ22536, recently identified as CASC15, is a putative mediator of neural growth and differentiation and a tumor suppressor in neuroblastoma (Russell et al., 2015), and in melanoma is linked to disease progression and phenotype switching between proliferative and invasive states (Lessard et al., 2015). Other diagnostic genes lack well-defined roles in melanoma; however, in other cancer types, a number exhibit aberrant expression (Gao et al., 2015; Jiang et al., 2008; Makiyama et al., 2005) and/or methylation (Jones et al., 2013; Kikuchi et al., 2013; Lai et al., 2008; Li et al., 2015; Semaan et al., 2016; Song et al., 2015; Wimmer et al., 2002; Yu et al., 2010; Zhao et al., 2013), function in apoptosis (Baras et al., 2009; Baras et al., 2011; Causeret et al., 2016) or differentiation (Zha et al., 2012), or are diagnostic (Semaan et al., 2016; Song et al., 2015; Xing et al., 2015), prognostic (Dietrich et al., 2013; Galluzzi et al., 2013; Qiu et al., 2015; Zheng et al., 2015; Zhou et al., 2014) or predictive biomarkers (Tada et al., 2011).

Our 40-CpG classifier for melanoma diagnosis may have advantages over other available approaches for melanoma diagnosis. In current clinical pathology practice, immunostains (e.g., Ki67, HMB45, p16) can aid pathologists' interpretation of melanocytic lesions, but single stains have low diagnostic accuracy (Uguen et al., 2015); combination staining may have higher accuracy but requires pathologist interpretation and lacks independent validation. Copy number analyses by comparative genomic hybridization (CGH) show that most melanomas, but few nevi, harbor numerous chromosomal changes (Bastian et al., 2000; Bauer and Bastian, 2006); however, CGH requires more tissue than is typically available from melanocytic samples. Fluorescence in situ hybridization detection of specific chromosomal changes is viewed directly on slides, using little tissue, but unlike CGH examines a limited number of chromosomes and requires technical expertise for interpretation (Busam, 2013). These currently utilized tests suffer from unclear diagnostic accuracy across the broad spectrum of melanoma and nevus subtypes (Ivan and Prieto, 2010) and limited independent validation. The Myriad MyPath Melanoma mRNA expression-based test showed reasonably high diagnostic accuracy (sensitivity and specificity >90%) for melanoma, but has a failure rate as high as 25% in FFPE archival samples (versus <3% in this study) (Clarke et al., 2015; Ko et al., 2017). The 40-CpG melanoma classifier is an approach that combines high accuracy across diverse melanocytic subtypes, technical robustness, and the ability to reliably screen early, small melanomas.

A strength of this study is that the 40-CpG melanoma classifier was developed from a genome-wide methylation platform allowing unbiased selection of loci. Notably, some of the identified loci may function in the neoplastic transition toward melanoma. Further, we utilized melanomas with a wide range of different AJCC tumor stages, including thin T1a melanomas, and diverse subtypes of both melanomas and nevi, such as dysplastic nevi, considered to be potential precursor lesions. For classification of melanoma or nevus in the training and validation sets, we required complete diagnostic consensus among three expert dermatopathologists and the original pathology report, crucial for achieving a highly accurate diagnostic classifier. Moreover, the classifier probes include only those with larger methylation differences between melanomas and nevi, which allows more reliable detection of these differences. Since the classifier was developed using FFPE samples similar to those typically found in clinical practice and requires amounts of DNA that can be recovered from most melanocytic samples, we expect the technology can be translated to clinical practice. Limitations of the study are its retrospective nature with potential sample selection bias. Another limitation is the absence of long-term follow-up of all patients.

In summary, our diagnostic 40-CpG melanoma classifier showed high accuracy in the validation set comprised of varied melanoma and nevus subtypes and was independently validated in public sample sets. Due to the robust nature of the assay, the 40-CpG melanoma classifier should be reliable on typical clinical samples. The assay also may have some advantages over other technologies due to its high diagnostic accuracy, need for less DNA, and robust methodology. However, additional studies are needed to further validate the performance of the classifier and optimize classifier score thresholds among larger numbers of samples, including rare melanocytic subtypes, especially in prospective studies with long-term follow-up.

### 6.10. Materials and Methods

Patients and Tissues

FFPE primary melanomas, nevi, and uncertain samples were assembled from the pathology archives of the University of North Carolina (UNC) Hospitals or from the University of Rochester (UR) Medical Center based on original diagnoses abstracted from pathology reports and diagnosed between 2001 and 2012. The Institutional Review Boards at UNC and the UR approved the study. Melanomas were chosen to span AJCC tumor stages and included common and less common subtypes (e.g., Spitzoid, nevoid, and desmoplastic melanomas). Nevi were chosen to include intradermal melanocytic nevi, including those with congenital pattern, compound melanocytic nevi with mild to severe dysplasia, Spitz and blue nevi, and other uncommon nevi (e.g. deep penetrating nevus, pigmented spindle cell nevus, and proliferative nodule in congenital pattern nevus). In addition, melanocytic proliferations of uncertain malignant potential were selected. Age, sex, race, and anatomic site were abstracted from the medical chart. Histopathological review of all samples was conducted independently by three expert dermatopathologists to assign diagnoses of melanoma or nevus or to identify uncertain samples. One dermatopathologist conducted a centralized histopathological review for histopathological pigment and adjacent solar elastosis of all the melanocytic lesions, for the histopathological subtype of nevi, and for histopathological subtype, Breslow thickness, mitoses, ulceration, and tumor infiltrating lymphocytes of the melanomas. Details of the histopathology are provided in Table 1. Details on the interobserver review are provided in the Supplementary Methods online.

DNA Preparation and Bisulfite Treatment

Melanocytic lesions were manually microdissected using H&E slides as guides, and DNA was prepared as described (Thomas et al., 2004; Thomas et al., 2007). Sodium bisulfite modification of 250-300 ng DNA from each FFPE tissue was performed using the EZ DNA Methylation Lightning kit (Zymo Research, Orange, Calif.) according to the manufacturer's protocol.

Infinium HumanMethylation450 BeadChip Analysis

Bisulfite-modified DNA (120 ng) was processed through the Illumina Infinium HD FFPE Restore protocol according to the manufacturer's instructions, and Illumina Infinium HumanMethylation450 BeadChip (450K) array analysis was performed in the Mammalian Genotyping Core at UNC. Details on methylation array analysis and data preprocessing are provided in the Supplementary Methods online. The final dataset contained 383,229 probes and 203 samples (89 melanomas, 73 nevi, 41 uncertain, and 12 controls). Methylation data were deposited to Gene Expression Omnibus under accession number GSE120878.

Statistical Analyses

To develop a diagnostic classifier distinguishing melanomas from nevi, melanomas and nevi with diagnostic consensus were split into training (67% of each sample class) and validation (the remaining 33%) sets. Multiple predictive models based on different probe sets were tested for their ability to distinguish melanomas from nevi; these included accounting for effects of age and limiting probes to the most differentially methylated. For each probe set, Monte-Carlo cross validation with 100 iterations was performed on training samples using the ElasticNet algorithm implemented in R package glmnet (Zou and Hastie, 2005) to obtain optimal parameters (alpha and the number of probes) that best differentiate melanomas. In each iteration, ⅔ of the training set was randomly selected to build the elastic model and to predict on the rest of the ⅓ in the training set. Based on the average AUC across 100 iterations, we determined the number of probes to be included in the final model. Classifier scores were calculated using the β value of selected probes in the final model. Heatmaps were generated to illustrate methylation at the diagnostic probe set, and PCA was performed to illustrate the segregation of melanomas and nevi. Additional details of model development and validation are provided in the Supplementary Methods online.

Independent Validation in Published Methylation Datasets

Illumina 450K methylation data for TCGA melanomas were downloaded from the Broad Institute Firehose web portal (http://firebrowse.org/) (version 2016012800). Illumina 450K methylation data for melanomas and nevi from the study of Wouters et al (Wouters et al., 2017) were obtained from Gene Expression Omnibus (GEO) (accession number GSE86355). Illumina Infinium HumanMethylation27 (27K) methylation data for melanomas and nevi were downloaded from GEO (accession number GSE45266) from the study of Gao et al (Gao et al., 2013).

### 6.11. References Section 6.6-6.11

Alexandrescu D T, Kauffman C L, Jatkoe T A, Hartmann D P, Vener T, Wang H, et al. Melanoma-specific marker expression in skin biopsy tissues as a tool to facilitate melanoma diagnosis. J Invest Dermatol 2010; 130(7): 1887-92.
Andtbacka R H, Kaufman H L, Collichio F, Amatruda T, Senzer N, Chesney J, et al. Talimogene Laherparepvec Improves Durable Response Rate in Patients With Advanced Melanoma. J Clin Oncol 2015; 33(25):2780-8.
Arai E, Kanai Y. DNA methylation profiles in precancerous tissue and cancers: carcinogenetic risk estimation and prognostication based on DNA methylation status. Epigenomics 2010; 2(3):467-81.
Balch C M, Gershenwald J E, Soong S J, Thompson J F, Atkins M B, Byrd D R, et al. Final version of 2009 AJCC melanoma staging and classification. J Clin Oncol 2009; 27(36):6199-206.
Baras A, Yu Y, Filtz M, Kim B, Moskaluk C A. Combined genomic and gene expression microarray profiling identifies ECOP as an upregulated gene in squamous cell carcinomas independent of DNA amplification. Oncogene 2009; 28(32):2919-24.
Baras A S, Solomon A, Davidson R, Moskaluk C A. Loss of VOPP1 overexpression in squamous carcinoma cells induces apoptosis through oxidative cellular injury. Lab Invest 2011; 91(8):1170-80.
Bastian B C, LeBoit P E, Pinkel D. Mutations and copy number increase of HRAS in Spitz nevi with distinctive histopathological features. Am J Pathol 2000; 157(3):967-72.
Bastian B C, Olshen A B, LeBoit P E, Pinkel D. Classifying melanocytic tumors based on DNA copy number changes. Am J Pathol 2003; 163(5):1765-70.
Bauer J, Bastian B C. Distinguishing melanocytic nevi from melanoma by DNA copy number changes: comparative genomic hybridization as a research and diagnostic tool. Dermatol Ther 2006; 19(1):40-9.
Brochez L, Verhaeghe E, Grosshans E, Haneke E, Pierard G, Ruiter D, et al. Inter-observer variation in the histopathological diagnosis of clinically suspicious pigmented skin lesions. J Pathol 2002; 196(4):459-66.
Busam K J. Molecular pathology of melanocytic tumors. Semin Diagn Pathol 2013; 30(4):362-74.
Causeret F, Sumia I, Pierani A. Kremen1 and Dickkopf1 control cell survival in a Wnt-independent manner. Cell Death Differ 2016; 23(2):323-32.
Clarke J M, George D J, Lisi S, Salama A K S. Immune Checkpoint Blockade: The New Frontier in Cancer Treatment. Target Oncol 2018.
Clarke L E, Warf M B, Flake D D, 2nd, Hartman A R, Tahan S, Shea C R, et al. Clinical validation of a gene expression signature that differentiates benign nevi from malignant melanoma. J Cutan Pathol 2015; 42(4):244-52.
Conway K, Edmiston S N, Khondker Z S, Groben P A, Zhou X, Chu H, et al. DNA-methylation profiling distinguishes malignant melanomas from benign nevi. Pigment Cell Melanoma Res 2011; 24(2):352-60.
Dietrich D, Jung M, Puetzer S, Leisse A, Holmes E E, Meller S, et al. Diagnostic and prognostic value of SHOX2 and SEPT9 DNA methylation and cytology in benign, paramalignant and malignant pleural effusions. PLoS One 2013; 8(12):e84225.
Dye D E, Medic S, Ziman M, Coombe D R. Melanoma biomolecules: independently identified but functionally intertwined. Front Oncol 2013; 3:252.
Elmore J, Barnhill R, Elder D, Longton G, Pepe M, Reisch L, et al. Pathologists' diagnosis of invasive melanoma and melanocytic proliferations: observer accuracy and resproducibility study. BMJ 2017; 357:j2813.
Farmer E R, Gonin R, Hanna M P. Discordance in the histopathologic diagnosis of melanoma and melanocytic nevi between expert pathologists. Hum Pathol 1996; 27(6):528-31.
Fung M A. Terminology and management of dysplastic nevi: responses from 145 dermatologists. Arch Dermatol 2003; 139(10): 1374-5.
Furuta J, Umebayashi Y, Miyamoto K, Kikuchi K, Otsuka F, Sugimura T, et al. Promoter methylation profiling of 30 genes in human malignant melanoma. Cancer Sci 2004; 95(12):962-8. Galluzzi L, Goubar A, Olaussen K A, Vitale I, Senovilla L, Michels J, et al. Prognostic value of LIPC in non-small cell lung carcinoma. Cell Cycle 2013; 12(4):647-54.
Gao C, Pang M, Zhou Z, Long S, Dong D, Yang J, et al. Epidermal growth factor receptor-coamplified and overexpressed protein (VOPP1) is a putative oncogene in gastric cancer. Clin Exp Med 2015; 15(4):469-75.
Gao L, Smit M A, van den Oord J J, Goeman J J, Verdegaal E M, van der Burg S H, et al. Genome-wide promoter methylation analysis identifies epigenetic silencing of MAPK13 in primary cutaneous melanoma. Pigment Cell Melanoma Res 2013; 26(4):542-54.
Gao L, van den Hurk K, Moerkerk P T, Goeman J J, Beck S, Gruis N A, et al. Promoter CpG island hypermethylation in dysplastic nevus and melanoma: CLDN11 as an epigenetic biomarker for malignancy. J Invest Dermatol 2014; 134(12):2957-66.
Gerami P, Busam K, Cochran A, Cook M G, Duncan L M, Elder D E, et al. Histomorphologic assessment and interobserver diagnostic reproducibility of atypical spitzoid melanocytic neoplasms with long-term follow-up. Am J Surg Pathol 2014; 38(7):934-40.
Gerami P, Jewell S S, Morrison L E, Blondin B, Schulz J, Ruffalo T, et al. Fluorescence in situ hybridization (FISH) as an ancillary diagnostic tool in the diagnosis of melanoma. Am J Surg Pathol 2009; 33(8):1146-56.
Haqq C, Nosrati M, Sudilovsky D, Crothers J, Khodabakhsh D, Pulliam B L, et al. The gene expression signatures of melanoma progression. Proc Natl Acad Sci USA 2005; 102(17):6092-7.
Herman J G, Baylin S B. Gene silencing in cancer in association with promoter hypermethylation. N Engl J Med 2003; 349(21):2042-54.
Hodi F S, Chesney J, Pavlick A C, Robert C, Grossmann K F, McDermott D F, et al. Combined nivolumab and ipilimumab versus ipilimumab alone in patients with advanced melanoma: 2-year overall survival outcomes in a multicentre, randomised, controlled, phase 2 trial. Lancet Oncol 2016; 17(11):1558-68.
Hoon D S, Spugnardi M, Kuo C, Huang S K, Morton D L, Taback B. Profiling epigenetic inactivation of tumor suppressor genes in tumors and plasma from cutaneous melanoma patients. Oncogene 2004; 23(22):4014-22.
Ivan D, Prieto V G. Use of immunohistochemistry in the diagnosis of melanocytic lesions: applications and pitfalls. Future Oncol 2010; 6(7):1163-75.
Jiang X, Zhang W, Kayed H, Zheng P, Giese N A, Friess H, et al. Loss of ONECUT1 expression in human pancreatic cancer cells. Oncol Rep 2008; 19(1):157-63.
Jin S G, Xiong W, Wu X, Yang L, Pfeifer G P. The DNA methylation landscape of human melanoma. Genomics 2015; 106(6):322-30.
Jones A, Teschendorff A E, Li Q, Hayward J D, Kannan A, Mould T, et al. Role of DNA methylation and epigenetic silencing of HAND2 in endometrial cancer development. PLoS Med 2013; 10(11):e1001551.
Kikuchi Y, Tsuji E, Yagi K, Matsusaka K, Tsuji S, Kurebayashi J, et al. Aberrantly methylated genes in human papillary thyroid cancer and their association with BRAF/RAS mutation. Front Genet 2013; 4:271.
Ko J S, Matharoo-Ball B, Billings S D, Thomson B J, Tang J Y, Sarin K Y, et al. Diagnostic Distinction of Malignant Melanoma and Benign Nevi by a Gene Expression Signature and Correlation to Clinical Outcomes. Cancer Epidemiol Biomarkers Prev 2017; 26(7):1107-13.
Koh S S, Opel M L, Wei J P, Yau K, Shah R, Gorre M E, et al. Molecular classification of melanomas and nevi using gene expression microarray signatures and formalin-fixed and paraffin-embedded tissue. Mod Pathol 2009; 22(4):538-46.
Lai H C, Lin Y W, Huang T H, Yan P, Huang R L, Wang H C, et al. Identification of novel DNA methylation markers in cervical cancer. Int J Cancer 2008; 123(1):161-7.
Landow S M, Gjelsvik A, Weinstock M A. Mortality burden and prognosis of thin melanomas overall and by subcategory of thickness, SEER registry data, 1992-2013. J Am Acad Dermatol 2017; 76(2):258-63.
Lessard L, Liu M, Marzese D M, Wang H, Chong K, Kawas N, et al. The CASC15 Long Intergenic Noncoding RNA Locus Is Involved in Melanoma Progression and Phenotype Switching. J Invest Dermatol 2015; 135(10):2464-74.
Li C, Tang L, Zhao L, Li L, Xiao Q, Luo X, et al. OPCML is frequently methylated in human colorectal cancer and its restored expression reverses EMT via downregulation of smad signaling. Am J Cancer Res 2015; 5(5):1635-48.
Long G V, Flaherty K T, Stroyakovskiy D, Gogas H, Levchenko E, de Braud F, et al. Dabrafenib plus trametinib versus dabrafenib monotherapy in patients with metastatic BRAF V600E/K-mutant melanoma: long-term survival and safety analysis of a phase 3 study. Ann Oncol 2017; 28(7): 1631-9.
Makiyama K, Hamada J, Takada M, Murakawa K, Takahashi Y, Tada M, et al. Aberrant expression of HOX genes in human invasive breast carcinoma. Oncol Rep 2005; 13(4):673-9.
Medic S, Ziman M. PAX3 across the spectrum: from melanoblast to melanoma. Crit Rev Biochem Mol Biol 2009; 44(2-3):85-97.
Medic S, Ziman M. PAX3 expression in normal skin melanocytes and melanocytic lesions (naevi and melanomas). PLoS One 2010; 5(4):e9977.
Noisa P, Raivio T. Neural crest cells: from developmental biology to clinical interventions. Birth Defects Res C Embryo Today 2014; 102(3):263-74.
North J P, Garrido M C, Kolaitis N A, LeBoit P E, McCalmont T H, Bastian B C. Fluorescence in situ hybridization as an ancillary tool in the diagnosis of ambiguous melanocytic neoplasms: a review of 804 cases. Am J Surg Pathol 2014; 38(6):824-31.
Plass C. Cancer epigenomics. Hum Mol Genet 2002; 11(20):2479-88.
Qiu Z X, Zhao S, Mo X M, Li W M. Overexpression of PROM1 (CD133) confers poor prognosis in non-small cell lung cancer. Int J Clin Exp Pathol 2015; 8(6):6589-95.
Ribas A, Hamid O, Daud A, Hodi F S, Wolchok J D, Kefford R, et al. Association of Pembrolizumab With Tumor Response and Survival Among Patients With Advanced Melanoma. JAMA 2016; 315(15):1600-9.
Ribas A, Puzanov I, Dummer R, Schadendorf D, Hamid O, Robert C, et al. Pembrolizumab versus investigator-choice chemotherapy for ipilimumab-refractory melanoma (KEYNOTE-002): a randomised, controlled, phase 2 trial. Lancet Oncol 2015; 16(8):908-18.
Robert C, Long G V, Brady B, Dutriaux C, Maio M, Mortier L, et al. Nivolumab in previously untreated melanoma without BRAF mutation. N Engl J Med 2015; 372(4):320-30.
Russell M R, Penikis A, Oldridge D A, Alvarez-Dominguez J R, McDaniel L, Diamond M, et al. CASC15-S Is a Tumor Suppressor lncRNA at the 6p22 Neuroblastoma Susceptibility Locus. Cancer Res 2015; 75(15):3155-66.
Schachter J, Ribas A, Long G V, Arance A, Grob J J, Mortier L, et al. Pembrolizumab versus ipilimumab for advanced melanoma: final overall survival results of a multicentre, randomised, open-label phase 3 study (KEYNOTE-006). Lancet 2017; 390(10105):1853-62.
Semaan A, van Ellen A, Meller S, Bergheim D, Branchi V, Lingohr P, et al. SEPT9 and SHOX2 DNA methylation status and its utility in the diagnosis of colonic adenomas and colorectal adenocarcinomas. Clin Epigenetics 2016; 8:100.
Shaikh W R, Dusza S W, Weinstock M A, Oliveria S A, Geller A C, Halpern A C. Melanoma Thickness and Survival Trends in the United States, 1989 to 2009. J Natl Cancer Inst 2016; 108(1). Shain A H, Yeh I, Kovalyshyn I, Sriharan A, Talevich E, Gagnon A, et al. The Genetic Evolution of Melanoma from Precursor Lesions. N Engl J Med 2015; 373(20):1926-36.
Sharma B K, Manglik V, Elias E G. Immuno-expression of human melanoma stem cell markers in tissues at different stages of the disease. J Surg Res 2010; 163(1):e11-5.
Shoo B A, Sagebiel R W, Kashani-Sabet M. Discordance in the histopathologic diagnosis of melanoma at a melanoma referral center. J Am Acad Dermatol 2010; 62(5):751-6.
Siegel R L, Miller K D, Jemal A. Cancer statistics, 2018. CA Cancer J Clin 2018; 68(1):7-30.
Song L, Yu H, Li Y. Diagnosis of Lung Cancer by SHOX2 Gene Methylation Assay. Mol Diagn Ther 2015; 19(3):159-67.
Strauss R M, Elliott F, Affleck P, Boon A P, Newton-Bishop J A. A retrospective study addressed to understanding what predicts severe histological dysplasia/early melanoma in excised atypical melanocytic lesions. Br J Dermatol 2007; 157(4):758-64.
Tada Y, Yokomizo A, Shiota M, Tsunoda T, Plass C, Naito S. Aberrant DNA methylation of T-cell leukemia, homeobox 3 modulates cisplatin sensitivity in bladder cancer. Int J Oncol 2011; 39(3):727-33.
Talantov D, Mazumder A, Yu J X, Briggs T, Jiang Y, Backus J, et al. Novel genes associated with malignant melanoma but not benign melanocytic lesions. Clin Cancer Res 2005; 11(20):7234-42.
TCGA N. Genomic Classification of Cutaneous Melanoma. Cell 2015; 161(7):1681-96.
Thomas N E, Alexander A, Edmiston S N, Parrish E, Millikan R C, Berwick M, et al. Tandem BRAF mutations in primary invasive melanomas. J Invest Dermatol 2004; 122(5):1245-50.
Thomas N E, Edmiston S N, Alexander A, Millikan R C, Groben P A, Hao H, et al. Number of nevi and early-life ambient U V exposure are associated with BRAF-mutant melanoma. Cancer Epidemiol Biomarkers Prev 2007; 16(5):991-7.
Thomas N E, Slater N A, Edmiston S N, Zhou X, Kuan P F, Groben P A, et al. DNA methylation profiles in primary cutaneous melanomas are associated with clinically significant pathologic features. Pigment Cell Melanoma Res 2014; 27(6):1097-105.
Uguen A, Talagas M, Costa S, Duigou S, Bouvier S, De Braekeleer M, et al. A p16-Ki-67-HMB45 immunohistochemistry scoring system as an ancillary diagnostic tool in the diagnosis of melanoma. Diagn Pathol 2015; 10:195.
Veenhuizen K C, De Wit P E, Mooi W J, Scheffer E, Verbeek A L, Ruiter D J. Quality assessment by expert opinion in melanoma pathology: experience of the pathology panel of the Dutch Melanoma Working Party. J Pathol 1997; 182(3):266-72.
Whiteman D C, Baade P D, Olsen C M. More people die from thin melanomas (1 mm) than from thick melanomas (>4 mm) in Queensland, Australia. J Invest Dermatol 2015; 135(4):1190-3.
Wimmer K, Zhu Xx X X, Rouillard J M, Ambros P F, Lamb B J, Kuick R, et al. Combined restriction landmark genomic scanning and virtual genome scans identify a novel human homeobox gene, ALX3, that is hypermethylated in neuroblastoma. Genes Chromosomes Cancer 2002; 33(3):285-94.
Wouters J, Vizoso M, Martinez-Cardus A, Carmona F J, Govaere O, Laguna T, et al. Comprehensive DNA methylation study identifies novel progression-related and prognostic markers for cutaneous melanoma. Bmc Med 2017; 15(1):101.
Xing B L, Li T, Tang Z H, Jiao L, Ge S M, Qiang X, et al. Cumulative methylation alternations of gene promoters and protein markers for diagnosis of epithelial ovarian cancer. Genet Mol Res 2015; 14(2):4532-40.
Yu J, Ma X, Cheung K F, Li X, Tian L, Wang S, et al. Epigenetic inactivation of T-box transcription factor 5, a novel tumor suppressor gene, is associated with colon cancer. Oncogene 2010; 29(49):6464-74.
Zha Y, Ding E, Yang L, Mao L, Wang X, McCarthy B A, et al. Functional dissection of HOXD cluster genes in regulation of neuroblastoma cell proliferation and differentiation. PLoS One 2012; 7(8):e40728.
Zhao Y, Zhou H, Ma K, Sun J, Feng X, Geng J, et al. Abnormal methylation of seven genes and their associations with clinical characteristics in early stage non-small cell lung cancer. Oncol Lett 2013; 5(4):1211-8.
Zheng Y, Li Y F, Wang W, Chen Y M, Wang D D, Zhao J J, et al. High expression level of T-box transcription factor 5 predicts unfavorable survival in stage I and II gastric adenocarcinoma. Oncol Lett 2015; 10(4):2021-6.
Zhou F, Tao G, Chen X, Xie W, Liu M, Cao X. Methylation of OPCML promoter in ovarian cancer tissues predicts poor patient survival. Clin Chem Lab Med 2014; 52(5):735-42.
Zimmerer R M, Matthiesen P, Kreher F, Kampmann A, Spalthoff S, Jehn P, et al. Putative CD133+ melanoma cancer stem cells induce initial angiogenesis in vivo. Microvasc Res 2016; 104:46-54.
Zou H, Hastie T. Regularization and variable selection via the Elastic Net. Journal of the Royal Statistical Society 2005; Series B (Statistical Methodology)(67):301-20.

### 6.12. TERT Promoter Mutation Analysis

### 6.13. Background Section 6.12-6.18

Highly-recurrent somatic CT mutations have been reported in up to 55% of primary cutaneous melanomas in the promoter of the catalytic reverse transcriptase subunit of the telomerase (TERT) gene, the ribonucleoprotein complex that maintains telomere length [1-8]. Hayward et al. report TERT promoter mutations to be 54.7% (41/75) in primary cutaneous melanoma Nature 2017; 545(7653):175-180 supplemental data. The majority of these mutations are C to T transitions that occur at positions −124 or −146 upstream from the transcription start site. Mutations at these positions create identical 11-bp nucleotide stretches that contain a consensus-binding site for E-twenty-six (ETS) transcription factors, in the ternary complex factor (TCF) subfamily. Other TERT promoter mutations reported in melanomas and which also create ETS/TCF binding sites occur at positions −57 or −138/139 from the start site [2,4,5,7,9]. TERT promoter mutations at sites −124 or −146 were found to have increased TERT mRNA expression and to increase transcriptional activity of TERT promoter luciferase constructs in reporter assays [1,4,10,11]. Studies have also demonstrated that TERT promoter −138/−139CC>TT mutations in melanoma correlate with TERT overexpression [10]. Presence of TERT promoter mutations has been associated with worse survival from melanoma [8,12]. This effect was found to be modified by a common polymorphism rs2853669 within the TERT promoter that disrupts a preexisting noncanonical ETS2 site in the proximal region of the TERT promoter immediately adjacent to an E-box [12]. Further, TERT promoter mutations in Spitzoid melanocytic neoplasms were reported to predict aggressive clinical behavior [13].

Previous small studies indicate that TERT promoter mutations are rare in benign precursor nevi (moles); however, the studies have not been definitive as they were based on small numbers of nevi [2,14,15]. Horn et al. screened 25 melanocytic nevi and found only one carried a mutation in the TERT promoter at −101 bp, which did not create an ETS/TCF motif [2]. Vinagre et al. did not detect TERT promoter mutations in 9 benign nevi tested [14]. Requena et al. found that none of 15 Spitz/Reed nevi carried TERT promoter mutations; whereas, two of nine atypical Spitzoid tumors contained TERT promoter mutations [15].

Due to their frequent presence in melanomas but rarity in nevi, as candidate markers, TERT promoter mutations may be ideally-suited for melanoma diagnosis. In contrast, BRAF and NRAS mutations are frequently found in benign nevi, minimizing their diagnostic value. Thus, we analyzed for TERT promoter mutations a series of the melanocytic lesions we had previously profiled using 450K methylation analysis. This series of melanocytic lesions had undergone interobserver review by three dermatopathologists to classify the lesions histopathologically as melanoma, nevus, or melanocytic proliferations of uncertain diagnosis.

Further, we examined whether a cost-effective sequential screening algorithm of detection first of TERT promoter mutations followed by DNA methylation screening would be an accurate method of melanoma identification. We also report the associations of TERT promoter mutations in melanomas with melanoma clinicopathologic features and with the common single-nucleotide polymorphism rs2853669 (−245T>C) within the TERT promoter region.

### 6.14. Methods

TERT promoter mutational screening. DNA prepared from primary melanomas and nevi as previously described above were screened for TERT promoter mutation by sequencing of a 270-base pair amplicon of the TERT promoter that encompasses the main target region for mutations. This region was amplified (primer F: 5′-CCGGGCTCCCAGTGGATTCG; primer R: 5′-GCTTCCCACGTGCGCAGCAGGA)(SEQ ID NO: 477-478) using primers as previously described targeted to amplify from −270 to −50 bps from the start site within the promoter region of the TERT gene [16]. Several melanoma cell lines and tissues had been pre-screened to identify appropriate positive and negative controls. Samples were sequenced in the UNC DNA Sequencing Core Facility from the purified 270 bp TERT PCR product using cycle sequencing with fluorescently labeled Big Dye terminators (ABI) on an ABI 3730 DNA Analyzer. To eliminate mutational artifacts, we repeated sequencing of a separately amplified aliquot of DNA. Inner primers were designed for sequencing (primer F-in: 5′-CTCCCAGTGGATTCGCGGGCA; primer R-in: 5′-CCCACGTGCGCAGCAGGAC) (SEQ ID NO: 479-480).

### 6.15. Results

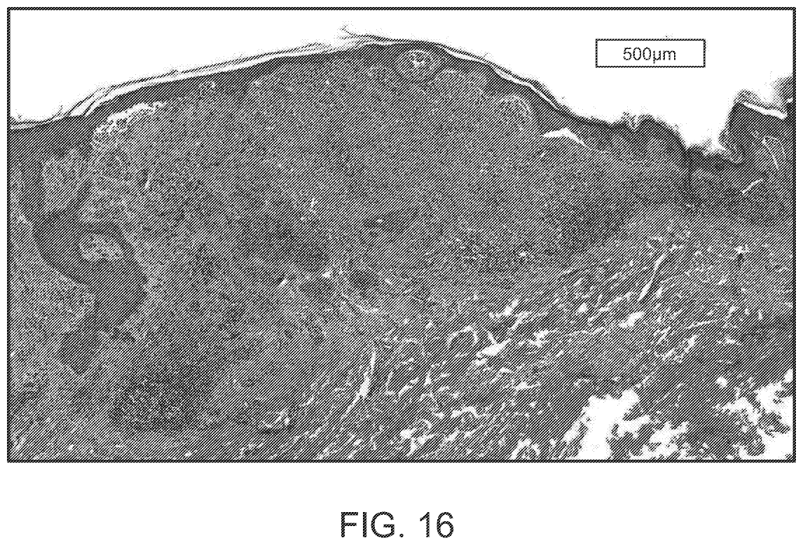

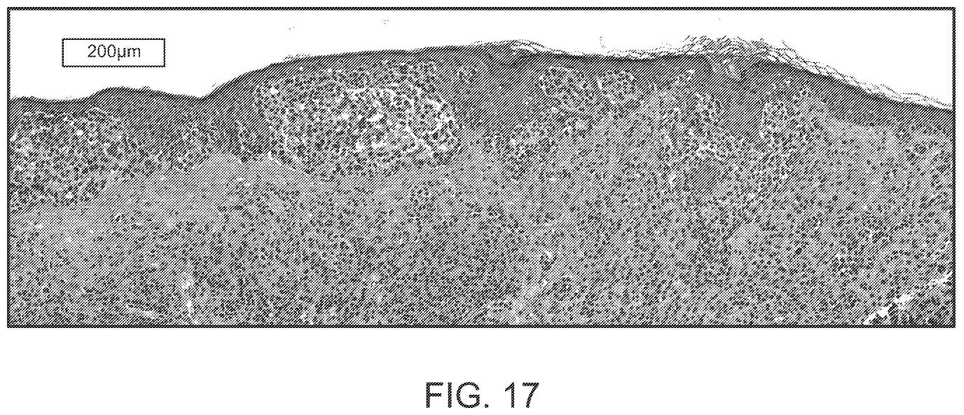

Frequency of TERT promoter mutations in melanocytic lesions. The following samples failed analysis for TERT promoter mutation and were excluded from analyses: melanomas (n=3), nevus (n=1), and melanocytic proliferations of uncertain diagnosis (n=1). As shown in Supp. TABLE S10, of the 86 successfully analyzed invasive melanomas, 67 (77.9%) had a TERT mutation that created a de novo confirmed functional ETS/TCF transcription factor binding site (at hotspot sites −124, −146 or −138/139). Of the remaining successfully analyzed melanomas: 4 (4.7%) had a TERT promoter mutation that created a de novo ETS/TCF binding site that has not been confirmed to be functional; 2 (2.3%) had a TERT promoter mutation that did not form an ETS/TCF site (‘other’ mutations); and 13 (15.2%) had no TERT promoter mutation. Examples of a TERT-positive and a TERT-negative melanoma are illustrated in FIGS. 16 and 17, respectively.

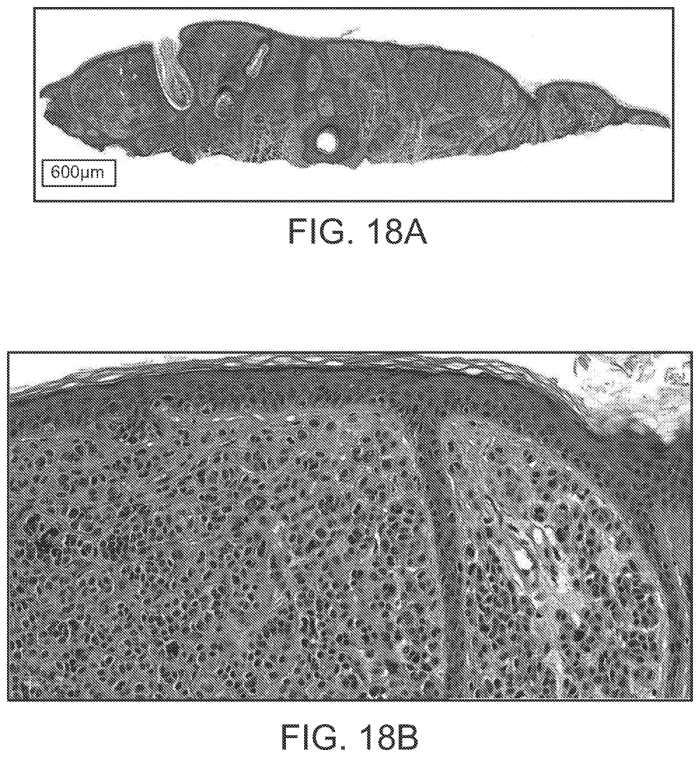

Of the 72 nevi, only 1 nevus (1.4%) had a TERT promoter mutation creating a confirmed functional ETS/TCF site. That one intradermal nevus (1.4%) from a 41 year old male, shown in FIG. 18A-18B, had a hotspot −124C>T TERT promoter mutation. 7 (9.7%) of nevi had ‘other’ mutations, and 64 (88.9%) had no TERT promoter mutations.

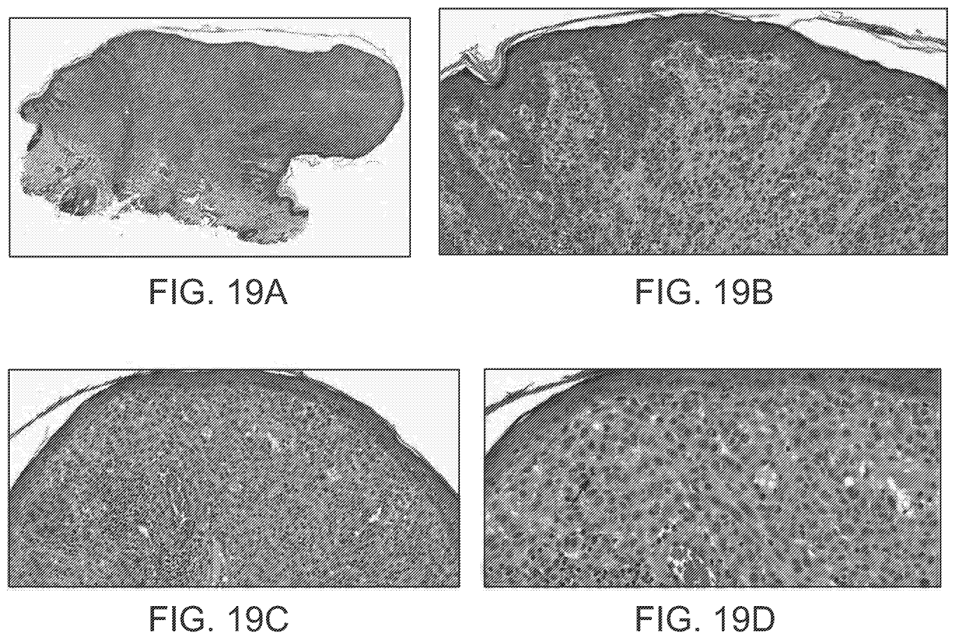

Of the 40 melanocytic proliferations of uncertain diagnosis (‘uncertain’ samples), 2 (5.0%) had a TERT promoter mutation creating a de novo confirmed functional ETS/TCF site (124C>T or 146C>T). An uncertain specimen that harbored a TERT promoter mutation at −146C>T is illustrated in FIG. 19A-19D. 1 (2.5%) had a TERT promoter mutation creating a de novo unconfirmed functional ETS/TCF site (156C>T); and 35 (87.5%) had no TERT promoter mutation (Supp. TABLE S10). Supp. TABLE S11 describes the characteristics of 86 primary melanomas, 72 nevi, and 40 melanocytic proliferations with uncertain diagnosis. On the original pathology report, the ‘uncertain’ sample with the 124C>T TERT promoter mutation was histologically described as an ‘atypical compound dysplastic nevus/thin invasive melanoma’ and 1 of 3 dermatopathologists called it uncertain on the interobserver review. The sample with the 146C>T TERT promoter mutation was described as ‘viewed by multiple pathologists with differing opinions’ and 2 of 3 dermatopathologists called it melanoma on the interobserver review. The sample with the 156C>T TERT promoter mutation was called a ‘melanoma’ on the original pathology report, and 1 of 3 dermatopathologists called it melanoma on the interobserver review.

TERT promoter mutations are highly specific for melanomas. Notably, the TERT promoter mutations creating de novo unconfirmed functional ETS/TCF sites were found only in melanomas and one ‘uncertain’ sample. Thus, we examined diagnostic accuracy for melanoma vs. nevi using two definitions for calling TERT promoter mutations ‘positive’. There were Definition 1: TERT positive if a de novo confirmed functional ETS/TCF site is present; and Definition 2: TERT positive if a de novo confirmed or un-confirmed functional ETS/TCF site is present (Supp. TABLE S10).

Using Definition 1, the ability of TERT mutation positivity as a test for melanoma vs. nevus had a diagnostic accuracy of 87.3% (95% CI, 81.1 to 92.1%) with a sensitivity of 77.9% and specificity of 98.6%. The positive predictive value (PPV) was 98.5% and negative predictive value (NPV) was 78.9%. Thus, occurrence of a confirmed functional TERT promoter mutation in a melanocytic sample should place this lesion under high suspicion for being a melanoma.

Using Definition 2, the ability of TERT mutation positivity as a test for melanoma vs. nevus had a diagnostic accuracy of 89.9% (95% CI, 84.1 to 94.1%) with a sensitivity of 82.6% and specificity of 98.6%. The PPV was 98.6% and NPV was 82.6%. Definition 2, which included as positive those samples with confirmed or unconfirmed TERT promoter mutations improved the diagnostic accuracy of the assay by improving the sensitivity.

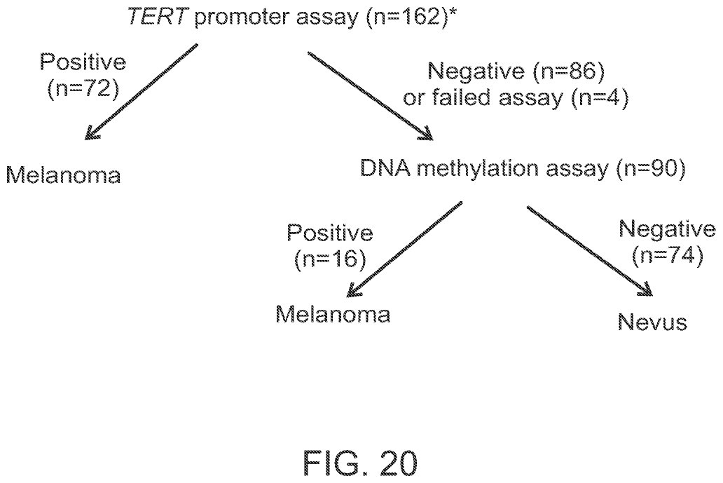

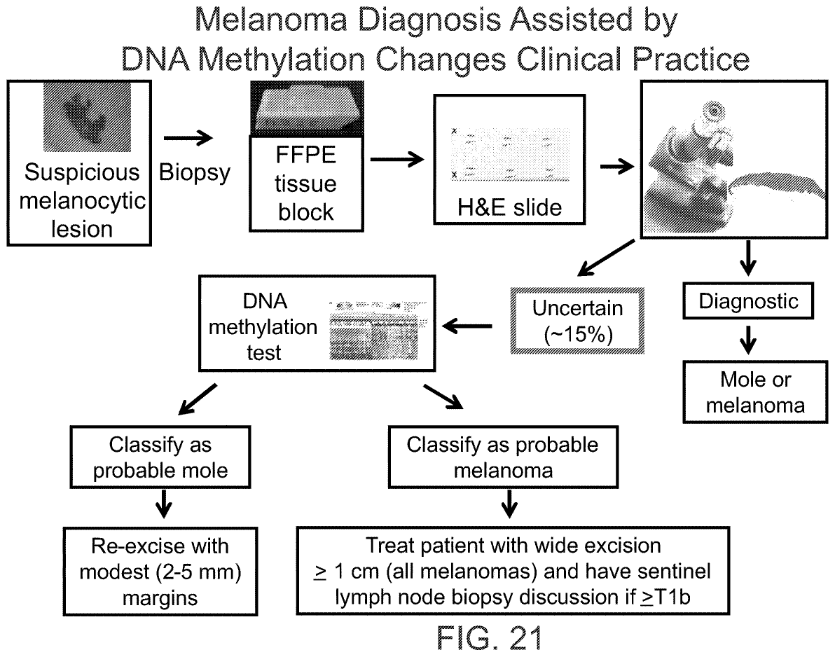

The combination of TERT promoter mutations and DNA methylation assays for diagnosis. We examined an algorithm for diagnosis of melanocytic lesions in which TERT promoter mutation assays are theoretically performed first followed by DNA methylation assays on cases negative for TERT promoter mutations (FIG. 20). If the sample is positive for TERT promoter mutation, the sample is designated as a melanoma but if it is negative or fails this assay, then the DNA methylation assay is performed. If the DNA methylation assay is positive, it is designated a melanoma, and if it is negative, then the sample is designated a nevus. For these two assays run in this manner, using Definition 1, the ability of TERT mutation positivity as a test for melanoma vs. nevus using these tests sequentially had a diagnostic accuracy of 98.2% (95% CI, 94.7-99.6%) with a sensitivity of 97.8% and specificity of 98.6%; the PPV was 98.9% and NPV was 97.3%. Using Definition 2, the ability of TERT mutation positivity using these tests sequentially as a test for melanoma vs. nevus had a diagnostic accuracy of 98.2% (95% CI, 94.7-99.6%) with a sensitivity of 97.8% and specificity of 98.6%; the PPV was 98.9% and NPV was 97.3%.

Relationship of TERT promoter mutations to clinicopathologic features. Using Definition 1, TERT promoter mutation positivity in melanomas was associated with older age at diagnosis (p=0.005), whites of European origin (p=0.02), histologic type (p=0.01), anatomic site (p<0.001), and the presence of solar elastosis (p=0.01) (Supp. TABLE S13). Notably, TERT promoter mutations were less common in acral lentiginous melanomas, with only 1 (16.7%) being TERT positive using Definition 1 or 2. TERT promoter mutations were also less likely to occur in melanomas on the lower extremities compared to other sites. There was no association with sex, presence of contiguous nevus, Breslow thickness, ulceration, mitoses, 2018 AJCC Stage, tumor infiltrating lymphocyte grade, regression, pigment, or presence of the rs2853669 single nucleotide polymorphism in the TERT promoter. The results were similar using Definition 2.

### 6.16. Discussion

We found the specificity of TERT promoter mutation for melanoma vs. nevus was 98.6%, with only 1 of 72 nevi harboring a mutation. These results indicate that a melanocytic lesion with a TERT promoter mutation should be viewed as melanoma unless strong evidence to the contrary exists. The sensitivity for melanoma ranged from 77.9 to 82.6% for Definitions 1 and 2, respectively. We found −124C>T, −146C>T and −138/−139CC>TT mutations in the melanomas, as reported in the literature [1-8]. However, we also found in melanomas additional mutations in the TERT promoter at 103C>T, 105_106CC>TT, 148C>T that form ETS/TCF sites and to our knowledge have not been reported in melanoma previously. We found these only to be present in melanomas and not nevi, indicating that they may be functional mutations. Including these additional mutations as positives in Definition 2 increased the assay sensitivity for melanoma to 82.6%. Further work on determining whether these mutations are functional seems warranted.

Presence of a TERT promoter mutation in melanocytic samples of uncertain potential may help to discriminate melanoma vs. nevi. We found 124C>T and 146C>T mutations in two different ‘uncertain’ samples. Further, we found a 156C>T mutation, which forms an ETS/TCF site, in another ‘uncertain’ sample. Heidenreich et al, [4] previously reported a 156C>T mutation in a cutaneous melanoma. Our data indicate that the presence of TERT promoter mutations in uncertain samples provides evidence that they are melanomas.

We examined an algorithm of performing TERT promoter assays first followed by examining TERT negative samples with DNA methylation profiling for purposes of diagnosing melanoma. This sequential assay was of interest as a cost saving measure to avoid the expense of methylation arrays. The sequential assays, as depicted in FIG. 1 with the results in Supp. TABLE S12, led to high diagnostic accuracy.

We found TERT promoter were associated with increased age at diagnosis similar to other studies [4,12,17]. We found TERT promoter mutations more frequently in melanomas from whites of European origin vs. other/unknown races; however, this needs to be examined in larger datasets with larger numbers of patients who are not whites of European origin. Notably Bal et al. found a low rate of TERT promoter mutations in melanomas in the Asian population [18]. We found only 16.7% of acral lentiginous melanomas harbored TERT promoter mutations consistent with the literature [4,5,7,8,12,18-20]. Similar to Heidenreich et al. [4], we found TERT mutations were associated with melanomas arising on sun-exposed anatomic sites (defined as presence of solar elastosis in our study). Unlike several other studies, we did not find an association with increased Breslow thickness, ulceration, tumor stage or mitotic rate [4,7,8,12]. We found no association of TERT promoter mutation in melanomas with regression, unlike other studies where negative [21] and positive [22] correlations were reported. Unlike Ofner et al. [9] but similar to Nagore et al. [12], we found no significant association of TERT promoter with the carrier status of the common single-nucleotide polymorphism rs2853669.

To our knowledge, this is the first study to examine the diagnostic accuracy of TERT promoter mutations for diagnosing melanoma. The strengths of the study include inclusion of a balanced number of melanoma of different tumor stages and histologic subtypes and a variety of nevus subtypes. The melanocytic samples underwent interob server review by three dermatopathologists to classify them as melanoma, nevi or melanocytic proliferations of uncertain diagnosis. The samples underwent rigorous TERT promoter mutational analysis with inclusion of the less common TERT promoter mutations in the analysis. Further, we are able to combine data on TERT promoter mutations and DNA methylation for the same samples. The inclusion of uncertain samples provides additional information on whether TERT promoter mutation can be found among those samples that are difficult to classify. Weaknesses of the study include a very limited number of samples from patients who are not whites of European origin and some samples from patients of unknown race.

### 6.17. Conclusions

Our results indicate that TERT promoter mutations may be useful in diagnosis of melanoma versus nevus when the diagnosis is uncertain histologically. Notably, our study indicates that less common TERT promoter mutations forming ETS/TCF sites are also diagnostic for melanoma, increasing the sensitivity of utilizing TERT promoter mutations for diagnosis.

However, large series of melanocytic samples need to be studied to confirm our results and determine diagnostic accuracy for less common subtypes and different races. Our results and that of others indicate that TERT promoter mutations in melanomas from races other than whites of European origin and in acral lentiginous melanomas are less frequent, making the sensitivity for diagnosing melanoma lower in these cases. Moreover, examination of TERT promoter mutations in melanocytic proliferations of uncertain diagnosis warrants additional study, in particular, among patients where long-term outcome is available, allowing better objective classification. Lastly, an algorithm for diagnosis of melanocytic lesions in which TERT promoter mutation assays are performed first followed by DNA methylation assays on cases negative for TERT promoter mutations seems promising as a cost-effective method with high diagnostic accuracy for melanoma.

### 6.18. TERT Mutation References (Section 2)

1. Huang F W, Hodis E, Xu M J, Kryukov G V, Chin L, Garraway L A. Highly recurrent TERT promoter mutations in human melanoma. Science 2013; 339(6122):957-9 doi 10.1126/science.1229259.
2. Horn S, Figl A, Rachakonda P S, Fischer C, Sucker A, Gast A, Kadel S, Moll I, Nagore E, Hemminki K, Schadendorf D, Kumar R. TERT promoter mutations in familial and sporadic melanoma. Science 2013; 339(6122):959-61 doi 10.1126/science.1230062.
3. Egberts F, Kruger S, Behrens H M, Bergner I, Papaspyrou G, Werner J A, Alkatout I, Haag J, Hauschild A, Rocken C. Melanomas of unknown primary frequently harbor TERT-promoter mutations. Melanoma Res 2014; 24(2):131-6 doi 10.1097/CMR.0000000000000048.
4. Heidenreich B, Nagore E, Rachakonda P S, Garcia-Casado Z, Requena C, Traves V, Becker J, Soufir N, Hemminki K, Kumar R. Telomerase reverse transcriptase promoter mutations in primary cutaneous melanoma. Nat Commun 2014; 5:3401 doi 10.1038/ncomms4401.
5. Hayward N K, Wilmott J S, Waddell N, Johansson P A, Field M A, Nones K, Patch A M, Kakavand H, Alexandrov L B, Burke H, Jakrot V, Kazakoff S, Holmes O, Leonard C, Sabarinathan R, Mularoni L, Wood S, Xu Q, Waddell N, Tembe V, Pupo G M, De Paoli-Iseppi R, Vilain R E, Shang P, Lau L M S, Dagg R A, Schramm S J, Pritchard A, Dutton-Regester K, Newell F, Fitzgerald A, Shang C A, Grimmond S M, Pickett H A, Yang J Y, Stretch J R, Behren A, Kefford R F, Hersey P, Long G V, Cebon J, Shackleton M, Spillane A J, Saw R P M, Lopez-Bigas N, Pearson J V, Thompson J F, Scolyer R A, Mann G J. Whole-genome landscapes of major melanoma subtypes. Nature 2017; 545(7653):175-80 doi 10.1038/nature22071.
6. Zehir A, Benayed R, Shah R H, Syed A, Middha S, Kim H R, Srinivasan P, Gao J, Chakravarty D, Devlin S M, Hellmann M D, Barron D A, Schram A M, Hameed M, Dogan S, Ross D S, Hechtman J F, DeLair D F, Yao J, Mandelker D L, Cheng D T, Chandramohan R, Mohanty A S, Ptashkin R N, Jayakumaran G, Prasad M, Syed M H, Rema A B, Liu Z Y, Nafa K, Borsu L, Sadowska J, Casanova J, Bacares R, Kiecka I J, Razumova A, Son J B, Stewart L, Baldi T, Mullaney K A, Al-Ahmadie H, Vakiani E, Abeshouse A A, Penson A V, Jonsson P, Camacho N, Chang M T, Won H H, Gross B E, Kundra R, Heins Z J, Chen H W, Phillips S, Zhang H, Wang J, Ochoa A, Wills J, Eubank M, Thomas S B, Gardos S M, Reales D N, Galle J, Durany R, Cambria R, Abida W, Cercek A, Feldman D R, Gounder M M, Hakimi A A, Harding J J, Iyer G, Janjigian Y Y, Jordan E J, Kelly C M, Lowery M A, Morris L G T, Omuro A M, Raj N, Razavi P, Shoushtari A N, Shukla N, Soumerai T E, Varghese A M, Yaeger R, Coleman J, Bochner B, Riely G J, Saltz L B, Scher H I, Sabbatini P J, Robson M E, Klimstra D S, Taylor B S, Baselga J, Schultz N, Hyman D M, Arcila M E, Solit D B, Ladanyi M, Berger M F. Mutational landscape of metastatic cancer revealed from prospective clinical sequencing of 10,000 patients. Nat Med 2017; 23(6):703-13 doi 10.1038/nm.4333.
7. Populo H, Boaventura P, Vinagre J, Batista R, Mendes A, Caldas R, Pardal J, Azevedo F, Honavar M, Guimaraes I, Manuel Lopes J, Sobrinho-Simoes M, Soares P. TERT Promoter Mutations in Skin Cancer: The Effects of Sun Exposure and X-Irradiation. J Invest Dermatol 2014 doi 10.1038/jid.2014.163.
8. Griewank K G, Murali R, Puig-Butille J A, Schilling B, Livingstone E, Potrony M, Carrera C, Schimming T, Moller I, Schwamborn M, Sucker A, Hillen U, Badenas C, Malvehy J, Zimmer L, Scherag A, Puig S, Schadendorf D. TERT promoter mutation status as an independent prognostic factor in cutaneous melanoma. J Natl Cancer Inst 2014; 106(9) doi 10.1093/jnci/dju246.
9. Ofner R, Ritter C, Heidenreich B, Kumar R, Ugurel S, Schrama D, Becker J C. Distribution of TERT promoter mutations in primary and metastatic melanomas in Austrian patients. J Cancer Res Clin Oncol 2017; 143(4):613-7 doi 10.1007/s00432-016-2322-1.
10. Lee S, Opresko P, Pappo A, Kirkwood J M, Bahrami A. Association of TERT promoter mutations with telomerase expression in melanoma. Pigment Cell Melanoma Res 2016; 29(3):391-3 doi 10.1111/pcmr.12471.
11. Huang D S, Wang Z, He X J, Diplas B H, Yang R, Killela P J, Meng Q, Ye Z Y, Wang W, Jiang X T, Xu L, He X L, Zhao Z S, Xu W J, Wang H J, Ma Y Y, Xia Y J, Li L, Zhang R X, Jin T, Zhao Z K, Xu J, Yu S, Wu F, Liang J, Wang S, Jiao Y, Yan H, Tao H Q. Recurrent TERT promoter mutations identified in a large-scale study of multiple tumour types are associated with increased TERT expression and telomerase activation. Eur J Cancer 2015; 51(8):969-76 doi 10.1016/j.ejca.2015.03.010.
12. Nagore E, Heidenreich B, Rachakonda S, Garcia-Casado Z, Requena C, Soriano V, Frank C, Traves V, Quecedo E, Sanjuan-Gimenez J, Hemminki K, Landi M T, Kumar R. TERT promoter mutations in melanoma survival. Int J Cancer 2016; 139(1):75-84 doi 10.1002/ijc.30042.
13. Lee S, Barnhill R L, Dummer R, Dalton J, Wu J, Pappo A, Bahrami A. TERT Promoter Mutations Are Predictive of Aggressive Clinical Behavior in Patients with Spitzoid Melanocytic Neoplasms. Sci Rep 2015; 5:11200 doi 10.1038/srep11200.
14. Vinagre J, Almeida A, Populo H, Batista R, Lyra J, Pinto V, Coelho R, Celestino R, Prazeres H, Lima L, Melo M, da Rocha A G, Preto A, Castro P, Castro L, Pardal F, Lopes J M, Santos L L, Reis R M, Cameselle-Teijeiro J, Sobrinho-Simoes M, Lima J, Maximo V, Soares P. Frequency of TERT promoter mutations in human cancers. Nat Commun 2013; 4:2185 doi 10.1038/ncomms3185.
15. Requena C, Heidenreich B, Kumar R, Nagore E. TERT promoter mutations are not always associated with poor prognosis in atypical spitzoid tumors. Pigment Cell Melanoma Res 2017; 30(2):265-8 doi 10.1111/pcmr.12565.
16. Scott G A, Laughlin T S, Rothberg P G. Mutations of the TERT promoter are common in basal cell carcinoma and squamous cell carcinoma. Mod Pathol 2014; 27(4):516-23 doi 10.1038/modpathol.2013.167.
17. Nagore E, Heidenreich B, Requena C, Garcia-Casado Z, Martorell-Calatayud A, Pont-Sanjuan V, Jimenez-Sanchez A I, Kumar R. TERT promoter mutations associate with fast-growing melanoma. Pigment Cell Melanoma Res 2016; 29(2):236-8 doi 10.1111/pcmr.12441.
18. Bai X, Kong Y, Chi Z, Sheng X, Cui C, Wang X, Mao L, Tang B, Li S, Lian B, Yan X, Zhou L, Dai J, Guo J, Si L. MAPK Pathway and TERT Promoter Gene Mutation Pattern and Its Prognostic Value in Melanoma Patients: A Retrospective Study of 2,793 Cases. Clin Cancer Res 2017; 23(20):6120-7 doi 10.1158/1078-0432.CCR-17-0980.
19. Liau J Y, Tsai J H, Jeng Y M, Chu C Y, Kuo K T, Liang C W. TERT promoter mutation is uncommon in acral lentiginous melanoma. J Cutan Pathol 2014 doi 10.1111/cup.12323.
20. Vazquez Vde L, Vicente A L, Carloni A, Berardinelli G, Soares P, Scapulatempo C, Martinho O, Reis R M. Molecular profiling, including TERT promoter mutations, of acral lentiginous melanomas. Melanoma Res 2016; 26(2):93-9 doi 10.1097/CMR.0000000000000222.
21. de Unamuno Bustos B, Murria Estal R, Perez Simo G, Oliver Martinez V, Llavador Ros M, Palanca Suela S, Botella Estrada R. Lack of TERT promoter mutations in melanomas with extensive regression. J Am Acad Dermatol 2016; 74(3):570-2 doi 10.1016/j.jaad.2015.10.003.
22. Macerola E, Loggini B, Giannini R, Garavello G, Giordano M, Proietti A, Niccoli C, Basolo F, Fontanini G. Coexistence of TERT promoter and BRAF mutations in cutaneous melanoma is associated with more clinicopathological features of aggressiveness. Virchows Arch 2015; 467(2):177-84 doi 10.1007/s00428-015-1784-x.

### 6.19. TERT Mutation Tables

SUPP. TABLE 10
TERT promoter mutation status in 86 primary melanomas, 72 nevi,
and 40 melanocytic proliferations of uncertain diagnosis*

| | | | | | Melanocytic | |
|---|---|---|---|---|---|---|
| | | | | | Proliferation | |
| | Melanomas | | Nevi | | Uncertain Diagnosis | |
| | (n = 86) | | (n = 72) | | (n = 40) | |
| Promoter Mutation Site | No.(%) | | No.(%) | | No.(%) | |
| Confirmed functional ETS/TCF binding | | | | | | |
| site^{a} | n = 67 | (77.9) | n = 1 | (1.4) | n = 2 | (5.0) |
| 124C > T^{b} | 29 | (33.7) | 1 | (1.4) | 1 | (2.5) |
| 124_{—}125CC > TT | 1 | (1.2) | | | | |
| 138_{—}139CC > TT | 4 | (4.7) | | | | |
| 146C > T^{c} | 33 | (38.4) | | | 1 | (2.5) |
| Unconfirmed ETS/TCF binding site^{d} | n = 4 | (4.7) | n = 0 | | n = 1 | (2.5) |
| 103C > T; 160C > T | 1 | (1.2) | | | | |
| 105_{—}106CC > TT | 1 | (1.2) | | | | |
| 148C > T | 1 | (1.2) | | | | |
| 148C > T; 161 C > T; 175C > T; 187C > T; 242C > T | 1 | (1.2) | | | | |
| 149C > T; 156C > T | | | | | 1 | (2.5) |
| Other mutations | n = 2 | (2.3) | n = 7 | (9.7) | n = 1 | (2.5) |
| 85C > T; 154C > T | | | | | 1 | (2.5) |
| 106C > T; 117C > T; 149C > T | | | 1 | (1.4) | | |
| 107C > T | 1 | (1.2) | | | | |
| 116C > T; 179C > T | | | 1 | (1.4) | | |
| 125C > T | | | 1 | (1.4) | | |
| 149C > T | | | 2 | (2.8) | | |
| 149C > T; 127C > T | | | 1 | (1.4) | | |
| 149C > T; 160C > T | | | | | 1 | (2.5) |
| 151C > T | 1 | (1.2) | | | | |
| 161C > T | | | 1 | (1.4) | | |
| No mutations | 13 | (15.2) | 64 | (88.9) | 35 | (87.5) |

*The following samples failed analysis for TERT promoter mutation and were excluded from analyses: melanomas (n = 3), nevus (n = 1), and melanocytic proliferations of uncertain diagnosis (n = 1).
^{a}Bolded mutations create ETS binding sites and are confirmed to be functional.
^{b}Six melanomas with 124C > T mutations had additional mutations: 124C > T; 101C > T (n = 2), 124C > T; 103C > T (n = 1), 124C > T; 126C > T (n = 1), 124C > T; 131C > T; 166C > T (n = 1), 124C > T; 148C > T (n = 1)
^{c}Twelve melanoma with 146C > T mutations had additional mutations: 146C > T117C > T107C > T (n = 1), 146C > T; 149C > T; 153G > A (n = 1), 146C > T; 165C > G (n = 1), 146C > T; 116C > T (n = 1), 146C > T; 125C > T (n = 2), 146C > T; 126C > T; 195C > A (n = 1), 146C > T; 127C > T (n = 1), 146C > T; 149C > T (n = 1), 146C > T; 150C > T (n − 1), 146C > T; 150C > T; 166C > T (n = 1), and 146C > T; 165C > T; 101C > T (n = 1).
^{d}Bolded mutations create ETS binding sites but are not yet confirmed to be functional.

SUPP. TABLE S11
Characteristics of 86 primary melanomas, 72 nevi, and
40 melanocytic proliferations with uncertain diagnosis

| | | | Melanocytic |
|---|---|---|---|
| | | | Proliferation |
| | Primary | | Uncertain |
| | Melanomas | Nevi | Diagnosis^{a} |
| Characteristic | N = 86 | N = 72 | N = 40 |

Laboratory processing of unstained FFPE tissue
sections

| University of North Carolina Pathology Laboratories | 81 | (94.2) | 66 | (91.7) | 40 | (100) |
|---|---|---|---|---|---|---|
| University of Rochester Pathology Laboratories | 5 | (5.8) | 6 | (8.3) | — | |
| Sex | | | | | | |
| Male | 55 | (64.0) | 35 | (48.6) | 16 | (40.0) |
| Female | 31 | (36.1) | 37 | (51.4) | 24 | (60.0) |
| Age at diagnosis of mole or primary melanoma, yrs | | | | | | |
| <65 | 44 | (51.2) | 66 | (91.7) | 35 | (87.5) |
| ≥65 | 42 | (48.8) | 6 | (8.3) | 5 | (12.5) |
| Race | | | | | | |
| Caucasian | 77 | (89.5) | 50 | (69.4) | 24 | (60.0) |
| Other/Unknown | 9 | (10.5) | 22 | (30.6) | 16 | (40.0) |
| Histologic subtype of primary melanoma | | | | | | |
| Superficial Spreading | 43 | (50.0) | — | | — | |
| Nodular | 12 | (14.0) | — | | — | |
| Lentigo maligna | 16 | (18.6) | — | | — | |
| Acral lentiginous | 6 | (7.0) | — | | — | |
| Other/unclassified^{b} | 9 | (10.5) | — | | — | |
| Anatomic site of mole or primary melanoma | | | | | | |
| Head/neck | 28 | (32.6) | 19 | (26.4) | 4 | (10.0) |
| Trunk | 27 | (31.4) | 37 | (51.4) | 23 | (57.5) |
| Upper extremities | 16 | (18.6) | 8 | (11.1) | 2 | (5.0) |
| Lower extremities | 15 | (17.4) | 8 | (11.1) | 11 | (27.5) |
| Solar Elastosis adjacent to the melanocytic lesion | | | | | | |
| Absent | 21 | (24.4) | 43 | (59.7) | 34 | (85.0) |
| Present | 59 | (68.6) | 9 | (12.5) | 5 | (12.5) |
| Indeterminate | 6 | (7.0) | 20 | (27.8) | 1 | (2.5) |
| Contiguous nevus | | | | | | |
| Absent | 75 | (87.2) | — | | — | |
| Present | 11 | (12.8) | — | | — | |
| Melanocytic nevus type | | | | | | |
| Intradermal | — | | 17 | (23.6) | — | |
| Common acquired | — | | 9 | (12.5) | — | |
| Congenital pattern | — | | 14 | (19.4) | — | |
| Dysplastic | — | | 14 | (19.4) | — | |
| Spitz | — | | 9 | (12.5) | — | |
| Other^{c} | — | | 9 | (12.5) | — | |
| Breslow thickness of primary melanoma, mm | | | | | | |
| 0.01 to 2.00 | 45 | (52.3) | — | | — | |
| >2.00 | 41 | (47.7) | — | | — | |
| Ulceration of primary melanoma | | | | | | |
| Absent | 52 | (60.5) | — | | — | |
| Present | 33 | (38.4) | — | | — | |
| Indeterminate | 1 | (1.2) | — | | — | |
| Mitoses of primary melanoma | | | | | | |
| Absent | 17 | (19.8) | — | | — | |
| Present | 69 | (80.2) | — | | — | |
| 2018 AJCC tumor stage at diagnosis | | | | | | |
| 1a/1b/2a | 38 | (44.2) | — | | — | |
| 2b/3a/3b/4a/4b | 48 | (55.8) | — | | — | |
| Tumor infiltrating lymphocyte (TIL) grade of primary melanoma | | | | | | |
| Absent | 20 | (23.3) | — | | — | |
| Present | 65 | (75.6) | — | | — | |
| Indeterminate | 1 | (1.2) | — | | — | |
| Pigment of the melanocytic lesion | | | | | | |
| Absent | 16 | (18.6) | 12 | (16.7) | 7 | (17.5) |
| Present | 70 | (81.4) | 60 | (83.3) | 33 | (82.5) |

| | | | | |
|---|---|---|---|---|
| Regression | | | | |
| Absent | 70 | (81.4) | — | — |
| Present | 16 | (18.6) | — | — |

^{a}Melanocytic proliferations were considered uncertain if there was interobserver disagreement between any of 3 dermatopathology readers or the pathology report diagnosis of nevus vs. melanoma or one of the dermatopathogists or pathology report described the specimen as having uncertain diagnosis.
^{b}Other types of melanoma include nevoid (n = 2), desmoplastic (n = 1), spindle cell (n = 1), Spitzoid (n = 1), unclassified (n = 4).
^{c}Other includes cellular blue nevus (n = 2), combined intradermal or sclerotic blue nevus, not cellular (n = 1), combined nevus with compound congenital pattern and deep penetrating nevus (n = 2), pigmented spindle cell nevus (n = 2), and proliferative nodule in congenital pattern nevus (n = 2).

### 6.20. TERT Mutation Tables (Continued)

Supp. TABLE S12. TERT Promoter Assay Alone, DNA Methylation Assay Alone and Both Assays Together with Sequential
TERT Promoter Muation Assay Followed by DNA Methylation if Negative Samples: Sensitivity, Specificity,
and Positive Predictive Value for Melanoma Among Prmary Melanocytic Lesions

| | | Sensitivity Analysis | | | Specificity Analysis | | | | |
|---|---|---|---|---|---|---|---|---|---|
| | | | No. | | | No. | | Positive | Negative |
| | Diagnostic | Sensitivity, | True | False | Specificity, | True | False | Predictive | Predictive |
| | Accuracy, | % | Posi- | Nega- | % | Nega- | Posi- | Value, % | Value, % |
| Assay | % (95% Cl) | (95% Cl) | tive | tive | (95% Cl) | tive | tive | (95% Cl) | (95% Cl) |
| DNA methylation assay alone | 98.8 | 97.8 | 87 | 2 | 100 | 73 | 0 | 100 | 97.8 |
| | (95.6-99.9) | | | | | | | | |
| Positives have a confirmed | 87.3 | 77.9 | 67 | 19 | 98.6 | 71 | 1 | 98.5 | 78.9 |
| functional ETS/TCF | (81.1-92.1) | | | | | | | | |
| binding site | | | | | | | | | |
| TERT promoter assay alone | 98.2 | 97.8 | 87 | 2 | 98.6 | 72 | 1 | 98.9 | 97.3 |
| TERT promoter assay | (94.7-99.6) | | | | | | | | |
| followed by DNA methylation | | | | | | | | | |
| of negatives and failures (using | | | | | | | | | |
| prediction score = 0) | | | | | | | | | |
| Positives have a confirmed or | 89.9 | 82.6 | 71 | 15 | 98.6 | 7 | 1 | 98.6 | 82.6 |
| unconfirmed ETS/TCF | (84.1-94.1) | | | | | | | | |
| binding site | | | | | | | | | |
| TERT promoter assay alone | 98.2 | 97.8 | 87 | 2 | 98.6 | 72 | 1 | 98.9 | 97.3 |
| TERT promoter assay followed by | (94.7-99.6) | | | | | | | | |
| DNA methylation of negatives and | | | | | | | | | |
| failures (using prediction score = 0) | | | | | | | | | |

SUPP. TABLE S13
Relationship of TERT positivity to clinicopathologic features in primary melanomas
from 86 patients*

| | | | | Confirmed Functional | | |
|---|---|---|---|---|---|---|
| | Confirmed Functional | | | and Unconfirmed | | |
| | TERT—neg | TERT—pos | | TERT—neg | TERT—pos | |
| | (n = 19) | (n = 67) | | (n = 15) | (n = 71) | |
| | n (%) | n (%) | P^{a} | n (%) | n (%) | P^{a} |
| Sex | | | | | | |
| Male | 13 (23.6) | 42 (76.4) | 0.79 | 9 (19.4) | 46 (80.7) | 0.77 |
| Female | 6 (19.4) | 25 (80.7) | | 6 (16.4) | 25 (83.6) | |
| Age, years | | | | | | |
| <65 | 15 (34.1) | 29 (65.9) | 0.005 | 12 (27.3) | 32 (72.7) | 0.02 |
| ≥65 | 4 (9.5) | 38 (90.5) | | 3 (7.1) | 39 (92.9) | |
| Race | | | | | | |
| Whites of European | 14 (18.2) | 63 (81.8) | 0.02 | 11 (14.3) | 66 (85.7) | 0.04 |
| origin | | | | | | |
| Other/Unknown | 5 (55.6) | 4 (44.4) | | 4 (44.4) | 5 (55.6) | |
| Histologic subtype | | | | | | |
| Superficial Spreading | 9 (20.9) | 34 (79.1) | 0.01 | 7 (16.3) | 36 (83.7) | <.001 |
| Nodular | 2 (16.7) | 10 (83.3) | | 2 (16.7) | 10 (83.3) | |
| Lentigo maligna | 2 (12.5) | 14 (87.5) | | 1 (6.3) | 15 (93.8) | |
| Acral lentiginous | 5 (83.3) | 1 (16.7) | | 5 (83.3) | 1 (16.7) | |
| Other/unclassified^{b} | 1 (11.1) | 8 (89.9) | | 0 | 9 (100.0) | |
| Site | | | | | | |
| Head/neck | 4 (14.3) | 24 (85.7) | <.001 | 2 (7.1) | 26 (92.9) | <.001 |
| Trunk | 6 (22.2) | 21 (77.8) | | 5 (18.5) | 22 (81.5) | |
| Upper extremities | 0 | 16 (100.0) | | 0 | 16 (100.0) | |
| Lower extremities | 9 (60.0) | 6 (40.0) | | 8 (53.3) | 7 (31.0) | |
| Solar elastosis | | | | | | |
| Absent | 9 (42.9) | 12 (57.1) | 0.01 | 8 (38.1) | 13 (61.9) | 0.008 |
| Present | 9 (15.3) | 50 (84.8) | | 7 (11.9) | 52 (88.1) | |
| Contiguous nevus | | | | | | |
| Absent | 17 (22.7) | 58 (77.3) | 1.00 | 13 (17.3) | 62 (82.7) | 1.00 |
| Present | 2 (18.2) | 9 (81.8) | | 2 (18.2) | 9 (81.8) | |
| Breslow thickness (mm) | | | | | | |
| 0.01 to 2.00 | 8 (17.8) | 37 (82.2) | 0.44 | 7 (15.6) | 38 (84.4) | 0.78 |
| >2.00 | 11 (26.8) | 30 (73.2) | | 8 (19.5) | 33 (80.5) | |
| Ulceration | | | | | | |
| Absent | 12 (23.1) | 40 (76.9) | 0.85 | 10 (19.2) | 42 (80.8) | 0.81 |
| Present | 7 (21.2) | 26 (78.8) | | 5 (15.2) | 30 (84.9) | |
| Indeterminant | 0 | 1 (100.0) | | 0 | 1 (100.0) | |
| Mitoses | | | | | | |
| Absent | 3 (17.7) | 14 (82.4) | 0.75 | 3 (17.7) | 14 (82.4) | 1.00 |
| Present | 16 (23.2) | 53 (76.8) | | 12 (17.4) | 57 (82.6) | |
| 2018 AJCC stage at | | | | | | |
| diagnosis | | | | | | |
| 1a/1b/2a | 7 (18.4) | 31 (81.6) | 0.60 | 6 (15.8) | 32 (84.2) | 0.78 |
| 2b/3a/3b/4a/4b | 12 (25.0) | 36 (75.0) | | 9 (18.8) | 39 (81.3) | |
| Tumor inflitrating lymphocyte grade | | | | | | |
| Absent | 5 (25.0) | 15 (75.0) | 0.76 | 4 (20.0) | 16 (80.0) | 0.75 |
| Present | 14 (21.5) | 51 (78.5) | | 11 (16.9) | 54 (83.1) | |
| Pigment | | | | | | |
| Absent | 3 (18.8) | 13 (81.3) | 1.00 | 2 (12.5) | 14 (87.5) | 0.73 |
| Present | 16 (22.9) | 54 (77.1) | | 13 (18.6) | 57 (81.4) | |
| Regression | | | | | | |
| Absent | 18 (25.7) | 52 (74.3) | 0.11 | 14 (20.0) | 56 (80.0) | 0.28 |
| Present | 1 (6.3) | 15 (93.8) | | 1 (6.3) | 15 (93.8) | |
| rs2853669 | | | | | | |
| Absent | 10 (55.6) | 39 (59.1) | 0.79 | 8 (16.3) | 41 (83.6) | 1.00 |
| Present | 8 (44.4) | 27 (40.9) | | 6 (17.1) | 29 (82.9) | |

Definitions:
AJCC, American Joint Committee on Cancer
*Melanomas (n = 3) that failed analysis for TERT promoter mutation were excluded from analysis.
^{a}P-values were derived from the Fisher's exact test.
^{b}Other types of melanoma include nevoid (n = 2), desmoplastic (n = 1), spindle cell (n = 1), Spitzoid (n = 1), unclassified (n = 4).

### 7. SEQUENCE LISTING

### Incorporation-by-Reference of Material Submitted Electronically

This application contains a sequence listing. It has been submitted electronically via EFS-Web as an ASCII text file entitled “150-25-PCT_2019-01-18_SEQ_LIST_ST25.txt”. The sequence listing is 85.4 kilobytes in size, and was created on Jan. 18, 2019. It is hereby incorporated by reference in its entirety.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world. It is to be understood that, while the disclosure has been described in conjunction with the detailed description, thereof, the foregoing description is intended to illustrate and not limit the scope. Other aspects, advantages, and modifications are within the scope of the claims set forth below. All publications, patents, and patent applications cited in this specification are herein incorporated by reference as if each individual publication or patent application were specifically and individually indicated to be incorporated by reference.

1. A method for detecting melanoma in a tissue sample which comprises:
(a) measuring a level of methylation of a plurality of regulatory elements differentially methylated in melanoma and benign nevi; and
(b) determining whether melanoma is present or absent in the tissue sample if there is
(i) hypermethylation of at least one regulatory element associated with a gene encoding ALX3, CCDC140, CCDC19, DYNC1I1, FLJ22536, HOXD12, LIPC, NBLA00301/HAND2, NRXN1, ONECUT1, PAX3/CCDC140, PROM1, RASGEF1C, SGEF, SHANK3, SHOX2, SIX6, TBX5, TLX3, and ZBTB38, and
(ii) hypomethylation of at least one regulatory element associated with a gene encoding ANKH, C3AR1, C5orf56, CACNA1C, CYTIP, EPB41L4A, FAIM3, GIMAP7, GOLIM4, KREMEN1, MAS1L, MBP, MYT1L, OPCML, SORCS2, TLR1, and VOPP1.

2. The method of claim 1, wherein the level of methylation is measured at single CpG site resolution.

3. (canceled)

4. (canceled)

5. (canceled)

6. A method for detecting melanoma in a tissue sample which comprises:
(a) measuring a level of methylation of a plurality of regulatory elements differentially methylated in melanoma and benign nevi; and
(b) determining whether melanoma is present or absent in the tissue sample if there is (i) hypermethylation of a CpG site cg02744046, cg02936049, cg03874199, cg05787556, cg06215569, cg06573459, cg07553475, cg07569216, cg08697503, cg08898055, cg09476130, cg12993163, cg13019491, cg13164157, cg14064356, cg16325502, cg16919569, cg17889682, cg18077971, cg18689332, cg18851100, cg19352038, and cg22322562, and (ii) hypomethylation of a CpG site cg00387964, cg02468320, cg03315407, cg03653573, cg04499514, cg07230581, cg07637837, cg08337633, cg08757862, cg10559416, cg12423733, cg15158847, cg15536663, cg15849098, cg17918270, cg18098839, and cg26967305.

7. The method of claim 1 further comprising measuring at least one DNA mutation in a TERT gene promoter region.

8. The method of claim 7, where the DNA mutation in the TERT gene promoter is 103C>T, 105_106CC>TT, 124C>T, 138_139CC>TT, 146C>T, 148C>T, or 156C>T.

9. (canceled)

10. (canceled)

11. (canceled)

12. (canceled)

13. (canceled)

14. (canceled)

15. (canceled)

16. (canceled)

17. The method of claim 1, wherein the tissue sample is a common nevi sample, a dysplastic nevi sample, or a benign atypical nevi sample.

18. (canceled)

19. (canceled)

20. The method of claim 1, wherein the tissue sample is a melanocytic lesion of unknown potential.

21. The method of claim 1, wherein the tissue sample is a formalin-fixed, paraffin-embedded sample.

22. The method of claim 1, wherein the tissue sample is a fresh-frozen sample.

23. The method of claim 1, wherein the tissue sample is a fresh tissue sample.

24. The method of claim 1, wherein the tissue sample is a dissected tissue, an excision biopsy, a needle biopsy, a punch biopsy, a shave biopsy, or a skin biopsy sample.

25. The method of claim 1, wherein the tissue sample is a lymph node biopsy sample.

26. The method of claim 1, wherein the level of methylation is measured using a bisulfate conversion-based microarray assay.

27. The method of claim 1, wherein the level of methylation is measured using a methylation specific polymerase chain reaction assay.

28. The method of claim 1, wherein the level of methylation is measured using a mass spectrometry assay.

29. The method of claim 1, wherein a plurality of regulatory elements differentially methylated are measured, and together they have a sensitivity of greater than 95% more preferably greater than 97%.

30. A method for treating a patient with a suspicious melanocytic lesion, the method comprising the steps of:
(a) determining whether the suspicious lesion is a melanoma by obtaining, or having obtained a biological sample from the patient, and performing, or having performed, a test the biological sample to determine if there is (i) hypermethylation of at least on regulatory element associated with a gene encoding ALX3, CCDC140, CCDC19, DYNC1I1, FLJ22536, HOXD12, LIPC, NBLA00301/HAND2, NRXN1, ONECUT1, PAX3/CCDC140, PROM1, RASGEF1C, SGEF, SHANK3, SHOX2, SIX6, TBX5, TLX3, and ZBTB38, and (ii) hypomethylation of at least one regulatory element associated with a gene encoding ANKH, C3AR1, C5orf56, CACNA1C, CYTIP, EPB41L4A, FAIM3, GIMAP7, GOLIM4, KREMEN1, MAS1L, MBP, MYT1L, OPCML, SORCS2, TLR1, and VOPP1;
(b) if the suspicious lesion is determined to be a melanoma treating the patient.

31. The method of claim 30 further comprising measuring at least one DNA mutation in a TERT gene promoter region.

32. The method of claim 31, where the DNA mutation in the TERT gene promoter is 103C>T, 105_106CC>TT, 124C>T, 138_139CC>TT, 146C>T, 148C>T, or 156C>T.

33. The method of claim 30, wherein the treatment is wide surgical excision (≥1 cm) of the suspicious melanocytic lesion.

34. (canceled)

35. (canceled)


SEQUENCE LISTING (480 sequences)

SEQ ID NO: 1 - DNA, 122 bp, Homo sapiens
tttataacttggtaagtgccagcgaactcgcctcctttacacccccgagtgccagccccgcgctctgcactgcgctttattcgctcgagcctattcagggactgtcactccggggccgcgag

SEQ ID NO: 2 - DNA, 122 bp, Homo sapiens
agaccacgagcaagtaagcacgttaatcaaagtgaaaggctcacccctcacgtctagctccgtccttctccagcctgtgcctgccagattatttcgggttcctcgtgtttgactcgtcaaag

SEQ ID NO: 3 - DNA, 122 bp, Homo sapiens
gccactctttctctgtctccgagtcttgggcctcccctttatttctttctgaagtctctccggagcccaagccaccccacacccaaaccccgcagctggatgggagtccaggccacttccct

SEQ ID NO: 4 - DNA, 122 bp, Homo sapiens
tatttataacttggtaagtgccagcgaactcgcctcctttacacccccgagtgccagccccgcgctctgcactgcgctttattcgctcgagcctattcagggactgtcactccggggccgcg

SEQ ID NO: 5 - DNA, 122 bp, Homo sapiens
tggctgtccaggcctgagtggagcgtgcccttgttagcttgaaagttctccctcgcagcccgtttggatgcgtgcgtctacagcccagtcgcactttggtgaccggcctgggctgtgaagca

SEQ ID NO: 6 - DNA, 122 bp, Homo sapiens
gacagccaggtaatctccgtcccgcctgcccgaccggggtcgcacgagcacaggcgcccacgccatgttggctgcccaaagggctcgccgcccaagccgggccagaaggcaggaggcggaaa

SEQ ID NO: 7 - DNA, 122 bp, Homo sapiens
atttcccttccccttttcttggttgtcgctcgctttctttggttttctttctcggtatttcgttgtcaaggccacccttgccgtcggatcccggggtgctgggtttctcccggccgctcgtt

SEQ ID NO: 8 - DNA, 122 bp, Homo sapiens
ttcgctggaagaaaatgattccgcttgtctccccaaagctgcagcggaaggtgactacttcgtgtgcggtcctgtccacggtgccctgggccgggtagacagtcactgaggcgcgagcagaa

SEQ ID NO: 9 - DNA, 122 bp, Homo sapiens
catctagagctgagtctcatttgtttttgagccggaggcttggtctccaagccctcccagcgtccacccgtctctctcctgccgggagttttctctcctaagagccggcagatgctggaggg

SEQ ID NO: 10 - DNA, 122 bp, Homo sapiens
ccgtagactccagcagcaggtcctgtcacagggttccgggcgggcactacgggcggagggcgacggcacgccagaggtgctgggcgtcgccaccagcccggccgccagtctatccagcaagg

SEQ ID NO: 11 - DNA, 122 bp, Homo sapiens
tagcccaagggagaccaaagactccaccttgagcatcgccctttggaggcgggcagagtccggccgcaggccacaaagcgatccccacccgaaggactccacaaggacagtcctttccttgc

SEQ ID NO: 12 - DNA, 122 bp, Homo sapiens
ttgtagctgagtgggtgaaacggcatcaccaacatttggccctgctgctccactgagagccggcgccgttcgcgggataattatcctgtagtcttctcacctccggagagaatgcaaggcgc

SEQ ID NO: 13 - DNA, 122 bp, Homo sapiens
agggaggagaaaggcgaagggaggaggtaacagcaggcgggcaactgtaggtaacctaagcggaaaacaaaccaggacgcatgcgcctctagagaacgggttttgaagatgcttcaaaggga

SEQ ID NO: 14 - DNA, 122 bp, Homo sapiens
gatgtaggcggtgctgaaatgaccggctttgaagaacctgcaggcaaagtttcgtccaatcgtctgagcctgtcctcttattcccggttgtaactaaatactgttgcgagcgcagccgaagc

SEQ ID NO: 15 - DNA, 122 bp, Homo sapiens
ctgctgaggggccagggaggcggcgcagatggctagggtaaggggggcgcagagcgaacccgtccactcctcactgtacacccccagtacagtggaaggagtgcgctcagccccgcgcctgg

SEQ ID NO: 16 - DNA, 122 bp, Homo sapiens
ccggcaccactcaaaaagtctcagcagtcgttgcttttcaatttgctcccctaacgagaccgcataggtaaacagacctccctctaacccccgaccgaaaaaaaggcttattttcatgcacg

SEQ ID NO: 17 - DNA, 122 bp, Homo sapiens
ggtggaggatgaggaggcggcctgggaccccgagtcagatctttggggtgagcacgaggacgtggtgtagggaagaggacgagtgagcagcgcctggctgtagggtcagagggcgcctggtc

SEQ ID NO: 18 - DNA, 122 bp, Homo sapiens
ggcgcgggcctgttctgtgagggagaaaacaagcgtcctatttaccacgagaatgaatatcgggctctgtgtgaaaatcccacttgctctgagatgtgtgaagccagcagggccagggacgc

SEQ ID NO: 19 - DNA, 122 bp, Homo sapiens
agccgcgtggagggacgaaaagatcaaccacccgatcgacgaggataggtttgatcttttcgattacctcagtgtgccagtgtatattcccggctgggcctagcgccctaagaaacttcgga

SEQ ID NO: 20 - DNA, 122 bp, Homo sapiens
ccacaagagactcctcaaggtgcgcagcatggtggagggcctaggaggacccctgggtcacgcaggggaggagagtgaggtcgataacgacgtggatagcccagggtctctgcggagaggct

SEQ ID NO: 21 - DNA, 122 bp, Homo sapiens
acctccagcgggcagttgccttgtgctggtggcttaggaaccggagcccgtcgctccaaccgttgcagctccacgctccagcccaaccgcggctctgaaggattgacccgccctggcgtgcc

SEQ ID NO: 22 - DNA, 122 bp, Homo sapiens
ggagtcgggtcaaggctggcctctgtgggagggggttgccggggtccccaggaacctctccgaaggcagcaccaccccccgcccagcgccctggctggtctcaccggcccttccgtccgcag

SEQ ID NO: 23 - DNA, 122 bp, Homo sapiens
ttgttttggtaaacaccttcacggccgcctggctctccttcccccgctcccattcggaatcgctctggccttataaatccgtgcgtcgtcatcataagggcagtgatcctggcagcgctgat

SEQ ID NO: 24 - DNA, 122 bp, Homo sapiens
tgtgctccctgggaagaaggttctccacatgctgagtagagtgtggttgctccattgggtcgatgccagctgcctttttgttcctccccacctctggcttatctgctaacgcccgttggaga

SEQ ID NO: 25 - DNA, 122 bp, Homo sapiens
ttttctaccacacagcgagcaaggccaacttccctaaactaagaatgctgagattcttttcgacttataatgttctgactgtctctctctgtttccccttacctcagaatttgtttaataga

SEQ ID NO: 26 - DNA, 122 bp, Homo sapiens
cctggctgtgtgtgtggcgtccagtgcgagtggtagccagacatcatgcccacctgccctcgagctgcttgcctgcagctggctccttactcacagatctgcatccatccggcgctggggag

SEQ ID NO: 27 - DNA, 122 bp, Homo sapiens
actaaccttttacataaaccagatgtttcttaaaatagcccagttaaatccacccttcctcgtggcatctgcttaccaccaaatgttcctccacttctgtattctcttgcttttgattactt

SEQ ID NO: 28 - DNA, 122 bp, Homo sapiens
tttcagcaccccaccccctcttcagttgaaggtagcaagccattcccacagtgggtggcccgcagggttatctgccacatcaaaaagagaggctttatggtactctaccaagcatccttaca

SEQ ID NO: 29 - DNA, 122 bp, Homo sapiens
cacgctgcttactcaggaacccttcacaatctggaactggaaagagatttctagcccccacgaggaacaaagcttgacgatgaggaaatgacaacctcccttgtttgctaactattctcagg

SEQ ID NO: 30 - DNA, 122 bp, Homo sapiens
tcatgaagtatggcaagaaaatttgctgagctttctcttcttctgttctctctctgtttccggcaataagttaagtcttatgctctagaccactatctgacctcacaggaagagtttcaaag

SEQ ID NO: 31 - DNA, 122 bp, Homo sapiens
catcgtaaggctgccggtgagtgtggagtaagagctatacgctggcccagcgcagaagtccgccaaattgccattgctgctgtgttgcaggagcctttgtaaagacattgtgaataaagatc

SEQ ID NO: 32 - DNA, 122 bp, Homo sapiens
aatcgacctcagttccgcaggatgaaggtgaccctgagccggcctcaggatgcagggaagcgcggacatacctcgaacccctttggaccgcgtgcgatgccgcttctcctcggtgtccacct

SEQ ID NO: 33 - DNA, 122 bp, Homo sapiens
gtgtaatctccgtggtcagtgaagcaatgctgtgcgcacagttctgtgttgtgcctctcccggggaaggggtgtatttggcctgtgccccaccctagccctccttcgtcttccctctttcac

SEQ ID NO: 34 - DNA, 122 bp, Homo sapiens
caaaaacccgtagaatgaacaccgtgcacacgcacacacacacacacacacacgtgcgcgcgcggcaaaaagaaacagctcatttcggagctgaggacaaggcgtgggaagaagacgcgttt

SEQ ID NO: 35 - DNA, 122 bp, Homo sapiens
tctaggcaatatttgggtcatttaataaggctcttttgcatccatcactataacctgaagcgaaaaatgtagctttggaaatggtgtttatagcaggcccatgggcaaaacgtttcaaccgg

SEQ ID NO: 36 - DNA, 122 bp, Homo sapiens
cctcaaaaggccccaggcctactgtggttttttctgagaggctcccagaaccaagtggcacgttggtttcctgtgcgtctgtgtctttgtgcctgtatctcgctgggggacttcacaggaag

SEQ ID NO: 37 - DNA, 122 bp, Homo sapiens
aggtcctgtcatggtcacctgtggcttgggccaattctcacttcccctgaagggcagctgcgtgtagggagcgggggctgcccaaagtttcactctgactggaggtaaacttaacatcattt

SEQ ID NO: 38 - DNA, 122 bp, Homo sapiens
ttttctgcttctccttttcagggtccagtctgtcccttcctctggaatggcagtttacaccggcagtttacacaggatggcagtatctctgggtgtaagaaacctcagaactttcccctgct

SEQ ID NO: 39 - DNA, 122 bp, Homo sapiens
acagataattcaagtgcaggtctgacagggggtgaccctgggtgaatcactcaattgctgcgagcctccatcttccgtctgcacgaggtattgttagaagcatctacttcctggcgatgttg

SEQ ID NO: 40 - DNA, 122 bp, Homo sapiens
cactagtgcctgttttcctgactctgacttcctgggtctcggcaccacagatagcttctgcgtttctctacaggagggaagaagcaatttccaattctgagcttcatgagggaggagaataa

SEQ ID NO: 41 - DNA, 50 bp, Homo sapiens
gagtgacagtccctgaataggctcgagcgaataaagcgcagtgcagagcg

SEQ ID NO: 42 - DNA, 50 bp, Homo sapiens
cggagctagacgtgaggggtgagcctttcactttgattaacgtgcttact

SEQ ID NO: 43 - DNA, 50 bp, Homo sapiens
cggagcccaagccaccccacacccaaaccccgcagctggatgggagtcca

SEQ ID NO: 44 - DNA, 50 bp, Homo sapiens
gtgacagtccctgaataggctcgagcgaataaagcgcagtgcagagcgcg

SEQ ID NO: 45 - DNA, 50 bp, Homo sapiens
cgggctgcgagggagaactttcaagctaacaagggcacgctccactcagg

SEQ ID NO: 46 - DNA, 50 bp, Homo sapiens
cgccatgttggctgcccaaagggctcgccgcccaagccgggccagaaggc

SEQ ID NO: 47 - DNA, 50 bp, Homo sapiens
cttttcttggttgtcgctcgctttctttggttttctttctcggtatttcg

SEQ ID NO: 48 - DNA, 50 bp, Homo sapiens
aaatgattccgcttgtctccccaaagctgcagcggaaggtgactacttcg

SEQ ID NO: 49 - DNA, 50 bp, Homo sapiens
cgctgggagggcttggagaccaagcctccggctcaaaaacaaatgagact

SEQ ID NO: 50 - DNA, 50 bp, Homo sapiens
cgccctccgcccgtagtgcccgcccggaaccctgtgacaggacctgctgc

SEQ ID NO: 51 - DNA, 50 bp, Homo sapiens
cggccgcaggccacaaagcgatccccacccgaaggactccacaaggacag

SEQ ID NO: 52 - DNA, 50 bp, Homo sapiens
gggtgaaacggcatcaccaacatttggccctgctgctccactgagagccg

SEQ ID NO: 53 - DNA, 50 bp, Homo sapiens
atcttcaaaacccgttctctagaggcgcatgcgtcctggtttgttttccg

SEQ ID NO: 54 - DNA, 50 bp, Homo sapiens
gctgaaatgaccggctttgaagaacctgcaggcaaagtttcgtccaatcg

SEQ ID NO: 55 - DNA, 50 bp, Homo sapiens
ctgagcgcactccttccactgtactgggggtgtacagtgaggagtggacg

SEQ ID NO: 56 - DNA, 50 bp, Homo sapiens
cggtctcgttaggggagcaaattgaaaagcaacgactgctgagacttttt

SEQ ID NO: 57 - DNA, 50 bp, Homo sapiens
cgtcctcgtgctcaccccaaagatctgactcggggtcccaggccgcctcc

SEQ ID NO: 58 - DNA, 50 bp, Homo sapiens
cgggctctgtgtgaaaatcccacttgctctgagatgtgtgaagccagcag

SEQ ID NO: 59 - DNA, 50 bp, Homo sapiens
cgattacctcagtgtgccagtgtatattcccggctgggcctagcgcccta

SEQ ID NO: 60 - DNA, 50 bp, Homo sapiens
cctcaaggtgcgcagcatggtggagggcctaggaggacccctgggtcacg

SEQ ID NO: 61 - DNA, 50 bp, Homo sapiens
cgggtcaatccttcagagccgcggttgggctggagcgtggagctgcaacg

SEQ ID NO: 62 - DNA, 50 bp, Homo sapiens
gggccggtgagaccagccagggcgctgggcggggggtggtgctgccttcg

SEQ ID NO: 63 - DNA, 50 bp, Homo sapiens
cgctctggccttataaatccgtgcgtcgtcatcataagggcagtgatcct

SEQ ID NO: 64 - DNA, 50 bp, Homo sapiens
cgacccaatggagcaaccacactctactcagcatgtggagaaccttcttc

SEQ ID NO: 65 - DNA, 50 bp, Homo sapiens
cagcgagcaaggccaacttccctaaactaagaatgctgagattcttttcg

SEQ ID NO: 66 - DNA, 50 bp, Homo sapiens
ggatggatgcagatctgtgagtaaggagccagctgcaggcaagcagctcg

SEQ ID NO: 67 - DNA, 50 bp, Homo sapiens
cataaaccagatgtttcttaaaatagcccagttaaatccacccttcctcg

SEQ ID NO: 68 - DNA, 50 bp, Homo sapiens
tggtagagtaccataaagcctctctttttgatgtggcagataaccctgcg

SEQ ID NO: 69 - DNA, 50 bp, Homo sapiens
tagcaaacaagggaggttgtcatttcctcatcgtcaagctttgttcctcg

SEQ ID NO: 70 - DNA, 50 bp, Homo sapiens
gcaagaaaatttgctgagctttctcttcttctgttctctctctgtttccg

SEQ ID NO: 71 - DNA, 50 bp, Homo sapiens
cgccaaattgccattgctgctgtgttgcaggagcctttgtaaagacattg

SEQ ID NO: 72 - DNA, 50 bp, Homo sapiens
cgcggacatacctcgaacccctttggaccgcgtgcgatgccgcttctcct

SEQ ID NO: 73 - DNA, 50 bp, Homo sapiens
tggtcagtgaagcaatgctgtgcgcacagttctgtgttgtgcctctcccg

SEQ ID NO: 74 - DNA, 50 bp, Homo sapiens
ttcccacgccttgtcctcagctccgaaatgagctgtttctttttgccgcg

SEQ ID NO: 75 - DNA, 50 bp, Homo sapiens
ttttgcccatgggcctgctataaacaccatttccaaagctacatttttcg

SEQ ID NO: 76 - DNA, 50 bp, Homo sapiens
cgtgccacttggttctgggagcctctcagaaaaaaccacagtaggcctgg

SEQ ID NO: 77 - DNA, 50 bp, Homo sapiens
cgcagctgcccttcaggggaagtgagaattggcccaagccacaggtgacc

SEQ ID NO: 78 - DNA, 50 bp, Homo sapiens
cggcagtttacacaggatggcagtatctctgggtgtaagaaacctcagaa

SEQ ID NO: 79 - DNA, 50 bp, Homo sapiens
cgcagcaattgagtgattcacccagggtcaccccctgtcagacctgcact

SEQ ID NO: 80 - DNA, 50 bp, Homo sapiens
ctcatgaagctcagaattggaaattgcttcttccctcctgtagagaaacg

SEQ ID NO: 81 - DNA, 20 bp, Homo sapiens
ggtaagggtggttttgataa

SEQ ID NO: 82 - DNA, 25 bp, Homo sapiens
taagagtgaaattttgttttaaaaa

SEQ ID NO: 83 - DNA, 30 bp, Homo sapiens
gaggttagatagtggtttagagtataagat

SEQ ID NO: 84 - DNA, 25 bp, Homo sapiens
tggggttttttgttattttggttgt

SEQ ID NO: 85 - DNA, 25 bp, Homo sapiens
gtagtaggtggggtagggtggtttt

SEQ ID NO: 86 - DNA, 29 bp, Homo sapiens
aaatttggtattaggattttttggtttat

SEQ ID NO: 87 - DNA, 28 bp, Homo sapiens
gaaatttttgtgttagtgtttttaagtg

SEQ ID NO: 88 - DNA, 30 bp, Homo sapiens
gttagtttatgaatttatgggtatttaaga

SEQ ID NO: 89 - DNA, 30 bp, Homo sapiens
aagtttggttaaagttaaagtggagagtag

SEQ ID NO: 90 - DNA, 30 bp, Homo sapiens
ggttatttaattatgtgttagttggagtga

SEQ ID NO: 91 - DNA, 25 bp, Homo sapiens
ttgtagagggaggggaaaggatgtt

SEQ ID NO: 92 - DNA, 30 bp, Homo sapiens
ttagtttgtggtgtagttgggtagatatta

SEQ ID NO: 93 - DNA, 25 bp, Homo sapiens
aagataaaggaatattaggtttggt

SEQ ID NO: 94 - DNA, 30 bp, Homo sapiens
ttaattttggaggttgagaatgattggaag

SEQ ID NO: 95 - DNA, 30 bp, Homo sapiens
tgttttgttttttaggtaatatttgggtta

SEQ ID NO: 96 - DNA, 24 bp, Homo sapiens
gaagtggaggaatatttggtggta

SEQ ID NO: 97 - DNA, 27 bp, Homo sapiens
taatttgatttggttttgttggtagtt

SEQ ID NO: 98 - DNA, 26 bp, Homo sapiens
attatggtttaattttaagagggatt

SEQ ID NO: 99 - DNA, 25 bp, Homo sapiens
ttggttttgttgttttattgagagt

SEQ ID NO: 100 - DNA, 27 bp, Homo sapiens
aaggaattttttagagtgattttttaa

SEQ ID NO: 101 - DNA, 25 bp, Homo sapiens
aattaggatatgttcgatttttcgt

SEQ ID NO: 102 - DNA, 25 bp, Homo sapiens
tgtagggttggtttgtataggtagg

SEQ ID NO: 103 - DNA, 30 bp, Homo sapiens
tttaagattttagggttagtggagggtaga

SEQ ID NO: 104 - DNA, 30 bp, Homo sapiens
tgtgtttagtaattatattggatttgaatg

SEQ ID NO: 105 - DNA, 30 bp, Homo sapiens
tttttgggaagttatagtaagaaaataaaa

SEQ ID NO: 106 - DNA, 25 bp, Homo sapiens
aggaatttaagttatttgaggtttt

SEQ ID NO: 107 - DNA, 27 bp, Homo sapiens
tttttgattgattaaggttttgaatat

SEQ ID NO: 108 - DNA, 27 bp, Homo sapiens
tttttattttaaagggaaaaatttgtt

SEQ ID NO: 109 - DNA, 23 bp, Homo sapiens
ggtgagtagttgggtttttattt

SEQ ID NO: 110 - DNA, 22 bp, Homo sapiens
agggtagtgagtttggttaggg

SEQ ID NO: 111 - DNA, 30 bp, Homo sapiens
aataattaaaatgtggagttttataagaga

SEQ ID NO: 112 - DNA, 25 bp, Homo sapiens
ggttaaggttggtttttgtgggagg

SEQ ID NO: 113 - DNA, 32 bp, Homo sapiens
ttgtatgaaaaggttttgagtaattaatagaa

SEQ ID NO: 114 - DNA, 24 bp, Homo sapiens
tggtggggtataatagtagggatt

SEQ ID NO: 115 - DNA, 30 bp, Homo sapiens
ggaaattgttgtggttaaatgatttatttt

SEQ ID NO: 116 - DNA, 26 bp, Homo sapiens
gatattttagtaacgcgaggatcggc

SEQ ID NO: 117 - DNA, 30 bp, Homo sapiens
aggggaaatagagagagatagttagaatat

SEQ ID NO: 118 - DNA, 25 bp, Homo sapiens
ggaagaatttaggttaggggtgcga

SEQ ID NO: 119 - DNA, 25 bp, Homo sapiens
ttagagtgaaattttgggtagtttt

SEQ ID NO: 120 - DNA, 28 bp, Homo sapiens
cattttaacctatttctaccactttaac

SEQ ID NO: 121 - DNA, 32 bp, Homo sapiens
tatttaatctactccctccctctttatccttt

SEQ ID NO: 122 - DNA, 25 bp, Homo sapiens
tcctaactctaacttcctaaatctc

SEQ ID NO: 123 - DNA, 25 bp, Homo sapiens
aacttcaacacctaaatatctccac

SEQ ID NO: 124 - DNA, 25 bp, Homo sapiens
aacccacaaaccacttcctctactc

SEQ ID NO: 125 - DNA, 30 bp, Homo sapiens
aaaaaaaactaaaataaaacacaaaccaaa

SEQ ID NO: 126 - DNA, 30 bp, Homo sapiens
caacaaaattaactaaaactacctaaccta

SEQ ID NO: 127 - DNA, 29 bp, Homo sapiens
aaaaaaataacttctattatcccctctac

SEQ ID NO: 128 - DNA, 30 bp, Homo sapiens
ttaaaaaacatctaaaactaaatctcattt

SEQ ID NO: 129 - DNA, 22 bp, Homo sapiens
ttaccttaacaaccactaaccc

SEQ ID NO: 130 - DNA, 30 bp, Homo sapiens
actctaccctcaaaaaaatataaacactct

SEQ ID NO: 131 - DNA, 28 bp, Homo sapiens
ctaccaaaatcactacccttataataac

SEQ ID NO: 132 - DNA, 26 bp, Homo sapiens
aacaaaccattcccacaataaataac

SEQ ID NO: 133 - DNA, 21 bp, Homo sapiens
ccatccaaaaacccctaaaaa

SEQ ID NO: 134 - DNA, 27 bp, Homo sapiens
aaaacaaaactctcaaaataaccaaac

SEQ ID NO: 135 - DNA, 30 bp, Homo sapiens
aaaaccacacacacaaaaatatttatcttt

SEQ ID NO: 136 - DNA, 30 bp, Homo sapiens
aaaaaataataaataatcattcccaataca

SEQ ID NO: 137 - DNA, 23 bp, Homo sapiens
ctcacacatctccaacaaaaaac

SEQ ID NO: 138 - DNA, 24 bp, Homo sapiens
ctacttcctataaaatcccccaac

SEQ ID NO: 139 - DNA, 30 bp, Homo sapiens
aaaaacatttctccatatttcattattata

SEQ ID NO: 140 - DNA, 30 bp, Homo sapiens
ctatatatacaattccccaactcaaatata

SEQ ID NO: 141 - DNA, 32 bp, Homo sapiens
ccaaaaatataattataacactctatattcga

SEQ ID NO: 142 - DNA, 27 bp, Homo sapiens
ttccacaaaaaaattaccaaaaaaata

SEQ ID NO: 143 - DNA, 29 bp, Homo sapiens
cccaaaaaaaaccaaaaactccaccttaa

SEQ ID NO: 144 - DNA, 25 bp, Homo sapiens
aaactattaatacacaacccaaccc

SEQ ID NO: 145 - DNA, 25 bp, Homo sapiens
tacactcaaatactcacacaaaacc

SEQ ID NO: 146 - DNA, 25 bp, Homo sapiens
ctatcccttcctctaaaataacaat

SEQ ID NO: 147 - DNA, 28 bp, Homo sapiens
aactaaaatatccccaacaaaatataac

SEQ ID NO: 148 - DNA, 30 bp, Homo sapiens
ccttaaaaaaaataccattatacataacct

SEQ ID NO: 149 - DNA, 20 bp, Homo sapiens
caattcctctaacccccaac

SEQ ID NO: 150 - DNA, 25 bp, Homo sapiens
aaccaaaaaacaactacaaaaaaaa

SEQ ID NO: 151 - DNA, 25 bp, Homo sapiens
tcaaaaccactaaccactaccctac

SEQ ID NO: 152 - DNA, 25 bp, Homo sapiens
aaaaaaaacaaaaaacccaaaaccc

SEQ ID NO: 153 - DNA, 25 bp, Homo sapiens
tcccctaaacaaccaaataatctcc

SEQ ID NO: 154 - DNA, 26 bp, Homo sapiens
catcctaaataaacaactcaaatatc

SEQ ID NO: 155 - DNA, 24 bp, Homo sapiens
tttacctctcaaaatacccccaca

SEQ ID NO: 156 - DNA, 25 bp, Homo sapiens
cgtaaaaacgaaaactaaccccgtt

SEQ ID NO: 157 - DNA, 26 bp, Homo sapiens
aacctacataatatccaatcaaaacc

SEQ ID NO: 158 - DNA, 25 bp, Homo sapiens
tatctacccgacccaaaacaccgta

SEQ ID NO: 159 - DNA, 30 bp, Homo sapiens
actatcaaaaaataatcatctcttacttca

SEQ ID NO: 160 - DNA, 28 bp, Homo sapiens
cattttaacctatttctaccactttaac

SEQ ID NO: 161 - DNA, 122 bp, Homo sapiens
ctttctttgcctgctggggtgattttggatgcaaaccttccgtgttaacgctctttcagacgtgctgttgaaagagtccaagtggacgaagatgttctttggagaaggccaggcctccctgt

SEQ ID NO: 162 - DNA, 122 bp, Homo sapiens; identical to SEQ ID NO: 39

SEQ ID NO: 163 - DNA, 122 bp, Homo sapiens
gagcaagaaaaatagtctataagtaggttgagggagggcatgtcaaatgtgtgtcatggccgagacacctccaaaaagccactgctgccgcctgagcagagcatgctgaaggctgtggttta

SEQ ID NO: 164 - DNA, 122 bp, Homo sapiens
tctctgggactcagtttccccaacggtgaatgaaggggtgaatctggaaggctgacattccgtactcagcaatgctgtcaccccctcagaaatccccagcctagcctgggggtggggtgggg

SEQ ID NO: 165 - DNA, 122 bp, Homo sapiens
ccctgaccccagcccccgcaaggccccttctgtgtacacttgactgaattttatgggcctcgtggatatgcatgggcttatctccagacatcataaattaaccacagctgatctcatccaga

SEQ ID NO: 166 - DNA, 122 bp, Homo sapiens
gatgcagttcaggagctgggggcgggcggcgcagccttcagcacctcagagggaccgggacgcaccagacactcccccagcccactcggccagagccccggacaggatgggtgcccgaccag

SEQ ID NO: 167 - DNA, 122 bp, Homo sapiens; identical to SEQ ID NO: 33

SEQ ID NO: 168 - DNA, 122 bp, Homo sapiens
ttccatttcttttcttttctcctctctctctctctttctctctgtctctcctccactccccgacccccaactaggatgcatcctgtaaagctccatctggtttgggggtgggaagtgggttt

SEQ ID NO: 169 - DNA, 122 bp, Homo sapiens; identical to SEQ ID NO: 2

SEQ ID NO: 170 - DNA, 122 bp, Homo sapiens; identical to SEQ ID NO: 40

SEQ ID NO: 171 - DNA, 122 bp, Homo sapiens; identical to SEQ ID NO: 14

SEQ ID NO: 172 - DNA, 122 bp, Homo sapiens
ggcccaattcccactcccccaaacacacacaagtacacactgactaaggcacagctagggcgggggcgggcagaaggccccttgggaggacgtggcgccacagctgcaatgggtgtgggggt

SEQ ID NO: 173 - DNA, 122 bp, Homo sapiens; identical to SEQ ID NO: 30

SEQ ID NO: 174 - DNA, 122 bp, Homo sapiens
ggcttgacttctcccacgccccatagacccggcaccgtgtaataactgggcccgtgtcctcgcctgaaaactgggggtcacacggcctgtcctgaagaactctgatgtgataaacaccatag

SEQ ID NO: 175 - DNA, 122 bp, Homo sapiens
tcatgggagaggtataggtccataagaaacaaagtcatcttaccaactggatatctgctccgagctgctgctgcttcctcttcctttttttggggggtgggggccgattttggaagggcaaa

SEQ ID NO: 176 - DNA, 122 bp, Homo sapiens; identical to SEQ ID NO: 8

SEQ ID NO: 177 - DNA, 122 bp, Homo sapiens; identical to SEQ ID NO: 7

SEQ ID NO: 178 - DNA, 122 bp, Homo sapiens; identical to SEQ ID NO: 34

SEQ ID NO: 179 - DNA, 122 bp, Homo sapiens
ttcccttagctcgccagaccctggctcgtgaattatttatgacccggcttctgggaccaccgcgacggctttcggagagcccgcctcccactgccggccgcggaggggctcaggcggcgctg

SEQ ID NO: 180 - DNA, 122 bp, Homo sapiens
aggtgtccgtcagcttctgcagtttcttgcggttcatgtctggcgctcagagaatcgggccgcggcgggggtctctgggcgcgcggctaccgagaccctcgcgggacccccgcgagccctgg

SEQ ID NO: 181 - DNA, 122 bp, Homo sapiens
cctttctctcagcactcaacctaaagtgttttccctctccctccccattattctctagcacgtgattccttcaaagccctacctttgtgatgaatgctgtgatgtgccaccctgacccccct

SEQ ID NO: 182 - DNA, 122 bp, Homo sapiens; identical to SEQ ID NO: 37

SEQ ID NO: 183 - DNA, 122 bp, Homo sapiens
cgtgggccttgggttgtgtgggataatcctgtcttattcctattgttccaatgttccatccggctactgctgcctctaacaaaactaacccagtttggaagataaattaagtcattagtcga

SEQ ID NO: 184 - DNA, 122 bp, Homo sapiens; identical to SEQ ID NO: 5

SEQ ID NO: 185 - DNA, 122 bp, Homo sapiens; identical to SEQ ID NO: 25

SEQ ID NO: 186 - DNA, 122 bp, Homo sapiens; identical to SEQ ID NO: 18

SEQ ID NO: 187 - DNA, 122 bp, Homo sapiens
cctcccttcctcccgctcatcctggcacccactgatctttccacacggcctccacagtttcgccttttcaccatgcccagttgatatcatgcagaatgtggcctctcaggttggcttcctgc

SEQ ID NO: 188 - DNA, 122 bp, Homo sapiens
accataatttttttaaaaattgaggatgatcacagcatcctaggagcttagaggttaccacggtgaccagagccaacattggccaagtttgtcgtggaacagccataccacctgtcctgaat

SEQ ID NO: 189 - DNA, 122 bp, Homo sapiens
caggctggccacttcagtgagaggctgtacccggagtttcgttcggaggggttcggggcgcgcccaatccttgtctggccacttgacgccctggcaggaagaatcctcccgccgccggctcc

SEQ ID NO: 190 - DNA, 122 bp, Homo sapiens
ggttaatgtaatacaggagtgtaagaatttgttctttccactaaagagaaaacaaggccacgcattcccaattatatgctcacaaagggcagccagggagtgcagttcttggcagcagcagg

SEQ ID NO: 191 - DNA, 122 bp, Homo sapiens
aatcatcaactgttccctggccctgttctgtgctgcatcctaagccaaatgtcacaatctcgagatggattaggggatgcggaggagggagaggaggccatgaggagcagaacagaggaggg

SEQ ID NO: 192 - DNA, 122 bp, Homo sapiens
ggcctgttttcacatcattctgatcatctctgtccttcgtcttcattttgctgtgcaactcggggagccgaggagaggtggcaaaaacagcggttgccgagacaaggcgcaggccttggcgc

SEQ ID NO: 193 - DNA, 122 bp, Homo sapiens
acatatcatccttggacaccaggcagtagaagtctgtgcgggcactgtagtttcgcgagccgagatccgagacgtccacttcgctgctccggctctctcccagcgagaccccactggtgtgc

SEQ ID NO: 194 - DNA, 122 bp, Homo sapiens
ataaaacagataaggagaaggctgtatctaggctgaatggctggccaatgttttcctctccgtcagtataaataaaatggatggaagaaaacacccctggatactatcaaatatgcctttca

SEQ ID NO: 195 - DNA, 122 bp, Homo sapiens
gcaggggaagaggggagacctggcacgtgcgggaggcctggagaagagacaaggaaacagcgtgagtatcgtaggcacataaagacctggctggggatattcagggagtggagggcaggctc

SEQ ID NO: 196 - DNA, 122 bp, Homo sapiens; identical to SEQ ID NO: 6

SEQ ID NO: 197 - DNA, 122 bp, Homo sapiens; identical to SEQ ID NO: 10

SEQ ID NO: 198 - DNA, 122 bp, Homo sapiens; identical to SEQ ID NO: 15

SEQ ID NO: 199 - DNA, 122 bp, Homo sapiens
atgccgtctggctttggccatgagaccctcgtgtgaccaggtgcgtgcctaagttagaatcgcccaggctaagttcgtgaaccccctggatgagggaggcccgactcccgcaaggagccctg

SEQ ID NO: 200 - DNA, 122 bp, Homo sapiens; identical to SEQ ID NO: 9

SEQ ID NO: 201 - DNA, 122 bp, Homo sapiens
ccatcttctcatgggagtttcagttttgtttcttgtgacctctatctccactgcactgttcggcaccaccccaaacacacccccaggctggttccacagagcagggtcttgctttccatccc

SEQ ID NO: 202 - DNA, 122 bp, Homo sapiens; identical to SEQ ID NO: 29

SEQ ID NO: 203 - DNA, 122 bp, Homo sapiens; identical to SEQ ID NO: 28

SEQ ID NO: 204 - DNA, 122 bp, Homo sapiens
tgacacaggaagtgggcgcatagcacttgctgtggaaatctgtgcctgcccccctgcctacgctggtgactcttgtcaggtaggaagttttcccatccgcaacatttccctagggagctcct

SEQ ID NO: 205 - DNA, 122 bp, Homo sapiens; identical to SEQ ID NO: 3

SEQ ID NO: 206 - DNA, 122 bp, Homo sapiens; identical to SEQ ID NO: 4

SEQ ID NO: 207 - DNA, 122 bp, Homo sapiens; identical to SEQ ID NO: 27

SEQ ID NO: 208 - DNA, 122 bp, Homo sapiens; identical to SEQ ID NO: 13

SEQ ID NO: 209 - DNA, 122 bp, Homo sapiens
gagaggctgctctgtgaggagcagtgttttccacagctgctctgagaattcagtagaaatcgagccatttgttactcagaagtctagctggatggcaagtggaaatcgttgcagcataaagg

SEQ ID NO: 210 - DNA, 122 bp, Homo sapiens; identical to SEQ ID NO: 1

SEQ ID NO: 211 - DNA, 122 bp, Homo sapiens
acattcaggaaacatgttgatgttgctgattgcaacatgctccttacacacaccagtgttcgagcacttgactcacaggaaatgctcctctgtctcaggcagatttcaggcatcaaacaggt

SEQ ID NO: 212 - DNA, 122 bp, Homo sapiens; identical to SEQ ID NO: 16

SEQ ID NO: 213 - DNA, 122 bp, Homo sapiens
tattcacccaggatatgttattgtgtgcttagccttggactttgttgcttgtgtgttggacgcctaatagtcttgacagctaaataggcttttgtaagcgagagtggtaaagtccaacatgt

SEQ ID NO: 214 - DNA, 122 bp, Homo sapiens
aacccttcacaatctggaactggaaagagatttctagcccccacgaggaacaaagcttgacgatgaggaaatgacaacctcccttgtttgctaactattctcaggctaaaaagagagtgctg

SEQ ID NO: 215 - DNA, 122 bp, Homo sapiens
ctgtggcctcgcacaggcggacagacgggcagcagggatctccaaccgggccccggagcacgaaccactacgcaatcgtcacagcatgtgctaccttccgtggtgttctgggaatctaaatc

SEQ ID NO: 216 - DNA, 122 bp, Homo sapiens
cataactggtctattctgttctctttttaaaacagagccaagattttcttcttcactcctcgcttggtggctcccagccagaggccggcagtggtgggaggctcgctctgggtgcacagacg

SEQ ID NO: 217 - DNA, 122 bp, Homo sapiens
tgggacgcctggccaaatgcggggcggtctctcgcgggccattggcttgggccaccgttccgagtcagctcctaggatttccccaggctttgcggcccctttgtgggtctaggccagcgcct

SEQ ID NO: 218 - DNA, 122 bp, Homo sapiens
actgagtcatgagaccaactcagagaccacagatgcaaatgagccttgtggtttctggtacgctgcatacacatctggacctacccaggaagccacaagagggaccaagtccaaggtctagc

SEQ ID NO: 219 - DNA, 122 bp, Homo sapiens
gtcccactgcccattctgattcacatcccccaattcctgatcatgtttgtctacgtctggcgatacctgaccagaagacgtccttatcttctatcctctccctttcctttgagggagaagtc

SEQ ID NO: 220 - DNA, 50 bp, Homo sapiens
gctggggtgattttggatgcaaaccttccgtgttaacgctctttcagacg

SEQ ID NO: 221 - DNA, 50 bp, Homo sapiens; identical to SEQ ID NO: 79

SEQ ID NO: 222 - DNA, 50 bp, Homo sapiens
ttcagcatgctctgctcaggcggcagcagtggctttttggaggtgtctcg

SEQ ID NO: 223 - DNA, 50 bp, Homo sapiens
cccaggctaggctggggatttctgagggggtgacagcattgctgagtacg

SEQ ID NO: 224 - DNA, 50 bp, Homo sapiens
cgaggcccataaaattcagtcaagtgtacacagaaggggccttgcggggg

SEQ ID NO: 225 - DNA, 50 bp, Homo sapiens
cgcaccagacactcccccagcccactcggccagagccccggacaggatgg

SEQ ID NO: 226 - DNA, 50 bp, Homo sapiens; identical to SEQ ID NO: 73

SEQ ID NO: 227 - DNA, 50 bp, Homo sapiens
cacccccaaaccagatggagctttacaggatgcatcctagttgggggtcg

SEQ ID NO: 228 - DNA, 50 bp, Homo sapiens; identical to SEQ ID NO: 42

SEQ ID NO: 229 - DNA, 50 bp, Homo sapiens; identical to SEQ ID NO: 80

SEQ ID NO: 230 - DNA, 50 bp, Homo sapiens; identical to SEQ ID NO: 54

SEQ ID NO: 231 - DNA, 50 bp, Homo sapiens
actcccccaaacacacacaagtacacactgactaaggcacagctagggcg

SEQ ID NO: 232 - DNA, 50 bp, Homo sapiens; identical to SEQ ID NO: 70

SEQ ID NO: 233 - DNA, 50 bp, Homo sapiens
cgcctgaaaactgggggtcacacggcctgtcctgaagaactctgatgtga

SEQ ID NO: 234 - DNA, 50 bp, Homo sapiens
cggagcagatatccagttggtaagatgactttgtttcttatggacctata

SEQ ID NO: 235 - DNA, 50 bp, Homo sapiens; identical to SEQ ID NO: 48

SEQ ID NO: 236 - DNA, 50 bp, Homo sapiens; identical to SEQ ID NO: 47

SEQ ID NO: 237 - DNA, 50 bp, Homo sapiens; identical to SEQ ID NO: 74

SEQ ID NO: 238 - DNA, 50 bp, Homo sapiens
gccagaccctggctcgtgaattatttatgacccggcttctgggaccaccg

SEQ ID NO: 239 - DNA, 50 bp, Homo sapiens
gcttctgcagtttcttgcggttcatgtctggcgctcagagaatcgggccg

SEQ ID NO: 240 - DNA, 50 bp, Homo sapiens
gtggcacatcacagcattcatcacaaaggtagggctttgaaggaatcacg

SEQ ID NO: 241 - DNA, 50 bp, Homo sapiens; identical to SEQ ID NO: 77

SEQ ID NO: 242 - DNA, 50 bp, Homo sapiens
gttgtgtgggataatcctgtcttattcctattgttccaatgttccatccg

SEQ ID NO: 243 - DNA, 50 bp, Homo sapiens; identical to SEQ ID NO: 45

SEQ ID NO: 244 - DNA, 50 bp, Homo sapiens; identical to SEQ ID NO: 65

SEQ ID NO: 245 - DNA, 50 bp, Homo sapiens; identical to SEQ ID NO: 58

SEQ ID NO: 246 - DNA, 50 bp, Homo sapiens
cgccttttcaccatgcccagttgatatcatgcagaatgtggcctctcagg

SEQ ID NO: 247 - DNA, 50 bp, Homo sapiens
cggtgaccagagccaacattggccaagtttgtcgtggaacagccatacca

SEQ ID NO: 248 - DNA, 50 bp, Homo sapiens
gggaggattcttcctgccagggcgtcaagtggccagacaaggattgggcg

SEQ ID NO: 249 - DNA, 50 bp, Homo sapiens
cgcattcccaattatatgctcacaaagggcagccagggagtgcagttctt

SEQ ID NO: 250 - DNA, 50 bp, Homo sapiens
ttccctggccctgttctgtgctgcatcctaagccaaatgtcacaatctcg

SEQ ID NO: 251 - DNA, 50 bp, Homo sapiens
catcattctgatcatctctgtccttcgtcttcattttgctgtgcaactcg

SEQ ID NO: 252 - DNA, 50 bp, Homo sapiens
cggctcgcgaaactacagtgcccgcacagacttctactgcctggtgtcca

SEQ ID NO: 253 - DNA, 50 bp, Homo sapiens
cggagaggaaaacattggccagccattcagcctagatacagccttctcct

SEQ ID NO: 254 - DNA, 50 bp, Homo sapiens
cactccctgaatatccccagccaggtctttatgtgcctacgatactcacg

SEQ ID NO: 255 - DNA, 50 bp, Homo sapiens; identical to SEQ ID NO: 46

SEQ ID NO: 256 - DNA, 50 bp, Homo sapiens; identical to SEQ ID NO: 50

SEQ ID NO: 257 - DNA, 50 bp, Homo sapiens; identical to SEQ ID NO: 55

SEQ ID NO: 258 - DNA, 50 bp, Homo sapiens
tttggccatgagaccctcgtgtgaccaggtgcgtgcctaagttagaatcg

SEQ ID NO: 259 - DNA, 50 bp, Homo sapiens; identical to SEQ ID NO: 49

SEQ ID NO: 260 - DNA, 50 bp, Homo sapiens
gggagtttcagttttgtttcttgtgacctctatctccactgcactgttcg

SEQ ID NO: 261 - DNA, 50 bp, Homo sapiens; identical to SEQ ID NO: 69

SEQ ID NO: 262 - DNA, 50 bp, Homo sapiens; identical to SEQ ID NO: 68

SEQ ID NO: 263 - DNA, 50 bp, Homo sapiens
cgctggtgactcttgtcaggtaggaagttttcccatccgcaacatttccc

SEQ ID NO: 264 - DNA, 50 bp, Homo sapiens; identical to SEQ ID NO: 43

SEQ ID NO: 265 - DNA, 50 bp, Homo sapiens; identical to SEQ ID NO: 44

SEQ ID NO: 266 - DNA, 50 bp, Homo sapiens; identical to SEQ ID NO: 67

SEQ ID NO: 267 - DNA, 50 bp, Homo sapiens; identical to SEQ ID NO: 53

SEQ ID NO: 268 - DNA, 50 bp, Homo sapiens
cgagccatttgttactcagaagtctagctggatggcaagtggaaatcgtt

SEQ ID NO: 269 - DNA, 50 bp, Homo sapiens; identical to SEQ ID NO: 41

SEQ ID NO: 270 - DNA, 50 bp, Homo sapiens
catgttgatgttgctgattgcaacatgctccttacacacaccagtgttcg

SEQ ID NO: 271 - DNA, 50 bp, Homo sapiens; identical to SEQ ID NO: 56

SEQ ID NO: 272 - DNA, 50 bp, Homo sapiens
cgcctaatagtcttgacagctaaataggcttttgtaagcgagagtggtaa

SEQ ID NO: 273 - DNA, 50 bp, Homo sapiens
tttagcctgagaatagttagcaaacaagggaggttgtcatttcctcatcg

SEQ ID NO: 274 - DNA, 50 bp, Homo sapiens
cagaacaccacggaaggtagcacatgctgtgacgattgcgtagtggttcg

SEQ ID NO: 275 - DNA, 50 bp, Homo sapiens
attctgttctctttttaaaacagagccaagattttcttcttcactcctcg

SEQ ID NO: 276 - DNA, 50 bp, Homo sapiens
agacccacaaaggggccgcaaagcctggggaaatcctaggagctgactcg

SEQ ID NO: 277 - DNA, 50 bp, Homo sapiens
cgctgcatacacatctggacctacccaggaagccacaagagggaccaagt

SEQ ID NO: 278 - DNA, 50 bp, Homo sapiens
cgccagacgtagacaaacatgatcaggaattgggggatgtgaatcagaat

SEQ ID NO: 279 - DNA, 20 bp, Homo sapiens
ggtaagggtggttttgataa

SEQ ID NO: 280 - DNA, 25 bp, Homo sapiens
taagagtgaaattttgttttaaaaa

SEQ ID NO: 281 - DNA, 32 bp, Homo sapiens
tatttaggggagatagagatggtttaaatatg

SEQ ID NO: 282 - DNA, 30 bp, Homo sapiens
tattattgatggattatgtttagattgaag

SEQ ID NO: 283 - DNA, 30 bp, Homo sapiens
agagagagagagagagtttgtttagggata

SEQ ID NO: 284 - DNA, 30 bp, Homo sapiens; identical to SEQ ID NO: 83

SEQ ID NO: 285 - DNA, 25 bp, Homo sapiens
gtagtaggtggggtagggtggtttt

SEQ ID NO: 286 - DNA, 27 bp, Homo sapiens
tgtaagttatggaggtgggtttttttt

SEQ ID NO: 287 - DNA, 28 bp, Homo sapiens
gaaatttttgtgttagtgtttttaagtg

SEQ ID NO: 288 - DNA, 30 bp, Homo sapiens; identical to SEQ ID NO: 88

SEQ ID NO: 289 - DNA, 29 bp, Homo sapiens
aaatttggtattaggattttttggtttat

SEQ ID NO: 290 - DNA, 30 bp, Homo sapiens
ttttttaaattgggttagttttgttagagg

SEQ ID NO: 291 - DNA, 30 bp, Homo sapiens
gcgtacgagattagtaattgagattttatc

SEQ ID NO: 292 - DNA, 25 bp, Homo sapiens
ttttttatagttgaatttttgggaa

SEQ ID NO: 293 - DNA, 30 bp, Homo sapiens; identical to SEQ ID NO: 92

SEQ ID NO: 294 - DNA, 22 bp, Homo sapiens
ttggattttggaggagggatgc

SEQ ID NO: 295 - DNA, 25 bp, Homo sapiens
aagataaaggaatattaggtttggt

SEQ ID NO: 296 - DNA, 30 bp, Homo sapiens
tagatgttttttgaatagggtgattttttt

SEQ ID NO: 297 - DNA, 30 bp, Homo sapiens
gtatatttgatagtatttaggggtgttttt

SEQ ID NO: 298 - DNA, 24 bp, Homo sapiens
gggggaaggaatgagtagattagt

SEQ ID NO: 299 - DNA, 24 bp, Homo sapiens
gggggaaggaatgagtagattagt

SEQ ID NO: 300 - DNA, 30 bp, Homo sapiens
ttgtttataaaaggtaattttgattgaagg

SEQ ID NO: 301 - DNA, 24 bp, Homo sapiens
gaagtggaggaatatttggtggta

SEQ ID NO: 302 - DNA, 24 bp, Homo sapiens
gttttgtggaattagtttgggggt

SEQ ID NO: 303 - DNA, 27 bp, Homo sapiens
taatttgatttggttttgttggtagtt

SEQ ID NO: 304 - DNA, 27 bp, Homo sapiens
ttgagggatttagtaataggataaaaa

SEQ ID NO: 305 - DNA, 26 bp, Homo sapiens
ttgggtaggtttagatgtgtatgtag

SEQ ID NO: 306 - DNA, 28 bp, Homo sapiens
ttttattttttttaagtgtttttttaga

SEQ ID NO: 307 - DNA, 30 bp, Homo sapiens
gaggtggggttagaggaagtttttgatata

SEQ ID NO: 308 - DNA, 25 bp, Homo sapiens
aattaggatatgttcgatttttcgt

SEQ ID NO: 309 - DNA, 30 bp, Homo sapiens
tttggaaagggtttaattttaatttttttt

SEQ ID NO: 310 - DNA, 25 bp, Homo sapiens
tgatgggtaatgttgaaggtaagtt

SEQ ID NO: 311 - DNA, 26 bp, Homo sapiens
ttttttttgtttttaagtatttttag

SEQ ID NO: 312 - DNA, 30 bp, Homo sapiens
gtgtagttgttgggattttattaggttgag

SEQ ID NO: 313 - DNA, 30 bp, Homo sapiens; identical to SEQ ID NO: 104

SEQ ID NO: 314 - DNA, 27 bp, Homo sapiens
tttttattttaaagggaaaaatttgtt

SEQ ID NO: 315 - DNA, 27 bp, Homo sapiens
tttttgattgattaaggttttgaatat

SEQ ID NO: 316 - DNA, 30 bp, Homo sapiens
aggttgttttgtgaggagtagtgtttttta

SEQ ID NO: 317 - DNA, 30 bp, Homo sapiens
aaggtggagatttagggttattaaatttta

SEQ ID NO: 318 - DNA, 30 bp, Homo sapiens
tagtaatattggtggtagtagtagagaata

SEQ ID NO: 319 - DNA, 25 bp, Homo sapiens
tatgtttaaggaattttttttatta

SEQ ID NO: 320 - DNA, 23 bp, Homo sapiens
ggtgagtagttgggtttttattt

SEQ ID NO: 321 - DNA, 30 bp, Homo sapiens
gtgagatgatggttgtgttatgtgattata

SEQ ID NO: 322 - DNA, 22 bp, Homo sapiens
agggtagtgagtttggttaggg

SEQ ID NO: 323 - DNA, 26 bp, Homo sapiens
ttgttttgttttttatggtttttttt

SEQ ID NO: 324 - DNA, 30 bp, Homo sapiens
aaggtaatttgtaagtatgtattatgttga

SEQ ID NO: 325 - DNA, 30 bp, Homo sapiens
ttgtagttattttggaggtgatttaagtat

SEQ ID NO: 326 - DNA, 32 bp, Homo sapiens; identical to SEQ ID NO: 113

SEQ ID NO: 327 - DNA, 25 bp, Homo sapiens
taaggttgtgggtggttattttagg

SEQ ID NO: 328 - DNA, 24 bp, Homo sapiens
tggtggggtataatagtagggatt

SEQ ID NO: 329 - DNA, 25 bp, Homo sapiens
tggaggagttttaaaagtgtattat

SEQ ID NO: 330 - DNA, 30 bp, Homo sapiens; identical to SEQ ID NO: 115

SEQ ID NO: 331 - DNA, 30 bp, Homo sapiens
tgtttgatgtttgaaatttgtttgagatag

SEQ ID NO: 332 - DNA, 26 bp, Homo sapiens
gatattttagtaacgcgaggatcggc

SEQ ID NO: 333 - DNA, 30 bp, Homo sapiens; identical to SEQ ID NO: 117

SEQ ID NO: 334 - DNA, 25 bp, Homo sapiens
ggaagaatttaggttaggggtgcga

SEQ ID NO: 335 - DNA, 28 bp, Homo sapiens
gaatatttaggttgtttttatttttttt

SEQ ID NO: 336 - DNA, 25 bp, Homo sapiens
ttagagtgaaattttgggtagtttt

SEQ ID NO: 337 - DNA, 25 bp, Homo sapiens
ttttgggatttagtgttttgatttt

SEQ ID NO: 338 - DNA, 32 bp, Homo sapiens; identical to SEQ ID NO: 121

SEQ ID NO: 339 - DNA, 25 bp, Homo sapiens
tcctaactctaacttcctaaatctc

SEQ ID NO: 340 - DNA, 28 bp, Homo sapiens
aaaaaaacaacaaaaaattatcaaatcc

SEQ ID NO: 341 - DNA, 25 bp, Homo sapiens
ttattctctaaaactcaatttcccc

SEQ ID NO: 342 - DNA, 28 bp, Homo sapiens
caaaaactaaattcaacaacaaaaaaaa

SEQ ID NO: 343 - DNA, 25 bp, Homo sapiens
aacttcaacacctaaatatctccac

SEQ ID NO: 344 - DNA, 30 bp, Homo sapiens; identical to SEQ ID NO: 125

SEQ ID NO: 345 - DNA, 30 bp, Homo sapiens
ccaccatatccataatacaattcaaaaact

SEQ ID NO: 346 - DNA, 29 bp, Homo sapiens
aaaaaaataacttctattatcccctctac

SEQ ID NO: 347 - DNA, 30 bp, Homo sapiens; identical to SEQ ID NO: 128

SEQ ID NO: 348 - DNA, 30 bp, Homo sapiens; identical to SEQ ID NO: 126

SEQ ID NO: 349 - DNA, 24 bp, Homo sapiens
aaaaaaaattccaaatccaccccc

SEQ ID NO: 350 - DNA, 25 bp, Homo sapiens
aacacaacaacgaaccttaacacga

SEQ ID NO: 351 - DNA, 30 bp, Homo sapiens
aattaaataactttaaccccaacctatcta

SEQ ID NO: 352 - DNA, 26 bp, Homo sapiens
aacaaaccattcccacaataaataac

SEQ ID NO: 353 - DNA, 25 bp, Homo sapiens
aacgcctaaccaaatacgaaacgat

SEQ ID NO: 354 - DNA, 21 bp, Homo sapiens
ccatccaaaaacccctaaaaa

SEQ ID NO: 355 - DNA, 30 bp, Homo sapiens
ccattatcccttctaaaatacaaaatccat

SEQ ID NO: 356 - DNA, 30 bp, Homo sapiens
aaacttaaaaaacaaaacaatcatttttat

SEQ ID NO: 357 - DNA, 26 bp, Homo sapiens
aaccacttcaataaaaaactataccc

SEQ ID NO: 358 - DNA, 30 bp, Homo sapiens
aattcaaactaaccacttcaataaaaaact

SEQ ID NO: 359 - DNA, 25 bp, Homo sapiens
acaacaaattctaccaactcctccc

SEQ ID NO: 360 - DNA, 30 bp, Homo sapiens; identical to SEQ ID NO: 136

SEQ ID NO: 361 - DNA, 30 bp, Homo sapiens
ttccaaccttctctctataaccttaaaaaa

SEQ ID NO: 362 - DNA, 23 bp, Homo sapiens
ctcacacatctccaacaaaaaac

SEQ ID NO: 363 - DNA, 22 bp, Homo sapiens
acccatcccaaaccctatctac

SEQ ID NO: 364 - DNA, 25 bp, Homo sapiens
aacaaaaaattaaatccaaaaaaaa

SEQ ID NO: 365 - DNA, 26 bp, Homo sapiens
aacatctataacttaacttctcccac

SEQ ID NO: 366 - DNA, 25 bp, Homo sapiens
ccccaaaccatcccacaatactaaa

SEQ ID NO: 367 - DNA, 32 bp, Homo sapiens; identical to SEQ ID NO: 141

SEQ ID NO: 368 - DNA, 30 bp, Homo sapiens
acctttctctcaacactcaacctaaaatat

SEQ ID NO: 369 - DNA, 30 bp, Homo sapiens
aaaaaaaataactctattctcaccaacaaa

SEQ ID NO: 370 - DNA, 24 bp, Homo sapiens
aaaaaaatacacaacacaccttcc

SEQ ID NO: 371 - DNA, 27 bp, Homo sapiens
acaaaaaaacctaaccttctccaaaaa

SEQ ID NO: 372 - DNA, 25 bp, Homo sapiens
aaactattaatacacaacccaaccc

SEQ ID NO: 373 - DNA, 30 bp, Homo sapiens; identical to SEQ ID NO: 148

SEQ ID NO: 374 - DNA, 28 bp, Homo sapiens
aactaaaatatccccaacaaaatataac

SEQ ID NO: 375 - DNA, 30 bp, Homo sapiens
aaaaaaataaccatccaacatccactaaat

SEQ ID NO: 376 - DNA, 30 bp, Homo sapiens
atcaacaacaccttcttacttaatccatat

SEQ ID NO: 377 - DNA, 26 bp, Homo sapiens
aaaataaaaataaattccatttacaa

SEQ ID NO: 378 - DNA, 21 bp, Homo sapiens
ataaaaacccaactactcacc

SEQ ID NO: 379 - DNA, 20 bp, Homo sapiens
caattcctctaacccccaac

SEQ ID NO: 380 - DNA, 30 bp, Homo sapiens
catccaaaaacttctacaaaatttctcttt

SEQ ID NO: 381 - DNA, 25 bp, Homo sapiens
aaccaaaaaacaactacaaaaaaaa

SEQ ID NO: 382 - DNA, 24 bp, Homo sapiens
aaataaccaaatatcttcccaacc

SEQ ID NO: 383 - DNA, 25 bp, Homo sapiens
aaaaactaaaaaaaatccaaaaacc

SEQ ID NO: 384 - DNA, 28 bp, Homo sapiens
aaactaacaaaaaaacaaaactccttac

SEQ ID NO: 385 - DNA, 25 bp, Homo sapiens
tcccctaaacaaccaaataatctcc

SEQ ID NO: 386 - DNA, 30 bp, Homo sapiens
caaaaattataatcctattaaacaaaaaaa

SEQ ID NO: 387 - DNA, 26 bp, Homo sapiens
catcctaaataaacaactcaaatatc

SEQ ID NO: 388 - DNA, 30 bp, Homo sapiens
aatatatatccatcattaatatatcaatca

SEQ ID NO: 389 - DNA, 24 bp, Homo sapiens
tttacctctcaaaatacccccaca

SEQ ID NO: 390 - DNA, 25 bp, Homo sapiens
taactccttaaccctcccaaaatcc

SEQ ID NO: 391 - DNA, 25 bp, Homo sapiens
cgtaaaaacgaaaactaaccccgtt

SEQ ID NO: 392 - DNA, 26 bp, Homo sapiens
aacctacataatatccaatcaaaacc

SEQ ID NO: 393 - DNA, 25 bp, Homo sapiens
tatctacccgacccaaaacaccgta

SEQ ID NO: 394 - DNA, 27 bp, Homo sapiens
aacatcatttttcctacctaacataac

SEQ ID NO: 395 - DNA, 30 bp, Homo sapiens; identical to SEQ ID NO: 159

SEQ ID NO: 396 - DNA, 28 bp, Homo sapiens
cattttaacctatttctaccactttaac

SEQ ID NO: 397 - DNA, 20 bp, Homo sapiens
ggtaagggtggttttgataa

SEQ ID NO: 398 - DNA, 34 bp, Homo sapiens
agtttgggtgataagagtgaaattttgttttaaa

SEQ ID NO: 399 - DNA, 30 bp, Homo sapiens; identical to SEQ ID NO: 83

SEQ ID NO: 400 - DNA, 25 bp, Homo sapiens
tggggttttttgttattttggttgt

SEQ ID NO: 401 - DNA, 25 bp, Homo sapiens
gtagtaggtggggtagggtggtttt

SEQ ID NO: 402 - DNA, 30 bp, Homo sapiens
atgaaatttttgtgttagtgtttttaagtg

SEQ ID NO: 403 - DNA, 30 bp, Homo sapiens; identical to SEQ ID NO: 88

SEQ ID NO: 404 - DNA, 29 bp, Homo sapiens
aaatttggtattaggattttttggtttat

SEQ ID NO: 405 - DNA, 30 bp, Homo sapiens; identical to SEQ ID NO: 89

SEQ ID NO: 406 - DNA, 30 bp, Homo sapiens; identical to SEQ ID NO: 90

SEQ ID NO: 407 - DNA, 25 bp, Homo sapiens
ttgtagagggaggggaaaggatgtt

SEQ ID NO: 408 - DNA, 30 bp, Homo sapiens; identical to SEQ ID NO: 92

SEQ ID NO: 409 - DNA, 25 bp, Homo sapiens
aagataaaggaatattaggtttggt

SEQ ID NO: 410 - DNA, 30 bp, Homo sapiens; identical to SEQ ID NO: 94

SEQ ID NO: 411 - DNA, 30 bp, Homo sapiens; identical to SEQ ID NO: 95

SEQ ID NO: 412 - DNA, 24 bp, Homo sapiens
gaagtggaggaatatttggtggta

SEQ ID NO: 413 - DNA, 27 bp, Homo sapiens
taatttgatttggttttgttggtagtt

SEQ ID NO: 414 - DNA, 26 bp, Homo sapiens
attatggtttaattttaagagggatt

SEQ ID NO: 415 - DNA, 25 bp, Homo sapiens
ttggttttgttgttttattgagagt

SEQ ID NO: 416 - DNA, 27 bp, Homo sapiens
aaggaattttttagagtgattttttaa

SEQ ID NO: 417 - DNA, 25 bp, Homo sapiens
aattaggatatgttcgatttttcgt

SEQ ID NO: 418 - DNA, 25 bp, Homo sapiens
tgtagggttggtttgtataggtagg

SEQ ID NO: 419 - DNA, 30 bp, Homo sapiens; identical to SEQ ID NO: 103

SEQ ID NO: 420 - DNA, 30 bp, Homo sapiens; identical to SEQ ID NO: 104

SEQ ID NO: 421 - DNA, 30 bp, Homo sapiens; identical to SEQ ID NO: 105

SEQ ID NO: 422 - DNA, 25 bp, Homo sapiens
aggaatttaagttatttgaggtttt

SEQ ID NO: 423 - DNA, 27 bp, Homo sapiens
tttttattttaaagggaaaaatttgtt

SEQ ID NO: 424 - DNA, 30 bp, Homo sapiens
atttttttgattgattaaggttttgaatat

SEQ ID NO: 425 - DNA, 23 bp, Homo sapiens
ggtgagtagttgggtttttattt

SEQ ID NO: 426 - DNA, 22 bp, Homo sapiens
agggtagtgagtttggttaggg

SEQ ID NO: 427 - DNA, 30 bp, Homo sapiens; identical to SEQ ID NO: 111

SEQ ID NO: 428 - DNA, 25 bp, Homo sapiens
ggttaaggttggtttttgtgggagg

SEQ ID NO: 429 - DNA, 32 bp, Homo sapiens; identical to SEQ ID NO: 113

SEQ ID NO: 430 - DNA, 24 bp, Homo sapiens
tggtggggtataatagtagggatt

SEQ ID NO: 431 - DNA, 29 bp, Homo sapiens
gaaattgttgtggttaaatgatttatttt

SEQ ID NO: 432 - DNA, 26 bp, Homo sapiens
gatattttagtaacgcgaggatcggc

SEQ ID NO: 433 - DNA, 30 bp, Homo sapiens; identical to SEQ ID NO: 117

SEQ ID NO: 434 - DNA, 25 bp, Homo sapiens
ggaagaatttaggttaggggtgcga

SEQ ID NO: 435 - DNA, 26 bp, Homo sapiens
agttagagtgaaattttgggtagttt

SEQ ID NO: 436 - DNA, 25 bp, Homo sapiens
ttttgggatttagtgttttgatttt

SEQ ID NO: 437 - DNA, 32 bp, Homo sapiens; identical to SEQ ID NO: 121

SEQ ID NO: 438 - DNA, 25 bp, Homo sapiens
tcctaactctaacttcctaaatctc

SEQ ID NO: 439 - DNA, 25 bp, Homo sapiens
aacttcaacacctaaatatctccac

SEQ ID NO: 440 - DNA, 25 bp, Homo sapiens
aacccacaaaccacttcctctactc

SEQ ID NO: 441 - DNA, 30 bp, Homo sapiens; identical to SEQ ID NO: 125

SEQ ID NO: 442 - DNA, 29 bp, Homo sapiens
aaaaaaataacttctattatcccctctac

SEQ ID NO: 443 - DNA, 30 bp, Homo sapiens; identical to SEQ ID NO: 128

SEQ ID NO: 444 - DNA, 30 bp, Homo sapiens; identical to SEQ ID NO: 126

SEQ ID NO: 445 - DNA, 22 bp, Homo sapiens
ttaccttaacaaccactaaccc

SEQ ID NO: 446 - DNA, 30 bp, Homo sapiens; identical to SEQ ID NO: 130

SEQ ID NO: 447 - DNA, 28 bp, Homo sapiens
ctaccaaaatcactacccttataataac

SEQ ID NO: 448 - DNA, 26 bp, Homo sapiens
aacaaaccattcccacaataaataac

SEQ ID NO: 449 - DNA, 21 bp, Homo sapiens
ccatccaaaaacccctaaaaa

SEQ ID NO: 450 - DNA, 27 bp, Homo sapiens
aaaacaaaactctcaaaataaccaaac

SEQ ID NO: 451 - DNA, 30 bp, Homo sapiens; identical to SEQ ID NO: 135

SEQ ID NO: 452 - DNA, 30 bp, Homo sapiens; identical to SEQ ID NO: 136

SEQ ID NO: 453 - DNA, 23 bp, Homo sapiens
ctcacacatctccaacaaaaaac

SEQ ID NO: 454 - DNA, 24 bp, Homo sapiens
ctacttcctataaaatcccccaac

SEQ ID NO: 455 - DNA, 30 bp, Homo sapiens; identical to SEQ ID NO: 139

SEQ ID NO: 456 - DNA, 30 bp, Homo sapiens; identical to SEQ ID NO: 140

SEQ ID NO: 457 - DNA, 32 bp, Homo sapiens; identical to SEQ ID NO: 141

SEQ ID NO: 458 - DNA, 27 bp, Homo sapiens
ttccacaaaaaaattaccaaaaaaata

SEQ ID NO: 459 - DNA, 29 bp, Homo sapiens
cccaaaaaaaaccaaaaactccaccttaa

SEQ ID NO: 460 - DNA, 25 bp, Homo sapiens
aaactattaatacacaacccaaccc

SEQ ID NO: 461 - DNA, 25 bp, Homo sapiens
tacactcaaatactcacacaaaacc

SEQ ID NO: 462 - DNA, 25 bp, Homo sapiens
ctatcccttcctctaaaataacaat

SEQ ID NO: 463 - DNA, 30 bp, Homo sapiens; identical to SEQ ID NO: 148

SEQ ID NO: 464 - DNA, 28 bp, Homo sapiens
aactaaaatatccccaacaaaatataac

SEQ ID NO: 465 - DNA, 20 bp, Homo sapiens
caattcctctaacccccaac

SEQ ID NO: 466 - DNA, 25 bp, Homo sapiens
aaccaaaaaacaactacaaaaaaaa

SEQ ID NO: 467 - DNA, 25 bp, Homo sapiens
tcaaaaccactaaccactaccctac

SEQ ID NO: 468 - DNA, 25 bp, Homo sapiens
aaaaaaaacaaaaaacccaaaaccc

SEQ ID NO: 469 - DNA, 25 bp, Homo sapiens
tcccctaaacaaccaaataatctcc

SEQ ID NO: 470 - DNA, 26 bp, Homo sapiens
catcctaaataaacaactcaaatatc

SEQ ID NO: 471 - DNA, 24 bp, Homo sapiens
tttacctctcaaaatacccccaca

SEQ ID NO: 472 - DNA, 25 bp, Homo sapiens
cgtaaaaacgaaaactaaccccgtt

SEQ ID NO: 473 - DNA, 26 bp, Homo sapiens
aacctacataatatccaatcaaaacc

SEQ ID NO: 474 - DNA, 25 bp, Homo sapiens
tatctacccgacccaaaacaccgta

SEQ ID NO: 475 - DNA, 30 bp, Homo sapiens; identical to SEQ ID NO: 159

SEQ ID NO: 476 - DNA, 28 bp, Homo sapiens
cattttaacctatttctaccactttaac

SEQ ID NO: 477 - DNA, 20 bp, Homo sapiens
ccgggctcccagtggattcg

SEQ ID NO: 478 - DNA, 22 bp, Homo sapiens
gcttcccacgtgcgcagcagga

SEQ ID NO: 479 - DNA, 21 bp, Homo sapiens
ctcccagtggattcgcgggca

SEQ ID NO: 480 - DNA, 19 bp, Homo sapiens
cccacgtgcgcagcaggac